(19) United States
(12) Patent Application Publication
(10) Pub. No.: US 2003/0162777 A1
(43) Pub. Date: Aug. 28, 2003

(54) Novel N-acylated heterocycles

(75) Inventors: Amedeo Leonardi, Milan (IT); Gianni Motta, Barlassina (IT); Rodolfo Testa, Vignate (IT); Carlo Riva, Varese (IT); Jeffrey W. Corbett, Portage, MI (US); Per-Olof Andersson, White House Station, NJ (US)

Correspondence Address: Darby & Darby P.C., 805 Third Avenue, New York, NY, 10022, US

(73) Assignee: Recordati S.A.

(21) Appl. No.: 10/266,104

(22) Filed: Oct. 7, 2002

Related U.S. Application Data
(60) Provisional application No. 60/350,680, filed on Jan. 22, 2002.

(30) Foreign Application Priority Data
Oct. 5, 2001 (IT) MI 002060

(51) Int. Cl. A61K 31/541; A61K 31/519; A61K 31/496; A61K 31/498; A61K 31/502; A61K 31/4709

(52) U.S. Cl. 514/228.2; 514/252.16; 514/248; 514/249; 514/253.04; 514/253.06; 514/314; 514/264.11

(57) ABSTRACT

Described are compositions comprising a muscarinic receptor antagonist and an N-acylated heterocycle derivative having affinity for serotonergic receptors, and enantiomers, diastereoisomers, N-oxides, polymorphs, solvates and pharmaceutically acceptable salts thereof. The combination of a muscarinic receptor antagonist and an N-acylated heterocycle, or an enantiomer, diastereoisomer, N-oxide, polymorph, solvate or pharmaceutically acceptable salt thereof, is useful in the treatment of patients with neuromuscular dysfunction of the lower urinary tract and diseases related to 5-HT_{1A} receptors.

This application claims priority under 35 U.S.C. 119(e) of U.S. provisional application No. 60/350,680, filed Jan. 22, 2002, and priority under 35 U.S.C. 119 (a)-(d) of Italian application no. MI 2001A 002060, filed Oct. 5, 2001. The contents of each of the aforementioned applications are hereby incorporated herein by reference in their entirety.

### FIELD OF THE INVENTION

This invention relates to novel N-acylated heterocycle compounds having affinity for serotonergic (5HT_{1A}) receptors, pharmaceutical compositions thereof and uses for such compounds and compositions.

### BACKGROUND OF THE INVENTION

In mammals, micturition (urination) is a complex process that requires the integrated action of the bladder, its internal and external sphincters, the musculature of the pelvic floor and neurological control over these muscles at three levels (in the bladder wall or sphincter itself, in the autonomic centres of the spinal cord and in the central nervous system at the level of the pontine micturition centre (PMC) in the brainstem (pons) under the control of the cerebral cortex) (De Groat, Neurobiology of Incontinence, Ciba Foundation Symposium 151:27, 1990). Micturition results from contraction of the detrusor muscle, which consists of interlacing smooth-muscle fibres, under the control of the parasympathetic autonomic system originating from the sacral spinal cord. A simple voiding reflex is triggered by sensory nerves for pain, temperature and distension that run from the bladder to the sacral spinal cord. However, sensory tracts from the bladder reach the PMC too, generating nerve impulses that normally suppress the sacral spinal suppression of cortical inhibition of the reflex arc, and relaxing the muscles of the pelvic floor and external sphincter. Finally, the detrusor muscle contracts and voiding occurs. Abnormalities of lower-urinary tract function, e.g. dysuria, incontinence and enuresis, are common in the general population. Dysuria includes urinary frequency, nocturia and urgency, and may be caused by cystitis (including interstitial cystitis), prostatitis or benign prostatic hyperplasia (BPH) (which affects about 70% of elderly males), or by neurological disorders. Incontinence syndromes include stress incontinence, urgency incontinence, overflow incontinence and mixed incontinence. Enuresis refers to the involuntary passage of urine at night or during sleep.

Previously, treatment of neuromuscular dysfunction of the lower urinary tract involved administration of compounds that act directly on the bladder muscles, such as flavoxate, a spasmolytic drug (Ruffman, J. Int. Med. Res. 16:317, 1988) which is also active on the PMC (Guarneri et al., Drugs of Today, 30:91, 1994), or anticholinergic compounds such as oxybutynin (Andersson, Drugs 36:477, 1988) and tolterodine (Nilvebrant, Life Sci. 68(22-23): 2549, 2001). The use of α1-adrenergic receptor antagonists for the treatment of BPH is common too, but is based on a different mechanism of action (Lepor, Urology, 42:483, 1993). However, treatments that involve direct inhibition of the pelvic musculature (including the detrusor muscle) may have unwanted side effects, such as incomplete voiding or accommodation paralysis, tachycardia and dry mouth (Andersson, Drugs 35:477, 1988). Thus, it would be preferable to utilize compounds that act via the central nervous system to, for example, affect the sacral spinal reflex and/or the PMC inhibition pathways in a manner that restores normal functioning of the micturition mechanism. 1-(4-Fluorophenyl)-4-(6-trifluoromethyl-1,2,3,4-tetrahydroquinoline-2-ylmethyl)piperazine is described in WO 98/38194 as an intermediate in the synthesis of 2-iminothiazole[2,4,3-yl]quinoline having a glutaminergic antagonistic activity.

1-Phenyl-4-(1,2,3,4-tetrahydroquinoline-2-ylmethyl)piperazine is described as an antagonist of α-adrenergic receptors in Indian J. Exp. Biol. 10 (5): 368-370 (1972).

A series of 1-(aryl)-4-[2-(1,2,3,4-tetrahydroquinoline-2-yl)ethyl]piperazines is described in U.S. Pat. No. 3,983,121 as having a hypotensive activity.

1-Acetyl-2,β-(N-piperidinoethyl)-2H-indole-3-one is described in Chem. Pharm. Bull. 29:1900-1911 (1981).

Structurally complex N-acylated tetrahydroquinolines having a somatostatin receptor agonistic or antagonistic activity are disclosed in WO 99/52875.

WO 01/49678 discloses a class of phenylpiperazine derivatives which are described as having a high affinity for 5HT_{1A} receptor.

### SUMMARY OF THE INVENTION

The present invention concerns compounds of formula I:

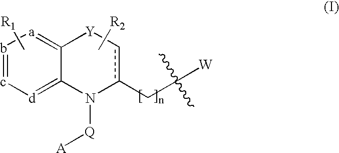

where:

W represents

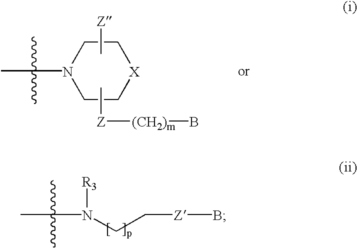

R₁ is one or more substituents selected from a group consisting of hydrogen, halogen, hydroxyl, alkyl, substituted alkyl, alkoxyl, substituted alkoxyl, nitro, aryl, substituted aryl, heterocycle, substituted heterocycle, alkenyl, substituted alkenyl, amino, alkylamino, dialkylamino, cyano, —SR₃, —C(O)R₃, —C(O)NR₃R₃, —NR₃C(O)R₃, —NR₃SO₂R₃, —NR₃C(O)OR₃ and —N(H)C(O)N(H)R₃;

R₃ is independently selected from a group consisting of hydrogen, alkyl, substituted alkyl, cycloalkyl, substituted cycloalkyl, aryl, substituted aryl, heterocycle and substituted heterocycle;

R₂ is one or two substituents selected from a group consisting of hydrogen, halogen, oxo, alkyl, substituted alkyl, alkenyl and substituted alkenyl groups;

Y represents a CH, CH₂, CR₂, CHR₂ group or a bond;

Q represents a carbonyl, thiocarbonyl or sulfonyl group;

A represents an alkyl, substituted alkyl, cycloalkyl, substituted cycloalkyl, alkenyl, substituted alkenyl, cycloalkenyl, substituted cycloalkenyl, aryl, substituted aryl, heterocycle, substituted heterocycle, alkylamino, substituted alkylamino, dialkylamino, substituted dialkylamino, cyclic amino, substituted cyclic amino, arylamino, substituted arylamino, arylalkylamino or substituted arylalkylamino group;

n is independently 1 or 2;

m is independently 0, 1 or 2;

p is independently 1, 2 or 3;

a, b, c and d are independently a carbon or nitrogen atom, or CH, CH₂ or NH group, with the proviso that no more than two of a, b, c and d may simultaneously be a nitrogen atom and/or NH,

X represents a bond, CH, CH₂, SO or SO₂ group or a carbon, nitrogen or sulphur atom and, when X is a nitrogen atom or CH group, the -Z-(CH₂)ₘ—B group is bound to said nitrogen atom or CH group, and when X is a carbon atom Z″ is not a hydrogen atom or oxo group and the Z-(CH₂)ₘ—B and Z″ groups are bound to said carbon;

Z represents a bond, an oxygen or sulphur atom or —CH(OH)—, —C(O)—NR₃C(O)—, —NR₃—C(O)—NR₃—, or —NR₃— group;

Z′ represents a bond or an oxygen or sulphur atom;

Z″ represents a hydrogen atom or hydroxyl, oxo, alkylcarbonyl or cyano group,

B represents a monocyclic aryl, substituted monocyclic aryl, bicyclic aryl, substituted bicyclic aryl, monocyclic heterocycle, substituted monocyclic heterocycle, bicyclic heterocycle or substituted bicyclic heterocycle;

[symbol] represents a single or double bond and, when Y═CH, the double bond is shifted so as to contain it; and

the enantiomers, diastereomers, N-oxides, crystalline forms, hydrates, solvates and pharmaceutically acceptable salts thereof, of the compounds of formula I.

Specific combinations of substituents are also contemplated.

In particular embodiments, the compounds of formula I are compounds having a formula illustrated below:

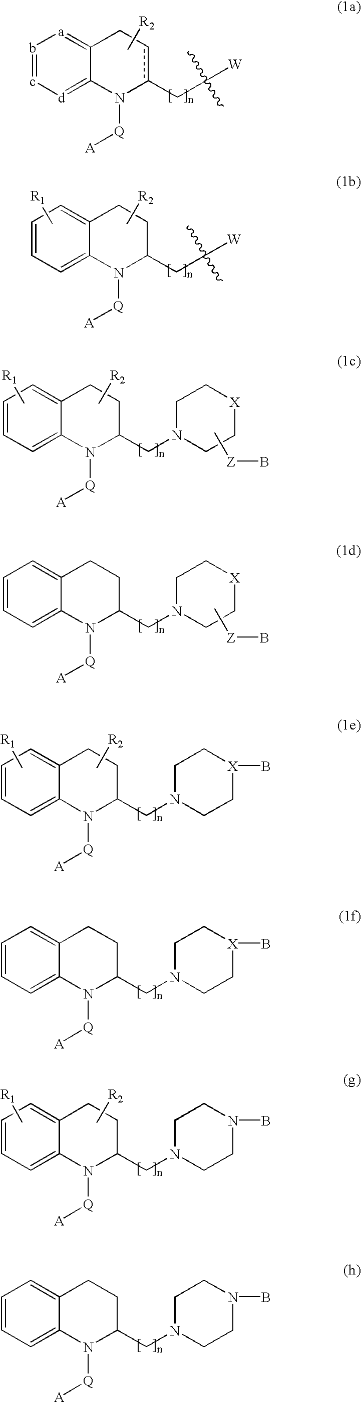

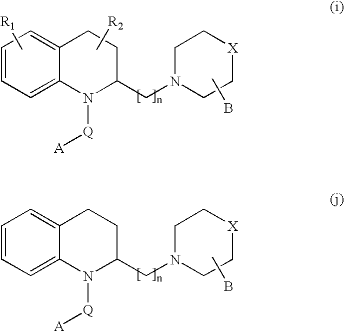

wherein R₁, R₂, Q, A, W, Z, B, n, a, b, c and d are as defined above. Accordingly, when the present disclosure sets forth or refers to a compound of formula I, the invention provides that each of the foregoing illustrated formulas may, without limitation, be substituted for formula I.

The invention also includes metabolites of the compounds of formula I having the same type of activity, hereinafter referred to as active metabolites.

The present invention also contemplates prodrugs which are metabolised in the body to generate the compounds of formula I.

In another embodiment, the present invention provides pharmaceutical compositions comprising compounds of formula I, enantiomers, diastereomers, N-oxides, crystalline forms, hydrates, solvates or pharmaceutically acceptable salts of such compounds of formula I, in admixture with pharmaceutically acceptable diluents or carriers such as those disclosed.

In another embodiment, the present invention provides compositions comprising compounds of formula I, enantiomers, diastereomers, N-oxides, crystalline forms, hydrates, solvates or pharmaceutically acceptable salts of such compounds of formula I, and an α1-adrenergic antagonist such as, for example and without limitation, prazosin, doxazosin, terazosin, alfuzosin and tamsulosin.

In another embodiment, the present invention provides compositions comprising compounds of formula I, enantiomers, diastereomers, N-oxides, crystalline forms, hydrates, solvates or pharmaceutically acceptable salts of such compounds of formula I, and a muscarinic receptor antagonist such as, for example and without limitation, oxybutynin, tolterodine, darifenacin and temiverine.

In yet another embodiment, the present invention provides the use of at least one compound of formula I in an amount effective for reducing the frequency of bladder contractions due to bladder distension by administering it to a mammal, including a human, in need of such treatment. Also, the present invention contemplates a method of administering a compound of formula I.

In yet another embodiment, the present invention provides the use of at least one compound of formula I in an amount effective for increasing urinary bladder capacity by administering it to a mammal, including a human, in need of such treatment.

In another embodiment, this invention provides the use of at least one compound of formula I in an amount effective for treating disorders of the urinary tract in a patient in need of such treatment to ameliorate at least one condition among urinary urgency, overactive bladder, increased urinary frequency, decreased urinary compliance (decreased bladder storage capacity), cystitis (including interstitial cystitis), incontinence, urine leakage, enuresis, dysuria, urinary hesitancy and difficulty in emptying the bladder.

For treating the above disorders, the compounds of the invention may be administered in combination with known antimuscarinic drugs such as oxybutynin, tolterodine, darifenacin and temiverine. Analogously, the compounds of the invention may be administered with α1-adrenergic antagonists, for the therapy of lower urinary tract symptoms, whether or not these are associated with BPH. Preferred α1-adrenergic antagonists suitable for administration in combination with a compound of the invention are prazosin, doxazosin, terazosin, alfuzosin and tamsulosin.

In yet another embodiment, the present invention covers the use of at least one compound of formula I in an amount effective for the treatment of central nervous system disorders due to serotonergic dysfunction. Such dysfunctions include anxiety, depression, hypertension, sleep/wake-cycle disorders, feeding, behaviour, sexual function and cognition disorders in mammals (particularly in humans) associated with stroke, injury, dementia, and originated by neurological development, attention-deficit hyperactivity disorders (ADHD), drug addiction, drug withdrawal, irritable-bowel syndrome. Treatment may be effected by delivering to the environment of a 5-HT_{1A serotonergic receptor, for example to the extracellular medium, or by systemically or locally administering to a mammal possessing such receptor, an amount of a compound of the invention effective to increase the duration of bladder quiescence with no contractions.}

In another embodiment the invention provides a method for reducing the activity of a 5HT_{1A} receptor comprising exposing said 5HT_{1A} receptor to an activity-lowering amount of the 5HT_{1A} receptor antagonist of a compound of formula I. In preferred embodiments the 5HT_{1A} receptor is present on the cell surface of a cell, more preferably a mammalian cell and, most preferably, a human cell.

The present invention refers to a method of administering a compound of the above formula with the previously-disclosed substituent patterns and combinations of such substituents.

### BRIEF DESCRIPTION OF DRAWINGS

FIG. 1. Time course of bladder volume capacity (BVC) and micturition pressure (MP) changes in rats after oral administration of vehicle (circles) or 3.0 mg/kg of the racemic compound ((±)1-(1-cyclohexanecarbonyl-1,2,3,4-tetrahydroquinoline-2-ylmethyl)-4-(4-indolyl)piperazine) of Example 1 (squares). Data represent the % changes versus basal values at different times from treatment. “n”=number of rats/group. Significance shown as P< . . . (between treatments: ANOVA (analysis of variance) of CONTRAST VARIABLES) indicates the difference between the trend observed in the control (vehicle) and treated groups. Asterisks (*=p<0.05, **p<0.01 and ***=p<0.001) indicate significance between the value observed at the time reported and the baseline value (within treatment).

FIG. 2. Time-course of bladder volume capacity (BVC) and micturition pressure (MP) changes in rats after oral administration of vehicle (circles) or 3.0 mg/kg of oxybutynin (squares). Data are expressed as in FIG. 1.

### DETAILED DESCRIPTION OF THE INVENTION

All cited patents, patent applications and literature references are hereby incorporated herein by reference in their entirety. In the case of inconsistencies, the present disclosure, including definitions, will prevail unless the context requires otherwise.

The present invention is related to compounds of formula I as disclosed above. The invention includes the enantiomers, diastereomers, N-oxides, crystalline forms, hydrates, solvates or pharmaceutically acceptable salts of these compounds, as well as active metabolites of these compounds having the same type of activity.

A “metabolite” of a compound disclosed herein is a derivative of a compound which is formed when the compound is metabolised. The term “active metabolite” refers to a biologically active derivative of a compound which is formed when the compound is metabolised. The term “metabolised” refers to the sum of the processes by which a particular substance is changed in the living body. In brief, all compounds present in the body are manipulated by enzymes within the body in order to derive energy and/or to remove them from the body. Specific enzymes produce specific structural alterations to the compound. For example, cytochrome P450 catalyses a variety of oxidative and reductive reactions while uridine diphosphate glucuronyltransferases catalyse the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9^{th} Edition, McGraw-Hill (1996), pages 11-17.

Metabolites of the compounds disclosed herein can be identified either by administration of compounds to a host and analysis of tissue samples from the host, or by incubation of compounds with hepatic cells in vitro and analysis of the resulting compounds. Both methods are well known in the art.

### Chemical Definitions

The following section is used to define the various substituents that are present in Formula I.

As used herein, the term “halo” or “halogen” refers to fluorine, chlorine, bromine and iodine.

As used herein, the term “hydroxyl” refers to a group —OH.

As used herein, the term “acyl” is a group —C(O)—R₃.

As used herein, the terms “oxo” and “keto” are synonymous and refer to a group ═O.

As used herein, the term “carbonyl” refers to a group —C(═O)—.

As used herein, the term “alkylcarbonyl” refers to a group —C(═O)-alkyl.

As used herein, the term “thiocarbonyl” refers to a group —C(═S)—.

As used herein, the term “alkylthio” refers to a group —S-alkyl.

As used herein, the term “sulfonyl” refers to a group —SO₂—.

As used herein, the term “nitro” refers to a group —NO₂.

As used herein, the term “amino” refers to a group —NH₂.

As used herein, the term “cyano” refers to a group —C≡N.

As used herein, the term “alkenyloxyl” refers to a group —O-alkenyl.

As used herein, the term “alkynyloxyl” refers to a group —O-alkynyl.

As used herein, the term “cycloalkoxyl” refers to a group —O-cycloalkyl.

As used herein, the term “aryloxyl” refers to a group —O-aryl.

As used herein, the term “aralkyl” refers to a group -alkyl-aryl.

As used herein, the term “arylalkoxyl” refers to a group —O-alkyl-aryl.

As used herein, the term “araloxylalkyl” refers to a group -alkyl-O-aryl.

As used herein, the term “cycloalkenyloxyl” refers to a group —O-cycloalkenyl.

As used herein, the term “cycloalkynyloxyl” refers to a group —O-cycloalkynyl.

As used herein, the term “heterocycloxyl” refers to a group —O-heterocycle.

As used herein, the terms “heterocyclealkyl” and “heterocyclicalkyl” are synonymous and refer to a group -alkyl-heterocycle.

As used herein, the term “heterocycloxylalkyl” refers to a group -alkyl-O-heterocycle.

As used herein, the term “alkylsulphonylamino” refers to a group —NH—S(O)₂-alkyl.

As used herein, the term “acyloxyl” refers to a group —O—C(═O)—R₃.

As used herein, the term “alkylaminocarbonyloxyl” refers to a group —O—C(═O)—NH-alkyl.

As used herein, the term “sulphonyloxyl” refers to a group —O—SO₂—R₃.

As used herein, the term “polyhaloalkylsulphonyloxyl” refers to a group —O—SO₂-polyhaloalkyl.

As used herein, the term “acylamino” refers to a group —NH—C(≡O)—R₃.

As used in this section, the term “cyanoamino” refers to a group —N(H)—C≡N.

As used herein, the term “acylalkylamino” refers to a group —N-alkyl-C(═O)—R₃.

As used herein, the term “ureido” refers to a group —NH—C(═O)—NH₂.

As used herein, the term “sulphonylamino” refers to a group —NH—SO₂—R₃.

As used herein, the term “sulphonylalkylamino” refers to a group —N-alkyl-SO₂—R₃.

As used herein, the term “arylsufonyl” refers to a group —SO₂-aryl.

As used herein, the term “alkylarylsulfonyl” refers to a group —SO₂-aryl-alkyl.

As used herein, the term “sulfamoyl” refers to a group —SO₂—NH₂.

As used herein, the term “substituted sulfamoyl” refers to a group —SO₂—NR₄R₅, wherein R₄ and R₅ are each independently a hydrogen atom or an alkyl group, or R₄ and R₅ are joined to form a monocyclic heterocycle comprising at least one nitrogen atom and optionally one or two additional heteroatoms selected independently from nitrogen, oxygen or sulphur.

As used herein, the term “alkyl” refers to a straight- or branched-chain hydrocarbon group having from 1 to 7 carbon atoms, preferably from 1 to 5 carbon atoms. Suitable alkyl groups include, but are not limited to, methyl, ethyl, n-propyl, isopropyl, n-butyl, sec-butyl, tert-butyl, pentyl, isopentyl, neopentyl, hexyl, isohexyl, heptyl, isoheptyl, and the like. The term “substituted alkyl” refers to an alkyl wherein at least one hydrogen is replaced by one or more substituents or groups independently selected for each position.

As used herein, the term “alkenyl” refers to linear or branched radicals of two to about twelve carbon atoms having at least one carbon-carbon double bond. Preferred alkenyl radicals are “lower alkenyl” radicals having two to about six carbon atoms. Examples of such radicals include ethenyl, n-propenyl, butenyl, and the like. The term “substituted alkenyl” refers to an alkenyl wherein at least one hydrogen is replaced by one or more substituents or groups independently selected for each position.

As used herein, the term “alkenyl” refers to linear or branched radicals of two to about twelve carbon atoms having at least one carbon-carbon triple bond. Preferred alkynyl radicals are “lower alkynyl” radicals having two to about six carbon atoms. Examples of such radicals include ethynyl, n-propynyl, butynyl, and the like. The term “substituted alkynyl” refers to an alkynyl wherein at least one hydrogen is replaced by one or more substituents or groups independently selected for each position.

As used herein, the term “alkoxyl” refers to a group —O-alkyl, wherein the alkyl moiety is defined above. In some embodiments, the preferred alkoxyl groups are those having from 1 to 6 carbon atoms. Alkoxyl may be substituted, for example, with at least one halogen, hydroxyl or cyano, preferably three halogens. Suitable alkoxyl groups include methoxyl, ethoxyl, n-propoxyl, i-propoxyl, butoxyl and the like. Suitable substituted alkoxyl groups include, as non-limiting examples, 2,2,2-trifluoroethoxyl, 2-hydroxyethoxyl and 2-cyanoethoxyl.

As used in this section, the term “cycloalkyl” refers to a mono or polycyclic non-aromatic hydrocarbon ring system having from 3 to 12 carbon atoms. Preferred monocyclic cycloalkyl groups are those having from 3 to 6 carbon atoms, for example cyclopropyl, cyclobutyl, cyclopentyl and cyclohexyl. “Substituted cycloalky” refers to a cycloalkyl wherein one or more hydrogen atom has been replaced with substituents or groups independently selected for each position.

As used in this part, the term “cycloalkenyl” for refers to a mono or polycyclic non-aromatic hydrocarbon ring system having from 5 to 12 carbon atoms, with at least one C═C group. “Substituted cycloalkenyl” refers to a cycloalkenyl wherein one or more hydrogen atom has been replaced with substituents or groups independently selected for each position.

As used in this part, the term “cycloalkynyl” refers to a mono or polycyclic non-aromatic hydrocarbon ring system having from 5 to 12 carbon atoms, with at least one C≡C group. “Substituted cycloalkynyl” refers to a cycloalkynyl wherein one or more hydrogen atom has been replaced with substituents or groups independently selected for each position.

As used herein the term “aryl” refers to a closed carbocyclic ring structure having from 6 to 12 carbon atoms comprising at least one unsaturated ring. The term “substituted aryl” refers to the instance wherein at least one hydrogen of said carbocyclic ring structure is substituted with one or more of the substituents described herein. As used herein the term “monocyclic aryl” refers to a closed aromatic carbocyclic ring structure having from 6 to 8 carbon atoms. An example of a monocyclic aryl group is phenyl. As used herein, the term “bicyclic aryl” refers to a closed bi-carbocyclic ring structure having from 9 to 12 carbon atoms. “Bicyclic aryl” encompasses the case wherein one ring of a bi-carbocyclic ring structure is saturated and the other ring is unsaturated or partially saturated and the case wherein both rings are saturated. Examples of bicyclic aryl groups include, without limitation, naphthyl and tetrahydronapthyl.

As used herein, the term “heterocycle” embraces saturated, partially saturated and unsaturated heteroatom-containing ring-shaped radicals having 5-12 atoms in the ring, where the heteroatoms may be selected from nitrogen, sulphur and oxygen. “Substituted heterocycle” refers to a heterocycle wherein one or more hydrogen atoms has been replaced with a substituent selected independently for each position. As used herein, the term “monocyclic heterocycle” refers to a closed saturated, partially saturated or unsaturated ring structure having from 5 to 7 atoms in the ring, in which one or more of the atoms in the ring is an atom other than carbon, such as oxygen, nitrogen or sulphur. Examples of monocyclic heterocycle groups groups include, without limitation, thiophene, pyridine, pyrimidine, imidazole, oxazole and thiazole. Examples of substituted heterocycles include, without limitation, thiophene, pyridine, pyrimidine, imidazole, oxazole and thiazole wherein one or more hydrogen atoms has been replaced with a substituent selected independently for each position. As used herein, the term “bicyclic heterocycle” refers to a closed saturated, partially saturated or unsaturated ring structure having from 9 to 12 atoms in the ring, in which one or more of the atoms in the ring is an atom other than carbon, such as oxygen, nitrogen or sulphur. Examples of bicyclic heterocycle groups and substituted heterocycle groups include, but are not limited to, indole, substituted indole (e.g., 2,3-dimethyl indole), quinoline, isoquinoline, benzothiophene, benzimidazole, benzodioxane, benzotriazole, benzofuran and 2,3-dihydrobenzofuran.

As used herein, the term “alkylamino” refers to an amino group in which the nitrogen atom of the amino (as defined above) is once substituted with an alkyl (as defined above). Preferably, the alkyl radical is 1 to 6 carbon atoms in length. The alkyl radical of an alkylamino may be substituted. Preferred substituents for the alkyl radical of an alkylamino group are, for example, hydroxyl, alkoxyl, arylalkoxyl, amino, acylamino and cyanoamino groups. Therefore, the term “substituted alkylamino” refers to an alkyl radical attached to an amino group wherein at least one hydrogen of the alkyl radical is replaced by one or more substituents independently selected for each position.

As used herein, the term “dialkylamino” refers to an amino group in which the N atom of the amino (as defined above) is twice substituted with alkyl (as defined above) radicals. Preferably, the alkyl radicals are independently 1 to 6 carbon atoms in length. One or both alkyl radicals of an alkylamino may be substituted. Preferred independent substituents for the alkyl radicals of dialkylamino group are, for example, hydroxyl, alkoxyl, arylalkoxyl, amino, acylamino and cyanoamino groups. Therefore, the term “substituted dialkylamino” refers to two alkyl radicals attached to an amino group wherein at least one hydrogen of one or both alkyl radicals is replaced by one or more substituents independently selected for each position.

As used herein, the term “alkenylamino” refers to an amino group in which the nitrogen atom of the amino (as defined above) is once substituted with an alkenyl (as defined above). Preferably, the alkenyl is 1 to 6 carbon atoms in length. Alkenylamino may be substituted, as set forth above for alkylamino. Preferred substituents for substituted alkenylamino groups are, for example, hydroxyl, alkoxyl, arylalkoxyl, amino, acylamino and cyanoamino.

As used herein, the term “dialkenylamino” refers to an amino group in which the N atom of the amino (as defined above) is twice substituted with alkenyl (as defined above). Preferably, the alkenyl is 1 to 6 carbon atoms in length. Dialkenylamino may be substituted, as set forth above for dialkylamino. Preferred substituents for substituted dialkenylamino groups are, for example, hydroxyl, alkoxyl, arylalkoxyl, amino, acylamino and cyanoamino.

As used herein, the term “alkynylamino” refers to an amino group in which the nitrogen atom of the amino (as defined above) is once substituted with an alkynyl (as defined above). Preferably, the alkynyl is 1 to 6 carbon atoms in length. Alkynylamino may be substituted, as set forth above for alkylamino. Preferred substituents for substituted alkynylamino groups are, for example, hydroxyl, alkoxyl, arylalkoxyl, amino, acylamino and cyanoamino.

As used herein, the term “dialkynylamino” refers to an amino group in which the N atom of the amino (as defined above) is twice substituted with alkynyl (as defined above). Preferably, the alkynyl is 1 to 6 carbon atoms in length. Dialkynylamino may be substituted, as set forth above for dialkylamino. Preferred substituents for substituted dialkynylamino groups are, for example, hydroxyl, alkoxyl, arylalkoxyl, amino, acylamino and cyanoamino.

As used herein, the term “cyclic amino” refers to an amino group in which the N atom of the amino (as defined above) is twice substituted with alkyl (as defined above) and the alkyl chains are connected to form a ring structure. Preferably, the alkyl is a group from 1 to 4 carbon atoms in length. In a preferred embodiment, the total number of carbon atoms present in the ring structure is from 4 to 6 carbon atoms. Additionally, the alkyl chains may be joined by a heteroatom, the formed ring structure therefore may optionally contain another heteroatom, such as oxygen, nitrogen or a sulphur atom. As used herein, “substituted cyclic amino” refers to cyclic amino wherein one or more hydrogen atom has been replaced independently with a substituent described herein. Preferred substituents for the cyclic amino group are alkyl, acyl, hydroxyl, alkoxyl, arylalkoxyl, amino, acylamino and cyanoamino groups.

As used herein, the terms “arylamino” and “diarylamino” refer respectively to one or two mono or bicyclic aromatic rings bound to a group NH and N atom, respectively, and the term “substituted” arylamino or diarylamino refers to the case wherein one or more hydrogen atoms on an aromatic ring of the arylamino or diarylamino has been replaced independently with a substituent described herein.

As used herein, the term “aroylamino” and used alone or in combination with other terms refers to a group —C(O)-aryl attached to an amino group, and the term “substituted” aroylamino refers to an aroylamino group wherein one or more hydrogen atoms on the aryl ring has been replaced independently with a substituent described herein.

As used herein, the term “arylalkylamino” refers to a mono or bicyclic aromatic ring, as defined herein, bound to an alkyl group as defined herein, preferably 1 to 6 carbon atoms in length, in turn bound to a group NH, and the term “substituted arylalkylamino” refers to an arylalkylamino group wherein one or more of the hydrogen atoms on the aryl ring has been replaced independently with a substituent described herein.

As used herein, the term “arylalkoxyl” refers to an alkoxyl substituted with an aryl moiety. Preferably, the arylalkoxyl has 1 to 2 carbon atoms in the alkoxyl moiety and the term “substituted arylalkoxyl” refers to an arylalkoxyl group wherein one or more of the hydrogen atoms on the aryl ring has been replaced independently with a substituent described herein.

As used herein, the term “haloalkyl” refers to radicals wherein any one or more of the alkyl carbon atoms is substituted with halo as defined above. Specifically embraced are monohaloalkyl and polyhaloalkyl radicals. A monohaloalkyl radical, for one example, may have either an iodo, bromo, chloro or fluoro atom within the radical. “Polyhaloalkyl” radicals are radicals comprising two or more of the same halo atoms or a combination of different halo atoms. In a preferred embodiment, the polyhaloalkyl is trifluoromethyl.

As used herein, the term “polyhaloalkoxyl” refers to an alkoxyl substituted with at least 2 halogen substituents. A preferred polyhaloalkoxyl is 2,2,2-trifluoroethoxyl.

As used herein, the term “alkoxylalkyl” refers to an alkyl radical in which at least one hydrogen atom has been substituted with any alkoxyl radical.

Variables A and B:

Variable A represents alkyl, substituted alkyl, alkenyl, substituted alkenyl, cycloalkyl, substituted cycloalkyl, cycloalkenyl, substituted cycloalkenyl, aryl, substituted aryl, heterocycle, substituted heterocycle, alkylamino, substituted alkylamino, dialkylamino, substituted dialkylamino, cyclic amino, substituted cyclic amino, arylamino, substituted arylamino, arylalkylamino or substituted arylalkylamino.

Variable B represents a monocyclic aryl, bicyclic aryl, monocyclic heterocycle, bicyclic heterocycle, substituted monocyclic aryl, substituted bicyclic aryl, substituted monocyclic heterocycle or substituted bicyclic heterocycle.

As used herein, the term “substituted” without further description refers to the instance where one or more hydrogen atoms on a radical are replaced independently with one or more atoms or groups. Atoms or groups that may be used as substituents of variables A and B include halogen, hydroxyl, oxo, nitro, cyano, alkyl, haloalkyl, polyhaloalkyl, alkylthio, alkoxyalkyl, alkenyl, alkynyl, cycloalkyl, cycloalkenyl, cycloalkynyl, alkoxyl, alkenyloxyl, alkynyloxyl, cycloalkoxyl, aryloxyl, substituted aryloxyl, cycloalkenyloxyl, cycloalkynyloxyl, arylalkoxyl, acyloxyl, alkylaminocarbonyloxyl, sulphonyloxyl, polyhaloalkylsulphonyloxyl, acyl, ureido, amino, alkylamino, dialkylamino, acylamino, diacylamino, N-alkyl-N-aroylamino, N-arylkyl-N-alkylsulphonylamino, alkylsulphonylamino, alkenylamino, dialkenylamino, arylamino, diarylamino, alkoxycarbonylamino, alkoxycarbonyl, acylamino, acylalkylamino, sulphonylamino, sulphonylalkylamino, cyanoamino, arylsufonyl, alkylarylsulfonyl, sulfamoyl, substituted sulfamoyl, aryl, substituted aryl, arylalkylamino, substituted arylalkylamino, heterocycle, substituted heterocycle, aralkyl, aryloxyalkyl, heterocycloxyalkyl, heterocyclicalkyl, wherein the terms substituted heterocycle, substituted aryl, substituted aryloxyl and substituted arylalkylamino refer respectively to a heterocyclic, aryl, aryloxyl or arylalkylamino group wherein one or more of the hydrogen atoms on a ring of the heterocyclic, aryl, aryloxyl or arylalkylamino group is replaced by one or more of the substituents recited herein, with the proviso that if variable A or B is substituted with a first substituted heterocycle, substituted aryl, substituted aryloxyl or substituted arylalkylamino and said first substituted heterocycle, substituted aryl, substituted aryloxyl or substituted arylalkylamino is substituted with a second substituted heterocycle, substituted aryl, substituted aryloxyl and substituted arylalkylamino, said second substituted heterocycle, substituted aryl, substituted aryloxyl and substituted arylalkylamino may not be substituted with a third substituted heterocycle, substituted aryl, substituted aryloxyl and substituted arylalkylamino.

Preferred alkyl groups that variable A represents are methyl, ethyl, propyl, butyl, pentyl, 1-ethylpropyl, isobutyl, neopentyl, tert-butyl and tert-pentyl.

When variable A is alkyl, preferred substituents for said alkyl group are hydroxyl; alkoxyl (e.g., methoxyl, isopropoxyl); aryloxyl (e.g., phenoxy); substituted aryloxyl, wherein the aryl group is substituted with one or more substituents selected from the group consisting of halogen (e.g., chlorine, fluorine) and alkoxyl (e.g., methoxyl) groups; arylalkoxyl (e.g., benzyloxyl); amino; alkylamino (e.g., methylamino); dialkylamino (e.g., dimethylamino), arylalkylamino (e.g., benzylamino), N-alkyl, N-acylamino; diacylamino; N-alkyl, N-aroylamino (e.g., N-methyl, N-benzoylamino); cyanoamino; cyano, ureido; aryl (e.g., phenyl); aryl (e.g., phenyl) substituted with one or more groups selected from the group consisting of halogen (e.g., fluorine, chlorine), polyhaloalkyl (e.g., trifluoromethyl), amino, alkylamino (e.g., methylamino or ethylamino), dialkylamino (e.g., N,N-dimethylamino or N-methyl, N-ethylamino) and alkoxyl (e.g., methoxy) groups; acyloxyl (e.g., acetyloxyl); cycloalkyl (e.g., cyclohexyl, cyclopropyl); heterocycle (e.g., pyridyl, thienyl, pyrrolidinyl, benzo[1,3]dioxolyl, indolyl); heterocycle substituted with at least one substituent selected from the group consisting of alkoxycarbonyl (e.g., tert-butoxycarbonyl), N-arylkyl, N-alkylsulphonylamino (e.g., N-benzyl, N-(methylsufonyl)amino), and alkoxylcarbonylamino (e.g., tert-butoxycarbonylamino) groups; N-arylkyl, N-alkylsulphonylamino (e.g., N-benzyl, N-(methylsufonyl)amino); and alkoxylcarbonylamino (e.g., tert-butoxycarbonylamino) groups.

Preferred cycloalkyl and substituted cycloalkyl groups that variable A represents are cyclopropyl, cyclobutyl, cyclopentyl, cyclohexyl, cycloheptyl, adamantyl and bicyclo[2.2.2]octyl groups. More preferred is where the cycloalkyl group is unsubstituted. Most preferably the cycloalkyl group that A represents is unsubstituted cyclohexyl.

A preferred substituent of the cycloalkyl of variable A is hydroxyl. A most preferred substituted cycloalkyl group that A represents is hydroxyhexyl.

A preferred cycloalkenyl group that variable A represents is a cyclohexenyl group.

Preferred aryl groups that variable A represents are phenyl and phenyl substituted with one or more substituents selected from the group consisting of alkyl (e.g., methyl), hydroxyl, alkoxyl (e.g., methoxyl, ethoxyl), dialkylamino (e.g., dimethylamino), cyano, halogen (chlorine, fluorine, di-fluoro) and polyhaloalkyl (e.g., trifluoromethyl). More preferably, when A represents substituted phenyl, the phenyl is mono-substituted.

Preferred alkenyl groups that A represents are ethenyl, propenyl and butenyl groups. Most preferably the alkenyl group that A represents is ethenyl. Preferred substituted alkenyl groups that A represents are arylalkenyl groups (e.g., phenylalkenyl). Most preferably the aralalkenyl group that A represents is phenylethenyl, wherein the phenyl group is optionally substituted with one or more substituent selected from the group consisting of halogen (e.g., fluorine), alkyl and alkoxyl groups.

Preferred heterocycle groups that A represents are morpholinyl, pyrrolidinyl, piperidyl, pyrrolyl, benzo[1,3]dioxolyl, furyl, isoxazolyl, tetrahydrofuryl, thienyl, pyridyl and indolyl groups.

Preferred substituents when variable A is a substituted heterocycle are alkyl (e.g., methyl, dimethyl), oxo, alkoxyl, halogen, acyl (e.g., acetyl), alkoxycarbonyl (e.g., tert-butoxycarbonyl), heterocycle (e.g., morpholinyl), heterocycle alkyl (e.g., triazolylmethyl), alkylarylsulfonyl (e.g., methylphenylsulfonyl), sulfamoyl and substituted sulfamoyl —SO₂NR₄R₅.

Preferred independent substituents on the amino group when variable A is alkylamino, dialkylamino, arylamino or arylalkylamino are methyl, ethyl, pentyl, tert-butyl, phenyl and benzyl groups and substituted phenyl or substituted benzyl wherein one of more hydrogen atom on the phenyl ring is substituted independently with substituents selected from the group consisting of halogen, alkyl and alkoxyl groups.

Preferred monocyclic aryl groups that B represents are phenyl and substituted phenyl. Preferred bicyclic aryl groups that B represents are naphthyl, tetrahydronaphthyl, substituted naphthyl and tetrahydronaphthyl.

Preferred monocyclic heterocyclic groups that B represents are pyrazinyl, pyridyl, pyrazolyl and thienyl.

Preferred bicyclic heterocyclic groups that B represents are indolyl, isoquinolyl, quinolyl, benzofuranyl, 2,3-dihydrobenzofuranyl, 2,3-dihydro-1,4-benzodioxinyl, 3,4-dihydro-2H-benzo[b][1,4]dioxepinyl, 2,1,3-benzothiadiazolyl, 2,1,3-benzoxadiazolyl, quinazolinyl, benzimidazolyl, benzo[1,3]dioxolyl and 3H-benzotriazolyl groups. More preferred bicyclic heterocyclic groups that B represents are indolyl, benzo[1,3]dioxolyl and dihydrobenzo[1,4]dioxinyl groups.

Preferred substituents when variable B represents a substituted monocyclic aryl, substituted bicyclic aryl, substituted monocyclic heterocycle or substituted bicyclic heterocycle group are halogen (e.g., chlorine, fluorine, bromine), hydroxyl, cyano, nitro, alkyl (e.g., methyl, ethyl, propyl, butyl, isopropyl); alkylthio (e.g., methylthio), alkylcarbonyl (e.g., acetyl), alkoxyl (e.g., methoxyl, ethoxyl, isopropoxyl, butoxyl); polyhaloalkyl (e.g., trifluoromethyl), polyhaloalkoxy (e.g., trifluoromethoxy, 2,2,2-trifluoroethoxy) alkoxylalkyl (e.g., methoxymethyl) and polyhaloalkylsulfonyloxyl (e.g., trifluoromethylsulfonyloxyl) groups.

Variable R₁

As used herein, variable R₁ is selected from the group consisting of hydrogen, halogen, hydroxyl, alkyl, substituted alkyl, alkoxyl, substituted alkoxyl, nitro, aryl, substituted aryl, heterocycle, substituted heterocycle, alkenyl, substituted alkenyl, amino, alkylamino, dialkylamino, cyano, —SR₃, —C(O)R₃, —C(O)NR₃R₃, —NR₃C(O)R₃, —NR₃SO₂R₃, —NR₃C(O)OR₃ and —N(H)C(O)N(H)R₃.

Preferably, R₁ represents hydrogen, halogen, hydroxyl, alkyl, alkoxyl, substituted alkoxyl, nitro, substituted alkyl, heterocycle or substituted heterocycle. Preferred heterocycles that R₁ represents are thienyl and isoxazole. Preferred substituted heterocycles that R₁ represents are heterocycles wherein one or more hydrogen has been replaced with an alkyl (e.g., methyl), alkylcarbonyl (e.g., acetyl) or alkoxyl (e.g., methoxy) group. Preferably substituted heterocycles that R₁ represents are substituted thienyl and substituted isoxazole. More preferably, R₁ represents hydrogen, hydroxyl, nitro, polyhaloalkoxyl (e.g., trifluoromethoxy), polyhaloalkyl (e.g., trifluoromethyl), alkyl (e.g., methyl), halogen (e.g., bromine, chlorine, fluorine) or alkoxyl (e.g., methoxy) groups, or a group wherein —NR₃C(O)OR₃ represents —NH—C(O)O-alkyl. More preferably R₁ is one or more substituents H, fluorine, chlorine, bromine, hydroxyl or methyl.

Variable R₂

As used herein, R₂ is selected from the group consisting of hydrogen, alkyl, substituted alkyl, alkenyl and substituted alkenyl groups. The definitions of these terms are encompassed by the definitions previously presented. Preferred groups that R₂ represents are H and alkyl (e.g., methyl).

Variable R₃

As used herein, variable R₃ is independently selected from a group consisting of hydrogen, alkyl, substituted alkyl, cycloalkyl, substituted cycloalkyl, aryl, substituted aryl, heterocycle and substituted heterocycle. Preferably, R₃ is hydrogen, alkyl, cycloalkyl, aryl or heterocycle.

Variables Y and “ [symbol] ”

Y represents a CH, CH₂, CR₂, CHR₂ group or a bond. Preferably, Y is CH₂.

The symbol “ [symbol] ” represents a single or double bond between carbon atoms. When Y is CH, the double bond is shifted so as to contain the Y group.

Variable X:

X represents a bond, CH, CH₂, SO or SO₂ group or a carbon, nitrogen or sulphur atom and, when X is a nitrogen atom or CH group, the -Z-(CH₂)ₘ—B group is bound to said nitrogen atom or CH group, and when X is a carbon atom Z″ is not a hydrogen atom and the Z-(CH₂)ₘ—B and Z″ groups are bound to said carbon;. Preferably, X is a nitrogen atom.

Variable Z:

As used herein, Z represents a valence bond, an oxygen or sulphur atom or a —CH(OH)—, —C(O)—NR₃—C(O)—, —NR₃—C(O)—NR₃— or —NR₃-group. Prefereably, Z represents a valence bond, or a —CH(OH)—, —C(O)—NR₃—C(O)—, —NR₃—C(O)—NR₃— or —NR₃-group. Most preferably, Z represents a valence bond.

Variable Z′:

As used herein, Z′ represents a bond or an oxygen or sulphur atom. Preferably Z′ represents a bond or an oxygen atom.

Variable Z″:

As used herein, Z″ represents a hydrogen atom or hydroxyl, oxo, alkylcarbonyl or cyano group. The preferred alkylcarbonyl group that Z″ represents is acetyl. Preferably, Z″ represents a hydrogen atom.

Variable Q:

As used herein, Q represents a carbonyl, thiocarbonyl or sulfonyl group. The preferred group that Q represents is carbonyl.

Variables n, m and p:

The value of n is 1 or 2. The preferred value of n is 1.

The value of m is 0, 1 or 2. The preferred value of m is 0.

The value of p is 1, 2 or 3. The preferred value of p is 2.

Variables a, b, c and d:

Variables a, b, c and d are independently a carbon or nitrogen atom, or CH, CH₂ or NH group, with the proviso that not more than two of a, b, c and d may simultaneously be a nitrogen atom and/or NH group. Preferably, each a, b, c and d are simultaneously carbon atom and/or CH group.

The following are independent preferences of formula I:

A is a member selected from the group consisting of (i) cycloalkyl; (ii) cycloalkyl substituted with hydroxyl; (iii) heterocycle; (iv) heterocycle substituted with one or more substituents selected from the group consisting of (C₁-C₇)-alkyl, oxo, (C₁-C₇)-alkoxyl, halogen, acetyl, (C₂-C₇)-alkoxycarbonyl, heterocycle, heterocyclicalkyl, alkylarylsulfonyl, sulfamoyl and substituted sulfamoyl —SO₂NR₄R₅, wherein R₄ and R₅ are each independently a hydrogen atom or a (C₁-C₄)-alkyl group, or R₄ and R₅ are joined to form a heterocycle comprising at least one nitrogen atom; (v) aryl; (vi) aryl substituted with one or more substituents selected from the group of hydroxyl, halogen, cyano, (C₁-C₇)-alkyl, (C₁-C₇)-alkoxyl, (C₁-C₄)-polyhaloalkyl, (C₁-C₄)-polyhaloalkoxyl, (C₁-C₅)-alkylamino and di-[(C₁-C₅)]-alkylamino (vii) alkyl; and (viii) substituted alkyl;

R₁ represents one or more substituent selected from the group consisting of hydrogen, hydroxyl, nitro, (C₁-C₅)-polyhaloalkoxyl, (C₁-C₅)-polyhaloalkyl, (C₁-C₇)-alkyl, halogen, (C₁-C₇)-alkoxyl, substituted (C₁-C₇)-alkoxyl, heterocycle, substituted heterocycle and —NH—C(O)O—(C₁-C₇)-alkyl groups;

R₂ is one or two substituents selected from the group consisting of H and (C₁-C₄)-alkyl groups;

B represents an aryl or heterocyclic selected from the group (ix) consisting of phenyl, naphthyl, tetrahydronaphthyl, pyrazinyl, pyridyl, pyrazolyl, thienyl, indolyl, isoquinolyl, quinolyl, benzofuranyl, 2,3-dihydrobenzofuranyl, 2,3-dihydro-1,4-benzodioxinyl, 3,4-dihydro-2H-benzo[b][1,4]dioxepinyl, 2,1,3-benzothiadiazolyl, 2,1,3-benzoxadiazolyl, quinazolinyl, benzimidazolyl, benzo[1,3]dioxolyl and 3H-benzotriazolyl groups, or a substituted aryl or heterocyclic of group (ix).

The following are non-limiting simultaneous preferences (A)-(K) of formula I:

(A) A is a member selected from the group consisting of (i) cycloalkyl; (ii) cycloalkyl substituted with hydroxyl; (iii) heterocycle; (iv) heterocycle substituted with one or more substituents selected from the group consisting of (C₁-C₇)-alkyl, oxo, (C₁-C₇)-alkoxyl, halogen, acetyl, (C₂-C₇)-alkoxycarbonyl, heterocycle, heterocyclicalkyl, alkylarylsulfonyl, sulfamoyl and substituted sulfamoyl —SO₂NR₄R₅, wherein R₄ and R₅ are each independently a hydrogen atom or a (C₁-C₄)-alkyl group, or R₄ and R₅ are joined to form a heterocycle comprising at least one nitrogen atom; (v) aryl; (vi) aryl substituted with one or more substituents selected from the group of hydroxyl, halogen, cyano, (C₁-C₇)-alkyl, (C₁-C₇)-alkoxyl, (C₁-C₄)-polyhaloalkyl, (C₁-C₄)-polyhaloalkoxyl, (C₁-C₅)-alkylamino and di-[(C₁-C₅)]-alkylamino (vii) alkyl; and (viii) substituted alkyl; and B represents an aryl or heterocyclic selected from the group (ix) consisting of phenyl, naphthyl, tetrahydronaphthyl, pyrazinyl, pyridyl, pyrazolyl, thienyl, indolyl, isoquinolyl, quinolyl, benzofuranyl, 2,3-dihydrobenzofuranyl, 2,3-dihydro-1,4-benzodioxinyl, 3,4-dihydro-2H-benzo[b][1,4]dioxepinyl, 2,1,3-benzothiadiazolyl, 2,1,3-benzoxadiazolyl, quinazolinyl, benzimidazolyl, benzo[1,3]dioxolyl and 3H-benzotriazolyl groups, or a substituted aryl or heterocyclic of group (ix);

(B) A is a member selected from the group consisting of (i) cycloalkyl; (ii) cycloalkyl substituted with hydroxyl; (iii) heterocycle; (iv) heterocycle substituted with one or more substituents selected from the group consisting of (C₁-C₇)-alkyl, oxo, (C₁-C₇)-alkoxyl, halogen, acetyl, (C₂-C₇)-alkoxycarbonyl, heterocycle, heterocyclicalkyl, alkylarylsulfonyl, sulfamoyl and substituted sulfamoyl —SO₂NR₄R₅, wherein R₄ and R₅ are each independently a hydrogen atom or a (C₁-C₄)-alkyl group, or R₄ and R₅ are joined to form a heterocycle comprising at least one nitrogen atom; (v) aryl; (vi) aryl substituted with one or more substituents selected from the group of hydroxyl, halogen, cyano, (C₁-C₇)-alkyl, (C₁-C₇)-alkoxyl, (C₁-C₄)-polyhaloalkyl, (C₁-C₄)-polyhaloalkoxyl, (C₁-C₅)-alkylamino and di-[(C₁-C₅)]-alkylamino (vii) alkyl; and (viii) substituted alkyl; B represents an aryl or heterocyclic selected from the group (ix) consisting of phenyl, naphthyl, tetrahydronaphthyl, pyrazinyl, pyridyl, pyrazolyl, thienyl, indolyl, isoquinolyl, quinolyl, benzofuranyl, 2,3-dihydrobenzofuranyl, 2,3-dihydro-1,4-benzodioxinyl, 3,4-dihydro-2H-benzo[b][1,4]dioxepinyl, 2,1,3-benzothiadiazolyl, 2,1,3-benzoxadiazolyl, quinazolinyl, benzimidazolyl, benzo[1,3]dioxolyl and 3H-benzotriazolyl groups, or a substituted aryl or heterocyclic of group (ix); and R₁ represents one or more substituent selected from the group consisting of hydrogen, hydroxyl, nitro, (C₁-C₅)-polyhaloalkoxyl, (C₁-C₅)-polyhaloalkyl, (C₁-C₇)-alkyl, halogen, (C₁-C₇)-alkoxyl, substituted (C₁-C₇)-alkoxyl, heterocycle, substituted heterocycle and —NH—C(O)O—(C₁-C₇)-alkyl groups;

(C) A is a member selected from the group consisting of (i) cycloalkyl; (ii) cycloalkyl substituted with hydroxyl; (iii) heterocycle; (iv) heterocycle substituted with one or more substituents selected from the group consisting of (C₁-C₇)-alkyl, oxo, (C₁-C₇)-alkoxyl, halogen, acetyl, (C₂-C₇)-alkoxycarbonyl, heterocycle, heterocyclicalkyl, alkylarylsulfonyl, sulfamoyl and substituted sulfamoyl —SO₂NR₄R₅, wherein R₄ and R₅ are each independently a hydrogen atom or a (C₁-C₄)-alkyl group, or R₄ and R₅ are joined to form a heterocycle comprising at least one nitrogen atom; (v) aryl; (vi) aryl substituted with one or more substituents selected from the group of hydroxyl, halogen, cyano, (C₁-C₇)-alkyl, (C₁-C₇)-alkoxyl, (C₁-C₄)-polyhaloalkyl, (C₁-C₄)-polyhaloalkoxyl, (C₁-C₅)-alkylamino and di-[(C₁-C₅)]-alkylamino (vii) alkyl; and (viii) substituted alkyl; B represents an aryl or heterocyclic selected from the group (ix) consisting of phenyl, naphthyl, tetrahydronaphthyl, pyrazinyl, pyridyl, pyrazolyl, thienyl, indolyl, isoquinolyl, quinolyl, benzofuranyl, 2,3-dihydrobenzofuranyl, 2,3-dihydro-1,4-benzodioxinyl, 3,4-dihydro-2H-benzo[b][1,4]dioxepinyl, 2,1,3-benzothiadiazolyl, 2,1,3-benzoxadiazolyl, quinazolinyl, benzimidazolyl, benzo[1,3]dioxolyl and 3H-benzotriazolyl groups, or a substituted aryl or heterocyclic of group (ix); and R₂ is one or two substituents selected from the group consisting of H and (C₁-C₄)-alkyl groups;

(D) A is a member selected from the group consisting of (i) cycloalkyl; (ii) cycloalkyl substituted with hydroxyl; (iii) heterocycle; (iv) heterocycle substituted with one or more substituents selected from the group consisting of (C₁-C₇)-alkyl, oxo, (C₁-C₇)-alkoxyl, halogen, acetyl, (C₂-C₇)-alkoxycarbonyl, heterocycle, heterocyclicalkyl, alkylarylsulfonyl, sulfamoyl and substituted sulfamoyl —SO₂NR₄R₅, wherein R₄ and R₅ are each independently a hydrogen atom or a (C₁-C₄)-alkyl group, or R₄ and R₅ are joined to form a heterocycle comprising at least one nitrogen atom; (v) aryl; (vi) aryl substituted with one or more substituents selected from the group of hydroxyl, halogen, cyano, (C₁-C₇)-alkyl, (C₁-C₇)-alkoxyl, (C₁-C₄)-polyhaloalkyl, (C₁-C₄)-polyhaloalkoxyl, (C₁-C₅)-alkylamino and di-[(C₁-C₅)]-alkylamino (vii) alkyl; and (viii) substituted alkyl; B represents an aryl or heterocyclic selected from the group (ix) consisting of phenyl, naphthyl, tetrahydronaphthyl, pyrazinyl, pyridyl, pyrazolyl, thienyl, indolyl, isoquinolyl, quinolyl, benzofuranyl, 2,3-dihydrobenzofuranyl, 2,3-dihydro-1,4-benzodioxinyl, 3,4-dihydro-2H-benzo[b][1,4]dioxepinyl, 2,1,3-benzothiadiazolyl, 2,1,3-benzoxadiazolyl, quinazolinyl, benzimidazolyl, benzo[1,3]dioxolyl and 3H-benzotriazolyl groups, or a substituted aryl or heterocyclic of group (ix); R₁ represents one or more substituent selected from the group consisting of hydrogen, hydroxyl, nitro, (C₁-C₅)-polyhaloalkoxyl, (C₁-C₅)-polyhaloalkyl, (C₁-C₇)-alkyl, halogen, (C₁-C₇)-alkoxyl, substituted (C₁-C₇)-alkoxyl, heterocycle, substituted heterocycle and —NH—C(O)O—(C₁-C₇)-alkyl groups; and R₂ is one or two substituents selected from the group consisting of H and (C₁-C₄)-alkyl groups;

(E) A is a member selected from the group consisting of (i) cycloalkyl; (ii) cycloalkyl substituted with hydroxyl; (iii) heterocycle; (iv) heterocycle substituted with one or more substituents selected from the group consisting of (C₁-C₇)-alkyl, oxo, (C₁-C₇)-alkoxyl, halogen, acetyl, (C₂-C₇)-alkoxycarbonyl, heterocycle, heterocyclicalkyl, alkylarylsulfonyl, sulfamoyl and substituted sulfamoyl —SO₂NR₄R₅, wherein R₄ and R₅ are each independently a hydrogen atom or a (C₁-C₄)-alkyl group, or R₄ and R₅ are joined to form a heterocycle comprising at least one nitrogen atom; (v) aryl; (vi) aryl substituted with one or more substituents selected from the group of hydroxyl, halogen, cyano, (C₁-C₇)-alkyl, (C₁-C₇)-alkoxyl, (C₁-C₄)-polyhaloalkyl, (C₁-C₄)-polyhaloalkoxyl, (C₁-C₅)-alkylamino and di-[(C₁-C₅)]-alkylamino (vii) alkyl; and (viii) substituted alkyl; and R₁ represents one or more substituent selected from the group consisting of hydrogen, hydroxyl, nitro, (C₁-C₅)-polyhaloalkoxyl, (C₁-C₅)-polyhaloalkyl, (C₁-C₇)-alkyl, halogen, (C₁-C₇)-alkoxyl, substituted (C₁-C₇)-alkoxyl, heterocycle, substituted heterocycle and —NH—C(O)O—(C₁-C₇)-alkyl groups;

(F) A is a member selected from the group consisting of (i) cycloalkyl; (ii) cycloalkyl substituted with hydroxyl; (iii) heterocycle; (iv) heterocycle substituted with one or more substituents selected from the group consisting of (C₁-C₇)-alkyl, oxo, (C₁-C₇)-alkoxyl, halogen, acetyl, (C₂-C₇)-alkoxycarbonyl, heterocycle, heterocyclicalkyl, alkylarylsulfonyl, sulfamoyl and substituted sulfamoyl —SO₂NR₄R₅, wherein R₄ and R₅ are each independently a hydrogen atom or a (C₁-C₄)-alkyl group, or R₄ and R₅ are joined to form a heterocycle comprising at least one nitrogen atom; (v) aryl; (vi) aryl substituted with one or more substituents selected from the group of hydroxyl, halogen, cyano, (C₁-C₇)-alkyl, (C₁-C₇)-alkoxyl, (C₁-C₄)-polyhaloalkyl, (C₁-C₄)-polyhaloalkoxyl, (C₁-C₅)-alkylamino and di-[(C₁-C₅)]-alkylamino (vii) alkyl; and (viii) substituted alkyl; and R₂ is one or two substituents selected from the group consisting of H and (C₁-C₄)-alkyl groups;

(G) A is a member selected from the group consisting of (i) cycloalkyl; (ii) cycloalkyl substituted with hydroxyl; (iii) heterocycle; (iv) heterocycle substituted with one or more substituents selected from the group consisting of (C₁-C₇)-alkyl, oxo, (C₁-C₇)-alkoxyl, halogen, acetyl, (C₂-C₇)-alkoxycarbonyl, heterocycle, heterocyclicalkyl, alkylarylsulfonyl, sulfamoyl and substituted sulfamoyl —SO₂NR₄R₅, wherein R₄ and R₅ are each independently a hydrogen atom or a (C₁-C₄)-alkyl group, or R₄ and R₅ are joined to form a heterocycle comprising at least one nitrogen atom; (v) aryl; (vi) aryl substituted with one or more substituents selected from the group of hydroxyl, halogen, cyano, (C₁-C₇)-alkyl, (C₁-C₇)-alkoxyl, (C₁-C₄)-polyhaloalkyl, (C₁-C₄)-polyhaloalkoxyl, (C₁-C₅)-alkylamino and di-[(C₁-C₅)]-alkylamino (vii) alkyl; and (viii) substituted alkyl; R₁ represents one or more substituent selected from the group consisting of hydrogen, hydroxyl, nitro, (C₁-C₅)-polyhaloalkoxyl, (C₁-C₅)-polyhaloalkyl, (C₁-C₇)-alkyl, halogen, (C₁-C₇)-alkoxyl, substituted (C₁-C₇)-alkoxyl, heterocycle, substituted heterocycle and —NH—C(O)O—(C₁-C₇)-alkyl groups; and R₂ is one or two substituents selected from the group consisting of H and (C₁-C₄)-alkyl groups;

(H) B represents an aryl or heterocyclic selected from the group (ix) consisting of phenyl, naphthyl, tetrahydronaphthyl, pyrazinyl, pyridyl, pyrazolyl, thienyl, indolyl, isoquinolyl, quinolyl, benzofuranyl, 2,3-dihydrobenzofuranyl, 2,3-dihydro-1,4-benzodioxinyl, 3,4-dihydro-2H-benzo[b][1,4]dioxepinyl, 2,1,3-benzothiadiazolyl, 2,1,3-benzoxadiazolyl, quinazolinyl, benzimidazolyl, benzo[1,3]dioxolyl and 3H-benzotriazolyl groups, or a substituted aryl or heterocyclic of group (ix); and R₁ represents one or more substituent selected from the group consisting of hydrogen, hydroxyl, nitro, (C₁-C₅)-polyhaloalkoxyl, (C₁-C₅)-polyhaloalkyl, (C₁-C₇)-alkyl, halogen, (C₁-C₇)-alkoxyl, substituted (C₁-C₇)-alkoxyl, heterocycle, substituted heterocycle and —NH—C(O)O—(C₁-C₇)-alkyl groups;

(I) B represents an aryl or heterocyclic selected from the group (ix) consisting of phenyl, naphthyl, tetrahydronaphthyl, pyrazinyl, pyridyl, pyrazolyl, thienyl, indolyl, isoquinolyl, quinolyl, benzofuranyl, 2,3-dihydrobenzofuranyl, 2,3-dihydro-1,4-benzodioxinyl, 3,4-dihydro-2H-benzo[b][1,4]dioxepinyl, 2,1,3-benzothiadiazolyl, 2,1,3-benzoxadiazolyl, quinazolinyl, benzimidazolyl, benzo[1,3]dioxolyl and 3H-benzotriazolyl groups, or a substituted aryl or heterocyclic of group (ix); and R₂ is one or two substituents selected from the group consisting of H and (C₁-C₄)-alkyl groups;

(J) B represents an aryl or heterocyclic selected from the group (ix) consisting of phenyl, naphthyl, tetrahydronaphthyl, pyrazinyl, pyridyl, pyrazolyl, thienyl, indolyl, isoquinolyl, quinolyl, benzofuranyl, 2,3-dihydrobenzofuranyl, 2,3-dihydro-1,4-benzodioxinyl, 3,4-dihydro-2H-benzo[b][1,4]dioxepinyl, 2,1,3-benzothiadiazolyl, 2,1,3-benzoxadiazolyl, quinazolinyl, benzimidazolyl, benzo[1,3]dioxolyl and 3H-benzotriazolyl groups, or a substituted aryl or heterocyclic of group (ix); R₁ represents one or more substituent selected from the group consisting of hydrogen, hydroxyl, nitro, (C₁-C₅)-polyhaloalkoxyl, (C₁-C₅)-polyhaloalkyl, (C₁-C₇)-alkyl, halogen, (C₁-C₇)-alkoxyl, substituted (C₁-C₇)-alkoxyl, heterocycle, substituted heterocycle and —NH—C(O)O—(C₁-C₇)alkyl groups; and R₂ is one or two substituents selected from the group consisting of H and (C₁-C₄)-alkyl groups; or

(K) R₁ represents one or more substituent selected from the group consisting of hydrogen, hydroxyl, nitro, (C₁-C₅)-polyhaloalkoxyl, (C₁-C₅)-polyhaloalkyl, (C₁-C₇)-alkyl, halogen, (C₁-C₇)-alkoxyl, substituted (C₁-C₇)-alkoxyl, heterocycle, substituted heterocycle and —NH—C(O)O—(C₁-C₇)-alkyl groups; and R₂ is one or two substituents selected from the group consisting of H and (C₁-C₄)-alkyl groups.

Further simultaneous preferences are as follows:

(L) Any one of preferences (A)-(K) wherein Y represents a CH₂ group.

(M) Any one of preferences (A)-(L) wherein X represents a nitrogen atom.

(N) Any one of preferences (A)-(M) wherein Q represents a carbonyl group.

(O) Any one of preferences (A)-(N) wherein Z″ represents a hydrogen atom.

(P) Any one of preferences (A)-(O) wherein Z represents a bond, Z″ represents a hydrogen atom and m=0.

Additional preferred independent preferences for Formula I are R₁ is hydrogen, R₂ is hydrogen, Y is CH₂, Q is carbonyl, n=1, A is cycloalkyl, n is 1, a, b, c and d are each CH, W is group (i), Z is a bond, X is nitrogen or CH group, m is 0 and B is phenyl, substituted phenyl, heterocycle or substituted heterocycle.

The present invention also specifically contemplates the simultaneous combinations:

where Y is CH₂ and n is 1; or

where Y is CH₂, a, b, c, and d are each CH, and n is 1; or

where Y is CH₂, a, b, c, and d are each CH, and n is 1, W is group (i), m is 0 and B is a substituted monocyclic aryl or unsubstituted bicyclic heterocycle; or

where Y is CH₂, a, b, c, and d are each CH, and n is 1, W is group (i), m is 0, B is a substituted monocyclic aryl or unsubstituted bicyclic heterocycle and X is a nitrogen atom; or

where Y is CH₂, a, b, c, and d are each CH, and n is 1, W is group (i), m is 0, B is a substituted monocyclic aryl or unsubstituted bicyclic heterocycle, X is a nitrogen atom, Q is carbonyl and A is cycloalkyl or substituted alkyl; or

where Y is CH₂, a, b, c, and d are each CH, and n is 1, W is group (i), m is 0, B is a substituted monocyclic aryl or unsubstituted bicyclic heterocycle, X is a nitrogen atom, Q is carbonyl and A is cycloalkyl or substituted alkyl, and R₁ and R₂ are hydrogen; or

where Y is CH₂, a, b, c, and d are each CH, and n is 1, W is group (i), m is 0, B is a substituted monocyclic aryl or unsubstituted bicyclic heterocycle, X is a nitrogen atom, Q is carbonyl and A is cycloalkyl or substituted alkyl, R₁ and R₂ are hydrogen and Z is a valence bond; or

where Y is CH₂, a, b, c, and d are each CH, and n is 1, W is group (i), m is 0, B is a substituted monocyclic aryl or unsubstituted bicyclic heterocycle, X is a nitrogen atom, Q is carbonyl and A is cycloalkyl or substituted alkyl, R₁ and R₂ are hydrogen, Z is a valence bond and Z″ is hydrogen.

The aforesaid combinations are also preferred for formulation into a pharmaceutical composition and for administration to a patient in need of treatment.

The present invention further encompasses, for example and without limition, the following particular compounds:

1-(1-Cyclohexanecarbonyl-1,2,3,4-tetrahydroquinoline-2-ylmethyl)-4-(4-indolyl)piperazine,

(+)-1-(1-Cyclohexanecarbonyl-1,2,3,4-tetrahydroquinoline-2-ylmethyl)-4-(4-indolyl)piperazine,

(−)-1-(1-Cyclohexanecarbonyl-1,2,3,4-tetrahydroquinoline-2-ylmethyl)-4-(4-indolyl)piperazine

1-(1-cyclohexanecarbonyl-1,2,3,4-tetrahydroquinoline-2-ylmethyl)-4-(2-methoxyphenyl)piperazine,

1-[2-(1-cyclohexanecarbonyl-1,2,3,4-tetrahydroquinoline-2-yl)ethyl]-4-(2-methoxyphenyl)piperazine,

1-(1-cyclohexanecarbonyl-1,2,3,4-tetrahydroquinoline-2-ylmethyl)-4-[2-(2,2,2-trifluoroethoxy)phenyl]piperazine,

1-(1-cyclohexanecarbonyl-6-fluoro-1,2,3,4-tetrahydroquinoline-2-ylmethyl)-4-[2-(2,2,2-trifluoroethoxy)phenyl]piperazine,

1-[1-(2-ethylbutanoyl)-1,2,3,4-tetrahydroquinoline-2-ylmethyl]-4-[2-(2,2,2-trifluoroethoxyphenyl]piperazine,

1-[1-(3-methoxypropionyl)-1,2,3,4-tetrahydroquinoline-2-ylmethyl]-4-[2-(2,2,2-trifluoroethoxy)phenyl]piperazine,

1-[1-(3-benzyloxypropionyl)-1,2,3,4-tetrahydroquinoline-2-ylmethyl]-4-[2-(2,2,2-trifluoroethoxy)phenyl]piperazine,

1-[1-(3-benzyloxypropionyl)-1,2,3,4-tetrahydroquinoline-2-ylmethyl]-4-(4-indolyl)piperazine,

1-[1-(3-hydroxypropionyl)-1,2,3,4-tetrahydroquinoline-2-ylmethyl]-4-[2-(2,2,2-trifluoroethoxy)phenyl]piperazine,

1-[1-(3-methoxypropionyl)-1,2,3,4-tetrahydroquinoline-2-ylmethyl]-4-(4-indolyl)piperazine,

1-[1-(3-isopropoxypropionyl)-1,2,3,4-tetrahydroquinoline-2-ylmethyl]-4-(4-indolyl)piperazine,

1-(1-acetyl-1,2,3,4-tetrahydroquinoline-2-ylmethyl)-4-(4-indolyl)piperazine,

1-[1-(4-morpholinocarbonyl)-1,2,3,4-tetrahydroquinoline-2-ylmethyl]-4-(4-indolyl)piperazine,

1-(1-cyclohexanecarbonyl-1,2,3,4-tetrahydroquinoline-2-ylmethyl)-4-[4-(1-methyl)indolyl]piperazine,

1-(1-cyclohexanecarbonyl-6-methoxy-1,2,3,4-tetrahydroquinoline-2-ylmethyl)-4-(4-indolyl)piperazine,

1-[1-(3-hydroxypropionyl)-1,2,3,4-tetrahydroquinoline-2-ylmethyl)-4-(4-indolyl)piperazine,

1-(1-cyclohexanecarbonyl-6-fluoro-1,2,3,4-tetrahydroquinoline-2-ylmethyl)-4-(4-indolyl)piperazine.

1-(1-Dimethylaminocarbonyl-1,2,3,4-tetrahydroquinoline-2-ylmethyl)-4-(4-indolyl)piperazine

1-(1-Ethylaminocarbonyl-1,2,3,4-tetrahydroquinoline-2-ylmethyl)-4-(4-indolyl)-piperazine

1-(1-Cyclohexanecarbonyl-1,2,3,4-tetrahydroquinoline-2-ylmethyl)-4-(1-isoquinolinyl)piperazine

1-(1-Cyclohexanecarbonyl-1,2,3,4-tetrahydroquinoline-2-ylmethyl)-4-(2-methoxyphenyl)piperidine

1-(7-Benzofuranyl)-4-(1-cyclohexanecarbonyl-1,2,3,4-tetrahydroquinoline-2-ylmethyl)piperazine

1-(2-Ethylbutanoyl-1,2,3,4-tetrahydroquinoline-2-ylmethyl)-4-(4-indolyl)-piperazine

1-(1-Cyclohex-3-enylcarbonyl-1,2,3,4-tetrahydroquinoline-2-ylmethyl)-4-(4-indolyl)-piperazine

1-(1-Cycloheptanecarbonyl-1,2,3,4-tetrahydroquinoline-2-ylmethyl)-4-(4-indolyl)-piperazine

1-(1-Cyclopentanecarbonyl-1,2,3,4-tetrahydroquinoline-2-ylmethyl)-4-(4-indolyl)-piperazine

1-(1-Benzoyl-1,2,3,4-tetrahydroquinoline-2-ylmethyl)-4-(4-indolyl)-piperazine

1-(4-Indolyl)-4-(1-pentanoyl-1,2,3,4-tetrahydroquinoline-2-ylmethyl)piperazine

1-(1-Cyclohexanecarbonyl-6-trifluoromethyl-1,2,3,4-tetrahydroquinoline-2-ylmethyl)-4-(4-indolyl)piperazine

1-(1-Cyclohexanecarbonyl-6-trifluoromethoxy-1,2,3,4-tetrahydroquinoline-2-ylmethyl)-4-(4-indolyl)piperazine

1-[1-(3-Benzylaminopropionyl)-1,2,3,4-tetrahydroquinoline-2-ylmethyl]-4-(4-indolyl)piperazine

1-(3-Aminopropionyl-1,2,3,4-tetrahydroquinoline-2-ylmethyl]-4-(4-indolyl)piperazine

1-(4-Indolyl)-[1-(3-methylaminopropionyl-1,2,3,4-tetrahydroquinoline-2-ylmethyl]-piperazine

1-[1-(3-Dimethylaminopropionyl)-1,2,3,4-tetrahydroquinoline-2-ylmethyl]-4-(4-indolyl)piperazine

1-(4-Indolyl)-4-(1-phenylaminocarbonyl-1,2,3,4-tetrahydroquinoline-2-ylmethyl)-piperazine

1-(1-Cyclohexanecarbonyl-6-methyl-1,2,3,4-tetrahydroquinoline-2-ylmethyl)-4-(4-indolyl)piperazine

1-(4-Indolyl)-4-(1-pyrrolidinecarbonyl-1,2,3,4-tetrahydroquinoline-2-ylmethyl)pipe-razine

1-(6-Bromo-1-cyclohexanecarbonyl-1,2,3,4-tetrahydroquinoline-2-ylmethyl)-4-(4-indolyl)piperazine

1-(1-Cyclohexanecarbonyl-1,2,3,4-tetrahydroquinoline-2-ylmethyl)-4-(4-fluoro-2-methoxyphenyl)piperazine

1-(1-Cyclohexanecarbonyl-1,2,3,4-tetrahydroquinoline-2-ylmethyl)-4-(2-methyl-4-indolyl)piperazine

1-(1-Cyclohexanecarbonyl-1,2,3,4-tetrahydroquinoline-2-ylmethyl)-4-phenylpiperazine

1-(1-Cyclohexanecarbonyl-1,2,3,4-tetrahydroquinoline-2-ylmethyl)-4-(2-ethoxyphe-nyl)piperazine

1-(1-Cyclohexanecarbonyl-1,2,3,4-tetrahydroquinoline-2-ylmethyl)-4-(2,5-dimethoxyphenyl)piperazine

1-(1-Cyclohexanecarbonyl-1,2,3,4-tetrahydroquinoline-2-ylmethyl)-4-(2,3-dihydro-benzofuran-7-yl)piperazine

1-(1-Cyclohexanecarbonyl-1,2,3,4-tetrahydroquinoline-2-ylmethyl)-4-(5-fluoro-2-methoxyphenyl)piperazine

1-(1-Cyclohexanecarbonyl-1,2,3,4-tetrahydroquinoline-2-ylmethyl)-4-(2-methylphe-nyl)piperazine

1-(1-Cyclohexanecarbonyl-1,2,3,4-tetrahydroquinoline-2-ylmethyl)-4-(2-trifluoromethylphenyl)piperazine

1-(1-Cyclohexanecarbonyl-1,2,3,4-tetrahydroquinoline-2-ylmethyl)-4-(2,4-dichloro-phenyl)piperazine

1-(1-Cyclohexanecarbonyl-1,2,3,4-tetrahydroquinoline-2-ylmethyl)-4-(2-hydroxy-phenyl)piperazine

1-(1-Cyclohexanecarbonyl-1,2,3,4-tetrahydroquinoline-2-ylmethyl)-4-(2-i-propoxy-phenyl)piperazine

1-(1-Cyclohexanecarbonyl-1,2,3,4-tetrahydroquinoline-2-ylmethyl)-4-(2-fluoro-5-methylphenyl)piperazine

1-(1-Cyclohexanecarbonyl-1,2,3,4-tetrahydroquinoline-2-ylmethyl)-4-(2,3-dimethyl-4-indolyl)piperazine

1-(1-Cyclohexanecarbonyl-1,2,3,4-tetrahydroquinoline-2-ylmethyl)-4-(2-chloro-5-fluorophenyl)piperazine

1-(1-Cyclohexanecarbonyl-1,2,3,4-tetrahydroquinoline-2-ylmethyl)-4-(5-fluoro-2-methylphenyl)piperazine

1-(2,3-Dihydro-1,4-benzodioxin-5-yl)-4-(1-cyclohexanecarbonyl-1,2,3,4-tetrahydroquinoline-2-ylmethyl)piperazine

1-(1-Cyclohexanecarbonyl-1,2,3,4-tetrahydroquinoline-2-ylmethyl)-4-(2-trifluoromethoxyphenyl)piperazine

1-(1-Cyclohexanecarbonyl-1,2,3,4-tetrahydroquinoline-2-ylmethyl)-4-(4-fluoro-2-methylphenyl)piperazine

1-(1-Cyclohexanecarbonyl-1,2,3,4-tetrahydroquinoline-2-ylmethyl)-4-(2,5-dichlorophenyl)piperazine

1-(1-Cyclohexanecarbonyl-1,2,3,4-tetrahydroquinoline-2-ylmethyl)-4-[4-fluoro-2-(2,2,2-trifluoroethoxyphenyl)]piperazine

1-(1-Cyclohexanecarbonyl-1,2,3,4-tetrahydroquinoline-2-ylmethyl)-4-(2-pyrimidinyl)piperazine

1-(1-Cyclohexanecarbonyl-1,2,3,4-tetrahydroquinoline-2-ylmethyl)-4-(8-quinolinyl)piperazine

1-(5-chloro-2-cyanophenyl)-4-(1-cyclohexanecarbonyl-1,2,3,4-tetrahydroquinoline-2-ylmethyl)piperazine

1-(5-Cyano-2-methoxyphenyl)-4-(1-cyclohexanecarbonyl-1,2,3,4-tetrahydroquinoline-2-ylmethyl)piperazine

1-(1-Acetyl-4-indolyl)-4-(1-cyclohexanecarbonyl-1,2,3,4-tetrahydroquinoline-2-ylmethyl)piperazine

1-(1-Cyclohexanecarbonyl-1,2,3,4-tetrahydroquinoline-2-ylmethyl)-4-(7-indolyl)piperazine

1-(3-Cyano-4-indolyl)-4-(1-cyclohexanecarbonyl-1,2,3,4-tetrahydroquinoline-2-ylmethyl)piperazine

1-(1-Cyclohexanecarbonyl-1,2,3,4-tetrahydroquinoline-2-ylmethyl)-4-(2,4-difluoro-benzyl)piperazine

1-(2-Bromobenzyl)-4-(1-cyclohexanecarbonyl-1,2,3,4-tetrahydroquinoline-2-ylmethyl)piperazine

1-(1-Cyclohexanecarbonyl-1,2,3,4-tetrahydroquinoline-2-ylmethyl)-4-(2,5-difluoro-benzyl)piperazine

1-(1-Cyclohexanecarbonyl-1,2,3,4-tetrahydroquinoline-2-ylmethyl)-4-(1-naphthyl)piperazine

1-(7-Bromo-4-indolyl)-4-(1-cyclohexanecarbonyl-1,2,3,4-tetrahydroquinoline-2-ylmethyl)piperazine

1-(1-Cyclohexanecarbonyl-1,2,3,4-tetrahydroquinoline-2-ylmethyl)-4-(3,4-dihydro-2H-benzo[b][1,4]dioxepin-6-yl)piperazine

1-(2-Chlorobenzyl)-4-(1-cyclohexanecarbonyl-1,2,3,4-tetrahydroquinoline-2-ylmethyl)piperazine

1-(1-Cyclohexanecarbonyl-1,2,3,4-tetrahydroquinoline-2-ylmethyl)-4-(6-methoxy-2-pyridinyl)piperazine

1-(1-Cyclohexanecarbonyl-1,2,3,4-tetrahydroquinoline-2-ylmethyl)-4-(2,5-dichloro-benzyl)piperazine

1-(4-Indolyl)-4-(1-piperidinocarbonyl-1,2,3,4-tetrahydroquinoline-2-ylmethyl)piperazine

1-[1-(3-Cyanopropionyl)-1,2,3,4-tetrahydroquinoline-2-ylmethyl]-4-(4-indolyl)pipe-razine

1-(1-Cyclohexanecarbonyl-8-fluoro-1,2,3,4-tetrahydroquinoline-2-ylmethyl)-4-(4 indolyl)piperazine

1-[1-(3-Acetylaminopropionyl)-1,2,3,4-tetrahydroquinoline-2-ylmethyl]-4-(4-indolyl)piperazine

1-[1-(3-Carbamoylaminopropionyl)-1,2,3,4-tetrahydroquinoline-2-ylmethyl]-4-(4-indolyl)piperazine

1-[1-(3-bis-Acetylaminopropionyl)-1,2,3,4-tetrahydroquinoline-2-ylmethyl]-4-(4-indolyl)piperazine

1-(6-Chloro-1-cyclohexanecarbonyl-1,2,3,4-tetrahydroquinoline-2-ylmethyl)-4-(4-indolyl)piperazine

(R)-1-(1-Cyclohexanecarbonyl-1,2,3,4-tetrahydroquinoline-2-ylmethyl)-4-(1-methyl-4-indolyl)piperazine

(Z)-1-[1-(4-Hydroxycyclohexanecarbonyl)-1,2,3,4-tetrahydroquinoline-2-ylmethyl]-4-(4-indolyl)piperazine

(E)-1-[1-(4-Hydroxycyclohexanecarbonyl)-1,2,3,4-tetrahydroquinoline-2-ylmethyl]-4-(4-indolyl)piperazine

1-(1-Cyclohexanecarbonyl-7-fluoro-1,2,3,4-tetrahydroquinoline-2-ylmethyl)-4-(4-indolyl)piperazine

1-(1-Cyclohexanecarbonyl-6-phenyl-1,2,3,4-tetrahydroquinoline-2-ylmethyl)-4-(4-indolyl)piperazine

1-(1-Cyclohexanecarbonyl-2,3-dihydroindole-2-ylmethyl)-4-(4-indolyl)piperazine

1-(1-Cyclohexanecarbonyl-8-methoxy-1,2,3,4-tetrahydroquinoline-2-ylmethyl)-4-(4-indolyl)piperazine

1-(1-Cyclohexanecarbonyl-6-hydroxy-1,2,3,4-tetrahydroquinoline-2-ylmethyl)-4-(4-indolyl)piperazine

1-(6-Bromo-1-cyclohexanecarbonyl-1,2,3,4-tetrahydroquinoline-2-ylmethyl)-4-(4-fluoro-2-methoxyphenyl)piperazine

1-(1-Cyclohexanecarbonyl-8-hydroxy-1,2,3,4-tetrahydroquinoline-2-ylmethyl)-4-(4-indolyl)piperazine

1-(1-Cyclohexanecarbonyl-6-methyl-1,2,3,4-tetrahydroquinoline-2-ylmethyl)-4-(4-fluoro-2-methoxyphenyl)piperazine

1-[1-(3-Cyanominopropionyl)-1,2,3,4-tetrahydroquinoline-2-ylmethyl]-4-(4-indolyl)piperazine

1-(5-Chloro-1-cyclohexanecarbonyl-1,2,3,4-tetrahydroquinoline-2-ylmethyl)-4-(4-fluoro-2-methoxyphenyl)piperazine

1-[5-Chloro-1-(4-methoxybenzoyl)-1,2,3,4-tetrahydroquinoline-2-ylmethyl]-4-(4-fluoro-2-methoxyphenyl)piperazine

1-(7-Chloro-1-cyclohexanecarbonyl-1,2,3,4-tetrahydroquinoline-2-ylmethyl)-4-(4-fluoro-2-methoxyphenyl)piperazine

3-Benzyl-1-(1-cyclohexanecarbonyl-1,2,3,4-tetrahydroquinoline-2-ylmethyl)piperidine

3-Benzyl-1-(1-cyclohexanecarbonyl-1,2,3,4-tetrahydroquinoline-2-ylmethyl)piperidine

1-(4-Chloro-2-1-propoxyphenyl)-4-(1-cyclohexanecarbonyl-1,2,3,4-tetrahydroquinoline-2-ylmethyl)piperazine

1-(5-Chloro-2-fluorophenyl)-4-(1-cyclohexanecarbonyl-1,2,3,4-tetrahydroquinoline-2-ylmethyl)piperazine

1-[4-(2,1,3-Benzothiadiazolyl)]-4-(1-cyclohexanecarbonyl-1,2,3,4-tetrahydroquinoline-2-ylmethyl)piperazine.

1-(1-Cyclohexanecarbonyl-1,2,3,4-tetrahydroquinoline-2-ylmethyl)-4-(2-ethoxy-4-fluorophenyl)piperazine

1-(1-Cyclohexanecarbonyl-1,2,3,4-tetrahydroquinoline-2-ylmethyl)-4-(2-methoxy-4-hydroxyphenyl)piperazine

1-(1-Cyclohexanecarbonyl-1,2,3,4-tetrahydroquinoline-2-ylmethyl)-4-(7-methoxy-4-indolyl)piperazine.

1-(1-Cyclohexanecarbonyl-1,2,3,4-tetrahydroquinoline-2-ylmethyl)-4-(2-pyrazinyl)piperazine

1-(2-Cyano-4-nitrophenyl)-4-(1-cyclohexanecarbonyl-1,2,3,4-tetrahydroquinoline-2-ylmethyl)piperazine

1-[4-(2,1,3-Benzoxadiazolyl)]-4-(1-cyclohexanecarbonyl-1,2,3,4-tetrahydroquinoline-2-ylmethyl)piperazine.

1-(1-Cyclohexanecarbonyl-1,2,3,4-tetrahydroquinoline-2-ylmethyl)-4-(2-methoxy-5-trifluoromethylphenyl)piperazine.

1-(1-Cyclohexanecarbonyl-1,2,3,4-tetrahydroquinoline-2-ylmethyl)-4-[1-(1,2,3,4-tetrahydronaphthyl)]piperazine.

1-(7-Chloro-2,3-dihydro-1,4-benzodioxin-5-yl)-4-(1-cyclohexanecarbonyl-1,2,3,4-tetrahydroquinoline-2-ylmethyl)piperazine.

1-(1-Cyclohexanecarbonyl-1,2,3,4-tetrahydroquinoline-2-ylmethyl)-4-(4-pyridinyl)piperazine.

1-(1-Cyclohexanecarbonyl-1,2,3,4-tetrahydroquinoline-2-ylmethyl)-4-[4-(6,7-dime-thoxyquinazolinyl)]piperazine

1-(1-Cyclohexanecarbonyl-6-nitro-1,2,3,4-tetrahydroquinoline-2-ylmethyl-4-(4-fluoro-2-methoxyphenyl)piperazine

1-(1-Cyclohexanecarbonyl-1,2,3,4-tetrahydroquinoline-2-ylmethyl)-4-(1-ethyl-4-indolyl)piperazine

1-(2-Bromo-5-methoxybenzyl)-4-(1-cyclohexanecarbonyl-1,2,3,4-tetrahydroquinoline-2-ylmethyl)piperazine

1-(4-Chloro-2-methoxyphenyl)-4-(1-cyclohexanecarbonyl-1,2,3,4-tetrahydroquinoline-2-ylmethyl)piperazine.

1-(4-Chloro-2-ethoxyphenyl)-4-(1-cyclohexanecarbonyl-1,2,3,4-tetrahydroquinoline-2-ylmethyl)piperazine

1-(1-Cyclohexanecarbonyl-1,2,3,4-tetrahydroquinoline-2-ylmethyl)-4-(4-fluoro-2-propoxyphenyl)piperazine

1-(1-Cyclohexanecarbonyl-1,2,3,4-tetrahydroquinoline-2-ylmethyl)-4-(7-methyl-4-indolyl)piperazine

1-(1-Cyclohexanecarbonyl-1,2,3,4-tetrahydroquinoline-2-ylmethyl)-4-(2-methoxyphenoxy)piperidine

1-[6-(5-Acetyl-2-thienyl)-1-cyclohexanecarbonyl-1,2,3,4-tetrahydroquinoline-2-ylmethyl)-4-(4-fluoro-2-methoxyphenyl)piperazine

1-[1-Cyclohexanecarbonyl-6-(3,5-dimethylisoxazol-4-yl)-1,2,3,4-tetrahydroquinoli-ne-2-ylmethyl]-4-(4-fluoro-2-methoxyphenyl)piperazine

1-(1-Cyclohexanecarbonyl-7-methyl-1,2,3,4-tetrahydroquinoline-2-ylmethyl)-4-(4-fluoro-2-methoxyphenyl)piperazine

Cis-1-(1-Cyclohexanecarbonyl-4-methyl-1,2,3,4-tetrahydroquinoline-2-ylmethyl)-4-(4-fluoro-2-methoxyphenyl)piperazine

1-(1-Cyclohexanecarbonyl-8-methyl-1,2,3,4-tetrahydroquinoline-2-ylmethyl)-4-(4-fluoro-2-methoxyphenyl)piperazine

1-(4-Indolyl)-4-[1-(2-pyrrolylcarbonyl)-1,2,3,4-tetrahydroquinoline-2-ylmethyl]piperazine

1-[1-(4-Dimethylaminobenzoyl)-1,2,3,4-tetrahydroquinoline-2-ylmethyl]-4-(4-indolyl)piperazine

1-(4-Indolyl)-4-[1-(2-phenylacetyl)-1,2,3,4-tetrahydroquinoline-2-ylmethyl]piperazine

1-[1-(4-Cyanobenzoyl)-1,2,3,4-tetrahydroquinoline-2-ylmethyl]-4-(4-indolyl)piperazine

1-[1-(2-Chlorobenzoyl)-1,2,3,4-tetrahydroquinoline-2-ylmethyl]-4-(4-indolyl)piperazine

1-[1-(4-Chlorobenzoyl)-1,2,3,4-tetrahydroquinoline-2-ylmethyl]-4-(4-indolyl)piperazine

1-[1-(3-Chlorobenzoyl)-1,2,3,4-tetrahydroquinoline-2-ylmethyl]-4-(4-indolyl)piperazine

1-[1-(5-Benzo[1,3]dioxolylcarbonyl)-1,2,3,4-tetrahydroquinoline-2-ylmethyl]-4-(4-indolyl)piperazine

1-(1-Cyclopropylcarbonyl-1,2,3,4-tetrahydroquinoline-2-ylmethyl)-4-(4-indolyl)piperazine

1-(4-Indolyl)-4-[1-(2-methylpropionyl)-1,2,3,4-tetrahydroquinoline-2-ylmethyl]piperazine

1-(4-Indolyl)-4-[1-(2-methoxyacetyl)-1,2,3,4-tetrahydroquinoline-2-ylmethyl]piperazine

1-(2-Cyclopropylacetyl-1,2,3,4-tetrahydroquinoline-2-ylmethyl)-4-(4-indolyl)piperazine

1-(4-Indolyl)-4-[1-(3-methylbutanoyl)-1,2,3,4-tetrahydroquinoline-2-ylmethyl]piperazine

1-(2-Furoyl-1,2,3,4-tetrahydroquinoline-2-ylmethyl)-4-(4-indolyl)piperazine

1-(4-Indolyl)-4-[1-(5-isoxazolylcarbonyl)-1,2,3,4-tetrahydroquinoline-2-ylmethyl]piperazine

1-(4-Indolyl)-4-[1-(3-tetrahydrofurancarbonyl)-1,2,3,4-tetrahydroquinoline-2-ylmethyl]piperazine

1-(4-Indolyl)-4-[1-(2-tetrahydrofurancarbonyl)-1,2,3,4-tetrahydroquinoline-2-ylmethyl]piperazine

1-[1-(3,3-Dimethylbutanoyl)-1,2,3,4-tetrahydroquinoline-2-ylmethyl]-4-(4-indolyl)piperazine

1-[1-(2-Acetoxyacetyl)-1,2,3,4-tetrahydroquinoline-2-ylmethyl]-4-(4-indolyl)piperazine

1-(4-Indolyl)-4-[1-(2-thienylcarbonyl)-1,2,3,4-tetrahydroquinoline-2-ylmethyl]piperazine

1-(4-Indolyl)-4-[1-(3-thienylcarbonyl)-1,2,3,4-tetrahydroquinoline-2-ylmethyl]piperazine

1-[1-(2-Cyclohexylacetyl)-1,2,3,4-tetrahydroquinoline-2-ylmethyl]-4-(4-indolyl)piperazine

1-(4-Indolyl)-4-[1-(3-trifluoromethylbenzoyl)-1,2,3,4-tetrahydroquinoline-2-ylmethyl]piperazine

1-(4-Indolyl)-4-[1-(4-trifluoromethylbenzoyl)-1,2,3,4-tetrahydroquinoline-2-ylmethyl]piperazine

1-(4-Indolyl)-4-[1-(3-phenylpropionyl)-1,2,3,4-tetrahydroquinoline-2-ylmethyl]piperazine

1-(4-Indolyl)-4-[1-(2-methoxybenzoyl)-1,2,3,4-tetrahydroquinoline-2-ylmethyl]piperazine

1-(4-Indolyl)-4-[1-(4-methoxybenzoyl)-1,2,3,4-tetrahydroquinoline-2-ylmethyl]piperazine

1-[1-[2-(4-Fluorophenyl)acetyl]-1,2,3,4-tetrahydroquinoline-2-ylmethyl]-4-(4-indolyl)piperazine

1-[1-(2,6-Difluorobenzoyl)-1,2,3,4-tetrahydroquinoline-2-ylmethyl]-4-(4 indolyl)piperazine

1-(4-Indolyl)-4-[1-[2-(2-methoxyphenyl)acetyl]-1,2,3,4-tetrahydroquinoline-2-ylmethyl]piperazine

1-[1-[2-(4-Chlorophenyl)acetyl]-1,2,3,4-tetrahydroquinoline-2-ylmethyl]-4-(4-indolyl)piperazine

1-(4-Indolyl)-4-[1-[2-(4-trifluoromethylphenyl)acetyl]-1,2,3,4-tetrahydroquinoline]-2-ylmethyl]piperazine

1-(4-Indolyl)-4-[1-(2-pyridylcarbonyl)-1,2,3,4-tetrahydroquinoline-2-ylmethyl]piperazine

1-(4-Indolyl)-4-[1-(3-pyridylcarbonyl)-1,2,3,4-tetrahydroquinoline-2-ylmethyl]piperazine

1-(4-Indolyl)-4-[1-(4-pyridylcarbonyl)-1,2,3,4-tetrahydroquinoline-2-ylmethyl]piperazine

1-[1-(3,5-Dimethyl-4-isoxazolylcarbonyl)-1,2,3,4-tetrahydroquinoline-2-ylmethyl]-4-(4-indolyl)piperazine

1-[1-(2,2-Dimethylpropionyl)-1,2,3,4-tetrahydroquinoline-2-ylmethyl]-4-(4-indolyl)piperazine

1-(4-Indolyl)-4-[1-[2-(3-pyridyl)acetyl]-1,2,3,4-tetrahydroquinoline-2-ylmethyl]piperazine

1-[1-(1-Acetylpiperidine-4-carbonyl)-1,2,3,4-tetrahydroquinoline-2-ylmethyl]-4-(4-indolyl)piperazine

1-(4-Indolyl)-4-[1-[2-(2-thienylacetyl)]-1,2,3,4-tetrahydroquinoline-2-ylmethyl]piperazine

1-(4-Indolyl)-4-[1-[2-(3-thienylacetyl)]-1,2,3,4-tetrahydroquinoline-2-ylmethyl]piperazine

1-(4-Indolyl)-4-[1-[3-(3-pyridylpropionyl)]-1,2,3,4-tetrahydroquinoline-2-ylmethyl]piperazine

1-(4-Indolyl)-4-[L 1-(2-phenoxyacetyl)-1,2,3,4-tetrahydroquinoline-2-ylmethyl]piperazine

1-(4-Indolyl)-4-[l 1-[2-(4-methoxyphenylacetyl)]-1,2,3,4-tetrahydroquinoline-2-ylmethyl]piperazine

1-(4-Indolyl)-4-[1-(2-phenylmethoxyacetyl)-1,2,3,4-tetrahydroquinoline-2-ylmethyl]piperazine

1-[1-[2-(2-Chlorophenylacetyl)]-1,2,3,4-tetrahydroquinoline-2-ylmethyl]-4-(4-indo-lyl)piperazine

1-[1-[2-(N-Benzoyl-N-methylamino)acetyl]-1,2,3,4-tetrahydroquinoline-2-ylmethyl]-4-(4-indolyl)piperazine

1-(4-Indolyl)-4-[1-(1-methyl-3-indolylcarbonyl)-1,2,3,4-tetrahydroquinoline-2-ylmethyl]piperazine

1-[1-[2-(4-Dimethylaminophenylacetyl)]-1,2,3,4-tetrahydroquinoline-2-ylmethyl-4-(4-indolyl)piperazine

1-[1-[2-(5-Benzo[1,3]dioxolyl)acetyl]-1,2,3,4-tetrahydroquinoline-2-ylmethyl]-4-(4-indolyl)piperazine

1-[1-[2-(2-Chlorophenoxy)acetyl]-1,2,3,4-tetrahydroquinoline-2-ylmethyl]-4-(4-indo-lyl)piperazine

1-[1-[2-(4-Chlorophenoxy)acetyl]-1,2,3,4-tetrahydroquinoline-2-ylmethyl]-4-(4-indolyl)piperazine

1-(4-Indolyl)-4-[1-[2-(4-morpholinyl)pyridine-5-carbonyl]-1,2,3,4-tetrahydroquinoline-2-ylmethyl]piperazine

1-(4-Indolyl)-4-[1-(5-methylisoxazole-4-carbonyl)-1,2,3,4-tetrahydroquinoline-2-ylmethyl]piperazine

1-(4-Indolyl)-4-[1-[2-(5-oxopyrrolidin-2yl)acetyl]-1,2,3,4-tetrahydroquinoline-2-ylmethyl]piperazine

1-(4-Indolyl)-4-[1-(2-methyl-5-piperidin-1-ylsulfonylfuran-3-carbonyl)-1,2,3,4-tetrahydroquinoline-2-ylmethyl]piperazine

1-(4-Indolyl)-4-[1-(2-methyl-5-morpholin-4-ylsulfonylfuran-3-carbonyl)-1,2,3,4-tetrahydroquinoline-2-ylmethyl]piperazine

1-(4-Indolyl)-4-[-[1-(4-methylphenylsulphonyl)pyrrole-3-carbonyl]-1,2,3,4-tetrahydroquinoline-2-ylmethyl]piperazine

1-(4-Indolyl)-4-[1-(2-methyl-5-dimethylaminolsulfonylfuran-3-carbonyl)-1,2,3,4-tetrahydroquinoline-2-ylmethyl]piperazine

1-(4-Indolyl)-4-[1-(2-methyl-5-pyrrolidin-1-ylsulfonylfuran-3-carbonyl)-1,2,3,4-tetrahydroquinoline-2-ylmethyl]piperazine

1-[(1-Adamantylcarbonyl)-1,2,3,4-tetrahydroquinoline-2-ylmethyl]-4-(4-indolyl)piperazine

1-(4-Indolyl)-4-[1-(3-phenoxypropionyl)-1,2,3,4-tetrahydroquinoline-2-ylmethyl]piperazine

1-(4-Indolyl)-1-[1-(4-phenoxybutanoyl)-1,2,3,4-tetrahydroquinoline-2-ylmethyl]piperazine

1-(4-Indolyl)-4-[1-[2-(2-fluorophenylacetyl)]-1,2,3,4-tetrahydroquinoline-2-ylmethyl]piperazine

1-(4-Indolyl)-4-[1-[2-(2-trifluoromethylphenylacetyl)]-1,2,3,4-tetrahydroquinoline-2-ylmethyl]piperazine

1-[1-(2-Bicyclo[2.2.2]octylcarbonyl)-1,2,3,4-tetrahydroquinoline-2-ylmethyl]-4-(4-indolyl)piperazine

1-(4-Indolyl)-1-[1-(4-phenylbutanoyl)-1,2,3,4-tetrahydroquinoline-2-ylmethyl]piperazine

1-(4-Indolyl)-4-[1-(3-methoxybenzoyl)-1,2,3,4-tetrahydroquinoline-2-ylmethyl]piperazine

1-[1-(4-Hydroxybenzoyl)-1,2,3,4-tetrahydroquinoline-2-ylmethyl]-4-(4-indolyl)piperazine

1-[1-(4-Ethoxybenzoyl)-1,2,3,4-tetrahydroquinoline-2-ylmethyl]-4-(4-indolyl)piperazine

1-(4-Indolyl)-4-[1-(4-methylbenzoyl)-1,2,3,4-tetrahydroquinoline-2-ylmethyl]piperazine

1-[1-[2-(N-Benzyl-N-methanesulphonylamino)acetyl]-1,2,3,4-tetrahydroquinoline-2-ylmethyl]-4-(4-indolyl)piperazine

1-(4-Indolyl)-4-[1-[(2S)-5-oxopyrrolidine-2-ylcarbonyl]-1,2,3,4-tetrahydroquinoline-2-ylmethyl]piperazine

1-(4-Indolyl)-4-[1-[(2R)-5-oxopyrrolidine-2-yl)carbonyl]-1,2,3,4-tetrahydroquinoline-2-ylmethyl]piperazine

1-(4-Indolyl)-4-[1-(2-methylphenylaminocarbonyl)-1,2,3,4-tetrahydroquinoline-2-ylmethyl]piperazine

1-(4-Indolyl)-4-[1-(3-methylphenylaminocarbonyl)-1,2,3,4-tetrahydroquinoline-2-ylmethyl]piperazine

1-[1-(4-Fluorophenylaminocarbonyl)-1,2,3,4-tetrahydroquinoline-2-ylmethyl)-4-(4-indolyl)piperazine

1-(4-Indolyl)-4-[1-(4-methylphenylaminocarbonyl)-1,2,3,4-tetrahydroquinoline-2-ylmethyl]piperazine

1-(4-Indolyl)-4-[1-[(1S)-1-phenylethylaminocarbonyl]-1,2,3,4-tetrahydroquinoline-2-ylmethyl]piperazine

1-(4-Indolyl)-1-[1-[(1R)-1-phenylethylaminocarbonyl]-1,2,3,4-tetrahydroquinoline-2-ylmethyl)piperazine (diastereoisomer A)

1-(4-Indolyl)-1-[1-[(1R)-1-phenylethylaminocarbonyl]-1,2,3,4-tetrahydroquinoline-2-ylmethyl]piperazine (1:1 diastereoisomers mixture)

1-(4-Indolyl)-4-[1-(1-methylethylaminocarbonyl)-1,2,3,4-tetrahydroquinoline-2-ylmethyl]piperazine

1-(4-Indolyl)-4-[1-methylaminothiocarbonyl-1,2,3,4-tetrahydroquinoline-2-ylmethyl)piperazine

1-(1-Benzylaminocarbonyl-1,2,3,4-tetrahydroquinoline-2-ylmethyl)-4-(4-indolyl)piperazine

1-(4-Indolyl)-4-[1-(2-phenylethylaminocarbonyl)-1,2,3,4-tetrahydroquinoline-2-ylmethyl]piperazine

1-(4-Indolyl)-4-(1-pentylaminocarbonyl-1,2,3,4-tetrahydroquinoline-2-ylmethyl]piperazine

1-[1-(1,1-Dimethylethylaminocarbonyl)-1,2,3,4-tetrahydroquinoline-2-ylmethyl]-1-(4-indolyl)piperazine

1-(4-Indolyl)-4-[1-(4-methoxyphenylaminocarbonyl)-1,2,3,4-tetrahydroquinoline-2-ylmethyl]piperazine

1-(4-Indolyl)-4-[1-(4-methoxyphenylmethylaminocarbonyl)-1,2,3,4-tetrahydroquinoline-2-ylmethyl]piperazine

1-(4-Indolyl)-1-[1-phenylsulphonyl-1,2,3,4-tetrahydroquinoline-2-ylmethyl]piperazine

1-(4-Indolyl)-4-[1-(2-thienylsulphonyl)-1,2,3,4-tetrahydroquinoline-2-ylmethyl]piperazine

1-(4-Indolyl)-4-[1-(4-methoxyphenylsulphonyl)-1,2,3,4-tetrahydroquinoline-2-ylmethyl]piperazine

1-(4-Indolyl)-4-[1-(4-methylphenylsulphonyl)-1,2,3,4-tetrahydroquinoline-2-ylmethyl]piperazine

1-[1-(4-Fluorophenylsulphonyl)-1,2,3,4-tetrahydroquinoline-2-ylmethyl]-4-(4-indolyl)piperazine

1-[1-(4-Cyanophenylsulphonyl)-1,2,3,4-tetrahydroquinoline-2-ylmethyl]-4-(4-indolyl)piperazine

1-(4-Indolyl)-4-(1-phenylmethylsulphonyl-1,2,3,4-tetrahydroquinoline-2-ylmethyl)piperazine

1-[1-(2-tert-Butoxycarbonylamino)acetyl-1,2,3,4-tetrahydroquinoline-2-ylmethyl]-4-(4-indolyl)piperazine

1-[1-(3-tert-Butoxycarbonylamino)propionyl-1,2,3,4-tetrahydroquinoline-2-ylmethyl]-4-(4-indolyl)piperazine

1-[1-[2-(1-tert-Butoxycarbonylindol-3-yl)acetyl]-1,2,3,4-tetrahydroquinoline-2-ylmethyl]-4-(4-indolyl]piperazine

1-[1-[4-(1-tert-Butoxycarbonylpiperidino)carbonyl]-1,2,3,4-tetrahydroquinoline-2-ylmethyl]-4-(4-indolyl)piperazine

1-[1-(1-tert-Butoxycarbonyl-(2R)-pyrrolidin-2yl)carbonyl-1,2,3,4-tetrahydroquinoline-2-ylmethyl]-4-(4-indolyl)piperazine

1-[1-(1-tert-Butoxycarbonyl-(2S)-pyrrolidin-2yl)carbonyl-1,2,3,4-tetrahydroquinoline-2-ylmethyl]-4-(4-indolyl)piperazine

1-(1-Cyclohexanecarbonyl-1,2,3,4-tetrahydroquinolin-2-ylmethyl)-2-(1-hydroxy-1-phenylmethyl)piperidine

1-(1-Cyclohexanecarbonyl-1,2,3,4-tetrahydroquinolin-2-ylmethyl)-4-(2,6-dimethylphenyl)piperidine

2-(4-Chlorophenyl)-1-(1-cyclohexanecarbonyl-1,2,3,4-tetrahydroquinolin-2-ylmethyl)piperidine

1-(1-Cyclohexanecarbonyl-1,2,3,4-tetrahydroquinolin-2-ylmethyl)-4-phenylpiperidine

4-Benzoy-1-(1-Cyclohexanecarbonyl-1,2,3,4-tetrahydroquinolin-2-ylmethyl)piperidine

1-(1-Cyclohexanecarbonyl-1,2,3,4-tetrahydroquinolin-2-ylmethyl)-4-(2-nitrophenyl)piperazine

1-(1-Cyclohexanecarbonyl-1,2,3,4-tetrahydroquinolin-2-ylmethyl)-4-(3-trifluoromethylpyridin-2-yl)piperazine

1-(1-Cyclohexanecarbonyl-1,2,3,4-tetrahydroquinolin-2-ylmethyl)-4-(4-fluoro-2-nitrophenyl)piperazine

1-(1-Cyclohexanecarbonyl-1,2,3,4-tetrahydroquinolin-2-ylmethyl)-4-(2,3-dichlorophenyl)piperazine

1-(1-Cyclohexanecarbonyl-1,2,3,4-tetrahydroquinolin-2-ylmethyl)-4-(3-cyanopyridin-2-yl)piperazine

1-(1-Cyclohexanecarbonyl-1,2,3,4-tetrahydroquinolin-2-ylmethyl)-4-(3,4-dichlorophenyl)piperazine

1-(1-Cyclohexanecarbonyl-1,2,3,4-tetrahydroquinolin-2-ylmethyl)-4-(2-ethylphenyl)piperazine

1-(4-Chloro-3-trifluoromethylphenyl)-4-(1-cyclohexanecarbonyl-1,2,3,4-tetrahydroquinolin-2-ylmethyl)piperazine

1-(2-Cyanophenyl)-4-(1-cyclohexanecarbonyl-1,2,3,4-tetrahydroquinolin-2-ylmethyl)piperazine

1-(1-Cyclohexanecarbonyl-1,2,3,4-tetrahydroquinolin-2-ylmethyl)-4-(2,4-dimethoxyphenyl)piperazine

1-(1-Cyclohexanecarbonyl-1,2,3,4-tetrahydroquinolin-2-ylmethyl)-4-(2-methylthiophenyl)piperazine

1-(1-Cyclohexanecarbonyl-1,2,3,4-tetrahydroquinolin-2-ylmethyl)-4-(3,4-dimethoxyphenyl)piperazine

1-(1-Cyclohexanecarbonyl-1,2,3,4-tetrahydroquinolin-2-ylmethyl)-4-(3-hydroxyphenyl)piperazine

1-(1-Cyclohexanecarbonyl-1,2,3,4-tetrahydroquinolin-2-ylmethyl)-4-(2,4-difluorophenyl)piperazine

1-(5-Chloro-2-methoxyphenyl)-4-(1-cyclohexanecarbonyl-1,2,3,4-tetrahydroquinolin-2-ylmethyl)piperazine

1-(1-Cyclohexanecarbonyl-1,2,3,4-tetrahydroquinolin-2-ylmethyl)-4-(6-methylpyridin-2-yl)piperazine

1-(1-Cyclohexanecarbonyl-1,2,3,4-tetrahydroquinolin-2-ylmethyl)-4-(4-methylphenyl)piperazine

1-(1-Cyclohexanecarbonyl-1,2,3,4-tetrahydroquinolin-2-ylmethyl)-4-(4-trifluoromethylphenyl)piperazine

1-(3-Chlorophenyl)-4-(1-cyclohexanecarbonyl-1,2,3,4-tetrahydroquinolin-2-ylmethyl)piperazine

1-(2-Chlorophenyl)-4-(1-cyclohexanecarbonyl-1,2,3,4-tetrahydroquinolin-2-ylmethyl)piperazine

1-(1-Cyclohexanecarbonyl-1,2,3,4-tetrahydroquinolin-2-ylmethyl)-4-(3-methoxyphenyl)piperazine

1-(1-Cyclohexanecarbonyl-1,2,3,4-tetrahydroquinolin-2-ylmethyl)-4-(4-fluorophenyl)piperazine

1-(4-Acetylphenyl)-4-(1-cyclohexanecarbonyl-1,2,3,4-tetrahydroquinolin-2-ylmethyl)piperazine

1-(1-Cyclohexanecarbonyl-1,2,3,4-tetrahydroquinolin-2-ylmethyl)-4-(4-hydroxyphenyl)piperazine

1-(5-Chloro-2-methylphenyl)-4-(1-cyclohexanecarbonyl-1,2,3,4-tetrahydroquinolin-2-ylmethyl)piperazine

1-(1-Cyclohexanecarbonyl-1,2,3,4-tetrahydroquinolin-2-ylmethyl)-4-(3-methylphenyl)piperazine

1-(1-Cyclohexanecarbonyl-1,2,3,4-tetrahydroquinolin-2-ylmethyl)-4-(2-nitro-4-trifluoromethylphenyl)piperazine

1-(1-Cyclohexanecarbonyl-1,2,3,4-tetrahydroquinolin-2-ylmethyl)-4-(5-trifluoromethylpyridin-2-yl)piperazine

1-(1-Cyclohexanecarbonyl-1,2,3,4-tetrahydroquinolin-2-ylmethyl)-4-(3-chloro-5-trifluoromethylpyridin-2-yl)piperazine

1-(5-Cyanopyridin-2-yl)-4-(1-cyclohexanecarbonyl-1,2,3,4-tetrahydroquinolin-2-ylmethyl)piperazine

1-(1-Cyclohexanecarbonyl-1,2,3,4-tetrahydroquinolin-2-ylmethyl)-4-(2-methylquinolin-4-yl)piperazine

1-(1-Cyclohexanecarbonyl-1,2,3,4-tetrahydroquinolin-2-ylmethyl)-4-(phenylmethyl)piperidine

1-(Benzofuran-3-yl)-4-(1-Cyclohexanecarbonyl-1,2,3,4-tetrahydroquinolin-2-ylmethyl)piperidine

1-(1-Cyclohexanecarbonyl-1,2,3,4-tetrahydroquinolin-2-ylmethyl)-4-(4-fluorophenyl)piperidine

1-(1-Cyclohexanecarbonyl-1,2,3,4-tetrahydroquinolin-2-ylmethyl)-4-(3-fluorophenyl)piperidine

1-(1-Cyclohexanecarbonyl-1,2,3,4-tetrahydroquinolin-2-ylmethyl)-4-(2-fluorophenyl)piperidine

4-(4-Chlorophenyl)-1-(1-cyclohexanecarbonyl-1,2,3,4-tetrahydroquinolin-2-ylmethyl)piperidine

1-(1-Cyclohexanecarbonyl-1,2,3,4-tetrahydroquinolin-2-ylmethyl)-4-(4-trifluoromethylphenyl)piperidine

1-(1-Cyclohexanecarbonyl-1,2,3,4-tetrahydroquinolin-2-ylmethyl)-4-(3-trifluoromethylphenyl)piperidine

1-(1-Cyclohexanecarbonyl-1,2,3,4-tetrahydroquinolin-2-ylmethyl)-4-[5-(2-furyl)-2H-pyrazol-3-yl]piperidine

1-(1-Cyclohexanecarbonyl-1,2,3,4-tetrahydroquinolin-2-ylmethyl)-4-(4-methoxyphenyl)piperidine

1-(1-Cyclohexanecarbonyl-1,2,3,4-tetrahydroquinolin-2-ylmethyl)-4-(3-methoxyphenyl)piperidine

1-(1-Cyclohexanecarbonyl-1,2,3,4-tetrahydroquinolin-2-ylmethyl)-4-(4-fluorobenzoyl)piperidine

1-(1-Cyclohexanecarbonyl-1,2,3,4-tetrahydroquinolin-2-ylmethyl)-4-(4-phenylmethylaminocarbonylamino)piperidine

1-(1-Cyclohexanecarbonyl-1,2,3,4-tetrahydroquinolin-2-ylmethyl)-2-(4-methoxyphenylmethyl)piperidine

1-(1-Cyclohexanecarbonyl-1,2,3,4-tetrahydroquinolin-2-ylmethyl)-2-(4-fluorophenylmethyl)piperidine

1-(1-Cyclohexanecarbonyl-1,2,3,4-tetrahydroquinolin-2-ylmethyl)-4-(2,3-dichlorophenyl)piperidine

1-(1-Cyclohexanecarbonyl-1,2,3,4-tetrahydroquinolin-2-ylmethyl)-4-(2,6-difluorophenyl)piperidine

4-(2-Chloro-6-fluorophenyl)-1-(1-cyclohexanecarbonyl 1,2,3,4-tetrahydroquinolin-2-ylmethyl)piperidine

1-(1-Cyclohexanecarbonyl-1,2,3,4-tetrahydroquinolin-2-ylmethyl)-4-(2,5-dimethylphenyl)piperidine

1-(1-Cyclohexanecarbonyl-1,2,3,4-tetrahydroquinolin-2-ylmethyl)-4-(3-methylphenyl)piperidine

1-(1-Cyclohexanecarbonyl-1,2,3,4-tetrahydroquinolin-2-ylmethyl)-4-(2-methylphenyl)piperidine

1-(1-Cyclohexanecarbonyl-1,2,3,4-tetrahydroquinolin-2-ylmethyl)-4-(3-fluoro-2-methylphenyl)piperidine

1-(1-Cyclohexanecarbonyl-1,2,3,4-tetrahydroquinolin-2-ylmethyl)-4-(3,5-difluorophenyl)piperidine

1-(1-Cyclohexanecarbonyl-1,2,3,4-tetrahydroquinolin-2-ylmethyl)-4-(2,5-difluorophenyl)piperidine

1-(1-Cyclohexanecarbonyl-1,2,3,4-tetrahydroquinolin-2-ylmethyl)-4-(3,5-dimethylphenyl)piperidine

4-(3-Bromophenyl)-1-(1-cyclohexanecarbonyl-1,2,3,4-tetrahydroquinolin-2-ylmethyl)piperidine

4-(2-Bromophenyl)-1-(1-cyclohexanecarbonyl-1,2,3,4-tetrahydroquinolin-2-ylmethyl)piperidine

4-(4-Butoxyphenyl)-1-(1-cyclohexanecarbonyl-1,2,3,4-tetrahydroquinolin-2-ylmethyl)piperidine

1-(1-Cyclohexanecarbonyl-1,2,3,4-tetrahydroquinolin-2-ylmethyl)-4-(2-fluorophenyl)piperazine

1-(1-Cyclohexanecarbonyl-1,2,3,4-tetrahydroquinolin-2-ylmethyl)-4-(6-chloropyridin-2-yl)piperazine

1-(1-Cyclohexanecarbonyl-1,2,3,4-tetrahydroquinolin-2-ylmethyl)-4-(3,5-dichloropyridin-4-yl)piperazine

1-(4-Aminophenyl)-4-(1-cyclohexanecarbonyl-1,2,3,4-tetrahydroquinolin-2-ylmethyl)piperazine

4-(3-Chlorophenyl)-1-(1-cyclohexanecarbonyl-1,2,3,4-tetrahydroquinolin-2-ylmethyl)piperidine

4-(2-Chlorophenyl)-1-(1-cyclohexanecarbonyl-1,2,3,4-tetrahydroquinolin-2-ylmethyl)piperidine

1-(1-Cyclohexanecarbonyl-1,2,3,4-tetrahydroquinolin-2-ylmethyl)-2-(pyridin-2-yl)-pyrrolidine

1-(1-Cyclohexanecarbonyl-1,2,3,4-tetrahydroquinolin-2-ylmethyl)-2-(2-thienyl)pyrrolidine

1-(1-Cyclohexanecarbonyl-1,2,3,4-tetrahydroquinolin-2-ylmethyl)-2-phenylpyrrolidine

1-(1-Cyclohexanecarbonyl-1,2,3,4-tetrahydroquinolin-2-ylmethyl)-2-(2,4-dimethoxy)phenylpyrrolidine

1-(1-Cyclohexanecarbonyl-1,2,3,4-tetrahydroquinolin-2-ylmethyl)-2-(pyridin-3-yl)-pyrrolidine

1-(1-Cyclohexanecarbonyl-1,2,3,4-tetrahydroquinolin-2-ylmethyl)-2-phenylmethylpyrrolidine

1-(1-Cyclohexanecarbonyl-1,2,3,4-tetrahydroquinolin-2-ylmethyl)-4-(3,5-dimethoxyphenyl)piperazine

1-(1-Cyclohexanecarbonyl-1,2,3,4-tetrahydroquinolin-2-ylmethyl)-2-(2-furyl)pyrrolidine

1-(1-Cyclohexanecarbonyl-1,2,3,4-tetrahydroquinolin-2-ylmethyl)-2-(4-methoxyphenylmethyl)pyrrolidine

1-(1-Cyclohexanecarbonyl-1,2,3,4-tetrahydroquinolin-2-ylmethyl)-2-(4-chlorophenyl)pyrrolidine

1-(1-Cyclohexanecarbonyl-1,2,3,4-tetrahydroquinolin-2-ylmethyl)-2-(2-indolyl)pyrrolidine

1-(1-Cyclohexanecarbonyl-1,2,3,4-tetrahydroquinolin-2-ylmethyl)-4-(2-trifluoromethylphenyl)piperidine

N-(1-Cyclohexanecarbonyl-1,2,3,4-tetrahydroquinolin-2-ylmethyl)-2-(2-methoxyphenoxy)ethylmethylamine

N-(1-Cyclohexanecarbonyl-1,2,3,4-tetrahydroquinolin-2-ylmethyl)-N-(2,3-dihydro-benzo[1,4]dioxin-2-ylmethyl)methylamine

N-(1-Cyclohexanecarbonyl-1,2,3,4-tetrahydroquinolin-2-ylmethyl)-2-(2-chlorophenoxy)ethylmethylamine

N-(1-Cyclohexanecarbonyl-1,2,3,4-tetrahydroquinolin-2-ylmethyl)-2-[2-(2,2,2-trifluoroethoxy)phenoxy]ethylmethylamine

N-(1-Cyclohexanecarbonyl-1,2,3,4-tetrahydroquinolin-2-ylmethyl)-2-[4-(1H)-indolyloxy]ethylmethylamine

N-(1-Cyclohexanecarbonyl-1,2,3,4-tetrahydroquinolin-2-ylmethyl)-2-(8-quinolyloxy)ethylmethylamine

1-(4-Cchloro-2-nitrophenyl)-4-(1-cyclohexanecarbonyl-1,2,3,4-tetrahydroquinolin-2-ylmethyl)piperazine

1-(1-Cyclohexanecarbonyl-1,2,3,4-tetrahydroquinolin-2-ylmethyl)-4-(2-trifluoromethylquinolin-4-yl)piperazine

4-(1-Benzimidazolyl)-1-(1-cCyclohexanecarbonyl-1,2,3,4-tetrahydroquinolin-2-ylmethyl)piperidine

1-(1-Cyclohexanecarbonyl-1,2,3,4-tetrahydroquinolin-2-ylmethyl)-2-(4-methoxyphenyl)piperidine

1-(1-Cyclohexanecarbonyl-1,2,3,4-tetrahydroquinolin-2-ylmethyl)-4-(2,6-dichlorophenyl)piperidine

1-(1-Cyclohexanecarbonyl-1,2,3,4-tetrahydroquinolin-2-ylmethyl)-4-(2-pyridyl)piperazine

1-(1-Cyclohexanecarbonyl-1,2,3,4-tetrahydroquinolin-2-ylmethyl)-4-(4-quinolyl)piperazine

1-(1-Cyclohexanecarbonyl-1,2,3,4-tetrahydroquinolin-2-ylmethyl)-4-(7-methoxy-4-quinolyl)piperazine

1-(4-Fluoro-2-methoxyphenyl)-4-[1-(2-methylphenylaminocarbonyl)-1,2,3,4-tetrahydroquinolin-6-methyl-2-ylmethyl]piperazine

1-(4-Fluoro-2-methoxyphenyl)-4-[1-(4-methylphenylaminocarbonyl)-1,2,3,4-tetrahydroquinolin-6-methyl-2-ylmethyl]piperazine

1-[1-(1,1-Dimethylethylaminocarbonyl)-1,2,3,4-tetrahydroquinolin-6-methyl-2-ylmethyl]-4-(4-fluoro-2-methoxyphenyl)piperazine

1-(4-Fluoro-2-methoxyphenyl)-4-(1-pentylaminocarbonyl-1,2,3,4-tetrahydroquinolin-6-methyl-2-ylmethyl)piperazine

1-(4-Fluoro-2-methoxyphenyl)-4-[1-(2-thienylacetyl)-1,2,3,4-tetrahydroquinolin-6-methyl-2-ylmethyl]piperazine

1-(4-Fluoro-2-methoxyphenyl)-4-[1-(3-thienylacetyl)-1,2,3,4-tetrahydroquinolin-6-methyl-2-ylmethyl]piperazine

1-(1-Cyclohexylacetyl-1,2,3,4-tetrahydroquinolin-6-methyl-2-ylmethyl)-4-(4-fluoro-2-methoxyphenyl)piperazine

1-[1-(4-Cyanobenzoyl)-1,2,3,4-tetrahydroquinolin-6-methyl-2-ylmethyl]-4-(4-fluoro-2-methoxyphenyl)piperazine

1-(4-Fluoro-2-methoxyphenyl)-4-[1-(4-methoxybenzoyl)-1,2,3,4-tetrahydroquinolin-6-methyl-2-ylmethyl)piperazine

1-[1-(Benzo[1,3]dioxol-5-ylcarbonyl)-1,2,3,4-tetrahydroquinolin-6-methyl-2-ylmethyl]-4-(4-fluoro-2-methoxyphenyl)piperazine

1-[1-[2-(2-Chlorophenyl)acetyl]-1,2,3,4-tetrahydroquinolin-6-methyl-2-ylmethyl]-4-(4-fluoro-2-methoxyphenyl)piperazine

1-(4-Fluoro-2-methoxyphenyl)-4-[1-(1-methyl-1H-indol-3-ylcarbonyl)-1,2,3,4-tetrahydroquinolin-6-methyl-2-ylmethyl]piperazine

1-(1-Cycloheptanecarbonyl-1,2,3,4-tetrahydroquinolin-6-methyl-2-ylmethyl)-4-(4-fluoro-2-methoxyphenyl)piperazine

1-(4-Fluoro-2-methoxyphenyl)-4-(1-phenylsulphonyl-1,2,3,4-tetrahydroquinolin-6-methyl-2-ylmethyl)piperazine

1-(4-Fluoro-2-methoxyphenyl)-4-(1-phenylmethylsulphonyl-1,2,3,4-tetrahydroquinolin-6-methyl-2-ylmethyl)piperazine

1-(4-Fluoro-2-methoxyphenyl)-4-[1-(4-methoxyphenylsulphonyl)-1,2,3,4-tetrahydroquinolin-6-methyl-2-ylmethyl]piperazine

1-(4-Fluoro-2-methoxyphenyl)-4-[1-(4-methylphenylsulphonyl)-1,2,3,4-tetrahydroquinolin-6-methyl-2-ylmethyl]piperazine

1-[1-4-Cyanophenylsulphonyl)-6-methyl-1,2,3,4-tetrahydroquinolin-2-ylmethyl)-4-(4-fluoro-2-methoxyphenyl]piperazine

1-[1-4-Fluorophenylsulphonyl)-6-methyl-1,2,3,4-tetrahydroquinolin-2-ylmethyl)-4-(4-fluoro-2-methoxyphenyl]piperazine

1-(4-Fluoro-2-methoxyphenyl)-4-[1-(3-methylbutanoyl)-1,2,3,4-tetrahydroquinolin-2-ylmethyl]piperazine

1-(3,3-Dimethylbutanoyl-1,2,3,4-tetrahydroquinolin-2-ylmethyl)-4-(4-fluoro-2-methoxyhenyl)piperazine

1-(1-Cyclopropylacetyl-1,2,3,4-tetrahydroquinolin-2-ylmethyl)-4-(4-fluoro-2-methoxyhenyl)piperazine

1-(4-Fluoro-2-methoxyphenyl)-4-[1-(2-pyrrolylcarbonyl)-1,2,3,4-tetrahydroquinolin-2-ylmethyl]piperazine

1-(4-Fluoro-2-methoxyphenyl)-4-[1-(2-furoyl)-1,2,3,4-tetrahydroquinolin-2-ylmethyl]piperazine

1-(4-Fluoro-2-methoxyphenyl)-4-[1-(4-methoxybenzoyl)-1,2,3,4-tetrahydroquinolin-2-ylmethy]piperazine

1-[1-(4-Cyanobenzoyl)-1,2,3,4-tetrahydroquinolin-2-ylmethy]-4-(4-fluoro-2-methoxyphenyl)piperazine

1-[1-(1-Acetylpiperidin-4-ylcarbonyl)-1,2,3,4-tetrahydroquinolin-2-ylmethy]-4-(4-fluoro-2-methoxyphenyl)piperazine

1-(4-Fluoro-2-methoxyphenyl)-4-[1-(2-thienylacetyl)-1,2,3,4-tetrahydroquinolin-2-ylmethyl]piperazine

1-(4-Fluoro-2-methoxyphenyl)-4-[1-(3-thienylacetyl)-1,2,3,4-tetrahydroquinolin-2-ylmethyl]piperazine

1-(1-Cyclobutylcarbonyl-1,2,3,4-tetrahydroquinolin-2-ylmethy)-4-(4-fluoro-2-methoxyphenyl)piperazine

1-(4-Fluoro-2-methoxyphenyl)-4-[1-(4-phenoxybutanoyl)-1,2,3,4-tetrahydroquinolin-2-ylmethyl]piperazine

1-(1-Benzenesulphonyl-1,2,3,4-tetrahydroquinolin-2-ylmethy)-4-(4-fluoro-2-methoxyphenyl)piperazine

1-(4-Fluoro-2-methoxyphenyl)-4-(1-phenylmethylsulphonyl-1,2,3,4-tetrahydroquinolin-2-ylmethyl]piperazine

1-(4-Fluoro-2-methoxyphenyl)-4-[1-(2-methylphenylaminocarbonyl)-1,2,3,4-tetrahydroquinolin-2-ylmethyl]piperazine

1-[1-(1,1-Dimethyethylaminocarbonyl)-1,2,3,4-tetrahydroquinolin-2-ylmethy]-4-(4-fluoro-2-methoxyphenyl)piperazine

1-(1-Isoquinolinyl)-4-[1-(3-methylbutanoyl)-1,2,3,4-tetrahydroquinolin-2-ylmethyl]piperazine

1-[1-(3,3-Dimethylbutanoyl)-1,2,3,4-tetrahydroquinolin-2-ylmethyl]-4-(1-isoquinolinyl)piperazine

1-[1-(2-Cyclopropylacetyl)-1,2,3,4-tetrahydroquinolin-2-ylmethyl]-4-(1-isoquinolinyl)piperazine

1-(1-Isoquinolinyl)-4-[1-(2-pyrrolylcarbonyl)-1,2,3,4-tetrahydroquinolin-2-ylmethyl]piperazine

1-[1-(2-Furancarbonyl)-1,2,3,4-tetrahydroquinolin-2-ylmethyl]-4-(1-isoquinolinyl)piperazine

1-(1-Isoquinolinyl)-4-[1-(4-methoxybenzoyl)-1,2,3,4-tetrahydroquinolin-2-ylmethyl]piperazine

1-[1-(4-Cyanobenzoyl)-1,2,3,4-tetrahydroquinolin-2-ylmethyl]-4-(1-isoquinolinyl)piperazine

1-[1-(1-Acetyl-4-piperidinecarbonyl)-1,2,3,4-tetrahydroquinolin-2-ylmethyl]-4-(1-isoquinolinyl)piperazine

1-(1-Isoquinolinyl)-4-[1-(2-thienyacetyl)-1,2,3,4-tetrahydroquinolin-2-ylmethyl]piperazine

1-(1-Isoquinolinyl)-4-[1-(3-thienyacetyl)-1,2,3,4-tetrahydroquinolin-2-ylmethyl]piperazine

1-(1-Cyclobutanecarbonyl-1,2,3,4-tetrahydroquinolin-2-ylmethyl)-4-(1-isoquinolinyl)piperazine

1-(1-Isoquinolinyl)-4-[1-(4-phenoxybutanoyl)-1,2,3,4-tetrahydroquinolin-2-ylmethyl]piperazine

1-(1-Benzenesulphonyl-1,2,3,4-tetrahydroquinolin-2-ylmethyl)-4-(1-isoquinolinyl)piperazine

1-(1-Isoquinolinyl)-4-(1-phenylmethylsulphonyl-1,2,3,4-tetrahydroquinolin-2-ylmethyl)piperazine

1-(1-Isoquinolinyl)-4-[1-(2-methylphenylaminocarbonyl)-1,2,3,4-tetrahydroquinolin-2-ylmethyl)piperazine

1-(1-Isoquinolinyl)-4-[1-(1,1-Dimethyethylaminocarbonyl)-1,2,3,4-tetrahydroquinolin-2-ylmethyl]piperazine

1-(2,3-Dihydrobenzo[1,4]dioxin-5-yl)-4-[1-(3-methylbutanoyl)-1,2,3,4-tetrahydroquinolin-2-ylmethyl]piperazine

4-[1-(2-Cyclopropylacetyl)-1,2,3,4-tetrahydroquinolin-2-ylmethyl]-1-(2,3-dihydrobenzo[1,4]dioxin-5-yl)piperazine

1-(2,3-Dihydrobenzo[1,4]dioxin-5-yl)-4-[1-(2-pyrrolylcarbonyl)-1,2,3,4-tetrahydroquinolin-2-ylmethyl]piperazine

1-(2,3-Dihydrobenzo[1,4]dioxin-5-yl)-4-[1-(2-furanecarbonyl)-1,2,3,4-tetrahydroquinolin-2-ylmethyl]piperazine

1-(2,3-Dihydrobenzo[1,4]dioxin-5-yl)-4-[1-(4-methoxybenzoyl)-1,2,3,4-tetrahydroquinolin-2-ylmethyl]piperazine

1-[1-(4-Cyanobenzoyl)-1,2,3,4-tetrahydroquinolin-2-ylmethyl]-4-(2,3-dihydrobenzo[1,4]dioxin-5-yl)piperazine

1-(2,3-Dihydrobenzo[1,4]dioxin-5-yl)-4-[1-[2-(2-thienyl)acetyl]-1,2,3,4-tetrahydroquinolin-2-ylmethyl]piperazine

1-(2,3-Dihydrobenzo[1,4]dioxin-5-yl)-4-[1-[2-(3-thienyl)acetyl]-1,2,3,4-tetrahydroquinolin-2-ylmethyl]piperazine

1-(1-Cyclobutanecarbonyl)-1,2,3,4-tetrahydroquinolin-2-ylmethyl]-4-(2,3-dihydrobenzo[1,4]dioxin-5-yl)piperazine

1-(2,3-Dihydrobenzo[1,4]dioxin-5-yl)-4-[1-(4-phenoxybutanoyl-1,2,3,4-tetrahydroquinolin-2-ylmethyl]piperazine

1-(1-Benzenesulphonyl-1,2,3,4-tetrahydroquinolin-2-ylmethyl)-4-(2,3-dihydrobenzo[1,4]dioxin-5-yl)piperazine

1-(2,3-Dihydrobenzo[1,4]dioxin-5-yl)-4-(1-phenylmethanesulphonyl-1,2,3,4-tetrahydroquinolin-2-ylmethyl)piperazine

1-(2,3-Dihydrobenzo[1,4]dioxin-5-yl)-4-[1-(2-methylphenylaminocarbonyl)-1,2,3,4-tetrahydroquinolin-2-ylmethyl]piperazine

1-(2,3-Dihydrobenzo[1,4]dioxin-5-yl)-4-[1-(1,1-dimethylethylaminocarbonyl)-1,2,3,4-tetrahydroquinolin-2-ylmethyl]piperazine

1-(2,3-Dihydrobenzo[1,4]dioxin-5-yl)-4-[1-(3,3-dimethylbutanoyl)-1,2,3,4-tetrahydroquinolin-2-ylmethyl]piperazine

1-[1-(1-acetyl-4-piperidinecarbonyl)-1,2,3,4-tetrahydroquinolin-2-ylmethyl]-4-(2,3-dihydrobenzo[1,4]dioxin-5-yl)piperazine

1-(2,3-Dihydrobenzo[1,4]dioxin-5-yl)-4-[1-(2,2-dimethylbutanoyl)-1,2,3,4-tetrahydroquinolin-2-ylmethyl]piperazine

1-(2,3-Dihydrobenzo[1,4]dioxin-5-yl)-4-[1-(1-trifluoromethylcyclopropanecarbonyl)-1,2,3,4-tetrahydroquinolin-2-ylmethyl]piperazine

1-[1-(1-Bicyclo[2.2.2]oct-2-ylcarbonyl)-1,2,3,4-tetrahydroquinolin-2-ylmethyl]-4-(2,3-dihydrobenzo[1,4]dioxin-5-yl)piperazine

1-[1-(1-Cyclopentanecarbonyl)-1,2,3,4-tetrahydroquinolin-2-ylmethyl]-4-(2,3-dihydrobenzo[1,4]dioxin-5-yl)piperazine

1-(2,3-Dihydrobenzo[1,4]dioxin-5-yl)-4-[1-(4-fluorobenzoyl)-1,2,3,4-tetrahydroquinolin-2-ylmethyl]piperazine

1-[1-(Benzo[1,3]dioxol-5-ylcarbonyl)-1,2,3,4-tetrahydroquinolin-2-ylmethyl]-4-(2,3-dihydrobenzo[1,4]dioxin-5-yl)piperazine

1-(2,3-Dihydrobenzo[1,4]dioxin-5-yl)-4-[1-(3-phenylpropenoyl)-1,2,3,4-tetrahydroquinolin-2-ylmethyl]piperazine

1-(2,3-Dihydrobenzo[1,4]dioxin-5-yl)-4-[1-[3-(4-fluorophenyl)propenoyl]-1,2,3,4-tetrahydroquinolin-2-ylmethyl]piperazine

1-(2,3-Dihydrobenzo[1,4]dioxin-5-yl)-4-[1-(3-methyl-2-thiophenecarbonyl)-1,2,3,4-tetrahydroquinolin-2-ylmethyl]piperazine

1-(2,3-Dihydrobenzo[1,4]dioxin-5-yl)-4-[1-(3-furanecarbonyl)-1,2,3,4-tetrahydroquinolin-2-ylmethyl]piperazine

1-(2,3-Dihydrobenzo[1,4]dioxin-5-yl)-4-[1-(2-methyl-3-furanecarbonyl)-1,2,3,4-tetrahydroquinolin-2-ylmethyl]piperazine

1-(2,3-Dihydrobenzo[1,4]dioxin-5-yl)-4-[1-[5-methyl-4-(1,2,4-triazol-1-ylmethyl)-1-furanecarbonyl)-1,2,3,4-tetrahydroquinolin-2-ylmethyl]piperazine

1-(2,3-Dihydrobenzo[1,4]dioxin-5-yl)-4-[1-(5-methyl-4-isoxazolecarbonyl)-1,2,3,4-tetrahydroquinolin-2-ylmethyl]piperazine

1-[1-(4-Acetyl-2-pyrrolecarbonyl)-1,2,3,4-tetrahydroquinolin-2-ylmethyl]-4-(2,3-dihydrobenzo[1,4]dioxin-5-yl)piperazine

N-[2-(2-Methoxyphenoxy)ethyl]-N-[1-(3-methylbutanoyl)-1,2,3,4-tetrahydroquinolin-2-ylmethyl]methylamine

N-[1-(3,3-Dimethylbutanoyl)-1,2,3,4-tetrahydroquinolin-2-ylmethyl]-N-[2-(2-methoxyphenoxy)ethyl]methylamine

N-[1-(2-Cyclopropylacetyl)-1,2,3,4-tetrahydroquinolin-2-ylmethyl]-N-[2-(2-methoxyphenoxy)ethyl]methylamine

N-[2-(2-Methoxyphenoxy)ethyl]-N-[1-(2-pyrrolylcarbonyl)-1,2,3,4-tetrahydroquinolin-2-ylmethyl]methylamine

N-[1-(2-Furoyl)-1,2,3,4-tetrahydroquinolin-2-ylmethyl]-N-[2-(2-methoxyphenoxy)ethyl]methylamine

N-[1-(4-Methoxybenzoyl)-1,2,3,4-tetrahydroquinolin-2-ylmethyl]-N-[2-(2-methoxyphenoxy)ethyl]methylamine

N-[1-(1-Acetyl-4-piperidinecarbonyl)-1,2,3,4-tetrahydroquinolin-2-ylmethyl]-N-[2-(2-methoxyphenoxy)ethyl]methylamine

N-[2-(2-Methoxyphenoxy)ethyl]-N-[1-[2-(2-thienyl)acetyl]-1,2,3,4-tetrahydroquinolin-2-ylmethyl]methylamine

N-[2-(2-Methoxyphenoxy)ethyl]-N-[1-[2-(3-thienyl)acetyl]-1,2,3,4-tetrahydroquinolin-2-ylmethyl]methylamine

N-(1-Cyclobutanecarbonyl)-1,2,3,4-tetrahydroquinolin-2-ylmethyl]-N-[2-(2-methoxyphenoxy)ethyl]methylanine

N-[2-(2-Methoxyphenoxy)ethyl]-N-[1-(4-phenoxybutanoyl-1,2,3,4-tetrahydroquinolin-2-ylmethyl]methylamine

N-(1-Benzenesulphonyl-1,2,3,4-tetrahydroquinolin-2-ylmethyl)-N-[2-(2-methoxyphenoxy)ethyl]methylamine

N-[2-(2-Methoxyphenoxy)ethyl]-N-(1-phenylmethanesulphonyl-1,2,3,4-tetrahydroquinolin-2-ylmethyl)methylamine

N-[2-(2-Methoxyphenoxy)ethyl]-N-[1-(2-methylphenylaminocarbonyl)-1,2,3,4-tetrahydroquinolin-2-ylmethyl]methylamine

N-[1-(1,1-Dimethylethyl)-1,2,3,4-tetrahydroquinolin-2-ylmethyl]-N-[2-(2-methoxyphenoxy)ethyl]methylamine

1-(1-Cyclohexanecarbonyl-5-fluoro-1,2,3,4-tetrahydroquinoline-2-ylmethyl)-4-(4-fluoro-2-methoxyphenyl)piperazine.

1-(1-Cyclohexanecarbonyl-1,2,3,4-tetrahydroquinoline-2-ylmethyl)-4-(2-triluoromethanesulphonyloxyphenyl)piperazine.

1-(1-Cyclohexanecarbonyl-1,2,3,4-tetrahydroquinoline-2-ylmethyl)-4-(1-isopropyl-4-indolyl)piperazine

1-(6-tert-Butoxycarbonylamino-1-cyclohexanecarbonyl-1,2,3,4-tetrahydroquinoline-2-ylmethyl)-4-(4-fluoro-2-methoxyphenyl)piperazine

1-(1-Cyclohexanecarbonyl-1,2,3,4-tetrahydroquinoline-2-ylmethyl)-4-(1-methoxymethyl-4-indolyl)piperazine

1-(1-Cyclohexanecarbonyl-2-methyl-1,2,3,4-tetrahydroquinoline-2-ylmethyl)-4-(4-fluoro-2-methoxyphenyl)piperazine

1-(1-Cyclopentylcarbonyl-6-methyl-1,2,3,4-tetrahydroquinoline-2-ylmethyl]-4-(4-fluoro-2-methoxyphenyl)piperazine

1-(6-Amino-1-cyclohexanecarbonyl-1,2,3,4-tetrahydroquinoline-2-ylmethyl)-4-(4-fluoro-2-methoxyphenyl)piperazine

1-(1-Cyclohexanecarbonyl-1,2,3,4-tetrahydroquinoline-2-ylmethyl)-4-(2-methoxy-4-methylphenyl)piperazine

1-(Benzo[1,3]dioxol-5-ylmethyl)-4-(1-cyclohexanecarbonyl-1,2,3,4-tetrahydroquinoline-2-ylmethyl)piperazine

N-(1-Cyclohexanecarbonyl-1,2,3,4-tetrahydroquinoline-2-ylmethyl)-N-[2-(2-methoxyphenoxy)]ethylamine

N-(1-Cyclohexanecarbonyl-1,2,3,4-tetrahydroquinoline-2-ylmethyl)-N-[3-(2-methoxyphenoxy)propylmethylamine]

4-Cyano-1-(1-cyclohexanecarbonyl-1,2,3,4-tetrahydroquinoline-2-ylmethyl)-4-phenylpiperidine

4-Benzyl-1-(1-cyclohexanecarbonyl-1,2,3,4-tetrahydroquinoline-2-ylmethyl)-4-hydroxypiperidine

4-Benzyloxy-1-(1-cyclohexanecarbonyl-1,2,3,4-tetrahydroquinoline-2-ylmethyl)piperidine

4-Benzoylamino-1-(1-cyclohexanecarbonyl-1,2,3,4-tetrahydroquinoline-2-ylmethyl)piperidine

4-Acetyl-1-(1-cyclohexanecarbonyl-1,2,3,4-tetrahydroquinoline-2-ylmethyl)-4-phenylpiperidine

1-(Benzo[1,3]dioxol-4-yl)-4-(1-cyclohexanecarbonyl-1,2,3,4-tetrahydroquinoline-2-ylmethyl)piperazine

N-[2-(3-Chlorophenoxy)ethyl]-N-(1-cyclohexanecarbonyl-1,2,3,4-tetrahydroquinoline-2-yl)methylamine

N-[2-(4-Chlorophenoxy)ethyl]-N-(1-cyclohexanecarbonyl-1,2,3,4-tetrahydroquinoline-2-yl)methylamine

N-Benzyl-N-(1-cyclohexanecarbonyl-1,2,3,4-tetrahydroquinoline-2-ylmethyl)-N-[2-(2-methoxyphenoxy)ethyl]amine

N-[(1-Cyclohexanecarbonyl-1,2,3,4-tetrahydroquinoline-2-yl)methyl]-N-(3-phenylpropyl)methylamine

1-[4-Cyano-2-trifluoromethoxyphenyl)-4-(1-cyclohexanecarbonyl-1,2,3,4-tetrahydroquinoline-2-ylmethyl)piperazine

1-(1-Cyclohexanecarbonyl-1,2,3,4-tetrahydroquinoline-2-ylmethyl)-4-(4-fluoro-2-methoxyphenylamino)piperidine

1-(3H-Benzotriazol-4-yl)-4-(1-cyclohexanecarbonyl-1,2,3,4-tetrahydroquinoline-2-ylmethyl)piperazine

1-(1H-Benzoimidazol-4-yl)-4-(1-cyclohexanecarbonyl-1,2,3,4-tetrahydroquinoline-2-ylmethyl)piperazine

N-(1-Cyclohexanecarbonyl-1,2,3,4-tetrahydroquinoline-2-ylmethyl)-N-[2-(2-pyridyloxy)ethylamine]

N-(1-Cyclohexanecarbonyl-1,2,3,4-tetrahydroquinoline-2-ylmethyl)-N-[2-(2-pyridyloxy)ethylmethylamine]

N-(1-Cyclohexanecarbonyl-1,2,3,4-tetrahydroquinoline-2-ylmethyl)-N-[3-(2-methoxyphenyl)propylmethylamine]

1-(4-Benzoyl)-4-(1-cyclohexanecarbonyl-1,2,3,4-tetrahydroquinoline-2-ylmethyl)piperazine

1-(1-Cyclohexanecarbonyl-1,2,3,4-tetrahydroquinoline-2-ylmethyl)-3-oxo-4-phenylpiperazine

1-(4-Fluoro-2-methoxyphenyl)-4-(1-hexanoyl-1,2,3,4-tetrahydroquinoline-2-ylmethyl)piperazine

1-(2,3-Dihydrobenzo[1,4]dioxin-5-yl)-4-(1-pentafluoropropionyl-1,2,3,4-tetrahydroquinolin-2-ylmethyl)piperazine

1-(2,3-Dihydrobenzo[1,4]dioxin-5-yl)-4-[1-(3,3,3-trifluoropropionyl)-1,2,3,4-tetrahydroquinolin-2-ylmethyl)piperazine

(Z)-1-(2,3-Dihydrobenzo[1,4]dioxin-5-yl)-4-[1-(4-hydroxycyclohexanecarbonyl)-1,2,3,4-tetrahydroquinolin-2-ylmethyl]piperazine

1-(1-Cyclohexanecarbonyl-6-fluoro-1,2,3,4-tetrahydroquinoline-2-ylmethyl)-4-(4-fluoro-2-methoxyphenyl)piperazine

### Pharmaceutical Compositions

The invention further provides pharmaceutical compositions comprising a compound of formula I or an enantiomer, diastereomer, N-oxide, crystalline form, hydrate, solvate, active metabolite or pharmaceutically acceptable salt thereof The pharmaceutical composition may also include optional additives, such as a pharmaceutically acceptable carrier or diluent, a flavorant, a sweetener, a preservative, a dye, a binder, a suspending agent, a dispersing agent, a colorant, a disintegrant, an excipient, a film forming agent, a lubricant, a plasticizer, an edible oil or any combination of two or more of the foregoing.

Suitable pharmaceutically acceptable carriers or diluents include, but are not limited to, ethanol; water; glycerol; propylene glycol, aloe vera gel; allantoin; glycerin; vitamin A and E oils; mineral oil; PPG2 myristyl propionate; magnesium carbonate; potassium phosphate; vegetable oil; animal oil; and solketal.

Suitable binders include, but are not limited to, starch; gelatin; natural sugars, such as glucose, sucrose and lactose; corn sweeteners; natural and synthetic gums, such as acacia, tragacanth, vegetable gum, and sodium alginate; carboxymethylcellulose; hydroxypropylmethylcellulose; polyethylene glycol; povidone; waxes; and the like.

Suitable disintegrants include, but are not limited to, starch, e.g., corn starch, methyl cellulose, agar, bentonite, xanthan gum, sodium starch glycolate, crosspovidone and the like.

Suitable lubricants include, but are not limited to, sodium oleate, sodium stearate, sodium stearyl fumarate, magnesium stearate, sodium benzoate, sodium acetate, sodium chloride and the like.

A suitable suspending agent is, but is not limited to, bentonite, ethoxylated isostearyl alcohols, polyoxyethylene sorbitol and sorbitan esters, microcrystalline cellulose, aluminum metahydroxide, agar-agar and tragacanth, or mixtures of two or more of these substances, and the like.

Suitable dispersing and suspending agents include, but are not limited to, synthetic and natural gums, such as vegetable gum, tragacanth, acacia, alginate, dextran, sodium carboxymethylcellulose, methylcellulose, polyvinyl-pyrrolidone and gelatin.

Suitable film forming agents include, but are not limited to, hydroxypropylmethylcellulose, ethylcellulose and polymethacrylates.

Suitable plasticizers include, but are not limited to, polyethylene glycols of different molecular weights (e.g., 200-8000 Da) and propylene glycol.

Suitable colorants include, but are not limited to, ferric oxide(s), titanium dioxide and natural and synthetic lakes.

Suitable edible oils include, but are not limited to, cottonseed oil, sesame oil, coconut oil and peanut oil.

Examples of additional additives include, but are not limited to, sorbitol, talc, stearic acid, dicalcium phosphate and polydextrose.

### Unit Dosage Forms

The pharmaceutical composition may be formulated as unit dosage forms, such as tablets, pills, capsules, caplets, boluses, powders, granules, sterile parenteral solutions, sterile parenteral suspensions, sterile parenteral emulsions, elixirs, tinctures, metered aerosol or liquid sprays, drops, ampoules, autoinjector devices or suppositories. Unit dosage forms may be used for oral, parenteral, intranasal, buccal, sublingual or rectal administration, or for administration by inhalation or insufflation, transdermal patches, and a lyophilized composition. In general, any delivery of active ingredients that results in systemic availability of them can be used. Preferably the unit dosage form is an oral dosage form, most preferably a solid oral dosage form, therefore the preferred dosage forms are tablets, pills, caplets and capsules. Parenteral preparations (e.g., injectable preparations and preparations for powder jet systems) also are preferred.

Solid unit dosage forms may be prepared by mixing an active agent of the present invention with a pharmaceutically acceptable carrier and any other desired additives as described above. The mixture is typically mixed until a homogeneous mixture of the active agents of the present invention and the carrier and any other desired additives is formed, i.e., until the active agent is dispersed evenly throughout the composition. In this case, the compositions can be formed as dry or moist granules.

Dosage forms can be formulated as, for example, “immediate release” dosage forms. “Immediate release” dosage forms are typically formulated as tablets that release at least 70%-90% of the active ingredient within 30-60 min when tested in a drug dissolution test, e.g., U.S. Pharmacopeia standard <711>. In certain embodiments, immediate dosage forms release at 75% of active ingredient in 45 min.

Dosage forms can also be formulated as, for example, “controlled release” dosage forms. “Controlled,” “sustained,” “extended” or “time release” dosage forms are equivalent terms that describe the type of active agent delivery that occurs when the active agent is released from a delivery vehicle at an ascertainable and manipulatable rate over a period of time, which is generally on the order of minutes, hours or days, typically ranging from about sixty minutes to about 3 days, rather than being dispersed immediately upon entry into the digestive tract or upon contact with gastric fluid. A controlled release rate can vary as a function of a multiplicity of factors. Factors influencing the rate of delivery in controlled release include the particle size, composition, porosity, charge structure, and degree of hydration of the delivery vehicle and the active ingredient(s), the acidity of the environment (either internal or external to the delivery vehicle), and the solubility of the active agent in the physiological environment, i.e., the particular location along the digestive tract. Typical parameters for dissolution test of controlled release forms are found in U.S. Pharmacopeia standard <724>.

Dosage forms can also be formulated to deliver active agent in multiphasic stages whereby a first fraction of an active ingredient is released at a first rate and at least a second fractions of active ingredient is released at a second rate. In a preferred embodiment, a dosage form can be formulated to deliver active agent in a biphasic manner, comprising a first “immediate release phase”, wherein a fraction of active ingredient is delivered at a rate set forth above for immediate release dosage forms, and a second “controlled release phase,” wherein the remainder of the active ingredient is released in a controlled release manner, as set forth above for controlled release dosage forms.

Tablets, caplets or pills can be coated or otherwise prepared so as to form a unit dosage form that has delayed and/or sustained action, such as controlled release and delayed release unit dosage forms. For example, the tablet or pill can comprise an inner dosage and an outer dosage component, the latter being in the form of a layer or envelope over the former. The two components can be separated by an enteric layer which serves to resist disintegration in the stomach and permits the inner component to pass intact into the duodenum or to be delayed in release.

Biodegradable polymers for controlling the release of the active agents include, but are not limited to, polylactic acid, polyepsilon caprolactone, polyhydroxybutyric acid, polyorthoesters, polyacetals, polydihydropyrans, polycyanoacrylates and crosslinked or amphipathic block copolymers of hydrogels.

For liquid dosage forms, the active substances or their physiologically acceptable salts are dissolved, suspended or emulsified, optionally with the usually employed substances such as solubilizers, emulsifiers or other auxiliaries. Solvents for the active combinations and the corresponding physiologically acceptable salts can include water, physiological salt solutions or alcohols, e.g. ethanol, propanediol or glycerol. Additionally, sugar solutions such as glucose or mannitol solutions may be used. A mixture of the various solvents mentioned may be used in the present invention too.

A transdermal dosage form is contemplated by the present invention too. Transdermal forms may be a diffusion transdermal system (transdermal patch) using either a fluid reservoir or a drug-in-adhesive matrix system. Other transdermal dosage forms include, but are not limited to, topical gels, lotions, ointments, transmucosal systems and devices, and iontophoretic (electrical diffusion) delivery systems. Transdermal dosage forms may be used for delayed release and sustained release of the active agents of the present invention.

The pharmaceutical compositions and unit dosage forms of the present invention for parenteral administration, and in particular by injection, typically include a pharmaceutically acceptable carrier, as described above. A preferred liquid carrier is vegetable oil. Injection may be, for example, intravenous, epidural, intrathecal, intramuscular, intraluminal, intratracheal or subcutaneous.

The active agents can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The active agents of the present invention may also be coupled with soluble polymers such as targetable drug carriers. Such polymers include, but are not limited to, polyvinylpyrrolidone, pyran copolymers, polyhydroxypropylmethacrylamidophenol, polyhydroxyethylaspartamidophenol, and polyethylenoxypolylysine substituted with palmitoyl residues.

### Administration

The pharmaceutical composition or unit dosage forms of the present invention may be administered by a variety of routes, such as, without limitation, oral, buccal, enteral, parenteral, intravenous, intramuscular subcutaneous, transdermal, transmucosal (including rectal and buccal) and by inhalation routes.

Preferably, the oral or transdermal route is used (i.e., with solid or liquid formulations or skin patches, respectively).

The pharmaceutical composition or unit dosage forms comprising an effective amount of the present invention may be administered to an animal, preferably a human, in need of treatment of neuromuscular dysfunction of the lower urinary tract described by E. J. McGuire in “Campbell's UROLOGY”, 5^{th} Ed. 616-638, 1986, W. B. Saunders Company, and patients affected by any physiological dysfunction related to impairment of 5-HT_{1A} receptor function. Such dysfunctions include, without limitation, central-nervous-system disorders such as depression, anxiety, eating disorders, sexual dysfunction, addiction and related problems.

As used herein, the term “effective amount” refers to an amount that results in measurable amelioration of at least one symptom or parameter of a specific disorder. In a preferred embodiment, the compound treats disorders of the urinary tract, such as urinary urgency, overactive bladder, increased urinary frequency, reduced urinary compliance (reduced bladder storage capacity), cystitis (including interstitial cystitis), incontinence, urine leakage, enuresis, dysuria, urinary hesitancy and difficulty in emptying the bladder, or central nervous system disorders due to serotonergic dysfunction (such as anxiety, depression, hypertension, sleep/wake cycle disorders, feeding behaviour, sexual function and cognition disorders in mammals (particularly a human) associated to stroke, injury, dementia and due to neurological development, disorders from hyperactivity related to an attention deficit (ADHD), drug addiction, drug withdrawal, irritable bowel syndrome.

The pharmaceutical composition or unit dosage form of the present invention may be administered according to a dosage and administration regimen defined by routine testing in the light of the guidelines given above in order to obtain optimal activity while minimising toxicity or side effects for a particular patient. However, such fine tuning of the therapeutic regimen is routine in the light of the guidelines given herein.

The dosage of the active agents of the present invention may vary according to a variety of factors such as underlying disease conditions, the individual's condition, weight, sex and age, and the mode of administration. An effective amount for treating a disorder can easily be determined by empirical methods known to those of ordinary skill in the art, for example by establishing a matrix of dosages and frequencies of administration and comparing a group of experimental units or subjects at each point in the matrix. The exact amount to be administered to a patient will vary depending on the state and severity of the disorder and the physical condition of the patient. A measurable amelioration of any symptom or parameter can be determined by a person skilled in the art or reported by the patient to the physician. It will be understood that any clinically or statistically significant attenuation or amelioration of any symptom or parameter of urinary tract disorders is within the scope of the invention. Clinically significant attenuation or amelioration means perceptible to the patient and/or to the physician.

For example, a single patient may suffer from several symptoms of dysuria simultaneously, such as, for non-limiting example, urgency and excessive frequency of urination or both, and these may be reduced using the methods of the present invention. In the case of incontinence, any reduction in the frequency or volume of unwanted passage of urine is considered a beneficial effect of the present method of treatment.

The amount of the agent to be administered can range between about 0.01 and about 25 mg/kg/day, preferably between about 0.1 and about 10 mg/kg/day and most preferably between 0.2 and about 5 mg/kg/day. It will be understood that the pharmaceutical formulations of the present invention need not necessarily contain the entire amount of the agent that is effective in treating the disorder, as such effective amounts can be reached by administration of a plurality of doses of such pharmaceutical formulations.

In a preferred embodiment of the present invention, the compounds are formulated in capsules or tablets, preferably containing 50 to 200 mg of the compounds of the invention, and are preferably administered to a patient at a total daily dose of 50 to 400 mg, preferably 150 to 250 mg and most preferably about 200 mg, for relief of urinary incontinence and dysfunctions under treatment with 5-HT_{1A} receptor ligand.

A pharmaceutical composition for parenteral administration prefereably contains from about 0.01% to about 100% by weight of the active agents of the present invention, based upon 100% weight of total pharmaceutical composition.

Generally, transdermal dosage forms contain from about 0.01% to about 100% by weight of the active agents versus 100% total weight of the dosage form.

The pharmaceutical composition or unit dosage form may be administered in a single daily dose, or the total daily dosage may be administered in divided doses. In addition, co-administration or sequential administration of another compound for the treatment of the disorder may be desirable. For example, the compounds of the invention may be administered in combination with known muscarinic receptor antagonists such as oxybutynin, tolterodine, darifenacin and temiverine. Analogously, the compounds of the invention may be administered with α1-adrenergic antagonists, for the therapy of lower urinary tract symptoms, whether or not these are associated with BPH. Examples of α1-adrenergic antagonists suitable for administration in combination with a compound of the invention are, without limitation, phentolamine, phenoxybenzamine, prazosin, alfuzosin, doxazosin, terazosin, tamsulosin (YM-617), olanzapine, mirtazapine (Remeron, 6-azamianserin, ORG 3770), chloroethylclonidine, WB4101, corynanthine, tolazoline, trimazosin, reserpine, labetalol, carvedilol, indoramin, RS 17053, urapidil, rauwolscine, RS 21361, piperoxan, ketanserin, tiodazosin, E-643, 5-methylurapidil (5Mu), (+) niguldipine, KMD 3213, BMY 7378, QAPB, trazodone, SNAP-1069), SK & F104856, RWJ-38063, RWJ-69736, RO-70-0004, RS-100329, thymoxamine (moxisylyte), benextramine, dihydroergocryptine, cyclazosin, Rec 15/2615, SKF 106686, benoxathian, mephendioxan, discretamine, SNAP 5089, spiperone, SNAP 8719, RS-97078, A-131701, furosemide, boldine, hexahydroisoquinolino [8,1-ab] carbazole (((+/−) IQC) (1a), bromotopsentin, tetrahydropapaverine, REC 15/2869, REC 15/3039, REC 15/2739 (SB 216469), REC 15/3011, WAY 100635, BE2254 (HEAT), AH11110A (AH1111OA), SNAP 5540, L765,314, BRL 48962, SL 89 0591, abanoquil, SK&F 105854, BRL 44408, imiloxan, SK&F 86468, SK&F 102229, dibenamine, EEDQ, SZL-49, ARC-239, spiroxatrine, amosulalol, naftopidil, SL 89.0951, raubasine, akuammigine, tetrahydroalstonine, yohimbine, yohimbol, apoyohimbine, bunazosin, napamezole, clonidine, clozapine, SNAP5257. Additional α1-adrenergic antagonists suitable for administration in combination with a compound of the invention are found, for example, in U.S. Pat. Nos. 5,798,362; 6,306,861; 6,365,591 and 6,403,594. Preferred α1-adrenergic antagonists suitable for administration in combination with a compound of the invention are prazosin, doxazosin, terazosin, alfuzosin and tamsulosin.

For combination treatment where the compounds are in separate dosage formulations, the compounds can be administered concurrently, or each can be administered at separate staggered times. For example, the compound of the invention may be administered in the morning and the antimuscarinic compound may be administered in the evening, or vice versa. Additional compounds may be administered at specific intervals too. The order of administration will depend upon a variety of factors including age, weight, sex and medical condition of the patient; the severity and aetiology of the disorders to be treated, the route of administration, the renal and hepatic function of the patient, the treatment history of the patient, and the responsiveness of the patient. Determination of the order of administration may be fine-tuned and such fine-tuning is routine in the light of the guidelines given herein.

Specific dosage forms and modes of administration can be found, without limitation, in U.S. Pat. Nos. 5,500,222; 5,512,293; 5,531,736; 5,533,971; 5,543,156; 5,573,776; 5,629,019; 5,641,504; 6,039,977; 6,096,339; 6,106,845; 6,130,200; and 6,174,547.

### Uses-Methods for Treatment

Without wishing to be bound by theory, it is believed that administration of 5-HT_{1A} receptor antagonists prevents unwanted activity of the sacral reflex and/or cortical mechanisms that control micturition. Thus, it is contemplated that a wide range of neuromuscular dysfunctions of the lower urinary tract can be treated using the compounds of the present invention, including without limitation dysuria, incontinence and enuresis (overactive bladder). Dysuria includes urinary frequency, nocturia, urgency, reduced urinary compliance (reduced bladder storage capacity), difficulty in emptying the bladder, i.e. a suboptimal volume of urine is expelled during micturition. Incontinence syndromes include stress incontinence, urgency incontinence and enuresis incontinence, as well as mixed forms of incontinence. Enuresis refers to the involuntary passage of urine at night or during sleep.

The compounds of the present invention may also be useful for the treatment of central nervous system disorders due to serotonergic dysfunction.

### Synthesis of the Compounds of the Invention Description

The compounds according to the invention may generally be prepared as follows:

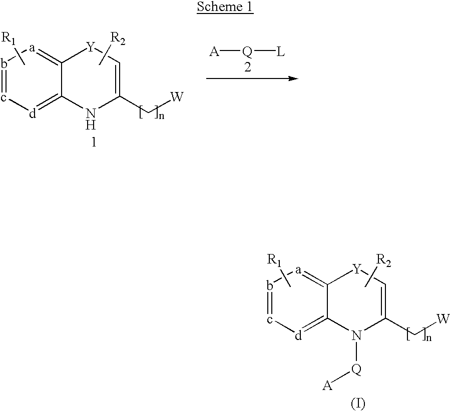

The compounds of the invention are obtained by direct condensation of compounds 1 with compounds 2 (Scheme 1). When A is alkyl, cycloalkyl, cycloalkenyl, aryl, heterocycle, (di)alkylamino or cyclic amino and L is a halogen atom, this step is the well-known reaction between an acyl, sulphonyl, thiocarbonyl or a carbamoyl chloride (e.g., chloroformamide) and amine 1. Chlorides 2 are generally commercially available items or are prepared by conventional procedures well documented in the literature and very well-known to those skilled in the art. Condensation is carried out as usual in an aprotic solvent (e.g. a chlorinated solvent or tetrahydrofuran or toluene) in the presence of an organic or inorganic base, such as triethylamine or diisopropylethylamine, as a proton scavenger at a temperature in the range of between −20° C. and the reflux of the solvent.

If compound 2 is a carboxylic acid (L=OH), condensation can be carried out in the presence of a condensing agent (e.g. dicyclohexylcarbodiimide or diethyl cyanophosphonate), optionally in the presence of a promoting agent (e.g. N-hydroxysuccinimide, 4-dimethylaminopyridine or N,N′-carbonyldiimidazole) in an aprotic or chlorinated solvent (e.g. N,N-dimethylformamide or chloroform) at between −10 and 140° C. (Albertson N. F., Org. React. 12, 205-218 (1962); Doherty A. M. et al., J. Med. Chem. 35, 2-14 (1992); Ishihara Y. et al., Chem Pharm. Bull. 39, 3236-3243 (1991)). In some cases the activated intermediate esters or amides (such as O-(N-succinimidyl) esters or N-acyl imidazolides) can be isolated and further reacted with compound 1 to be transformed into the corresponding amides (I) in an aprotic or chlorinated solvent at between 10 and 100° C. Another activated intermediate which can be used is the mixed anhydride of the carboxylic acid 2, obtainable by reacting 2 (L=OH) with an alkyl chloroformate (optionally supported on resin) in the presence of a tertiary amine (e.g., triethylamine or N-methylmorpholine), then reacting it with compound 1 at between 0 and 80° C.; optionally a promoting agent (e.g. 1-hydroxypiperidine) may be added before the amine addition (Albertson N. F., Org. React. 12, 157 (1962)). Alternatively, condensation can be carried out without a solvent at between 150 and 220° C. (Mitchell J. A. et al., J. Am. Chem. Soc. 53, 1879 (1931)) or in high-boiling ethereal solvents (e.g., diglyme). Less reactive derivatives of the carboxylic acid 2 can be used too, such as alkyl esters, which, in turn, can be converted into (I) in the presence of a condensing agent (e.g., trimethylaluminum) in an aprotic and/or chlorinated solvent (e.g., hexane, dichloromethane) at between −10 and 80° C., or without solvents at between 80 and 180° C. (Weinreb S. M. et al., Tetrahedron Lett. 4171 (1977); Lipton M. F. et al., Org. Synth. 59, 49 (1979)). An alternative procedure to prepare compounds (I) where A is alkylamino, dialkylamino, cyclic amino, arylamino or arylalkylamino, consists in the use of isocyanates, which are commercially available or prepared in situ or a priori by conventional procedures well known to those skilled in the art, or in the use of carbonyldiimidazole or phosgene or other phosgene-like compounds subsequently reacting them in a proper solvent (e.g., THF or toluene or a chlorinated solvent) with compound 1 followed by the proper amine (or vice-versa).

Intermediates 2 (L=OH, Cl) can be prepared by standard procedures well known to those skilled in the art and documented in the literature. Compounds (I) where A is an alkyl optionally substituted with one or more hydroxy, alkoxy, arylalkoxy, amino, acylamino, cyanoamino, aminocarbonyl, alkylaminocarbonyl groups can be alternatively prepared from compounds (I) where A-Q is HalAlkylQ or CH₂═CHCO (Ia) by simple nucleophilic substitutions or 1,4 additions (Michael reactions) with the proper reactive (e.g., sodium cyanamide, sodium cyanide, sodium alkoxides). The synthesis of compounds (I) where A contains an OH group or an amino or acylamino group requires a supplementary step of deprotection (and acylation). For example, compounds (I) with a masked or protected amino group or a protected hydroxy group can be obtained by reaction of (Ia) with sodium benzylate or sodium azide or potassium phthalimide or benzylamine or others (see, for example, T. W. Greene et al., Protective Groups in Organic Synthesis, 3^{rd} Ed., Wiley Interscience, New York). Compounds (I) are obtained after deprotection or reduction in the case of azido group by standard methods. The preferred method for the synthesis of compounds (I) where A contains an OH group consists anyway in the reaction of a O-protected O-alkylCOOH derivative (or its activated analogue) with compounds 1. Preferred protecting group is benzyl which can be removed by hydrogenolysis.

When R₁ and the substituents on ring B in compounds I represent reactive groups (e.g., hydroxyl or amino), these should be protected before the acylation of Scheme 1 and then deprotected following the known methods described, for example, in Greene et al., supra.

Preparation of Intermediates 1 can be carried out by several methods. The main procedures are described below.

The first procedure is detailed in Scheme 2 and includes nucleophilic substitution of the proper amine 14 on a 2-halomethylbicyclic 4 or 1,2-addition to a 2-vinylbicyclic 6 in a suitable solvent (e.g., acetonitrile, N,N-dimethylformamide, a chlorinated solvent, toluene or other protic or aprotic polar solvent) at a temperature of between 0° C. and the reflux temperature of the solvent in the presence or not of a base such as N,N-diisopropylethylamine, TEA, potassium carbonate, 1,8-diazabicycloundec-7-ene or others. Intermediates 4 can be prepared by halogenation of 3 with N-bromosuccinimide or N-chlorosuccinimide by conventional procedures well known to those skilled in the art and documented in the experimental part.

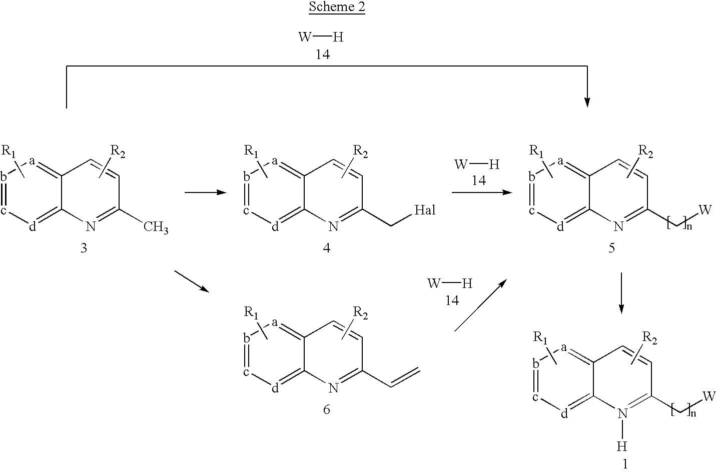

Those of compounds 3 (e.g., a, b, c, d=CH), which are not commercially available, can be prepared from the corresponding substituted anilines by the methods cited by O. Foye et al., J. Pharm. Sci. 68(3), 336-338, (1979) or by J. C. Hardy et al. WO 98/38194 or by other suitable methods disclosed in the literature.

Those of compounds 6 (e.g., a, b, c, d=CH), which are not commercially available can be prepared from the corresponding compounds 3 by the method of Buchmann G. et al., J. Prakt. Chem. 24(4), 101-112, (1964) or by other suitable methods.

An alternative procedure for the preparation of the compounds 5 of the invention where n is 2 is a reaction between the 2-methylbicyclic 3 and formaldehyde in the presence of an appropriate cyclic amine. This is the well-known Mannich reaction, which is generally carried out in the following manner, viz. the amine in the form of an acid addition salt is dissolved in a 40% aqueous formaldehyde solution and then added to an alcoholic solution of 2-methylbicyclic, and the resulting mixture is warmed. Alternative Mannich procedures that can be applied if necessary are available and described in the literature. The tertiary amine 5 is then converted to the desired intermediate 1 by standard hydrogenation methods in the presence of a catalyst such as platinum, platinum dioxide, palladium or rodhium or nickel, with or without a support such as charcoal or alumina.

This hydrogenation can also be carried out using nascent hydrogen generated by sodium, lithium or potassium metals and a lower alkanol, e.g. methanol, ethanol, propanol, n-butanol, at a temperature ranging between the room temperature and the boiling point of the solvent. Other methods of reduction of the pyridine ring can be used, as detailed in the literature (C. J. Moody, SYNLETT 9, 1029-1030, (1998); B. C. Ranu, Synth. Comm. 28(3), 485-492, (1998); P. Balczewsky, Synth. Comm. 20(18), 2815-2819, (1990); A Srikrishna, Tetrahedron 52(5), 1631-1636, (1996)).

The same synthetic approach can be used to prepare the intermediate with Y=bond corresponding to 5 from 2-methylbicyclic analogues (e.g., 2-methylindole), subsequently reducing them to the indoline derivatives corresponding to 1.

An alternative procedure to prepare compounds 1 is illustrated in Scheme 3.

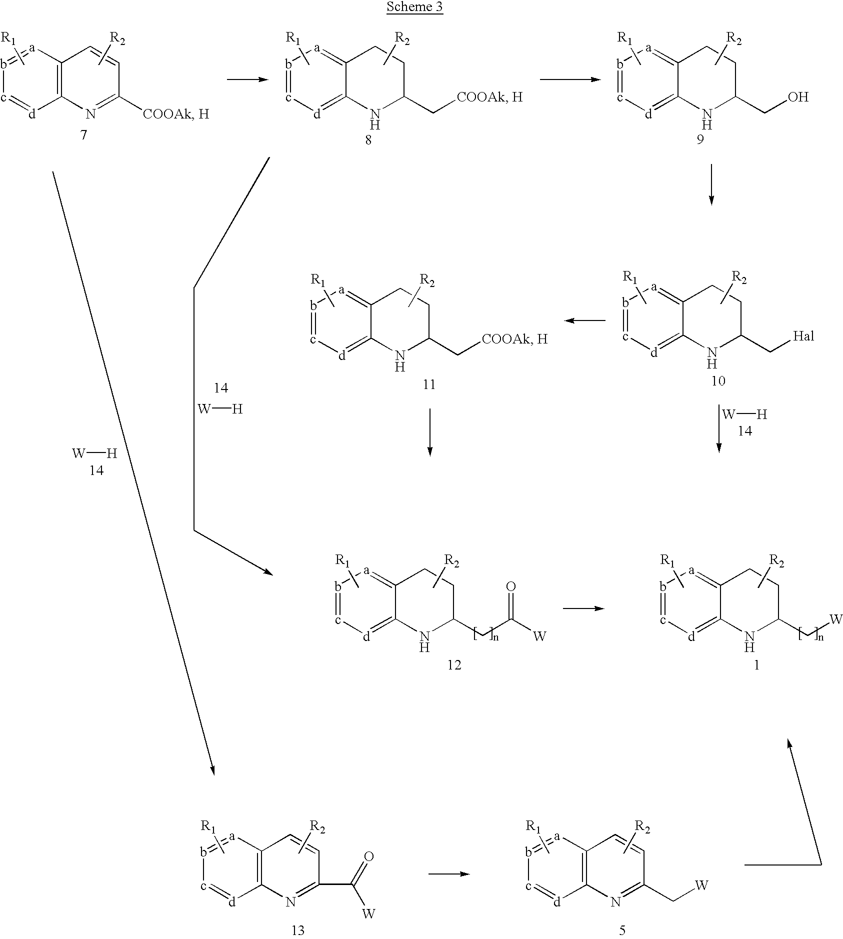

Intermediates 7 are commercially available or can be prepared by methods well known to those skilled in the art and cited in the literature, e.g., by Reissert reaction, treating the proper bicyclic unsubstituted at position 2 (e.g., quinoline) with cyanide ion or equivalent (e.g., trimethylsilylcyanide, Popp, F. D. Heterocycles, 23, 731, (1985); D. E. Portlock, U.S. Pat. No. 4,461,896; Renaud, A. et al, EP 322 263) or by haloform reaction from the proper 2-methylbicyclic by exhaustive halogenation followed by hydrolysis (Ejima et al U.S. Pat. No. 6,169,086) or by reacting a properly substituted aniline with dimethyl acetylenedicarboxylate in a high boiling solvent (e.g., diphenyl ether) affording compound 7 as a 4-hydroxy derivative which can be in turn converted into a 4-halo derivative (phosphorus oxychloride). Compound 7 can be reduced to the corresponding 1,2,3,4-tetrahydroderivatives (8) by the same hydrogenation procedures cited above and condensed by standard methods with the proper amines to afford amides 12 (see above for condensation reactions). The next step is a standard reduction with lithium aluminium hydride or borane or, where necessary, non standard reduction (e.g., conversion into a thioamide and reduction with boron and sodium hydride) to convert the amide carbonyl function to a methylene group. The reduction is accomplished by dissolving the amide in a solvent such as dioxane and then adding the organic solution slowly to a thick suspension of lithium aluminium hydride in anhydrous dioxane or tetrahydrofuran or 1,2-dimethoxyethane or other solvent. The catalytic hydrogenation step that reduces the Y-bearing hetero ring in the bicyclic can also be performed on the reduction products 5 obtained by the same procedure as above by reduction of intermediates 13, in turn obtained from 7. The carboxy group of intermediates 8 can also be reduced by standard fashions (see R. Nagata et al., J. Med. Chem. 37, 3956-3968 (1994)) to give alcohols 9 which can be converted in turn into halo derivatives 10 by standard halogenation procedures (SOCl₂, PBr₃ or others) or by Mitsunobu reaction (Nagata supra or carbon tetrachloride, triphenylphosphine and a chlorinated solvent or others, between room temperature and reflux). The compounds 10 can be reacted through classical nucleophilic-substitution methods, very well-known to those skilled in the art, with the proper amines to afford compounds 1. The compounds 10 can also be used in the homologation reactions which lead to formation of 11 (for example via cyanide nucleophilic substitution, hydrolysis and reesterification). The entire last reaction pathway can be carried out by conventional methods which are well documented in the literature (see for example R. Nagata et al.).

In addition to the above procedures, there are two similar related methods for preparing the same compounds. The first variation utilises a 2-formylbicyclic (obtained, for example, by selenium-dioxide oxidation of derivatives 3 (Scheme 2) as starting material instead of the corresponding 2-carboxylic acid. The 2-formyl compound can be simultaneously reacted with amine 14 and reduced via hydrogenation over Raney nickel, platinum oxide (Adams catalyst) or a catalyst made up of 5% palladium on barium sulphate. The combined reaction is generally carried out in a suitable solvent such as absolute alcohol. The last procedure can also be performed by using a classical reductive-amination method and a reducing agent such as sodium triacetoxyborohydride or sodium cyanoborohydride followed by hydrogenation of the pyridine nucleus. If R₁ is nitro, a nitration step should be introduced, for example, on compounds 10 or 11 (preferred) in which R₁ is H. The best mode to carry out the nitration step is to use a N-protected 11 (e.g., N-cyclohexylcarbonyl derivative 15 (see Scheme 4). The compounds of the invention in which R₁ is nitro can also be prepared by nitro-dediazosubstitution (Sandmeyer reaction) on diazonium salt from aniline compounds, as well as compounds where R₁ is halogen, which can be obtained from the corresponding compounds where R₁ is nitro by reduction to the amine, followed by diazotization and subsequent conversion to the halo compound via well-known methods.

If R₁ is Br, a bromination step (e.g., with N-bromosuccinimide in DMF) should be introduced, for example, on compounds 10 or 11 (preferred) in which R₁ is H. Bromination on compounds 15 (Scheme 4) requires harsher conditions (e.g., Br₂ with Fe catalysis in dichloromethane or other methods from the literature (see Nagata supra)). Starting reagents required for these reactions are either commercially available or prepared using standard techniques and normal organic-synthesis procedures that are well known in the art. Such techniques are disclosed in standard organic-synthesis textbooks and/or published in the literature.

The compounds I where Y is a bond can be prepared in analogy with the procedures detailed above for Y═CH₂ derivatives (see Nagata supra). Harsher conditions are required for the acylations that lead to compounds (I) when the heterocycle is an indole derivative. The use of strong bases is needed to deprotonate the indole NH group to make it a good nucleophile (see T. W. Greene et al., Protective Group in Organic Synthesis, 3^{rd} Ed., Wiley Interscience, New York, 1999). When B is not an N-protected indole, the last acylation step to form I must be carried out after protecting the B indole with a proper group such as a silyl or tertbutoxycarbonyl or other group (see Greene supra). The obtained protected compound I must then be deprotected by standard methods. Alternatively, the compounds I where B is indole or other potentially-reactive hetero ring can be prepared starting from, for example, a methyl or ethyl indole-2-carboxylate, N-acylating it and reducing the carboxylate group, avoiding harsh reaction conditions, for example by using CaBH₄ or hydrolyzing the ester moiety, and carrying out the reduction on the mixed anhydride obtained from indole-2-carboxylic acid and ethylchloroformate with boron sodium hydride. These N-acyl-2-indolylmethanol derivatives can be converted to the final compounds I having an indole as B, as described for intermediate 9 (Scheme 3).

The compounds where Y is a CH bearing a double bond can be obtained as described in U.S. Pat. No. 3,929,784 by the method of Reissert starting from a proper quinoline, for example, or other known method or its variations. For example, reacting a substituted quinoline with benzoyl chloride and potassium cyanide one can obtain 1-benzoyl-2-cyano(substituted)-1,2-dihydroquinoline, or by reaction with benzoyl chloride and diethyl malonate (CA76:59409) followed by hydrolysis/decarboxylation, one can prepare the 2-quinoline acetic derivative. Afterwards, classical reaction methodologies can afford the same type of intermediates described for the tetrahydro compounds.

An alternative procedure to obtain the compounds I of the invention, especially amenable when dealing with optically active compounds, is described in Scheme 4.

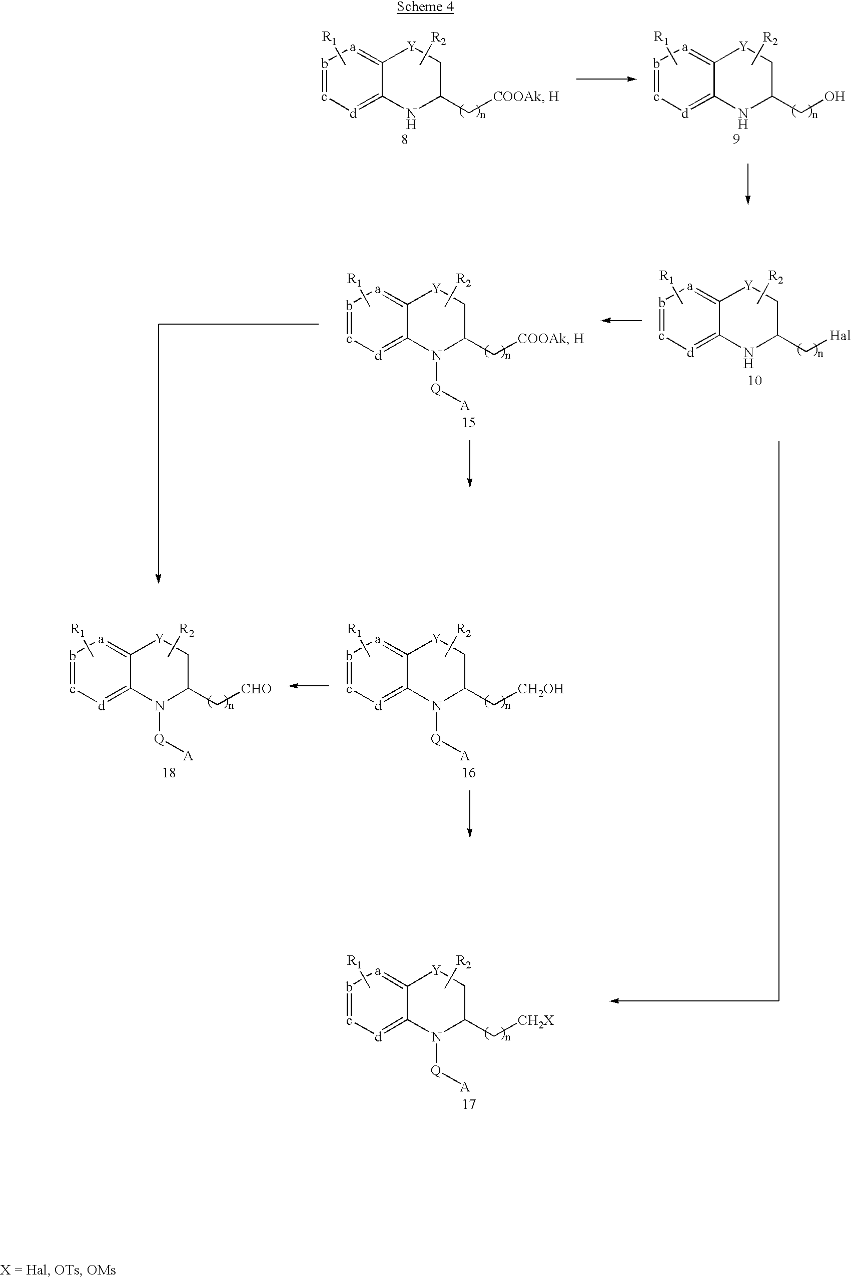

The NH group of Intermediates 8 and 11 can be acylated by standard fashions (see above for the acylation of Intermediates 1) to give polyfunctionalized compounds 15 which, in turn, can be selectively reduced by methods known from the literature (e.g. lithium borohydride, sodium borohydride in alcohols or water, sodium borohydride-lithium chloride or sodium borohydride-calcium chloride, calcium borohydride, borane-THF complex or borane-dimethyl sulphide complex or, only when 8 or 11 is a carboxylic acid, generating the mixed anhydride with a chloroformate and a base and reducing it with sodium borohydride). Alternatively alcohols 16 can be prepared by reacting Intermediates 9 with excess acylating reagent to afford the corresponding O,N-diacylated derivatives, which in turn can be selectively O-monohydrolized by known methods. The so obtained alcohols 16 can be converted into the halo derivative 17 by standard halogenation procedures (SOCl₂, PBr₃ or other) or by Mitsunobu reaction (R. Nagata or CCl₄, triphenylphosphine/chlorinated solvent or other, r.t.—reflux). 17 can be reacted through classical nucleophilic substitution methods very well known to those skilled in the art with the proper amine to afford compounds I. Compounds 17 can also be prepared carrying out the acylation reactions on intermediate 10.

An alternative procedure to obtain the compounds I of the invention consists in reacting in a reductive amination fashion the aldehyde 18 with amines 14. The Intermediate 18 can be generated by selectively reducing compounds 15 (e.g. with diisobutylaluminumhydride or by Rosemund reaction (Bull. Chem. Soc. Jpn. 58(11), 3337-45, (1985)) or other methods (J. Org. Chem. 64(24), 8962-8964, (1999) or J. Org. Chem. 51(5), 705-12, (1986)). Alternatively, alcohols 16 can be oxidized to compounds 18 by known methods (e.g., Swern's oxidation or DMSO-based oxidation methods (Synthesis, 857 (1990) or pyridinium dichromate or Martin's reagent or manganese dioxide).

Compounds 15 are very useful intermediate for the synthesis of compounds 15 where R₂ is a 2-alkyl group. This group can be introduced by a proton abstraction at the 2-position of the tetrahydroquinoline ring with a strong base (e.g., NaNH₂, NaH, BuLi etc.) and alkylation with the proper alkyl halogenide (see Bioorg. Med. Chem. Lett. 5, 1527, (1995)). Compounds (I) where R₁ is aryl or heteroaryl can be obtained by very well known Suzuki coupling of compounds (I) with R₁=Br. The chemical procedures are very well documented in the experimental part.

The N-oxides of compounds I may be synthesised by simple oxidation procedures known to those skilled in the art. The oxidation procedure described by Brougham P., Synthesis, 1015-1017 (1987), allows differentiation of the two nitrogen atoms of the piperazine ring, permitting both the N-oxides and N,N′-dioxide to be obtained.

The above compounds I, (tetrahydro or dihydroquinoline and 2,3-dihydroindole compounds), carry a stereocenter at position 2 of the hetero ring and can be obtained as two enantiomers.

Resolution of the racemic form of (I) can be carried out by fractional crystallization of the diastereoisomeric salt prepared by salification of (I) with an optically-active acid or by preparative-chiral-column chromatography methods as described in the experimental part.

The enantiomers of (I) can be obtained by stereospecific synthesis starting from the homochiral compounds 8 (Scheme 3). These can be obtained by known methods which include N-derivatization of the esters with chiral compounds, separation by chromatographic column or fractional crystallization or hydrolysis of the diastereomeric-acid mixture followed by fractional crystallization and deprotection (M. Paglialunga et al, J. Chem. Soc. Perkin Trans. I., 596-600, (1976)). Alternatively direct fractional crystallization of the salt of the N-protected acids 8 with optically-active bases can be performed (D. E. Portlock U.S. Pat. No. 4,461,896).

The homochiral compounds 8 (Scheme 3) can also be obtained by enzymatic resolution (S. Katayama et al., Tetrahedron Asymmetry 9, 4295-4299, (1998)).

Preparation of the intermediate cyclic amines 14, not yet known in the literature, uses synthesis procedures very well-known to those skilled in the art, which comprise the synthesis of a proper aniline through standard reactions and the subsequent cyclization with bis-(2-chloroethyl)amine to afford piperazine following the method of Prelog V. et al., Collect. Czech. Chem. Comm. 5, 497-502 (1933)) or its variations (Elworthy T. R., J. Med. Chem. 40, 2674-2687 (1997)).

Preparation of the intermediate aryl or heterocyclopiperidines not commercially available can be carried out by known methods which include reaction of aryl or heterocycle organometallic compounds with N-protected piperidones (e.g. carbobenzyloxy or tertbutoxycarbonyl piperidones or ethoxycarbonyl piperidones) to afford compounds 4-aryl or heterocyclopiperidinol which can be dehydrated and reduced or deoxygenated to compounds 14. Alternatively, N-protected piperidones (e.g. carbobenzyloxy and tertbutoxycarbonyl or ethoxycarbonyl piperidones) can be converted to their silyl enol ethers and reacted, by palladium-catalyzed reactions, with aryl or heterocycle organometallic compounds or aryl or heterocycloboronic acids or esters to afford the ene compounds which are then reduced to compounds 14.

The other required amines 14 can in turn be prepared by known methods.

The following additional description and the examples illustrate without limitations the invention and the manner and process of carrying it out to enable those skilled in the art to use the same, and set forth the best mode contemplated by the inventors of carrying out the invention.

Notes and Abbreviation

The main abbreviations or codes used in the experimental part of this patent application are listed below accompanied by their meanings: CHCl₃ stands for alcohol-free chloroform; CH₂Cl₂ stands for dichloromethane; CCl₄ stands for carbon tetrachloride; 1,2-DCE stands for 1,2-dichloroethane; THF stands for tetrahydrofuran; Et₂O stands for diethyl ether; EtOAc stands for ethyl acetate; EtOH stands for ethanol; MeOH stands for methanol; DMF stands for N,N-dimethyl formamide; MeCN stands for acetonitrile; DIPEA stands for N,N-diisopropylethylamine; TEA stands for triethylamine; Na₂SO₄ stands for sodium sulphate; Ph₃P stands for triphenylphosphine; NaOH stands for sodium hydroxide; H₂O stands for water; PtO₂ stands for platinum dioxide; NBS stands for N-bromosuccinimide; AIBN stands for azo-bis-isobutyronitrile.

An alternative procedure to obtain the compounds I of the invention, especially amenable when dealing with optically active compounds, is decribed in Scheme 4.

### Detailed Synthesis

Below are some examples which illustrate the invention as described herein, without in any way meaning to limit it. Product characterization was carried out in the following conditions: ¹H-NMR spectrum (200 MHz) (CDCl₃) if not otherwise specified or (400 MHz) (DMSO-d₆)

### EXAMPLE 1

01-(1-Cyclohexanecarbonyl-1,2,3,4-tetrahydroquinoline-2-ylmethyl)-4-(4-indolyl)piperazine,

(+)-1-(1-Cyclohexanecarbonyl-1,2,3,4-tetrahydroquinoline-2-ylmethyl)-4-(4-indolyl)piperazine,

(−)-1-(1-Cyclohexanecarbonyl-1,2,3,4-tetrahydroquinoline-2-ylmethyl)-4-(4-indolyl)piperazine

a) 2-Chloromethyl-1,2,3,4-tetrahydroquinoline (Compound 1A)

A solution of 0.13 g of 2-hydroxymethyl-1,2,3,4-tetrahydroquinoline (prepared as described in Nagata R. et al., J. Med. Chem. 37, 3956-3968 (1994)), 0.39 g of Ph₃P in 5 ml of CHCl₃ and 3 ml of CCl₄ was stirred at 5° C. for 6 hours. The mixture was evaporated to dryness and the residue was purified by flash chromatography (petroleum ether—ethyl acetate 97:3) to give 0.09 g (66%) of the title compound.

¹H-NMR (δ): 1.00-2.00 (b, 1H), 1.65-1.85 (m, 1H), 1.90-2.10 (m, 1H), 2.60-2.95 (m, 2H), 3.45-3.70 (m, 3H), 6.50-6.70 (m, 2H), 6.90-7.10 (m, 2H)

b) 1-(4-Indolyl)-4-(1,2,3,4-tetrahydroquinoline-2-ylmethyl)piperazine (Compound 1B)

0.13 g of Compound 1A and 0.20 g of 1-(4-indolyl)piperazine (WO 99/67237) in 1.5 ml of DMF was stirred at 100° C. under nitrogen atmosphere for 2 hours. The cooled mixture was poured into 20 ml of water and extracted with ethyl acetate (3×5 ml). The organic layer was dried on anhydrous sodium sulphate and evaporated to dryness. The residue was purified by flash chromatography (petroleum ether—ethyl acetate 7:3) to give 0.08 g (32%) of the title compound.

¹H-NMR (δ): 1.45-1.75 (m, 1H), 1.85-2.00 (m, 1H), 2.45-3.10 (m, 8H), 3.20-3.60 (m, 6H), 6.50-6.70 (m, 4H), 6.90-7.05 (m, 2H), 7.05-7.20 (m, 3H), 8.25 (bs, 1H)

c) 1-(1-Cyclohexanecarbonyl-1,2,3,4-tetrahydroquinoline-2-ylmethyl)-4-(4-indolyl)piperazine

A mixture of 0.07 g of Compound 1B, 0.05 ml of DIPEA, 0.04 ml of cyclohexanecarbonyl chloride and 1.5 ml of toluene was stirred at 20 to 25° C. under nitrogen atmosphere for 1 hour. The solution was washed with a 5% aqueous solution of sodium bicarbonate (3×3 ml) and 3 ml of water. The organic layer, dried on anhydrous sodium sulphate, was evaporated to dryness and the residue purified by flash chromatography (dichloromethane—methanol 95:5) to give 0.03 g (33%) of the title compound.

¹H-NMR (δ): 0.80-2.05 (m, 12H), 2.05-2.25 (m, 1H), 2.25-2.85 (m, 8H), 3.10-3.35 (m, 4H), 5.00-5.25 (m, 1H), 6.45-6.60 (m, 2H), 7.00-7.30 (m, 7H), 8.20 (bs, 1H)

Alternatively, the compound of Example 1 can be prepared by the following procedure:

d) 1-(4-Indolyl)-4-(quinoline-2-ylmethyl)piperazine (Compound 1C)

A mixture of 2-chloromethylquinoline (2.56 g), DIPEA (4.16 ml) and 1-(4-indolyl)piperazine (2.65 g) in DMF (4 ml) was heated at 120-130° C. for 3-4 hours. After cooling to room temperature, the mixture was diluted with water (50-60 ml); the liquids were decanted and extracted with diethyl ether (3×40 ml), washed with water, dried on sodium sulphate and filtered; the solid residue was dissolved in dichloromethane, washed with water, dried on sodium sulphate and filtered.

The combined organic layers were evaporated to dryness at reduced pressure. The crude was purified by flash chromatography eluting with ethyl acetate-petroleum ether 75:25 to give 2.87 g (70%) of the desired compound as a solid.

¹H-NMR (δ): 2.65-2.96 (m, 4H), 3.28-3.42 (m, 4H), 3.99 (s, 2H), 6.51-6.66 (m, 2H), 6.98-7.18 (m, 3H), 7.48-7.61 (m, 1H), 7.61-7.89 (m, 3H), 8.07-8.28 (m, 3H)

e) 1-(4-Indolyl)-4-(1,2,3,4-tetrahydroquinoline-2-ylmethyl)piperazine (Compound 1B)

A mixture of Compound 1C (2.87 g), PtO₂ (101 mg) and acetic acid (50 ml) was hydrogenated at a hydrogen pressure of 15 psi in a Parr apparatus at room temperature. The catalyst was separated by filtration and washed with methanol. The solvent was evaporated at reduced pressure, the residue was taken up with dichloromethane (20 ml) and alkalinised with 1N sodium hydroxide (pH>8). The aqueous extract was extracted twice with dichloromethane and the combined organic layers were dried on sodium sulphate, filtered and evaporated in vacuo. The crude was purified by flash chromatography eluting with dichloromethane—methanol 97:3 to give 2.22 g (76.5%) of the title compound.

f) 1-(1-Cyclohexanecarbonyl-1,2,3,4-tetrahydroquinoline-2-ylmethyl)-4-(4-indolyl)piperazine

Cyclohexanecarbonyl chloride (1.03 ml) was added to a solution of Compound 1B (2.22 g), TEA (1.33 ml) in dichloromethane (30 ml) cooled to 0° C., under nitrogen atmosphere. After 2-3 hours of stirring at room temperature, the resulting mixture was washed with 1N sodium hydroxide (pH>8) and the organic phase was dried on sodium sulphate, filtered and evaporated in vacuo.

The crude was purified by flash chromatography eluting with toluene-acetone 8:2 affording 2.41 g (81%) of the title compound as a pale yellow powder. Crystallisation from MeCN afforded the title compound as a white solid. M.p. 180-181° C.

Alternatively the crude compound was purified directly by crystallisation from MeCN affording the pure title compound.

Alternatively Compound 1B can be prepared by the synthetic procedure described below:

g) 1-(4-Indolyl)-4-(1,2,3,4-tetrahydroquinoline-2-ylcarbonyl)piperazine (Compound 1D)

TEA (0.48 ml) and diethyl cyanophosphonate (0.53 ml) were added to a stirred solution of 1,2,3,4-tetrahydroquinoline-2-carboxilic acid (0.594 g) and 1-(4-indolyl)piperazine (0.675 g) in DMF (24 ml) at 0° C. The reaction mixture was stirred at room temperature for 3 hours. It was diluted with H₂O (300 ml) and the white precipitate which formed was filtered and dried on sodium sulphate by dissolving in CH₂Cl₂, and the solution was evaporated to dryness in vacuo. The residue was purified by flash chromatography eluting with petroleum ether—EtOAc 1:1 to give 0.7 g (58%) of the title compound.

¹H-NMR (δ): 1.60-1.90 (m, 1H), 2.10-2.25 (m, 1H), 2.70-3.00 (m, 2H), 3.20-3.35 (m, 4H), 3.65-4.10 (m, 4H), 4.40 (dd, 1H), 4.50 (s, 1H), 6.51-6.75 (m, 4H), 6.95-7.30 (m, 5H), 8.25 (s, 1H)

h) 1-(4-Indolyl)-4-(1,2,3,4-tetrahydroquinoline-2-ylmethyl)piperazine (Compound 1B)

10M Borane-dimethylsulphide (0.21 ml) was added to a stirred solution of Compound 1D (0.25 g) in THF (5 ml) at 0° C. The solution was refluxed for 1 hour, cooled at room temperature and diluted with a saturated solution of ammonium chloride. The resulting solution was extracted with CH₂Cl₂ (20 ml), dried on sodium sulphate and evaporated in vacuo. The residue was purified by flash chromatography eluting with petroleum ether—EtOAc 1:1 to give 0.12 g (49%) of the title compound.

(+)-1-(1-Cyclohexanecarbonyl-1,2,3,4-tetrahydroquinoline-2-ylmethyl)-4-(4-indolyl)piperazine and

(−)-1-(1-Cyclohexanecarbonyl-1,2,3,4-tetrahydroquinoline-2-ylmethyl)-4-(4-indolyl)piperazine

The racemic compound of Example 1 was subjected to chiral-column chromatography (Chiralpack AD 0.46×25 cm; eluent 0.1% diethylamine in ethanol; flow 65 ml/min; λ=235 nM affording the two enantiomers:

Ex. (+)-1 α_{D}: +142° (c=0.5, CHCl₃)

Ex. (−)-1 α_{D}: −147° (c=0.57, CHCl₃)

The compound of Example (+)-1 was converted into the corresponding monomethanesulphonate salt by traditional methods and, after crystallisation from i-propanol and mixtures of i-propanol-water 4:1, 1:1 and 1:4, showed a DSC melting peak of 143° C.

Similarly, after crystallisation from mixtures of ethanol-water 4:1, 1:1 and 1:4, it showed a DSC melting peak of 151° C.

### EXAMPLE 2

1-(1-Cyclohexanecarbonyl-1,2,3,4-tetrahydroquinoline-2-ylmethyl)-4-(2-methoxyphenyl)piperazine

a) 2-[4-(2-Methoxyphenyl)-1-piperazinylmethyl]quinoline (Compound 2A)

The title compound was prepared as described for Compound 1C substituting 1-(4-indolyl)piperazine with 1-(2-methoxyphenyl)piperazine. Purification was carried out by flash chromatography eluting with petroleum ether-ethyl acetate 4:6 to give the title compound (63.6%).

¹H-NMR (δ): 2.69-2.86 (m, 4H), 2.98-3.27 (m, 4H), 3.85 (s, 2H), 3.97 (s, 3H), 6.79-7.04 (m, 4H), 7.49-7.58 (m, 1H), 7.63-7.79 (m, 3H), 8.03-8.21 (m, 2H)

b) 1-(1,2,3,4-Tetrahydroquinoline-2-ylmethyl)-4-(2-methoxyphenyl)piperazine (Compound 2B)

Compound 2B was obtained in the same way as described for Compound 1B (step e) but starting from Compound 2A instead of Compound 1C. The crude was purified by flash chromatography eluting with dichloromethane-methanol 97:3 to give the title compound (57%).

¹H-NMR (δ): 1.48-1.67 (m, 1H), 1.80-1.99 (m, 1H), 2.47 (m, 2H), 2.51-2.67 (m, 4H), 2.72-2.98 (m, 4H), 3.02-3.27 (m, 4H), 3.37-3.59 (m, 1H), 3.90 (m, 3H), 4.56-4.77 (b, 1H), 6.48-6.67 (m, 2H), 6.83-7.08 (m, 6H)

c) 1-(1-Cyclohexanecarbonyl-1,2,3,4-tetrahydroquinoline-2-ylmethyl)-4-(2-methoxyphenyl)piperazine

The title compound was synthesised by the alternative acylation procedure (step f) described for the compound of Example 1 starting from Compound 2B instead of Compound 1B. The crude was purified twice by flash chromatography eluting with dichloromethane-methanol 95:5 and then with toluene-acetone 85:15 affording the title compound (69%). M.p. 45.5° C. (dec.).

¹H-NMR (δ): 0.86-2.00 (m, 11H), 2.02-2.21 (m, 1H), 2.25-2.81 (m, 9H), 2.92-3.16 (m, 4H), 3.81 (s, 3H), 4.99-5.21 (m, 1H), 6.81-8.04 (m, 4H), 7.08-7.29 (m, 4H)

### EXAMPLE 3

1-[2-(1-Cyclohexanecarbonyl-1,2,3,4-tetrahydroquinoline-2-yl)ethyl]-4-(2-methoxyphenyl)piperazine

a) 1-[2-(1,2,3,4-Tetrahydroquinoline-2-yl)ethyl]-4-(2-methoxyphenyl)piperazine (Compound 3A)

Compound 3A was obtained in the same way as described for Compound 1B (step e) but starting from 1-[2-(2-quinolineyl)ethyl]-4-(2-methoxyphenyl)piperazine (U.S. Pat. No. 3,983,121) instead of Compound 1C. The crude was purified by flash chromatography eluting with dichloromethane-methanol 93:7 to give 0.115 g (43%) of the title compound.

¹H-NMR (δ): 1.54-2.03 (m, 4H), 2.51-2.72 (m, 4H), 2.73-2.97 (m, 4H), 3.01-3.40 (m, 4H), 3.31-3.49 (m, 1H), 3.89 (s, 3H), 6.47 (d, 1H), 6.51-6.62 (m, 1H), 6.81-7.09 (m, 6H)

b) 1-[2-(1-Cyclohexanecarbonyl-1,2,3,4-tetrahydroquinoline-2-yl)ethyl]-4-(2-methoxyphenyl)piperazine

The title compound was synthesised following the alternative acylation procedure (step f) described for the compound of Example 1 starting from Compound 3A instead of Compound 1B. The crude was purified twice by flash chromatography eluting first with dichloromethane-methanol 94:6, then with ethyl acetate-2N methanolic ammonia 98:2, affording 0.13 g of the title compound as a solid containing cyclohexanecarboxylic acid as the main impurity. This solid was dissolved in dichloromethane (5 ml) and washed with 1N sodium hydroxide (2×3 ml). The organic part was dried on sodium sulphate, filtered and evaporated at reduced pressure to give 0.091 g (62%) of the designated compound.

¹H-NMR (δ): 0.89-2.00 (m, 13H), 2.23-2.81 (m, 10H), 2.94-3.15 (m, 4H), 3.87 (s, 3H), 4.78-5.04 (m, 1H), 6.76-7.28 (m, 8H)

### EXAMPLE 4

1-(1-Cyclohexanecarbonyl-1,2,3,4-tetrahydroquinoline-2-ylmethyl)-4-[2-(2,2,2-trifluoroethoxy)phenyl]piperazine

a) 2-[4-[2-(2,2,2-Trifluoroethoxy)phenyl]-1-piperazinylmethyl]quinoline (Compound 4A)

Compound 4A was obtained in the same way as described for Compound 1C (procedure d) but using 1-[2-(2,2,2-trifluoroethoxyphenyl)]piperazine instead of 1-(4-indolyl)piperazine. The crude was purified by flash chromatography eluting with petroleum ether-ethyl acetate 55:45 to give the title compound (88%).

¹H-NMR (δ):2.60-2.83 (m, 4H), 3.06-3.27 (m, 4H), 3.91 (s, 2H), 4.40 (q, 4H), 6.88-7.09 (m, 4H), 7.46-7.62 (m, 1H), 7.66-7.85 (m, 3H), 8.03-8.21 (m, 2H)

b) 1-(1,2,3,4-Tetrahydroquinoline-2-ylmethyl)-4-[2-(2,2,2-trifluoroethoxy)phenyl]piperazine (Compound 4B)

The title compound was prepared as described for Compound 1B (procedure e) starting from Compound 4A instead of Compound 1C. The crude was purified by flash chromatography eluting with dichloromethane-methanol 97:3 to give the title compound (61%).

¹H-NMR (δ): 1.45-1.71 (m, 1H), 1.82-2.00 (m, 1H), 2.40-2.64 (m, 4H), 2.69-2.91 (m, 4H), 3.04-3.26 (m, 4H), 3.37-3.57 (m, 1H), 4.41 (q, 2H), 4.56-4.76 (br, 1H), 6.48-6.67 (m, 2H), 6.85-7.09 (m, 6H)

c) 1-(1-Cyclohexanecarbonyl-1,2,3,4-tetrahydroquinoline-2-ylmethyl)-4-[2-(2,2,2-trifluoroethoxy)phenyl]piperazine

The title compound was synthesised following the alternative acylation procedure described for the compound of Example 1 (step f) but starting from Compound 4B instead of Compound 1B. The crude was purified by flash chromatography eluting with petroleum ether-ethyl acetate 7:3 to give the title compound (89%). M.p. 46° C. (dec.).

¹H-NMR (δ): 0.81-1.99 (m, 12H), 2.01-2.19 (m, 1H), 2.24-2.78 (m, 8H), 2.92-3.13 (m, 4H), 4.39 (q, 2H), 4.99-5.20 (m, 1H), 6.84-7.28 (m, 8H)

### EXAMPLE 5

1-(1-Cyclohexanecarbonyl-6-fluoro-1,2,3,4-tetrahydroquinoline-2-ylmethyl)-4-[2-(2,2,2-trifluoroethoxy)phenyl]piperazine

a) 2-Bromomethyl-6-fluoroquinoline (Compound 5A)

A mixture of 6-fluoro-2-methylquinoline (3 g), NBS (1.65 g), AIBN (25 mg) and CCl₄ (10 ml) was refluxed for 5 hours, then additional NBS (0.825 g) was added and the mixture was refluxed for 5 hours. After cooling at room temperature, the solid was separated by filtration and the filtrate evaporated at reduced pressure. The crude was purified by flash chromatography eluting with petroleum ether-ethyl acetate 85:15 to give, as a first eluted product, 0.71 g (17%) of the title compound and 2.13 g of starting material.

¹H-NMR (δ): 4.71 (s, 2H), 7.36-7.65 (m, 3H), 8.00-8.19 (m, 2H)

b) 6-Fluoro-2-[4-[2-(2,2,2-trifluoroethoxy)phenyl]-1-piperazinylmethyl]quinoline (Compound 5B)

Compound 5B was obtained in the same way as described for Compound 1C (procedure d) but using 1-[2-(2,2,2-trifluoroethoxy)phenyl)]piperazine instead of 1-(4-indolyl)piperazine and Compound 5A instead of 2-chloromethylquinoline. The crude was purified by flash chromatography eluting with petroleum ether-ethyl acetate 6:4 to give the title compound (80%).

¹H-NMR (δ): 2.67-2.86 (m, 4H), 3.05-3.26 (m, 4H), 3.91 (s, 2H), 4.41 (q, 4H), 6.88-7.09 (m, 4H), 7.37-7.56 (m, 2H), 7.71 (d, 3H), 7.99-8.18 (m, 2H)

c) 1-(6-Fluoro-1,2,3,4-tetrahydroquinoline-2-ylmethyl)-4-[2-(2,2,2-trifluoroethoxy -phenyl]piperazine (Compound 5C)

The title compound was prepared as described for Compound 1B (procedure e) starting from Compound 5B instead of Compound 1C. The crude was purified by flash chromatography eluting with petroleum ether-ethyl acetate 8:2 to give the title compound (57%).

¹H-NMR (δ): 1.42-1.80 (m, 1H), 1.80-1.99 (m, 1H), 2.39-2.61 (m, 4H), 2.63-2.94 (m, 4H), 3.02-3.26 (m, 4H), 3.31-3.52 (m, 1H), 4.41 (q, 2H), 4.41-4.68 (br, 1H), 6.37-6.54 (m, 1H), 6.61-6.79 (m, 2H), 6.88-7.11 (m, 6H)

d) 1-(1-Cyclohexanecarbonyl-6-fluoro-1,2,3,4-tetrahydroquinoline-2-ylmethyl)-4-[2-(2,2,2-trifluoroethoxy)phenyl]piperazine

The title compound was synthesised following the alternative acylation procedure described for the compound of Example 1 (step f) but starting from Compound 5C instead of Compound 1B. The crude was purified by flash chromatography eluting with petroleum ether-ethyl acetate 7:3 affording the title compound (92%). M.p. 51° C.

¹H-NMR (δ): 0.80-1.99 (m, 11H), 2.00-2.17 (m, 1H), 2.21-2.77 (m, 9H), 2.91-3.12 (m, 4H), 4.40 (q, 2H), 4.98-5.22 (m, 1H), 6.83-7.16 (m, 8H)

### EXAMPLE 6

1-[1-(2-Ethylbutanoyl)-1,2,3,4-tetrahydroquinoline-2-ylmethyl]-4-[2-(2,2,2-trifluoroethoxy)phenyl]piperazine

The title compound was synthesised following the alternative acylation procedure described for the compound of Example 1 (step f) but starting from Compound 4B instead of Compound 1B and using 2-ethylbutanoyl chloride instead of cyclohexanecarbonyl chloride. The crude was purified by flash chromatography eluting with petroleum ether-ethyl acetate 75:25 affording the title compound (93%).

¹H-NMR (δ): 0.61 and 1.05 (2t, 6H), 1.21-1.67 (m, 5H), 1.74-1.93 (m, 1H), 2.00-2.19 (m, 1H), 2.23-2.84 (m, 8H), 2.92-3.24 (m, 4H), 4.41 (m, 2H), 5.08-5.29 (m, 1H), 6.87-7.29 (m, 8H)

### EXAMPLE 7

1-[1-(3-Methoxypropionyl)-1,2,3,4-tetrahydroquinoline-2-ylmethyl]-4-[2-(2,2,2-trifluoroethoxy)phenyl]piperazine

The title compound was synthesised following the alternative acylation procedure described for the compound of Example 1 (step f) but starting from Compound 4B instead of Compound 1B and using 3-methoxypropionyl chloride instead of cyclohexanecarbonyl chloride. The crude was purified by flash chromatography eluting with petroleum ether-ethyl acetate 4:6 affording the title compound (56.5%).

¹H-NMR (δ): 1.41-1.55 (m, 1H), 2.04-2.21 (m, 1H), 2.31-2.95 (m, 10H), 2.97-3.16 (m, 4H), 3.32 (s, 3H), 3.71 (t, 2H), 4.41 (q, 2H), 5.06-5.21 (m, 1H), 6.88-7.08 (m, 4H), 7.00-7.18 (m, 4H)

### EXAMPLE 8

1-[1-(3-Benzyloxypropionyl)-1,2,3,4-tetrahydroquinoline-2-ylmethyl]-4-[2-(2,2,2-trifluoroethoxy)phenyl]piperazine

The title compound was synthesised following the alternative acylation procedure described for the compound of Example 1 (step f) but starting from Compound 4B instead of Compound 1B and using 3-benzyloxypropionyl chloride instead of cyclohexanecarbonyl chloride. The crude was purified by flash chromatography eluting with toluene-acetone 7.5:21.5 affording the title compound (48%).

¹H-NMR (δ): 1.41-1.60 (m, 1H), 2.05-2.21 (m, 1H), 2.25-2.75 (m, 9H), 2.80-3.16 (m, 5H), 3.70-3.90 (m, 2H), 4.41 (q, 2H), 4.50 (s, 2H), 5.00-5.21 (m, 1H), 6.82-7.08 (m, 4H), 7.10-7.40 (m, 4H)

### EXAMPLE 9

1-[1-(3-Benzyloxypropionyl)-1,2,3,4-tetrahydroquinoline-2-ylmethyl]-4-(4-indolyl)piperazine

The title compound was synthesised following the alternative acylation procedure described for the compound of Example 1 (step f) using 3-benzyloxypropionyl chloride instead of cyclohexanecarbonyl chloride. The crude was purified by flash chromatography eluting with toluene-acetone 7.5:2.5 to give the title compound (58%).

¹H-NMR (δ): 1.41-1.65 (m, 1H), 2.10-2.30 (m, 1H), 2.32-3.00 (m, 10H), 3.10-3.30 (m, 4H), 3.72-3.92 (m, 2H), 4.50 (s, 2H), 5.05-5.25 (m, 1H), 6.45-6.65 (m, 2H), 7.00-7.40 (m, 12H), 8.15 (s, 1H)

### EXAMPLE 10

1-[1-(3-Hydroxypropionyl)-1,2,3,4-tetrahydroquinoline-2-ylmethyl]-4-[2-(2,2,2-trifluoroethoxy)phenyl]piperazine

10% Pd-C (0.05 mg) and ammonium formate (0.16 g) was added to a solution of the compound of Example 8 (0.202 g) in MeOH (8 ml). The reaction mixture was refluxed for 6 hours under nitrogen atmosphere. Additional 0.2 g of ammonium formate and 0.1 g of 10% Pd-C was added. The mixture was refluxed for 4 additional hours. The catalyst was filtered and evaporation in vacuo was carried out. The crude was purified by flash chromatography eluting with CHCl₃-MeOH 97:3 to obtain the title compound (48%).

¹H-NMR (δ): 1.30-2.00 (m, 2H), 2.04-2.21 (m, 1H), 2.25-2.80 (m, 9H), 2.80-3.00 (m, 1H), 3.00-3.15 (m, 4H), 3.75-4.00 (m, 2H), 4.40 (q, 2H), 5.05-5.31 (m, 1H), 6.82-7.08 (m, 4H), 7.10-7.35 (m, 4H)

### EXAMPLE 11

1-[1-(3-Methoxypropionyl)-1,2,3,4-tetrahydroquinoline-2-ylmethyl]-4-(4-indolyl)piperazine

The title compound was synthesised following the alternative acylation procedure described for the compound of Example 1 (step f) using 3-methoxypropionyl chloride instead of cyclohexanecarbonyl chloride. The crude was purified by flash chromatography eluting with petroleum ether-ethyl acetate 3:7 to give the title compound (44%). M.p. 62.8-66° C.

¹H-NMR (δ): 1.41-1.65 (m, 1H), 2.10-2.25 (m, 1H), 2.30-2.95 (m, 10H), 3.12-3.25 (m, 4H), 3.32 (s, 1H), 3.70 (t, 2H), 5.00-5.25 (m, 1H), 6.45-6.60 (m, 2H), 7.00-7.30 (m, 12H), 8.15 (s, 1H)

### EXAMPLE 12

1-[1-(3-Isopropoxypropionyl)-1,2,3,4-tetrahydroquinoline-2-ylmethyl]-4-(4-indolyl)piperazine

The title compound was synthesised following the alternative acylation procedure described for the compound of Example 1 (step f) using 3-isopropoxypropionyl chloride instead of cyclohexanecarbonyl chloride. The crude was purified by flash chromatography eluting with petroleum ether-ethyl acetate 1:1 to give the title compound (20%).

¹H-NMR (δ): 1.12 (d, 6H), 1.41-1.65 (m, 1H), 2.10-2.25 (m, 1H), 2.30-3.00 (m, 10H), 3.12-3.25 (m, 4H), 3.55 (heptet, 1H), 3.65-3.90 (m, 1H), 5.00-5.25 (m, 1H), 6.45-6.60 (M, 2H), 7.00-7.35 (m, 12H), 8.15 (s, 1H)

### EXAMPLE 13

1-[1-Acetyl-1,2,3,4-tetrahydroquinoline-2-ylmethyl]-4-(4-indolyl)piperazine

The title compound was synthesised following the alternative acylation procedure described for the compound of Example 1 (step f) using acetyl chloride instead of cyclohexanecarbonyl chloride and CHCl₃ instead of dichloromethane. The crude was purified by flash chromatography eluting with petroleum ether-ethyl acetate-2.5N methanolic ammonia 3:7:0.1 to give the title compound (53%). M.p. 187-189° C.

¹H-NMR (δ): 1.50-1.70 (m, 1H), 2.05-2.29 (m, 4H), 2.31-2.85 (m, 8H), 3.10-3.32 (m, 4H), 4.98-5.21 (m, 1H), 6.48-6.65 (m, 2H), 7.00-7.30 (m, 7H) 8.15, s, 1H

### EXAMPLE 14

1-[1-(4-Morpholinocarbonyl)-1,2,3,4-tetrahydroquinoline-2-ylmethyl]-4-(4-indolyl)piperazine

The title compound was synthesised following the alternative acylation procedure described for the compound of Example 1 (step f) using 4-morpholinocarbonyl chloride instead of cyclohexanecarbonyl chloride and toluene instead of dichloromethane, and refluxing for 2.5 hours. The crude was purified by flash chromatography eluting with petroleum ether-ethyl acetate-2.5N methanolic ammonia 8:2:0.01 to give the title compound (26%). M.p. 195-205° C.

¹H-NMR (δ): 1.90-2.21 (m, 2H), 2.35 (d, 1H), 2.55-2.90 (m, 7H), 3.12-3.42 (m, 8H), 3.48-3.75 (m, 4H), 4.38-4.50 (m, 1H), 6.48-6.60 (m, 2H), 6.85-7.20 (m, 7H), 8.15 (s, 1H)

### EXAMPLE 15

1-(1-Cyclohexanecarbonyl-1,2,3,4-tetrahydroquinoline-2-ylmethyl)-4-(1-methyl-4-indolyl)piperazine

The compound of example 1 (0.456 g) was added to a suspension of 50% NaH (0.130 g) in DMF (20 ml), and stirred for 20 minutes at room temperature and then 1 hour at 55° C.

Methyl iodide (0.185 ml) was added dropwise, stirred for 4 hours at room temperature, and poured into H₂O. The precipitate was filtered, dissolved in Et₂O, washed with H₂O, dried on sodium sulphate and evaporated in vacuo. The crude was purified by flash chromatography eluting with CH₂Cl₂-MeOH 9.7:0.3 to give 0.077 g of the title compound (16%). M.p. 66-70° C.

¹H-NMR (δ): 0.80-2.05 (m, 11H), 2.06-2.25 (m, 1H), 2.25-2.85 (m, 8H), 3.10-3.35 (m, 4H), 5.00-5.25 (m, 1H), 6.45-6.60 (m, 2H), 7.00-7.30 (m, 7H), 8.20 (b, 1H).

### EXAMPLE 16

1-(1-Cyclohexanecarbonyl-6-methoxy-1,2,3,4-tetrahydroquinoline-2-ylmethyl)-4-(4-indolyl)piperazine

a) 2-Bromomethyl-6-methoxyquinoline (Compound 16A)

Compound 16A was obtained in the same way as described for Compound 5A (procedure a) but using 6-methoxy-2-methylquinoline instead of 6-fluoro-2-methylquinoline. The crude was purified by flash chromatography eluting with petroleum ether—ethyl acetate 85:15 to give, as a first eluted product, 0.45 g (30%) of the title compound, followed by 0.39 g of starting material.

¹H-NMR (δ): 3.93 (s, 3H), 4.70 (s, 2H), 7.09 (d 1H), 7.37 (dd, 1H), 7.37 (d, 1H), 7.96 (d, 1H), 8.09 (d, 1H).

b) 6-Methoxy-2-[4-(4-indolyl)-1-piperazinylmethyl]quinoline (Compound 16B)

Compound 16B was obtained in the same way as described for Compound 1C (procedure d) but using Compound 16A instead of 2-chloromethylquinoline. The crude was purified by flash chromatography eluting with methylene chloride-methanol 95:5 to give the title compound (80%).

¹H-NMR (δ): 2.73-2.91 (m, 4H), 3.21-3.42 (m, 4H), 3.86-4.02 (m, 5H), 6.49-6.67 (m, 2H), 7.00-7.21 (m, 4H), 7.36 (d, 1H), 7.65(d, 1H), 7.91-8.10 (m, 2H), 8.12-8.33 (br, 1H).

c) 1-(6-Methoxy-1,2,3,4-tetrahydroquinoline-2-ylmethyl)-4-(4-indolyl)piperazine (Compound 16C)

The title compound was prepared as described for Compound 1B (procedure e) starting from Compound 16B instead of Compound 1C. The crude was purified by flash chromatography eluting with dichloromethane-methanol 97:3 to give the title compound (48%).

¹H-NMR (δ): 1.47-1.72 (m, 1H), 1.81-2.01 (m, 1H), 2.36-2.53 (m, 2H), 2.54-2.74 (m, 2H), 2.75-2.98 (m, 4H), 3.18-3.57 (m, 5H), 3.73 (s, 3H), 4.71-4.83 (sa, 1H), 6.47-6.63 (m, 5H), 7.02-7.18 (m, 3H), 8.11-8.26 (br, 1H)

d) 1-(1-Cyclohexanecarbonyl-6-methoxy-1,2,3,4-tetrahydroquinoline-2-ylmethyl]-4-(4-indolyl)piperazine

The title compound was synthesised following the alternative acylation procedure described for the compound of Example 1 (step f) but starting from Compound 16C instead of Compound 1B. The crude was purified by flash chromatography eluting with dichloromethane-methanol 97:3 to give the title compound (89%). M.p. 90-93° C.

¹H-NMR (δ): 0.75-2.00 (m, 11H), 2.01-2.21 (m, 1H), 2.22-2.84 (m, 9H), 3.11-3.34 (m, 4H), 3.76 (s, 3H), 5.01-5.23 (m, 1H), 6.46-6.64 (m, 2H), 6.70-6.83 (m, 2H), 6.95-7.19 (m, 4H), 8.07-8.22 (b, 1H).

### EXAMPLE 17

1-[1-(3-Hydroxypropionyl)-1,2,3,4-tetrahydroquinoline-2-ylmethyl]-4-(4-indolyl)piperazine

The title compound was prepared following the procedure described for the compound of Example 10, but using the compound of Example 9 instead of the compound of Example 8. The crude was purified by flash chromatography eluting with CHCl₃-MeOH 97:3 to give the title compound (63%). M.p. 58-61° C.

¹H-NMR (δ): 1.30-2.30 (br, 1H), 1.41-1.60 (m, 1H), 2.10-2.27 (m, 1H), 2.30-2.99 (m, 10H), 3.11-3.31 (m, 4H), 3.72-3.93 (m, 2H), 5.05-5.28 (m, 1H), 6.50-6.67 (m, 2H), 7.01-7.28 (m, 7H), 8.01-8.25 (br, 1H)

### EXAMPLE 18

1-(1-Cyclohexanecarbonyl-6-fluoro-1,2,3,4-tetrahydroquinoline-2-ylmethyl)-4-(4-indolyl)piperazine

a) 6-Fluoro-2-[4-(4-indolyl)-1-piperazinylmethyl]quinoline (Compound 18A)

Compound 18A was obtained in the same way as described for Compound 1C (procedure d) but using Compound 5A instead of 2-chloromethylquinoline. The crude was purified by flash chromatography eluting with petroleum ether-ethyl acetate 1:1 to give the title compound (63%).

¹H-NMR (δ): 2.65-2.91 (m, 4H), 3.12-3.41 (m, 4H), 3.93 (s, 2H), 6.49-6.62 (m, 2H), 7.00-7.21 (m, 3H), 7.38-7.52 (m, 2H), 7.65-7.80 (m, 1H), 7.93-8.12 (m, 2H), 8.15-8.30 (br, 1H)

b) 1-(6-Fluoro-1,2,3,4-tetrahydroquinoline-2-ylmethyl)-4-(4-indolyl)piperazine (Compound 18B)

The title compound was prepared as described for Compound 1B (procedure e) starting from Compound 18A instead of Compound 1C. The crude was purified by flash chromatography eluting with petroleum ether-ethyl acetate 6:4 to give the title compound (59%).

¹H-NMR (δ): 1.45-1.68 (m, 1H), 1.80-1.93 (m, 1H), 2.43 (d, 2H), 2.51-2.69 (m, 2H), 2.81-3.01 (m, 4H9, 3.12-3.33 (m, 4H), 3.34-3.51 (m, 1H), 4.20-5.00 (br, 1H), 6.40-6.73 (m, 5H), 7.00-7.19 (m, 3H), 8.07-8.27 (br, 1H)

c) 1-(1-Cyclohexanecarbonyl-6-fluoro-1,2,3,4-tetrahydroquinoline-2-ylmethyl)-4-(4-indolyl)piperazine

The title compound was synthesised following the alternative acylation procedure described for the compound of Example 1 (step f) but starting from Compound 18B instead of Compound 1B. The crude was purified by flash chromatography eluting with petroleum ether-ethyl acetate 6:4 to give the title compound (49%). M.p. 82-84° C.

¹H-NMR (δ): 0.75-1.93 (m, 11H), 1.95-2.20 (m, 1H), 2.28-2.83 (m, 9H), 3.07-3.30 (m, 4H), 5.02-5.23 (m, 1H), 6.42-6.70 (m, 2H), 6.81-7.28 (m, 6H), 8.04-8.22 (br, 1H)

### EXAMPLE 19

1-(1-Dimethylaminocarbonyl-1,2,3,4-tetrahydroquinoline-2-ylmethyl)-4-(4-indolyl)piperazine

The title compound was synthesised following the alternative acylation procedure described for the compound of Example 1 (step f) using N,N-dimethylaminocarbonyl chloride instead of cyclohexanecarbonyl chloride and toluene instead of dichloromethane, and refluxing for 2 hours. The crude was purified by flash chromatography eluting with petroleum ether-ethyl acetate-2.5 N methanolic ammonia 8:2:0.01 to give the title compound (39%). M.p. 190-227° C.

¹H-NMR (δ): 1.88-2.10 (m, 1H), 2.11-2.27 (m, 1H), 2.28-2.50 (m, 1H), 2.60-2.95 (m, 13H), 3.10-3.48 (m, 4H), 4.32-4.50 (s, 1H), 6.50-6.65 (m, 2H), 6.75-6.98 (m, 2H), 7.00-7.22 (m, 5H), 8.20 (bs, 1H).

### EXAMPLE 20

1-(1-Ethylaminocarbonyl-1,2,3,4-tetrahydroquinoline-2-ylmethyl)-4-(4-indolyl)piperazine

A solution of Compound 1B (0.35 g), ethy isocyanate (0.16 ml) in DMF (2 ml) was stirred at 100° C. for 2 h. Afterwards, the reaction mixture was poured into H₂O and extracted with EtOAc (2×30 ml). The combined extracts were washed with H₂O, dried (Na₂SO₄) and evaporated to dryness in vacuo. The crude was purified by flash chromatography eluting with petroleum ether-ethyl acetate-2.5 N methanolic ammonia 8:2:0.01 to give 0.22 g of the title compound (49.8%). M.p. 109-112° C.

¹H-NMR (δ): 1.18 (t, 3H), 1.58-1.78 (m, 1H), 2.11-2.34 (m, 1H), 2.36 (dd, 1H), 2.58-2.98 (m, 7H), 3.10-3.42 (m, 4H), 4.45-4.65 (s, 1H), 6.47-6.65 (m, 2H), 6.95-7.25 (m, 7H), 7.39 (d, 1H), 8.25 (bs, 1H)

### EXAMPLE 21

1-(1-Cyclohexanecarbonyl-1,2,3,4-tetrahydroquinoline-2-ylmethyl)-4-(1-isoquinolinyl)piperazine

a) 1-(1-Isoquinolinyl)-4-(1,2,3,4-tetrahydroquinoline-2-ylcarbonyl)piperazine (Compound 21A)

The title compound was synthesised following the procedure described for Compound 1D (step g) but using 1-(1-isoquinolinyl)piperazine (WO 0040554) instead of 1-(4-indolyl)piperazine. The residue was purified by flash chromatography eluting with petroleum ether—EtOAc 1:1 to give of the title compound (75.6%)

¹H-NMR (δ): 1.61-1.89 (m, 1H), 2.22-2.30 (m, 1H), 2.78-2.96 (m, 2H), 3.35-3.54 (m, 4H), 3.71-4.10 (m, 4H), 4.29 (dd, 1H), 4.41-4.68 (br, 1H), 6.59-6.76 (m, 2H), 6.95-7.13 (m, 2H), 7.32 (d, 1H), 7.50-7.71 (m, 2H), 7.80 (d, 1H), 8.04-8.23 (m, 2H).

b) 1-(1-Isoquinolinyl)-4-(1,2,3,4-tetrahydroquinoline-2-ylmethyl)piperazine (Compound 1B)

The title compound was synthesised following the procedure described for Compound 1B (step h). The residue was purified by flash chromatography eluting with petroleum ether—EtOAc 5.5:4.5 to give 0.12 g (73.6%) of the title compound.

¹H-NMR (δ): 1.41-1.80 (m, 1H), 1.81-1.99 (m, 1H), 2.50 (d, 2H), 2.58-2.76 (m, 2H), 2.78-3.01 (m, 4H), 3.39-3.58 (m, 4H), 4.59-4.90 (br, 1H), 6.48-6.67 (m, 2H), 6.87-7.08 (m, 2H), 7.16-7.31 (m, 1H), 7.47-7.66 (m, 2H), 7.78 (d, 1H), 8.04-8.22 (m, 2H).

c) 1-(1-Cyclohexanecarbonyl-1,2,3,4-tetrahydroquinoline-2-ylmethyl)-4-(1-isoquinolinyl)piperazine

The title compound was synthesised following the alternative acylation procedure described for the compound of Example 1 (step f) but starting from Compound 21B instead of Compound 1B. The crude was purified by flash chromatography eluting with petroleum ether-ethyl acetate 6:4 to give the title compound (88%). M.p. (63.9) 70.4-72° C.

¹H-NMR (δ): 0.86-2.01 (m, 11H), 2.10-2.31 (m, 1H), 2.33-2.87 (m, 9H), 3.12-3.56 (m, 4H), 4.94-5.29 (m, 1H), 7.08-7.27 (m, 5H), 7.41-7.67 (m, 2H), 7.26 (d, 1H), 8.00-8.19 (m, 2H).

### EXAMPLE 22

1-(1-Cyclohexanecarbonyl-1,2,3,4-tetrahydroquinoline-2-ylmethyl)-4-(2-methoxyphenyl)piperidine

a) 2-[4-(2-Methoxyphenyl)-1-piperidinylmethyl]quinoline (Compound 22A)

The title compound was prepared as described for Compound 1C substituting 1-(4-indolyl)piperazine with 1-(2-methoxyphenyl)piperidine. Purification was carried out by flash chromatography eluting with petroleum ether-ethyl acetate 6:4 to give the title compound (53%). Oil.

¹H-NMR (δ): 1.70-1.90 (m, 4H), 2.18-2.35 (m, 2H), 2.85-3.10 (m, 3H), 3.82 (s, 3H), 3.90 (s, 2H), 6.78-7.00 (m, 2H), 7.08-7.30 (m, 2H), 7.51 (t, 1H), 7.60-7.85 (m, 3H), 8.10 (t, 1H).

b) 1-(1,2,3,4-Tetrahydroquinoline-2-ylmethyl)-4-(2-methoxyphenyl)piperidine (Compound 22B)

Compound 22B was obtained in the same way as described for Compound 1B (step e) but starting from Compound 22A instead of Compound 1C. The crude was purified by flash chromatography eluting with CH₂Cl₂-2.5 N NH₃ in MeOH 100:1 to 100:2 affording the title compound (50.3%). Oil.

¹H-NMR (δ): 1.40-2.20 (m, 6H), 2.20-3.20 (m, 9H), 3.30-3.60 (m, 1H), 3.90-4.50 (br, 1H), 6.45-6.65 (m, 2H), 6.80-7.05 (m, 4H), 7.05-7.30 (m, 2H).

c) 1-(1-Cyclohexanecarbonyl-1,2,3,4-tetrahydroquinoline-2-ylmethyl)-4-(2-methoxyphenyl)piperidine

The title compound was synthesised by the alternative acylation procedure (step f) described for the compound of Example 1 starting from Compound 22B instead of Compound 1B. The crude was purified by flash chromatography eluting with dichloromethane-methanol 95:5 and then with CH₂Cl₂-2.5 N NH₃ in MeOH 100:2 to 100:3 affording the title compound (72%). Oil.

¹H-NMR (δ): 1.40-2.20 (m, 16H), 2.20-3.30 (m, 10H), 3.80 (s, 3H), 4.85-5.25 (br, 2H), 6.75-7.00 (m, 2H), 7.00-7.35 (m, 6H).

### EXAMPLE 23

1-(7-Benzofuranyl)-4-(1-cyclohexanecarbonyl-1,2,3,4-tetrahydroquinoline-2-ylmethyl)piperazine

a) 1-(7-Benzofuranyl)-4-(1,2,3.4-tetrahydroquinoline-2-ylcarbonyl)piperazine (Compound 23A)

The title compound was synthesised following the procedure described for Compound 1D (step g) but using 1-(7-benzofuranyl)piperazine instead of 1-(4-indolyl)piperazine. The residue was purified by flash chromatography eluting with petroleum ether—EtOAc 55:45 to give the title compound (69%).

¹H-NMR (δ): 1.60-1.86 (m, 1H), 2.10-2.30 (m, 1H), 2.74-3.01 (m, 2H), 3.26-3.45 (m, 4H), 3.62-4.08 (m, 4H), 4.27 (d, 1H), 4.39-4.46 (br, 1H), 6.62-6.81 (m, 2H), 6.86-7.05 (m, 2H), 7.11-7.32 (m, 2H), 7.62 (d, 1H).

b) 1-(7-Benzofuranyl)-4-(1,2,3,4-tetrahydroquinoline-2-ylmethyl)piperazine (Compound 23B)

The title compound was synthesised following the procedure described for Compound 1B (step h). The residue was purified by flash chromatography eluting with petroleum ether—EtOAc 8:2 to give the title compound (62%).

¹H-NMR (δ): 1.49-1.71 (m, 1H), 1.79-1.98 (m, 1H), 2.50 (d, 2H), 2.57-2.76 (m, 2H), 2.77-2.98 (m, 4H), 4.40-4.99 (br, 1H), 6.48-6.66 (m, 2H), 6.72-6.85 (m, 2H), 6.91-7.08 (m, 2H), 7.09-7.29 (m, 2H), 7.62 (s, 1H).

c) 1-(7-Benzofuranyl)-4-(1-cyclohexanecarbonyl-1,2,3,4-tetrahydroquinoline-2-ylmethyl)piperazine

The title compound was synthesised following the alternative acylation procedure described for the compound of Example 1 (step f) but starting from Compound 23B instead of Compound 1B. The crude was purified by flash chromatography eluting with petroleum ether-ethyl acetate 6:4 to give the title compound (61.5%). M.p. 99.9-104° C.

¹H-NMR (δ): 0.83-1.99 (m, 11H), 2.02-2.21 (m, 1H), 2.29-2.85 (m, 9H), 3.19-3.89 (m, 4H), 5.03-5.23 (m, 1H), 6.64-6.85 (m, 2H), 7.06-7.27 (m, 6H), 6.72-6.85 (m, 2H), 7.59 (d, 1H).

### EXAMPLE 24

### EXAMPLE 24A

1-(2-Ethylbutanoyl-1,2,3,4-tetrahydroquinoline-2-ylmethyl)-4-(4-indolyl)piperazine

The title compound was synthesised following the alternative acylation procedure described for the compound of Example 1 (step f) starting from Compound 1B using 2-ethtylbutanoyl chloride instead of cyclohexanecarbonyl chloride. Purification (flash chromatography): petroleum ether—EtOAc 6:4. Yield: 88%. M.p. 145-148° C.

¹H-NMR (δ): 0.61 (t, 3H), 1.05 (t, 3H), 1.20-1.70 (m, 4H), 1.71-1.98 (m, 1H), 2.03-2.25 (m, 1H), 2.30-2.90 (m, 9H), 3.10-3.32 (m, 4H), 5.18-5.35 (m, 1H), 6.48-6.63 (m, 2H), 7.00-7.32 (m, 7H), 8.12 (s, 1H).

The following compounds were synthesised following the above acylation procedure starting from Compound 1B and using the proper acyl or aroyl chloride instead of 2-ethylbutanoyl chloride. Flash chromatography purification with the given eluting mixtures afforded the title compounds.

### EXAMPLE 24A1

1-(1-Cyclohex-3-enylcarbonyl-1,2,3,4-tetrahydroquinoline-2-ylmethyl)-4-(4-indolyl)piperazine

From 1-cyclohex-3-enecarbonyl chloride. Purification: petroleum ether—EtOAc-2.5 N NH₃ in MeOH 6:4:0.15. Yield: 40%. M.p. 156-160° C.

¹H-NMR (δ): 1.32-2.83 (m, 14H), 2.85-3.10 (m, 1H), 5.00-5.25 (m, 1H), 5.48-5.65 (m, 1H), 5.66-5.81 (m, 1H), 6.48-6.61 (m, 2H), 7.00-7.30 (m, 7H), 8.14 (s, 1H).

### EXAMPLE 24A2

1-(1-Cycloheptanecarbonyl-1,2,3,4-tetrahydroquinoline-2-ylmethyl)-4-(4-indolyl)piperazine

From 1-cycloheptanecarbonyl chloride. Purification: petroleum ether—EtOAc-2.5 N NH₃ in MeOH 7:3:0.1. Yield: 30%. M.p. 167-173° C.

¹H-NMR (δ): 1.02-2.04 (m, 13H), 2.03-2.22 (m, 11H), 2.30-2.80 (m, 8H), 2.81-2.98 (m, 1H), 3.11-3.22 (m, 1H), 4.95-5.22 (m, 1H), 6.48-6.61 (m, 2H), 7.00-7.30 (m, 7H), 8.14 (s, 1H).

### EXAMPLE 24A3

1-(1-Cyclopentanecarbonyl-1,2,3,4-tetrahydroquinoline-2-ylmethyl)-4-(4-indolyl)piperazine

From 1-cyclopentanecarbonyl chloride. Purification: petroleum ether—EtOAc-2.5 N NH₃ in MeOH 6:4:0.2. Yield: 82%. M.p. 150-153° C.

¹H-NMR (δ): 1.30-2.25 (m, 10H), 2.32-2.82 (m, 8H), 2.96-3.32 (m, 5H), 3.11-3.22 (m, 1H), 4.98-5.22 (m, 1H), 6.45-6.61 (m, 2H), 7.00-7.27 (m, 7H), 8.14 (s, 1H).

### EXAMPLE 24A4

1-(1-Benzoyl-1,2,3,4-tetrahydroquinoline-2-ylmethyl)-4-(4-indolyl)piperazine

From benzoyl chloride. Purification: petroleum ether—EtOAc-2.5 N methanolic ammonia 6:4:0.2. Yield: 91% Oil

¹H-NMR (δ): 1.78-1.98 (m, 1H), 2.30 (dd, 1H), 2.34-2.55 (m, 1H), 2.66 (dd, 1H), 2.69-2.88 (m, 4H), 3.12-3.38 (m, 4H), 4.98-5.14 (m, 1H), 6.50-6.73 (m, 3H), 6.80-6.97 (m, 1H), 7.00-7.37 (m, 10H), 8.14 (s, 1H).

### EXAMPLE 24A5

1-(4-Indolyl)-4-(1-pentanoyl-1,2,3,4-tetrahydroquinoline-2-ylmethyl)piperazine

From pentanoyl chloride. Purification: petroleum ether-ethyl acetate-2N NH₃ in MeOH 6:4:0.1. Yield: 80%.

¹H-NMR (δ): 1.85 (s, 3H), 1.12-1.40 (m, 2H), 1.42-1.72 (m, 3H), 2.16 (dd, 1H), 2.27-2.82 (m, 10H), 3.10-3.22 (m, 4H), 4.98-5.20 (m, 1H), 6.48-6.60 (m, 2H), 6.80-6.97 (m, 1H), 7.00-7.30 (m, 7H), 8.14 (s, 1H).

### EXAMPLE 25

1-(1-Cyclohexanecarbonyl-6-trifluoromethyl-1,2,3,4-tetrahydroquinoline-2-ylmethyl)-4-(4-indolyl)piperazine

a) 2-Methyl-6-trifluoromethylquinoline (Compound 25A)

A mixture of 4-trifluoromethylaniline (3 g), chloranil (4.6 g), 37% HCl (4.77 ml) and n-BuOH (4.77 ml) was heated to reflux; then crotonaldehyde (1.89 ml) in n-BuOH (1.88 ml) was added dropwise and the reaction mixture was stirred at reflux for 40 min. After cooling to r.t., it was diluted with H₂O, extracted with Et₂O (2×30 ml). The aqueous layer was alkalinized with 37% NaOH (pH>9) cooling with an ice bath, extracted with Et₂O (3×60 ml), dried (Na₂SO₄) and evaporated to dryness in vacuo. The crude was purified by flash chromatography eluting with petroleum ether-ethyl acetate 1:1 to give 3.8 g (72%) of the title compound.

¹H-NMR (δ): 2.73 (s, 3H), 7.39 (d, 1H), 7.86 (dd, 1H), 8.02-8.21 (m, 3H).

b) 2-Bromomethyl-6-trifluoromethylquinoline (Compound 25B)

Compound 26B was obtained in the same way as described for Compound 5A (procedure a) but using 6-trifluoromethyl-2-methylquinoline (Compound 25A) instead of 6-fluoro-2-methylquinoline. The crude was purified by flash chromatography eluting with petroleum ether—ethyl acetate 8:2 to give, as a first eluted product, the title compound (44%), followed by a partial recovery of unreacted starting material.

¹H-NMR (δ): 4.71 (s, 2H), 7.69 (d, 1H), 7.91 (dd, 1H), 8.12-8.21 (m, 3H).

c) 6-Trifluoromethyl-2-[4-(4-indolyl)-1-piperazinylmethyl]quinoline (Compound 25C)

Compound 25C was obtained in the same way as described for Compound 1C (procedure d) but using Compound 25B instead of 2-chloromethylquinoline. The crude was purified by flash chromatography eluting with petroleum ether—ethyl acetate 55:45 to give the title compound (44%).

¹H-NMR (δ):2.78-2.92 (m, 4H), 3.18-3.37 (m, 4H), 3.96 (s, 2H), 6.49-6.68 (m, 2H), 7.03-7.22 (m, 3H), 7.26-7.97 (m, 2H), 8.08-8.27 (m, 4H).

d) 1-(6-Trifluoromethyl-1.2,3,4-tetrahydroquinoline-2-ylmethyl)-4-(4-indolyl piperazine (Compound 25D)

The title compound was prepared as described for Compound 1B (procedure e) starting from Compound 25C instead of Compound 1C. The crude was purified by flash chromatography eluting with petroleum ether—ethyl acetate 7:3 to give the title compound (46%).

¹H-NMR (δ): 1.43-1.66 (m, 1H), 1.84-2.01(m, 1H), 2.43-2.71 (m, 4H), 2.80-3.01 (m, 4H), 3.20-3.41 (m, 4H), 3.45-3.54 (m, 1H), 5.10 (bs, 3H), 6.48-6.68 (m, 3H), 7.06-7.24 (m, 5H), 8.19 (s, 1H).

e) 1-(1-Cyclohexanecarbonyl-6-tri fluoromethyl-1,2,3,4-tetrahydroquinoline-2-yl methyl]-4-(4-indolyl)piperazine

The title compound was synthesised following the alternative acylation procedure described for the compound of Example 1 (step f) but starting from Compound 26D instead of Compound 1B. The crude was purified by flash chromatography eluting with petroleum ether—ethyl acetate 6:4 to give the title compound (59%).

¹H-NMR (δ): 1.00-1.99 (m, 11H), 2.11-2.28 (m, 1H), 2.30-2.51 (m, 2H), 2.61-2.83 (m, 7H), 3.11-3.30 (m, 4H), 4.96-5.17 (m, 1H), 6.45-6.63 (m, 2H), 7.00-7.19 (m, 3H), 7.21-7.38 (m, 1H), 7.41-7.59 (m, 2H), 8.17 (bs, 1H).

### EXAMPLE 26

1-(1-Cyclohexanecarbonyl-6-trifluoromethoxy-1,2,3,4-tetrahydroquinoline-2-ylmethyl)-4-(4-indolyl)piperazine

a) 2-Methyl-6-trifluoromethoxyquinoline (Compound 26A)

The title compound was prepared according to the procedure for compound 25A but using 4-trifluoromethoxyaniline instead of 4-trifluoromethylaniline. The crude was purified by flash chromatography eluting with CH₂Cl₂ 1:1 to give compound 26A (42%).

b) 2-Bromomethyl-6-trifluoromethoxyquinoline (Compound 26B)

Compound 26B was obtained in the same way as described for Compound 5A (procedure a) but using compound 2-methyl-6-trifluoromethoxyquinoline 26A instead of 6-fluoro-2-methylquinoline. The crude was purified by flash chromatography eluting with petroleum ether —CH₂Cl₂ 7:3 to afford the title compound (44%) accompanied by a small amount of 2,2-dibromomethyl-6-trifluoromethoxyquinoline.

¹H-NMR (δ): 4.70 (s, 2H), 7.50-7.70 (m, 3H), 8.05-8.25 (m, 2H).

c) 6-Trifluoromethoxy-2-[4-(4-indolyl)-1-piperazinylmethyl]quinoline (Compound 26C)

Compound 26C was obtained in the same way as described for Compound 1C (procedure d) but using Compound 26B instead of 2-chloromethylquinoline. The crude was purified by flash chromatography eluting with petroleum ether—ethyl acetate 6:4 to 5:5 to give the title compound (44%).

¹H-NMR (δ): 2.70-2.93 (m, 4H), 3.20-3.45 (m, 4H), 3.95 (s, 2H), 6.50-6.65 (m, 2H), 7.00-7.20 (m, 3H), 7.45-7.70 (m, 2H), 7.75 (d, 1H), 8.05-8.30 (m, 3H).

d) 1-(6-Trifluoromethoxy-1,2,3,4-tetrahydroquinoline-2-ylmethyl)-4-(4-indolyl)piperazine (Compound 26D)

The title compound was prepared as described for Compound 1B (procedure e) starting from Compound 26C instead of Compound 1C. The crude was purified by flash chromatography eluting with petroleum ether—ethyl acetate 6:4 to give the title compound (29%).

¹H-NMR (δ): 1.40-1.70 (m, 1H), 1.80-2.00 (m, 1H), 2.40-2.52 (m, 2H), 2.52-2.72 (m, 2H), 2.72-3.00 (m, 4H), 3.15-3.38 (m, 4H), 3.38-3.55 (m, 1H), 4.78 (b, 1H), 6.40-6.65 (m, 3H), 6.75-6.80 (m, 2H), 7.05-7.25 (m, 3H), 8.15 (b, 1H).

e) 1-(1-Cyclohexanecarbonyl-6-trifluoromethoxy-1,2,3,4-tetrahydroquinoline-2-ylmethyl)-4-(4-indolyl)piperazine

The title compound was synthesised following the alternative acylation procedure described for the compound of Example 1 (step f) but starting from Compound 26D instead of Compound 1B. The crude was purified by flash chromatography eluting with petroleum ether—ethyl acetate 6:4 to give the title compound (50%).

¹H-NMR (δ): 1.70-2.02 (m, 11H), 2.02-2.25 (m, 1H), 2.25-2.50 (m, 2H), 2.50-2.85 (m, 7H), 3.05-3.35 (m, 4H), 6.45-6.65 (m, 2H), 7.00-7.25 (m, 6H), 7.00-7.25 (m, 6H), 8.15 (bs, 1H).

### EXAMPLE 27

1-[1-(3-Benzylaminopropionyl)-1,2,3,4-tetrahydroquinoline-2-ylmethyl]-4-(4-indolyl)piperazine

a) 1-(1-Acryloyl-1,2,3,4-tetrahydroquinoline-2-ylmethyl)-4-(4-indolyl)piperazine (Compound 27A)

The title compound was synthesised following the alternative acylation procedure described for the compound of Example 1 (step f) but using acryloyl chloride instead of cyclohexanecarbonyl chloride and carrying out the reaction at −10° C. The crude was purified by flash chromatography eluting with petroleum ether—ethyl acetate—2.5 N methanolic ammonia 5:5:0.2 to give the title compound (62%) as a yellow solid. M.p. 147-8° C.

¹H-NMR (δ): 1.60-1.82 (m, 1H), 2.25 (dd, 1H), 2.32-2.89 (m, 9H), 3.08-3.33 (m, 4H), 5.01-5.22 (m, 1H), 5.65 (d, 1H), 6.40-6.63 (m, 4H), 7.00-7.30 (m, 7H), 8.15 (s, 1H).

b) 1-[1-(3-Benzylaminopropionyl)-1,2,3,4-tetrahydroquinoline-2-ylmethyl]-4-(4-indolyl)piperazine

A mixture of Compound 27A (0.52 g), benzylamine (0.71 ml) was stirred at 110° C. for 1 h. After cooling to 20-25° C., the reaction mixture was diluted with H₂O, and the pasty precipitated which was formed was extracted with EtOAc (20 ml), washed with H₂O, dried (Na₂SO₄) and evaporated to dryness in vacuo. The crude was purified by flash chromatography eluting with ethyl acetate—2.5 N methanolic ammonia 95:5 to give the title compound (91%) as a yellow solid.

¹H-NMR (δ): 1.38-1.65 (m, 1H), 2.08-2.25 (m, 2H), 2.31-2.81 (m, 10H), 2.82-2.98 (m, 24H), 3.10-3.32 (m, 4H), 3.88 (s, 2H), 4.95-5.27 (br, 1H), 6.48-6.62 (m, 2H), 7.00-7.40 (m, 12H).

### EXAMPLE 28

1-(3-Aminopropionyl-1,2,3,4-tetrahydroquinoline-2-ylmethyl]-4-(4-indolyl)piperazine

A mixture of the compound of Example 27 (0.51 g), ammonium formate (0.32 g), 10% palladium on carbon (0.25 g) and MeOH (18 ml) was stirred at reflux for 1.5 h. Afterwards, it was filtered and evaporated to dryness in vacuo. The crude was purified by flash chromatography eluting with CHCl₃—2.5 N methanolic ammonia 9:1 to give 0.34 g of the title compound (80%) as a yellow solid.

¹H-NMR (δ): 1.38-1.78 (m, 3H), 2.06-2.23 (m, 1H), 2.25-2.88 (m, 12H), 2.89-3.08 (M, 2H), 3.10-3.32 (m, 2H), 5.00-5.30 (br, 1H), 6.47-6.63 (m, 2H), 7.00-7.33 (m, 7H).

### EXAMPLE 29

1-(4-Indolyl)-[1-(3-methylaminopropionyl-1,2,3,4-tetrahydroquinoline-2-ylmethyl]piperazine

The title compound was synthesised following the procedure reported for Compound 27 (step b) but using 40% aqueous methylamine instead of benzylamine and stirring at r.t. in DMF. The crude was purified by flash chromatography eluting with CHCl₃—2.5 N methanolic ammonia 92:8 to give the title compound (63%).

¹H-NMR (δ): 1.40-1.65 (in, 1H), 1.70 (s, 1H), 2.10-2.25 (m, 2H), 2.30-2.95 (m, 15H), 3.10-3.31 (m, 4H), 4.98-5.25 (br, 1H), 6.48-6.62 (m, 2H), 7.00-7.28 (m, 7H),

### EXAMPLE 30

1-[1-(3-Dimethylaminopropionyl)-1,2,3,4-tetrahydroquinoline-2-ylmethyl]-4-(4-indolyl)piperazine

The title compound was synthesised following the procedure reported for Compound 27 (step b) but using a solution of anhydrous dimethylamine in toluene and stirring at 100° C. for 2 h in toluene in a sealed reaction vessel. The crude was purified by flash chromatography eluting with EtOAc-2.5 N methanolic ammonia 9:1 to give the title compound (56%).

¹H-NMR (δ): 1.38-1.65 (m, 1H), 2.05-2.30 (m, 6H), 2.31-2.87 (m, 12H), 3.20-3.35 (m, 4H), 4.98-5.30 (br, 1H), 6.48-6.62 (m, 2H), 7.00-7.32 (m, 7H), 8.20 (s, 1H).

### EXAMPLE 31

1-(4-Indolyl)-4-(1-phenylaminocarbonyl-1,2,3,4-tetrahydroquinoline-2-ylmethyl)piperazine

The title compound was synthesised as described for the compound of Example 20 but using phenylisocyanate instead of ethyl isocyanate and stirring at reflux for 11 h in toluene. The crude was purified by flash chromatography eluting with EtOAc—petroleum ether—2.5 N methanolic ammonia 1:1:0.01 to give the title compound (69.1%).

¹H-NMR (δ): 1.60-1.815 (m, 1H), 2.20-2.40 (m, 1H), 2.50 (d, 1H), 2.62-3.08 (m, 7H), 3.10-3.50 (in, 4H), 4.48-4.72 (m, 1H), 6.48-6.70 (m, 2H), 6.95-7.40 (m, 9H), 7.42-7.58 (m, 3H), 8.25 (s, 1H).

### EXAMPLE 32

1-(1-Cyclohexanecarbonyl-6-methyl-1,2,3,4-tetrahydroquinoline-2-ylmethyl)-4-(4-indolyl)piperazine

a) 1-Benzoyl-2-cyano-6-methyl-1,2-dihydroquinoline (Compound 32A)

To a solution of 6-methylquinoline (2.83 ml) in CH₂Cl₂ (25.5 ml) was added a solution of potassium cyanide (4.1 g) in water (10.25 ml) followed by dropwise addition of benzoyl chloride (4.86 ml). The reaction was stirred at r.t. for 4 h, then the organic layer was separated and the aqueous layer extracted with CH₂Cl₂ (30 ml). The combined organic layers were washed with H₂O, 1 N HCl, H₂O, 1N NaOH, dried, evaporated to dryness in vacuo. The crude was crystallized from EtOH affording 1.84 g of the title product (32%). A second amount (0.86 g; 15%) was obtained by flash chromatography purification of the mother liquors eluting with petroleum ether—EtOAc 8:2.

¹H-NMR (δ): 2.30 (s, 3H), 6.05-6.14 (m, 1H), 6.21 (d, 1H), 6.48 (d, 1H), 6.70-6.89 (m, 2H), 7.03 (d, 1H), 7.27-7.46 (m, 5H).

b) 6-Methylquinoline-2-carboxylic acid (Compound 32B)

A suspension of 2.7 g of Compound 32A in 48% HBr (3 ml), AcOH (3 ml), and H₂O(17 ml) was stirred at reflux for 40-45′. The solid which precipitated after cooling to r.t was taken up with 32% aq. NH₃ to pH=8-9 at 50° C.; afterwards, AcOH was added to lower the pH to about 4. After filtration the title compound was obtained (1.69 g; 90%).

¹H-NMR (δ): 2.53 (s; 3H) 7.70 (d, 1H), 7.86 (d, 1H), 8.01-8.13 (m, 2H), 8.42 (d, 1H), 12.75-13.50 (br, 1H).

c) 6-Methyl-1,2,3,4-tetrahydroquinoline-2-carboxylic acid (Compound 32C)

The title compound was prepared as described for Compound 1B (step e) starting from Compound 32B instead of Compound 1D. The crude was treated with 37% HCl and taken up with MeCN (5 ml) to afford, after cooling to 0° C. and filtration, Compound 32C (53%).

¹H-NMR (δ): 1.94-2.15 (m, 1H), 2.16-2.32 (m, 4H), 2.57-2.91 (m, 2H), 4.15 (dd, 1H), 6.81-7.01 (m, 3H), 7.25-7.69 (br, 2H), 9.31-10.5 (br, 1H)

d) 1-(4-Indolyl)-4-(6-methyl-1,2,3,4-tetrahydroquinoline-2-ylcarbonyl)piperazine (Compound 32D)

The title compound was synthesised following the procedure described for Compound 1D (step g) but using Compound 32C instead of 1,2,3,4-tetrahydroquinoline-2-carboxylic acid. The residue was purified by flash chromatography eluting with petroleum ether—EtOAc 1:1 to give the title compound (73%).

¹H-NMR (δ): 1.61-1.86 (m, 1H), 2.08-2.30 (m, 4H), 2.68-2.96 (m, 2H), 3.18-3.36 (m, 4H), 3.67-4.10 (m, 4H), 4.23 (dd, 1H), 4.45-4.51 (br, 1H), 6.51-6.69 (m, 3H), 6.78-6.93 (m, 2H), 7.05-7.22 (m, 3H), 8.15-8.38 (br, 1H).

e) 1-(4-Indolyl)-4-(6-methyl-1,2,3,4-tetrahydroquinoline-2-ylmethyl)piperazine (Compound 32E)

The title compound was synthesised following the procedure described for Compound 1B (step h). The residue was purified by flash chromatography eluting with petroleum ether—EtOAc 7:3 to give the title compound (35%).

¹H-NMR (δ): 1.48-1.71 (m, 1H), 1.82-1.97 (m, 1H), 2.21 (s, 3H), 2.50 (d, 2H), 2.55-2.74 (m, 2H), 2.76-2.99 (m, 4H), 3.19-3.37 (m, 4H), 3.38-3.56 (m, 1H), 4.33-4.69 (br, 1H), 6.43-6.69 (m, 3H), 6.74-6.89 (m, 2H), 7.01-7.21 (m, 3H), 8.07-8.26 (br, 2H).

f) 1-(1-Cyclohexanecarbonyl-6-methyl-1,2,3,4-tetrahydroquinoline-2-ylmethyl)-4-(4-indolyl)piperazine

The title compound was synthesised following the alternative acylation procedure described for the compound of Example 1 (step f) but starting from Compound 32E instead of Compound 1B. The crude was purified by flash chromatography eluting with petroleum ether—ethyl acetate 1:1 to give the title compound (88%).

¹H-NMR (δ): 0.86-2.01; (m; 111H), 2.04-2.21 (m; 1H), 2.29-2.86 (m; 2H), 3.11-3.31 (m; 4H), 4.99-5.22 (m; 1H), 6.48-6.62(m; 2H), 6.96-7.16 (m; 6H), 8.05-8.24 (br; 1H).

### EXAMPLE 33

1-(4-Indolyl)-4-(1-pyrrolidinecarbonyl-1,2,3,4-tetrahydroquinoline-2-ylmethyl)piperazine

The title compound was synthesised following the alternative acylation procedure described for the compound of Example 1 (step f) using 4-pyrrolidinocarbonyl chloride instead of cyclohexanecarbonyl chloride and chloroform instead of dichloromethane, and refluxing for 8 hours. The crude was purified by flash chromatography eluting with petroleum ether—ethyl acetate—2.5N methanolic ammonia 8:2:0.01 to give the title compound (10%).

¹H-NMR (δ): 1.51-2.21 (m, 4H), 2.22-2.41 (m, 1H), 2.58-295 (m, 9H), 3.51-3.69 (m, 2H), 4.34-4.59 (m, 1H), 6.49-6.63 (m, 2H), 6.79-7.28 (m, 7H), 8.14 (bs, 1H)

### EXAMPLE 34

1-(6-Bromo-1-cyclohexanecarbonyl-1,2,3,4-tetrahydroquinoline-2-ylmethyl)-4-(4-indolyl)piperazine

a) 6-Bromo-2-methoxycarbonyl-1,2,3,4-tetrahydroquinoline (Compound 34A)

A solution of 0.36 g of N-bromosuccinimide in 3 ml of anhydrous DMF was dropped into a solution of 0.37 g of 2-methoxycarbonyl-1,2,3,4-terahydroquinoline stirred at 0-5° C. After 2.5 h at 0-5° C. and 20 h at r.t. the mixture was diluted with H₂O (50 ml) and extracted with Et₂O (3×20 ml); the organic layer was washed with H₂O (3×10 ml) and dried (anhydrous Na₂SO₄). The solvent was evaporated and the residue was purified by flash chromatography (petroleum ether—EtOAc 9:1) to afford 0.29 g (55.5%) of Compound 34A as an ivory solid.

¹H-NMR (δ): 1.85-2.38 (2m, 2H), 2.60-2.90(m, 2H), 3.78(s, 3H), 3.90-4.10 (m, 1H), 4.38 (s, 1H), 6.45 (d, 1H), 7.00-7.15 (m, 2H).

b) 6-Bromo-2-hydroxymethyl-1,2,3,4-tetrahydroquinoline (Compound 34B)

A mixture of 0.27 g of Compound 34A, 5 ml of anhydrous THF and 0.6 ml of 2 M LiBH₄ in THF was stirred at r.t. for 6.5 h, diluted with H₂O (50 ml) and extracted with Et₂O (3×20 ml). The organic layer was dried (anhydrous Na₂SO₄) and evaporated to dryness in vacuo to afford 0.21 g (85.1%) of the title compound as an oil.

¹H-NMR (δ): 1.45-2.00 (2m, 3H), 2.60-2.95 (m, 2H), 3.20-3.85 (2m, 3H), 4.00-4.55 (br, 1H), 6.40 (d, 1H), 6.95-7.10 (m, 2H)

c) 6-Bromo-2-iodomethyl-1,2,3,4-tetrahydroquinoline (Compound 34C)

Into a stirred mixture of 0.31 g of Compound 34B, 0.20 g of imidazole, 0.40 g of Ph₃P and 3 ml of toluene—MeCN (5:1) was added at 0-5° C. during 15 min, 0.36 g of ₂. The mixture was stirred at 0-5° C. for 15′, then at r.t. for 1 h, treated with aq. Na₂S₂O₃ and extracted with EtOAc (3×20 ml); the organic layer was washed with brine, dried (anhydrous Na₂SO₄) and evaporated to dryness in vacuo to afford 0.76 g of a mixture of Compound 34C and Ph₃PO (NMR) and used without further purification in the next reactionstep.

¹H-NMR (δ): 1.63-2.15 (2m, 1H), 2.58-2.90 (m, 2H), 3.10-3.35 (2m, 2H), 3.35-3.55 (m, 1H), 4.05-4.40 (b, 1H), 6.40 (d, 1H), 6.95-7.20 (m, 2H), 7.40-7.80(m, 8H).

d) 6-Bromo-2-[4-(4-indolyl)-1-piperazinyl]methyl]-1,2,3,4-tetrahydroquinoline (Compound 34D)

A stirred mixture of 0.76 g of Compound 34C, 0.6 ml of anhydrous DMF, 0.45 ml of DIPEA and 0.29 g (1.39 mmol) of 1-(4-indolyl)piperazine was heated at 115° C. for 4 h, diluted with H₂O (50 ml) and extracted with Et₂O. The organic layer was washed with H₂O (2×10 ml), dried (anhydrous Na₂SO₄) and evaporated to dryness in vacuo. The residue was purified by flash chromatography (petroleum ether—EtOAc 7:3) to afford 0.27 g (50.7%) of the title compound as a dense oil.

¹H-NMR (δ): 1.40-1.70 (m, 1H), 1.80-2.00 (m, 1H), 2.35-3.00 (m, 8H), 3.10-3.70 (m, 5H), 4.55-4.90(b, 1H), 6.40 (d, 1H), 6.50-6.70 (m, 2H), 6.95-7.25 (m, 5H), 8.05-8.35 (b, 1H).

e) 1-(6-Bromo-1-cyclohexanecarbonyl-1,2,3,4-tetrahydroquinoline-2-ylmethyl)-4-(4-indolyl)piperazine

The title compound was prepared from Compound 34D following the alternative acylation procedure described for the compound of Example 1 (step f) using 4-pyrrolidinocarbonyl chloride instead of cyclohexanecarbonyl chloride and chloroform instead of dichloromethane, and refluxing for 8 hours. The residue was purified by flash chromatography (petroleum ether—EtOAc 70:30) affording the title compound (65%) as an ivory vitreous solid.

¹H-NMR (δ): 0.85-2.00 (m, 12H), 2.00-2.20 (m, 1H), 2.20-2.85 (m, 8H), 3.00-3.45 (m, 4H), 4.80-5.20 (m, 1H), 6.45-6.65 (m, 2H), 6.85-7.20 (m, 4H), 7.30-7.05 (m, 2H), 8.15 (br, 1H).

### EXAMPLE 35

### EXAMPLE 35A

1-(1-Cyclohexanecarbonyl-1,2,3,4-tetrahydroquinoline-2-ylmethyl)-4-(4-fluoro-2-methoxyphenyl)piperazine

a) 1-Cyclohexanecarbonyl-2-hydroxymethyl-1,2,3,4-tetrahydroquinoline(Compound 35A-A)

Into a solution of methyl 1-cyclohexanecarbonyl-1,2,3,4-tetrahydroquinoline-2-carboxylate (19 g) in anhydrous THF (210 ml) stirred at 0° C. was dropped lithium borohydride (31.5 ml of a 2M sol. in THF). The reaction mixture was stirred at 0° C.—r.t for 6 h; afterwards, it was cautiously acidified by adding 2N HCl and extracted with EtOAc (2×500 ml). The organic layers were washed with H₂O, dried (Na₂SO₄), evaporated to dryness in vacuo and purified by flash chromatography eluting with petroleum ether—EtOAc 6:4 to give 11.8 g (69%) of the title compound.

¹H-NMR (δ): 0.80-2.00 (m, 11H), 2.30-2.85 (m, 4H), 3.32 (dd, 1H), 3.60 (dd, 1H), 3.95-4.20 (br, 1H), 4.70-4.92 (m, 1H), 7.05-7.35 (m, 4H)

b) 1-Cyclohexanecarbonyl-1,2,3.4-tetrahydroquinoline-2-carbaldehyde (Compound 35A-B)

To a solution of freshly distilled oxalyl chloride (5.65 ml) stirred at −60° C. in anhydrous CH₂Cl₂ (100 ml) was added anhydrous DMSO (9.2 ml) during 15 min. A solution of Compound 35A-A (11.8 g) in CH₂Cl₂ (100 ml) was dropped and, after 5 min stirring at the same temperature, TEA (35 ml) was added dropwise. The reaction mixture was allowed to warm to 0° C. during 3 h.; afterwards, it was quenched by adding H₂O, alkalinised with 1 M NaOH and extracted with CH₂Cl₂ (2×200 ml). The organic layers were washed with H₂O, dried (Na₂SO₄), evaporated to dryness in vacuo and purified by flash chromatography eluting with petroleum ether—EtOAc 7:3 to give 11.2 g (95%) of the title compound.

¹H-NMR (δ): 0.82-2.08 (m, 11H), 2.28-2.82 (m, 4H), 2.83-3.03 (m, 1H), 5.18 (dd, 1H, 7.08-7.32 (m, 4H), 9.50 (s, 1H)

c) 1-(1-Cyclohexanecarbonyl-1,2,3,4-tetrahydroquinoline-2-ylmethyl)-4-(4-fluoro-2-methoxyphenyl)piperazine (Ex. 35A!

To a solution 1-cyclohexanecarbonyl-1,2,3,4-tetrahydroquinoline-2-carbaldehyde (0.14 g), 1-(4-fluoro-2-methoxyphenyl)piperazine (0.17 g) in CHCl₃ (5 ml) at r.t., were added sodium triacetoxyborohydride (0.21 g) and acetic acid (0.14 ml). The solution was stirred at r.t. for 3 h, maintained overnight resting, poured into H₂O (30 ml), alkalinised with 1 N NaOH and extracted with EtOAc (2×30 ml). The combined organic layers were washed with H₂O, dried (Na₂SO₄) and the solvent was evaporated under vacuum. The crude was purified by flash chromatography eluting with EtOAc-petroleum ether-2N NH₃ in MeOH 1:1:0.01 to give 0.18 g (74%) of the title compound.

¹H-NMR (δ): 0.82-2.18 (m, 11H), 2.21-2.85 (m, 9H), 2.86-3.10 (m, 4H), 3.83 (s, 3H), 4.92-5.12 (m, 1H), 6.486-6.66 (m, 2H), 6.78-6.90 (m, 2H), 6.98-7.30 (m, 4H).

The following compounds were similarly synthesised (Ex. 35A—step c) in a parallel synthesis fashion using dichloromethane instead of chloroform and using the proper piperazine instead of 1-(4-fluoro-2-methoxyphenyl)piperazine. After having alkalinised, diluted with CH₂Cl₂ and removed the aqueous layer the organic layer was evaporated to dryness in vacuo and the crude purified by flash chromatography (eluent shown):

### EXAMPLE 35A1

1-(1-Cyclohexanecarbonyl-1,2,3,4-tetrahydroquinoline-2-ylmethyl)-4-(2-methyl-4-indolyl)piperazine

From 1-(2-methyl-4-indolyl)piperazine.

Flash chromatography: CH₂Cl₂— MeOH: 95-5. Yield: 89%.

¹H-NMR (δ): 0.79-2.01 (m, 11H), 2.05-2.22 (m, 1H), 2.30-2.88 (m, 12H), 3.00-3.32 (m, 4H), 4.95-5.25 (m, 1H), 6.20 (s, 1H), 6.55 (d, 1H), 6.90-7.32 (m, 6H), 7.78-8.00 (br, 1H).

The starting piperazine was obtained as following:

a) 2-Methyl-7-aminoindole (Compound 35A1-A)

To a solution of 2-methyl-7-nitroindole (N. Moskalev et al, Tetrahedron Letters 40, 5395-5398, (1999)) (1.4 g) and Ni-Raney (40 mg) in MeOH (40 ml) was added hydrazine hydrate (4.8 ml) and the resulting suspension stirred for 4 h. The catalyst was filtered off and the solvents evaporated under vacuum. The residue was dissolved in CH₂Cl₂ (80 ml) and washed with H₂O (50 ml). The organic layer was dried (Na₂SO₄) and the residue was purified by flash chromatography (CH₂Cl₂-2N NH₃ in MeOH 97:3) to give 0.62 g (53%) of the title compound.

¹H-NMR (δ): 2.48 (s, 3H), 3,21-4.32 (br, 2H), 6.05 (s, 1H), 6.41 (dd, 1H), 6.78 (dd, 1H), 6.95 (dd, 1H), 7.75-7.93 (br 1H)

b) 1-(2-Methyl-4-indolyl)piperazine (Compound 35A1-B)

A mixture of Compound 35A1-A (0.62 g), bis(2-chloroethyl)amine hydrochloride (0.7 6 g), potassium iodide (0.35 g), DIPEA (0.8 ml) in 1,2-dichlorobenzene (5 ml) and n-hexanol (0.5 ml) was heated at 190° C. for 3 h. After cooling at r.t., EtOAc (30 ml) and 1M NaOH (20 ml) was added; the organic layer was separated and dried (Na₂SO₄)and evaporated to dryness. The crude was purified by flash chromatography (CH₂Cl₂-2N NH₃ in MeOH 97:3) to give 0.43 g (47%) of the title compound.

¹H-NMR (δ): 2.46 (s, 3H), 3.02 (m, 8H), 6.31 (s 1H), 6.55 (dd, 1H), 6.92-7.08 (m, 2H), 7.83-8.01 (br 1H).

### EXAMPLE 35A2

1-(1-Cyclohexanecarbonyl-1,2,3,4-tetrahydroquinoline-2-ylmethyl)-4-phenylpiperazine

From 1-phenylpiperazine (commercial)

Flash chromatography: petroleum ether—EtOAc 70:30. Yield: 43%.

¹H-NMR (δ): 0.82-2.18 (m, 11H), 2.02-2.18 (m, 1H), 2.20-2.82 (m, 9H), 3.00-3.22 (m, 4H), 5.00-5.25 (m, 1H), 6.75-6.98 (m, 3H), 6.99-7.32 (m, 6H).

### EXAMPLE 35A3

1-(1-Cyclohexanecarbonyl-1,2,3,4-tetrahydroquinoline-2-ylmethyl)-4-(2-ethoxyphenyl)piperazine

From 1-(2-ethoxyphenyl)piperazine (commercial)

Flash chromatography: petroleum ether—EtOAc 70:30. Yield: 74%.

¹H-NMR (δ): 0.78-2.18 (m, 12H), 1.45 (t, 3H), 2.28-2.85 (m, 9H), 2.90-3.22 (m, 4H), 4.08 (q, 2H), 4.95-5.25 (m, 1H), 6.75-7.02 (m, 4H), 7.03-7.32 (m, 4H).

### EXAMPLE 35A4

1-(1-Cyclohexanecarbonyl-1,2,3,4-tetrahydroquinoline-2-ylmethyl)-4-(2,5-dimethoxyphenyl)piperazine

From 1-(2,5-dimethoxyphenyl)piperazine (J. Med. Chem. 29, 630, 1986) Flash chromatography: petroleum ether—EtOAc 50:50. Yield: 56%.

¹H-NMR (δ): 0.75-2.20 (m, 12H), 2.25-2.80 (m, 9H), 2.88-3.15 (m, 4H), 3.78;3,80 (2s, 6H), 4.95-5.25 (m, 1H), 6.40-7.06 (m, 2H), 6,78 (d, 1H), 7.03-7.32 (m, 4H).

### EXAMPLE 35A5

1-(1-Cyclohexanecarbonyl-1,2,3,4-tetrahydroquinoline-2-ylmethyl)-4-(2,3-dihydrobenzofuran-7-yl)piperazine

From 1-(2,3-dihydrobenzofuran-7-yl)piperazine (F. Kerrigan et al., Tetrahedron Letters, 39, 2219-2222, (1998)).

Flash chromatography: petroleum ether—EtOAc 70:30. Yield: 86%.

¹H-NMR (δ): 0.75-2.20 (m, 12H), 2.20-2.80 (m, 9H), 2.90-3.30 (m, 6H), 4.58 (t, 2H), 4.90-5.25 (m, 1H), 6.40-6.90 (m, 3H), 6,95-7.20 (m, 4H).

### EXAMPLE 35A6

1-(1-Cyclohexanecarbonyl-1,2,3,4-tetrahydroquinoline-2-ylmethyl)-4-(5-fluoro-2-methoxyphenyl)piperazine

From 1-(5-fluoro-2-methoxyphenyl)piperazine (U.S. Pat. No. 4,585,773,1986)

Flash chromatography: petroleum ether—EtOAc 6:4. Yield: 32%.

¹H-NMR (δ): 0.75-1.99(m,l 1H), 2.01-2.18 (m, 1H), 2.28-2.80 (m, 9H), 2.89-3.11 (m, 4H), 3.81 (s, 3H), 4.92-5.21 (m, 1H), 6.55-6.78 (m, 3H) 7.05-7.25 (m, 4H).

### EXAMPLE 35A7

1-(1-Cyclohexanecarbonyl-1,2,3,4-tetrahydroquinoline-2-ylmethyl)-4-(2-methylphenyl)piperazine

From 1-(2-methylphenyl)piperazine(commercial).

Flash chromatography: EtOAc 8:2. Yield: 25%.

¹H-NMR (δ): 0.80-1.99 (m, 11H), 2.00-2.21 (m, 1H), 2.22-2.95 (m, 18H), 4.96-5.22 (m, 1H), 6.95-7.06 (m, 2H), 7.08-7.20 (m, 6H)

### EXAMPLE 35A8

1-(1-Cyclohexanecarbonyl-1,2,3,4-tetrahydroquinoline-2-ylmethyl)-4-(2-trifluoromethylphenyl)piperazine

From 1-(2-trifluoromethylphenyl)piperazine (commercial) ¹H-NMR (δ): 0.8-2.2 (m, 13H), 2.23-2.95 (m, 12H), 4.95-5.2 (m, 1H), 7.00-7.65 (m, 8H).

### EXAMPLE 35A9

1-(1-Cyclohexanecarbonyl-1,2,3,4-tetrahydroquinoline-2-ylmethyl)-4-(2,4-dichlorophenyl)piperazine

From 1-(2,4-dichlorophenyl)piperazine (commercial)

Flash chromatography: EtOAc 7:3. Yield: 77%.

¹H-NMR (δ): 0.8-2.2 (m, 13H), 2.25-3.1 (m, 12H), 5.0-5.25 (m, 1H), 7.00-7.65 (m, 7H).

### EXAMPLE 35A10

1-(1-Cyclohexanecarbonyl-1,2,3,4-tetrahydroquinoline-2-ylmethyl)-4-(2-hydroxyphenyl)piperazine

From 1-(2-hydroxyphenyl)piperazine (commercial)

Flash chromatography: petroleum ether—EtOAc 7:3. Yield: 82%.

¹H-NMR (δ): 0.72-2.12 (m, 13H), 2.23-2.85 (m, 13H), 4.96-5.23 (m, 1H), 6.75-6.95 (m, 2H), 7.01-7.30 (m, 6H).

### EXAMPLE 35A11

1-(1-Cyclohexanecarbonyl-1,2,3,4-tetrahydroquinoline-2-ylmethyl)-4-(2-i-propoxyphenyl)piperazine

From 1-(2-i-propoxyphenyl)piperazine (Martin, G. E. et al., J. Med. Chem. 32, 1052-1056, (1989)).

Flash chromatography: petroleum ether—EtOAc 7:3. Yield: 77%.

¹H-NMR (δ): 0.76-2.11 (m, 18H), 2.21-2.84 (m, 9H), 2.89-3.14 (m, 4H), 4.49-4.69 (m, 1H), 4.94-5.21 (m, 1H), 6.80-6.92 (m, 4H), 7.03-7.29 (m, 4H).

### EXAMPLE 35A12

1-(1-Cyclohexanecarbonyl-1,2,3,4-tetrahydroquinoline-2-ylmethyl)-4-(2-fluoro-5-methylphenyl)piperazine

From 1-(2-fluoro-5-methylphenyl)piperazine (WO01/05765, 2001)

Eluent: petroleum ether—EtOAc 7:3. Yield: 46%.

¹H-NMR (δ): 0.78-2.18 (m, 12H), 2.20-2.85 (m, 12H), 2.95-3.15 (m, 4H), 4.97-5.23 (m, 1H), 6.62-6.80 (m, 1H), 6.63-6.92 (m, 2H), 7.02-7.25 (m, 4H).

### EXAMPLE 35A13

1-(1-Cyclohexanecarbonyl-1,2,3,4-tetrahydroquinoline-2-ylmethyl)-4-(2,3-dimethyl-4-indolyl)piperazine

From 1-(2,3-dimethyl-4-indolyl)piperazine

Flash chromatography: petroleum ether—EtOAc 6:4. Yield: 56%.

¹H-NMR (δ): 0.78-2.20 (m, 1H), 2.21-2.82 (m, 16H), 2.84-3.22 (m, 4H), 5.01-5.26 (m, 1H), 6.58-6.71 (m, 1H), 6.92-7.02 (m, 2H), 7.04-7.32 (m, 4H), 7.65-7.81 (br, 1H).

The starting piperazine was obtained as following:

a) 2,3-Dimethyl-7-aminoindole (Compound 35A13-A)

The title compound was prepared as described for Compound 35A1-A starting from 2,3-dimethyl-7-nitroindole (N. Moskalev et al, Tetrahedron Letters 40, 5395-5398, (1999)) instead of 2-methyl-7-nitroindole. The crude was purified by flash chromatography eluting with petroleum ether-ethyl acetate 7:3 to give the title compound (70%).

¹H-NMR (δ): 2.31 (s, 3H), 2.49 (s, 3H), 3,85-4.21 (br, 2H), 6.27 (dd, 11H), 6.70 (dd, 1H), 6.94 (dd, 1H), 7.48-7.73 (br 1H)

b) 1-(2,3-Dimethyl-4-indolyl)piperazine (Compound 35A13-B)

The title compound was prepared as described for Compound 35A1-B starting from Compound 35A13-A instead of Compound 35A1-A. The crude was purified by flash chromatography eluting with CH₂Cl₂-2N NH₃ in MeOH 97:3 to give the title compound (43%).

¹H-NMR (δ): 2.30 (s, 3H), 2.43 (s, 3H), 2.95-3.21 (m, 8H), 6.31 (s 1H), 6.62-6.75 (m, 1H), 6.91-7.09 (m, 2H), 7.63-7.86 (br 1H).

### EXAMPLE 35A14

1-(1-Cyclohexanecarbonyl-1,2,3,4-tetrahydroquinoline-2-ylmethyl)-4-(2-chloro-5-fluorophenyl)piperazine

1-(2-chloro-5-fluorophenyl)piperazine (WO 01/05765, 2001)

Flash chromatography: petroleum ether—EtOAc 8:2. Yield: 25%.

¹H-NMR (δ): 0.8-2.2 (m, 13H), 2.22-2.75 (m, 8H), 2.8-3.2 (m, 4H), 4.94-5.21 (m, 1H), 6.60-6.80 (m, 2H), 7.00-7.30 (m, 5H).

### EXAMPLE 35A15

1-(1-Cyclohexanecarbonyl-1,2,3,4-tetrahydroquinoline-2-ylmethyl)-4-(5-fluoro-2-methylphenyl)piperazine

From 1-(5-fluoro-2-methylphenyl)piperazine.

Flash chromatography: petroleum ether—EtOAc 8:2. Yield: 18%.

¹H-NMR (δ): 0.8-2.15 (m, 13H), 2.15-2.25 (s, 3H) 2.30-2.85 (m, 12H), 4.94-5.21 (m, 1H), 6.58-6.78 (m, 2H), 7.00-7.30 (m, 5H).

The starting piperazine was prepared as following:

a) 1-(5-Fluoro-2-methylphenyl)piperazine (Compound 35A15-A)

The title compound was prepared as described for Compound 35A1-B starting from 1-(5-fluoro-2-methyl)aniline instead of Compound 35A1-A. The crude was taken up with hot EtOAc, cooled at r.t. and filtered to afford the title comopound as hydrochloride.

### EXAMPLE 35A16

1-(2,3-Dihydro-1,4-benzodioxin-5-yl)-4-(1-cyclohexanecarbonyl-1,2,3,4-tetrahydroquinoline-2-ylmethyl)piperazine

From 1-(2,3-Dihydro-1,4-benzodioxin-5-yl)piperazine (F. Kerrigan et al., Tetrahedron Letters, 39, 2219-2222, (1998)).

Flash chromatography: petroleum ether—EtOAc 4:6. Yield: 22%.

¹H-NMR (δ): 0.8-2.2 (m, 13H), 2.25-2.80 (m, 8H) 2.65-3.25 (m, 4H), 4.16-4.38 (m, 4H), 4.95-5.20 (m, 1H), 6.48-6.80 (m, 3H), 7.00-7.30 (m, 4H).

### EXAMPLE 35A17

1-(1-Cyclohexanecarbonyl-1,2,3,4-tetrahydroquinoline-2-ylmethyl)-4-(2-trifluoromethoxyphenyl)piperazine

From 1-(2-trifluoromethoxyphenyl)piperazine (EP 0711757, 1996)

Flash chromatography: petroleum ether—EtOAc 7:3. Yield: 35%

¹H NMR (δ): 0.65-2.80 (m, 21H), 2.90-3.15 (m, 4H), 5.10 (bs, 1H), 6.95 (m, 2H), 7.05-7.30 (m, 6H)

### EXAMPLE 35A1

1-(1-Cyclohexanecarbonyl-1,2,3,4-tetrahydroquinoline-2-ylmethyl)-4-(4-fluoro-2-methylphenyl)piperazine

From 1-(4-fluoro-2-methylphenyl)piperazine (WO01/29015, 2001)

Flash chromatography: petroleum ether—EtOAc 7:3. Yield: 87%.

¹H NMR (δ): 0.70-2.20 (m, 13H), 2.20 (s, 3H), 2.20-2.90 (m, 12H), 5.10 (bs, 1H), 6.75-7.00 (m, 3H), 7.00-7.30 (m, 4H)

### EXAMPLE 35A19

1-(1-Cyclohexanecarbonyl-1,2,3,4-tetrahydroquinoline-2-ylmethyl)-4-(2,5-dichlorophenyl)piperazine

From 1-(2,5-dichlorophenyl)piperazine (commercial)

Flash chromatography: petroleum ether—EtOAc 8:2. Yield: 55%.

¹H-NMR (δ): 0.70-2.85 (m, 22H), 2.85-3.15 (m, 4H), 5.10 (bs, 1H), 6.85-7.00 (m, 2H), 7.00-7.35 (m, 4H)

### EXAMPLE 35A20

1-(1-Cyclohexanecarbonyl-1,2,3,4-tetrahydroquinoline-2-ylmethyl)-4-[4-fluoro-2-(2,2,2-trifluoroethoxyphenyl)]piperazine

From 1-[4-fluoro-2-(2,2,2-trifluoroethoxyphenyl)]piperazine (EP 0748800, 1996)

Flash chromatography: petroleum ether—EtOAc 4:6. Yield: 28%.

¹H-NMR (δ): 0.70-2.20 (m, 13H), 2.20-2.85 (m, 8H), 2.85-3.15 (m, 4H), 4.40 (q, 2H), 5.10 (bs, 1H), 6.55-6.80 (m, 2H), 6.80-6.95 (m, 1H), 6.95-7.35 (m, 4H)

### EXAMPLE 35A21

1-(1-Cyclohexanecarbonyl-1,2,3,4-tetrahydroquinoline-2-ylmethyl)-4-(2-pyrimidinyl)piperazine

From 1-(2-pyrimidinyl)piperazine (commercial)

Flash chromatography: petroleum ether—EtOAc 4:6. Yield: 13%.

¹H-NMR (δ): 0.70-2.30 (m, 21H), 3.60-3.90 (m, 4H), 5.10 (bs, 1H), 6.45 (dd, 1H), 7.00-7.30 (m, 4H), 8.30 (dd, 2H)

### EXAMPLE 35A22

1-(1-Cyclohexanecarbonyl-1,2,3,4-tetrahydroquinoline-2-ylmethyl)-4-(8-quinolinyl)piperazine

From 1-(8-quinolinyl)piperazine (WO 00/40554, 2000)

Flash chromatography: petroleum ether-EtOAc 50:50. Yield: 70%.

¹H-NMR (δ): 0.79-2.25 (m, 13H), 2.26-3.50 (m, 8H), 3.22-3.49 (m, 4H), 5.00-5.31 (m, 1H), 7.05-7.48 (m, 7H), 8.09 (dd, 1H), 8.82 (dd, 1H)

### EXAMPLE 35A23

1-(5-chloro-2-cyanophenyl)-4-(1-cyclohexanecarbonyl-1,2,3,4-tetrahydroquinoline-2-ylmethyl)piperazine

1-(5-chloro-2-cyanophenyl)piperazine (WO 01/05765, 2001)

Flash chromatography: petroleum ether-EtOAc 50:50. Yield: 80%.

¹H-NMR (δ): 0.79-2.01 (m, 12H), 2.02-2.21 (m, 1H), 2.20-2.81 (m, 8H), 3.08-3.29 (m, 4H), 5.00-5.23 (m, 1H), 6.80-7.00 (m, 2H), 7.05-7.30 (m, 4H), 7.48 (d, 1H)

### EXAMPLE 35A24

1-(5-Cyano-2-methoxyphenyl)-4-(1-cyclohexanecarbonyl-1,2,3,4-tetrahydroquinoline-2-ylmethyl)piperazine

From 4-(5-cyano-2-methoxyphenyl)piperazine (WOO1/29022, 2001)

Flash chromatography: petroleum ether-EtOAc 50:50. Yield: 63%.

¹H-NMR (δ): 0.75-2.01 (m, 12H), 2.02-2.18 (m, 1H), 2.22-2.81 (m, 8H), 2.92-3.12 (m, 4H), 3.89 (s, 3H), 4.95-5.19 (m, 1H), 6.85 (d, 1H), 7.04-7.32 (m, 6H)

### EXAMPLE 35A25

1-(1-Acetyl-4-indolyl)-4-(1-cyclohexanecarbonyl-1,2,3,4-tetrahydroquinoline-2-ylmethyl)piperazine

From 1-(1-acetyl-4-indolyl)piperazine

Flash chromatography: petroleum ether—EtOAc 50:50. Yield: 63.2%.

¹H-NMR (δ): 0.75-2.25 (m, 12H), 2.25-2.90 (m, 9H), 3.00-3.40 (m, 4H), 4.90-5.30 (m, 1H), 6.45-6.65 (m, 2H), 6,95-7.30 (m, 7H).

The starting piperazine was prepared as following:

a) 1-(1-tert-Butoxycarbonyl)-4-(1-acetyl-4-indolyl)piperazine (Compound 35A25-A)

A suspension of 0.6 g of 1-(1-tert-butoxycarbonyl)-4-(4-indolyl)piperazine (WO99/67237, 1999) and 0.24 g of 60% NaH oil dispersion in 6 ml of anhydrous DMF was stirred for 30 min. at r.t. under nitrogen stream then for 1 h at 55° C. Afterwards, a solution of 0.29 ml of acetyl chloride in 3 ml of DMF was dropped; stirring was continued for 6 h at 55° C. After cooling, the reaction mixture was diluted with 60 ml of H₂O, extracted with Et₂O (3×30 ml), washed and dried (Na₂SO₄) and evaporated to dryness in vacuo. The residue was purified by flash chromatography (Et₂O— petroleum ether 40:60) affording 0.55 g (79.3%) of the title compound.

¹H-NMR (δ): 2.63 (s, 3H), 3.18-3.40 (m, 8H), 6.73-6.90 (m 2H), 7.12-7.38 (m, 1H), 7.81 (d, 1H), 8.00 (dd, 1H), 9.40 (br, 2H), 9.80-11.00 (br, 1H).

The starting piperazine was prepared as follows:

b) 1-(1-Acetyl-4-indolyl)piperazine (Compound 35A25-B)

A solution of 0.34 g of Compound 35A25-A in 3 ml of MeOH and 15 ml of a 2 N solution of HCl in Et₂O was kept overnight resting. The precipitated solid was filtered affording 0.29 g of the title compound as a dihydrochloride.

¹H-NMR (δ): 1.50 (s, 9H), 2.62 (s, 3H), 2.95-3.22, 3.50-3.80 (2m, 8H), 6.65 (d 1H), 6.80 (dd, 1H), 7.18-7.33 (m, 1H), 7.40 (d, 1H), 8.14 (dd, 1H).

### EXAMPLE 35A26

1-(1-Cyclohexanecarbonyl-1,2,3,4-tetrahydroquinoline-2-ylmethyl)-4-(7-indolyl)piperazine

From 1-(7-indolyl)piperazine (WO 94/15919, 1994)

Flash chromatography: petroleum ether—EtOAc 50:50. Yield: 72.4%

¹H-NMR (δ): 0.75-2.25 (m, 12H), 2.25-2.90 (m, 9H), 2.90-3.20 (m, 4H), 4.90-5.30 (m, 1H), 6.48-6.68 (m, 1H), 6.80 (dd, 1H), 6,95-7.40 (m, 7H).

### EXAMPLE 35A27

1-(3-Cyano-4-indolyl)-4-(1-cyclohexanecarbonyl-1,2,3,4-tetrahydroquinoline-2-ylmethyl)piperazine

From 1-(3-cyano-4-indolyl)piperazine (WO99/67237, 1999)

Flash chromatography: petroleum ether—EtOAc 30:70. Yield: 35.19%.

¹H-NMR (δ): 0.80-2.20 (m, 12H), 2.20-2.90 (m, 9H), 2.90-3.20 (m, 4H), 4.90-5.30 (m, 1H), 6.65-6.85 (m, 1H), 7.00-7.35 (m, 6H), 7.70 (d, 1H), 8.82 (b, 1H).

### EXAMPLE 35A28

1-(1-Cyclohexanecarbonyl-1,2,3,4-tetrahydroquinoline-2-ylmethyl)-4-(2,4-difluorobenzyl)piperazine

From 1-(2,4-difluorobenzyl)piperazine.

Flash chromatography: petroleum ether—EtOAc 70:30. Yield: 58.5%.

¹H-NMR (δ): 0.80-2.15 (m, 12H), 2.15-2.80 (m, 13H), 3.30 (s, 2H), 4.90-5.20 (m, 1H), 6.65-6.95 (m, 2H), 6.65-7.45 (m, 5H).

The starting piperazine was prepared as follows:

a) 1-(2,4-Difluorobenzyl)piperazine (Compound 35A28-A)

The title product was prepared following the general procedure described for benzyl piperazines in CA2188484.

¹H-NMR (δ): 2.26-2.55 (m, 4H), 2.79-2.95 (m, 4H), 3.52 (s, 2H), 6.71-6.88 (m, 2H), 7.22-7.41 (m, 1H).

### EXAMPLE 35A29

1-(2-Bromobenzyl)-4-(1-cyclohexanecarbonyl-1,2,3,4-tetrahydroquinoline-2-ylmethyl)piperazine

From 1-(2-bromobenzyl)piperazine.

Flash chromatography: petroleum ether—EtOAc 50:50. Yield:81.9%.

¹H-NMR (δ): 0.70-2.10 (m, 12H), 2.10-3.00 (m, 13H), 3.40-3.80 (m, 2H), 4.90-5.20 (m, 1H), 6.90-7.40 (m, 6H), 7.40-7.65 (m, 2H)

The starting piperazine was prepared as follows:

a) 1-(2-Bromobenzyl)piperazine (Compound 35A29-A)

The title product was prepared following the general procedure described for benzyl piperazines in CA2188484.

¹H-NMR (δ): 2.41-2.55 (m, 4H), 2.62-2.98 (m, 4H), 3.64 (s, 2H), 6.71-6.88 (m, 2H), 7.13 (dd, 1H), 7.25(dd, 1H), 7.41-7.56 (m, 2H).

### EXAMPLE 35A30

1-(1-Cyclohexanecarbonyl-1,2,3,4-tetrahydroquinoline-2-ylmethyl)-4-(2,5-difluorobenzyl)piperazine

From 1-(2,5-difluorobenzyl)piperazine (CA2188484).

Eluent: Petroleum Ether-EtOAc 1:1. Yield: 52%.

¹H-NMR (δ):0.8-2.11(m, 12H), 2.12-2.83(m, 13H), 3.52 (s, 2H), 4.95-5.12 (m, 1H), 6.80-7.00 (m, 2H), 7.01-7.32 (m, 5H).

### EXAMPLE 35A31

1-(1-Cyclohexanecarbonyl-1,2,3,4-tetrahydroquinoline-2-ylmethyl)-4-(1-naphthyl)piperazine

From 1-(1-naphthyl)piperazine.

Flash chromatography: petroleum ether—EtOAc 7:3. Yield: 83.7%

¹H-NMR (δ): 0.81-2.15 (m, 11H), 2.10-2.29 (m, 1H), 2.32-2.91 (m, 9H), 3.00-3.28 (m, 4H), 4.98-5.25 (m, 1H), 7.01-7.32- (m, 5H), 7.34-7.59 (m; 4H), 7.71-7.92 (m; 1H), 8.08-8.27 (m; 1H).

The starting piperazine was prepared as follows:

a) 1-Benzyl-4-(1-naphthyl)piperazine (Compound 35A31-A)

A reaction flask was charged with cesium carbonate (11 g), BINAP (0.22 g) and palladium acetate (53 mg), under N₂ atmosphere; afterwards, was added 1-bromonaphthalene (5 g), 1-benzylpiperazine (5 ml) and toluene (70 ml) and the suspension was stirred at reflux for 20 h. After cooling to r.t., the mixture was diluted with diethyl ether (20 ml), filtered through a Celite bed and concentrated in vacuo. The residue was purified by flash chromatography eluting with petroleum ether—ethyl acetate 9:1 to give 4.16 g (57%) of the title compound.

¹H-NMR (δ): 2.67-2.84 (m; 4H), 3.07-3.24 (m; 4H), 3.66 (s; 2H), 7.09 (dd, 1H), 7.24-7.61 (m; 9H), 7.76-7.88 (m; 1H), 8.12-8.30 (m; 1H)

b) 1-Naphthylpiperazine (Compound 35A31-B)

To a solution of Compound 35A31-A (4.16 g) in MeOH (250 ml) were added 10% Pd-C (0.65 g) and ammonium formiate (6.12 g) and the mixture stirred at reflux under N₂ atmosphere for 5 h. After cooling to r.t., the catalyst was filtered off, the solvent was evaporated to dryness and the residue dissolved in CH₂Cl₂. The solution was washed with aqueous 5% NaHCO₃, dried (Na₂SO4), filtered and evaporated at reduced pressure to give 2.5 g (85%) of the title compound.

¹H-NMR (δ): 1.71 (bs; 1H), 2.92-3.36 (m; 8H), 7.09 (dd; 1H), 7.38-7.58 (m; 4H), 7.74-7.92 (m; 1H), 8.12-8.31 (m; 1H)

### EXAMPLE 35A32

1-(7-Bromo-4-indolyl)-4-(1-cyclohexanecarbonyl-1,2,3,4-tetrahydroquinoline-2-ylmethyl)piperazine

From 1-(7-Bromo-4-indolyl)piperazine.

Flash chromatography: petroleum ether-EtOAc 50:50. Yield: 59%.

¹H-NMR (δ): 0.79-2.21 (m, 13H), 2.22-2.84 (m, 8H), 2.87-3.24 (m, 4H), 4.99-5.23 (m, 1H), 6.61 (d, 1H), 7.03-7.30 (m, 7H), 8.25-8.47 (m, 1H)

The starting piperazine was prepared as following:

a) 1-tert-Butoxycarbonyl-4-(7-bromo-4-indolyl)piperazine (Compound 35A32-A)

To a solution of 1.53 g of 1-tert-butoxycarbonyl-4-(4-indolyl)piperazine in 20 ml of THF was added 0.89 g of NBS. The mixture was stirred for 6 h at r.t., then quenched with water, extracted with EtOAc (2×30 ml). The organic layer was washed with water, dried (Na₂SO₄) and evaporated to dryness in vacuo. The residue was purified by flash chromatography (petroleum ether—EtOAc 8:2) to afford the title compound (0.71 g; 37%).

¹H-NMR (δ): 1.49 (s, 9H), 3.04-3.34 (m, 4H), 3.55-3.78 (m, 4H), 6.40-6.58 (m, 1H), 6.59-6.68 (m, 1H), 7.15-7.30 (m, 2H), 8.35 (bs, 1H)

b) 1-(7-Bromo-4-indolyl)piperazine (Compound 35A32-B)

The title compound was prepared following the same procedure described for Compound 35A25-B but starting from compound 35A32-A instead of Compound 35A25-A.

The crude was used without further purification.

¹H-NMR (δ): 1.90 (bs, 1H), 3.01-3.24 (m, 8H), 3.55-3.78 (m, 4H), 6.49 (d, 1H), 6.61 (s, 1H), 7.12-7.30 (m, 2H), 8.35 (bs, 1H)

### EXAMPLE 35A33

1-(1-Cyclohexanecarbonyl-1,2,3,4-tetrahydroquinoline-2-ylmethyl)-4-(3,4-dihydro-2H-benzo[b][1,4]dioxepin-6-yl)piperazine

From 1-(3,4-dihydro-2H-1,5-benzodioxepin-6-yl)piperazine (J. Med Chem, 31, 1934-1940, (1988)).

Flash chromatography petroleum ether—ethyl acetate 7:3.

¹H-NMR (δ): 0.81-1.89 (m, 11H), 1.91-2.01 (m, 1H), 2.03-2.82 (m, 13H), 2.91-3.11 (m, 4H), 4.16-4.26(m, 2H), 4.98-5.25 (m, 1H), 6.52-6.71 (m; 2H), 6.81 (t; 1H), 7.06-7.18 (m; 4H).

### EXAMPLE 35A34

1-(2-Chlorobenzyl)-4-(1-cyclohexanecarbonyl-1,2,3,4-tetrahydroquinoline-2-ylmethyl)piperazine

From 1-(2-chlorobenzyl)piperazine (CA2188484))

Eluent: Petroleum Ether-EtOAc 1:1. Yield: 64%.

¹H-NMR (δ):0.9-2.98(m, 25H), 3.52 (s, 2H), 4.95-5.12 (m, 1H), 6.80-7.11(m, 8H).

### EXAMPLE 35A35

1-(1-Cyclohexanecarbonyl-1,2,3,4-tetrahydroquinoline-2-ylmethyl)-4-(6-methoxy-2-pyridinyl)piperazine

1-(6-Methoxy-2-pyridinyl)piperazine (Reignier et al. Arzneim.Forsch (Drug Res) 24, 12 (1974)).

Flash chromatography petroleum ether—ethyl acetate 7:3. Yield: 84.8%

¹H-NMR (δ):0.8-2.2 (m, 13H), 2.20-2.73(m, 8H), 3.35-4.05 (m, 4H), 3.79-3.91 (s, 3H), 4.95-5.12 (m, 1H), 6.05-6.19 (m, 2H), 7.01-7.42 (m, 5H).

### EXAMPLE 35A36

1-(1-Cyclohexanecarbonyl-1,2,3,4-tetrahydroquinoline-2-ylmethyl)-4-(2,5-dichlorobenzyl)piperazine

From 1-(2,5-dichlorobenzyl)piperazine (CA 2188484).

Flash chromatography petroleum ether—ethyl acetate 8:2. Yield: 76.8%

¹H-NMR (δ):0.8-2.20 (m, 13H), 2.30-2.85(m, 12H), 3.52 (s, 2H), 4.95-5.12 (m, 1H), 7.05-7.30 (m, 6H), 7.45 (m 1H).

### EXAMPLE 36

1-(4-Indolyl)-4-(1-piperidinocarbonyl-1,2,3,4-tetrahydroquinoline-2-ylmethyl)piperazine

The title compound was synthesised following the alternative acylation procedure described for the compound of Example 1 (step f) using 1-piperidinocarbonyl chloride instead of cyclohexanecarbonyl chloride and chloroform instead of dichloromethane, and refluxing for 8 hours in the presence of TEA The crude was purified by flash chromatography eluting with petroleum ether—ethyl acetate-2.5N methanolic ammonia 8:2:0.01 to give the title compound (10%).

¹H-NMR (δ):1.31-2.48 (m, 9H), 2.53-2.91 (m, 7H), 3.08-3.41 (m, 10H), 4.29-4.48 (m, 1H), 6.47-6.63 (m, 2H), 6.80-7.00 (m, 2H), 7.01-7.21 (m, 5H), 8.10-8.28 (bs, 1H)

### EXAMPLE 37

1-[1-(3-Cyanopropionyl)-1,2,3,4-tetrahydroquinoline-2-ylmethyl]-4-(4-indolyl)piperazine

To a solution of 0.37 g of Compound 27A in 5 mL of 1-methyl-2-pyrrolidinone, was added 0.14 g of sodium cyanide and the mixture was stirred at 130° C. for 3 hours, cooled at r.t., poured into H₂O and extracted with EtOAc. The organic layer was washed with water, dried (Na₂SO₄) and evaporated to dryness in vacuo. The residue was purified by flash chromatography (petroleum ether-EtOAc 2:8) to afford the title compound (0.14 g; 36%).

¹H-NMR (δ): 1.38-1.64 (m, 1H), 2.10-3.08 (m, 13H), 3.08-3.32 (m, 4H), 5.01-6.24 (m, 1H), 6.47-6.61 (m, 2H), 7.01-7.31 (m, 5H), 8.10-8.28 (bs, 1H)

### EXAMPLE 38

1-(1-Cyclohexanecarbonyl-8-fluoro-1,2,3,4-tetrahydroquinoline-2-ylmethyl)-4-(4-indolyl)piperazine

a) 8-fluoro-2-methylquinoline (Compound 38A)

Compound 38A was obtained with the same method as described for Compound 25A, but using 2-fluoroaniline instead of 4-trifluoromethylaniline. The crude was purified by flash chromatography eluting with petroleum ether—ethyl acetate 7:3, affording 3.5 g (80%) of the title compound.

¹H-NMR (δ): 2.81 (s; 3H), 7.28-7.46 (m; 3H), 7.50-7.62(m; 1H), 8.08 (d; 1H)

b) 2-Bromomethyl-8-fluoroquinoline (Compound 38B)

The title compound was prepared as described for Compound 5A, starting from Compound 38A instead of 6-fluoro-2-methylquinoline. The crude was purified by flash chromatography eluting with petroleum ether—ethyl acetate 9:1 to give, as a first eluted product, 1.9 g (36%) of the title compound and then 2.16 g of starting material.

¹H-NMR (CDCl₃; 67): 4.78 (s; 2H), 7.37-7.72 (m; 4H), 8.22 (d; 1H).

c) 8-Fluoro-2-[4-(4-indolyl)-1-piperazinylmethyl]quinoline (Compound 38C)

Compound 38C was obtained in the same way as described for Compound 1C (procedure d) but using Compound 38B instead of 2-chloromethylquinoline. The crude was purified by flash chromatography eluting with petroleum ether—ethyl acetate 1:1 to give the title compound (75%).

¹H-NMR (; 67): 2.74-2.95 (m; 4H), 3.21-3.42 (m; 4H), 4.02 (s; 2H), 6.49-6.68 (m; 2H), 7.00-7.19 (m; 3H), 7.34-7.52 (m; 2H), 7.61 (d; 1H), 7.80 (d; 1H), 8.12-8.29 (m, 2H)

d) 1-(8-Fluoro-1,2,3,4-tetrahydroquinoline-2-ylmethyl)-4-(4-indolyl)piperazine (Compound 38D)

The title compound was prepared as described for Compound 1B (procedure e) starting from Compound 38C instead of Compound 1C. The crude was purified by flash chromatography eluting with petroleum ether—ethyl acetate 1:1 to give the title compound (57%).

¹H-NMR (δ): 1.49-1.75 (m; 1H), 1.87-2.08 (m; 1H), 2.51 (d; 2H), 2.59-3.02 (m; 6H), 3.21-3.60 (m; 5H), 3.73-3.95 (br; 1H), 6.41-6.80 (m; 5H), 7.04-7.22 (m; 3H), 8.08-8.28 (br; 1H).

e) 1-(1-Cyclohexanecarbonyl-8-fluoro-1,2,3,4-tetrahydroquinoline-2-ylmethyl)-4-(4-indolyl)piperazine

The title compound was synthesised following the alternative acylation procedure described for the compound of Example 1 (step f) but starting from Compound 38D instead of Compound 1B. The crude was purified by flash chromatography eluting with toluene—acetone 8:2 affording the title compound (70%).

¹H-NMR (iδ): 0.82-1.99 (m; 11H), 2.03-2.25 (m; 1H), 2.28-2.91 (m; 9H), 3.08-3.44 (m; 4H); 4.96-5.22 (m; 1H), 6.45-6.77 (m; 2H), 6.96-7.29 (m; 6H), 8.05-8.26 (br, 1H).

### EXAMPLE 39

1-[1-(3-Acetylaminopropionyl)-1,2,3,4-tetrahydroquinoline-2-ylmethyl]-4-(4-indolyl)piperazine

A mixture of 0.16 g of the compound of Ex. 28, 0.058 ml of TEA, 0.03 ml of acetyl chloride and 3.8 ml of CH₂Cl₂ was stirred at 20 to 25° C. under nitrogen atmosphere for 2 hour. The solution was washed with a 0.5 N NaOH (1×10 ml) and 15 ml of water. The organic layer, dried on anhydrous sodium sulphate, was evaporated to dryness and the residue purified by flash chromatography (EtOAc—2 N NH₃ sol. in methanol 93:7) to give 0.13 g (75%) of the title compound and the compound of Example 41 (0.027 g).

¹H-NMR (δ): 1.40-1.65 (m, 1H), 1.95 (s, 3H), 2.10-2.95 (m, 11H), 3.10-3.30 (m, 4H), 3.30-3.370 (m, 2H), 5.10 (b, 1H), 6.40-6.60 (m, 3H), 7.00-7.25 (m, 7H), 8.20 (s, 1H)

### EXAMPLE 40

1-[1-(3-Carbamoylaminopropionyl)-1,2,3,4-tetrahydroquinoline-2-ylmethyl]-4-(4-indolyl)piperazine

A mixture of 0.17 g of the compound of Ex. 28, 0.067 ml of trimethylsilylisocyanate and 4 ml of THF was stirred at 20 to 25° C. under nitrogen atmosphere for 2 hour. After overnight resting, the solution was taken up with a 1 N NaOH (1×10 ml) and extracted with EtOAc (2×10 ml) which was washed with 10 ml of water. The organic layer, dried on anhydrous sodium sulphate, was evaporated to dryness and the residue purified by flash chromatography (EtOAc—2 N NH₃ sol. in methanol 92:8) to give 0.13 g (68%) of the title compound.

¹H-NMR (δ): 1.35-1.65 (m, 1H), 2.10-2.90 (m, 11H), 3.10-3.25 (m, 4H), 3.25-3.65 (m, 2H), 4.35 (b, 1H), 5.10 (b, 1H), 5.35 (b, 1H), 6.45-6.65 (m, 2H), 7.00-7.30 (m, 7H), 8.15 (s, 1H)

### EXAMPLE 41

1-[1-(3-bis-Acetylaminopropionyl)-1,2,3,4-tetrahydroquinoline-2-ylmethyl]-4-(4-indolyl)piperazine

The title product was obtained during the synthesis of the compound of Example 39.

¹H-NMR (δ): 1.35-1.65 (m, 1H), 2.05-3.00 (m, 18H), 3.10-3.30 (m, 4H), 3.80-4.20 (m, 2H), 5.10 (b, 1H), 6.45-6.60 (m, 2H), 7.00-7.30 (m, 7H), 8.15 (s, 1H)

### EXAMPLE 42

1-(6-Chloro-1-cyclohexanecarbonyl-1,2,3,4-tetrahydroquinoline-2-ylmethyl)-4-(4-indolyl)piperazine

a) 6-Chloro-2-hydroxymethyl-1,2,3,4-tetrahydroquinoline (Compound 42A)

A mixture of 1.24 g of 6-chloro-1,2,3,4-tetrahydroquinoline-2-carboxylic acid, 30 ml of anhydrous THF and 6 ml of 10 M BH₃.Me₂S in THF was stirred at r.t. for 3 h under nitrogen. After overnight standing at r.t., 5 ml of MeOH was dropped into the mixture, and after 15′ was added 5 ml of 2N HCl, The stirred mixture was heated at 60° C. for 1 h, then evaporated to dryness in vacuo. The residue was treated with 2 N NaOH (30 ml) and extracted with CHCl₃ (2×40 ml). The organic layer was dried (anhydrous Na₂SO₄) and evaporated to dryness in vacuo; the residue was purified by flash chromatography (CHCl₃—MeOH 100:1) to afford 0.35 g (35.3%) of Compound 42A as a thick oil.

¹H-NMR (δ): 1.50-2.10 (m, 3H), 2.55-2.95 (m, 2H), 3.30-3.50 (m, 1H), 3.50-3.85 (m, 2H), 3.85-4.70 (b, 1H), 6.45 (d, 1H), 6.80-7.05 (m, 2H)

b) 6-Chloro-2-iodomethyl-1,2,3,4-tetrahydroquinoline (Compound 42B)

The title compound was synthesized following the procedure described for compound 34C but using as a starting material compound 42A instead of compound 34B.

¹H-NMR (δ):1.63-2.15 (2m, 2H), 2.55-2.90 (m, 2H), 3.05-3.38 (2m, 2H), 3.38-3.55 (m, 1H), 3.85-4.40 (b, 1H), 6.45 (d, 1H), 6.90-7.00 (m, 2H)

c) 6-Chloro-2-[4-(4-indolyl)-1-piperazinyl]methyl]-1,2,3,4-tetrahydroquinoline (Compound 42C)

The title compound was synthesized following the procedure described for compound 34D but using as a starting material compound 42B instead of compound 34C.

he residue was purified by flash chromatography (petroleum ether—EtOAc 70:30) to afford Compound 42B (55.8%) as a yellow oil.

¹H-NMR (δ): 1.40-1.75 (m, 1H), 1.80-2.05 (m, 1H), 2.35-3.00 (m, 8H), 3.10-3.65 (m, 5H), 4.50-4.95 (b, 1H), 6.35 (d, 1H), 6.50-6.70 (m, 2H), 6.85-7.00 (m, 2H), 7.05-7.25 (m, 3H), 8.05-8.30 (b, 1H)

d) 1-(6-Chloro-1-cyclohexanecarbonyl-1,2,3,4-tetrahydroquinoline-2-ylmethyl)-4-(4-indolyl)piperazine

The title compound was synthesised starting from Compound 42C following the procedure described for the of Example 1 (step f). The residue was purified by flash chromatography eluting with petroleum ether—EtOAc gradient from 60:40 to 40:60 affording the title compond (71.8%)

¹H-NMR (δ): 0.85-2.00 (m, 12H), 2.00-2.25 (m, 1H), 2.25-2.85 (m, 8H), 3.00-3.40 (m, 4H), 4.85-5.30 (m, 1H), 6.45-6.65 (m, 2H), 6.95-7.25 (m, 6H), 8.15 (b, 1H)

### EXAMPLE 43

(R)-1-(1-Cyclohexanecarbonyl-1,2,3,4-tetrahydroquinoline-2-ylmethyl)-4-(1-methyl-4-indolyl)piperazine

The title compound was prepared as described in Example 15 but using the compound of Example (+)1 instead of (−)1. Yield: 30%.

¹H-NMR (δ): 0.80-1.99 (m, 11H), 2.05-2.24 (m, 1H), 2.26-2.85 (m, 9H), 3.10-3.35 (m, 4H), 3.76 (s 3H) 5.00-5.25 (m, 1H), 6.45-6.60 (m, 2H), 7.00-7.30 (m, 7H).

### EXAMPLE 44

(Z)-1-[1-(4-Hydroxycyclohexanecarbonyl)-1,2,3,4-tetrahydroquinoline-2-ylmethyl]-4-(4-indolyl)piperazine

a) (Z)-1-[1-(4-Diphenyltertbutylsilyloxycyclohexanecarbonyl)-1,2,3,4-tetrahydroquinoline-2-ylmethyl]-4-(4-indolyl)piperazine (Compound 44A)

The title compound was synthesised following the acylation procedure described for the compound of Example 1 (step f) starting from Compound 1B but using cis-4-diphenyltertbutylsilyloxycyclohexanecarbonyl chloride, (prepared as described in EP352909), instead of cyclohexanecarbonyl chloride. Flash chromatography eluent: petroleum ether—EtOAc 6:4. Yield: 87%.

¹H-NMR (δ): 0.86-1.86 (m, 18H), 1.95-2.78 (m, 10H), 3.11-3.41 (m, 4H), 3.82-4.01 (m, 1H), 5.05-5.28 (m, 1H), 6.48-6.62 (m, 2H), 7.00-7.46 (m, 11H), 7.53-7.71 (m, 6H), 8.02-.8.25 (br 1H).

b) (Z)-1-[-(4-Hydroxycyclohexanecarbonyl)-1,2,3,4-tetrahydroquinoline-2-ylmethyl]-4-(4-indolyl)piperazine

To a solution of compound 44A (0.2 g) in 20 ml of THF was added tetrabutylammonium fluoride and the resulting suspension stirred at r.t for 3 days. Afterwards the solvent was evaporated under vacuum; the residue was dissolved in CH₂Cl₂ (40 ml) and washed with H₂O (30 ml). The organic layer was dried (Na₂SO₄) and the crude was purified by flash chromatography (CH₂Cl₂— MeOH 95:5) to give the title compound (63%).

¹H-NMR (δ): 1.10-1.99 (m, 9H), 2.01-2.22 (m, 2H), 2.33.-2.88 (m, 9H), 3.12-3.33 (m, 4H), 3.82-4.01 (m, 1H), 5.05-5.29 (m, 1H), 6.48-6.68 (m, 2H), 7.07-7.29 (m, 7H), 8.03-.8.29 (br 1H).

### EXAMPLE 45

(E)-1-[1-(4-Hydroxycyclohexanecarbonyl)-1,2,3,4-tetrahydroquinoline-2-ylmethyl]-4-(4-indolyl)piperazine

a) E)-1-[1-(4-Diphenyltertbutylsilyloxycyclohexanecarbonyl)-1,2,3,4-tetrahydroquinoline-2-ylmethyl]-4-(4-indolyl)piperazine (Compound 45A)

The title compound was synthesised following the same method described for compound 44A but using trans-4-diphenyltertbutylsilyloxycyclohexanecarbonyl chloride (prepared as described in EP352909), instead of cis-4-diphenyltertbutylsilyloxycyclohexanecarbonyl chloride. Flash chromatography eluent: petroleum ether—EtOAc 6:4. Yield: 87%.

¹H-NMR (δ): 0.89-1.87 (m, 18H), 1.93-2.75 (m, 10H), 3.14-3.47 (m, 4H), 3.51-3.71 (m, 1H), 5.05-5.28 (m, 1H), 6.50-6.60 (m, 2H), 7.02-7.48 (m, 11H), 7.52-7.70 (m, 6H), 8.03-.8.24 (br 1H).

b) (E)-1-[1-(4-Hydroxycyclohexanecarbonyl)-1,2,3,4-tetrahydroquinoline-2-ylmethyl]-4-(4-indolyl)piperazine

The title compound was synthesised following the procedure described for the compound 44 but starting from compound 45A (44%).

¹H-NMR (δ): 1.00-2.05 (m, 9H), 2.07-2.27 (m, 2H), 2.38.-2.88 (m, 9H), 3.15-3.38 (m, 4H), 3.50-3.71 (m, 1H), 5.06-5.32 (m, 1H), 6.51-6.70 (m, 2H), 7.07-7.31 (m, 7H), 8.05-.8.32 (br 1H).

### EXAMPLE 46

1-(1-Cyclohexanecarbonyl-7-fluoro-1,2,3,4-tetrahydroquinoline-2-ylmethyl)-4-(4-indolyl)piperazine

a) 7-Fluoro-2-methylquinoline (Compound 46A)

Compound 46A was obtained with the same method as described for Compound 25A, but using 3-fluoroaniline instead of 4-trifluoromethylaniline. The crude was purified by flash chromatography eluting with petroleum ether—ethyl acetate 75:25, affording 2.6 g (61.1%) of the title compound.

¹H-NMR (CDCl₃; δ): 2.74 (s; 3H), 7.17-7.36 (m; 2H), 7.67 (dd; 1H), 7.71-7.84 (m; 1H), 8.06 (d; 1H).

b) 2-Bromomethyl-7-fluoroquinoline (Compound 46B)

The title compound was prepared as described for Compound 5A, starting from Compound 46A instead of 6-fluoro-2-methylquinoline. The crude was purified by flash chromatography eluting with petroleum ether—ethyl acetate 85:15 to give, as a first eluted product, 1.95 g (61%) of the title compound and then 1.14 g of starting material 46A.

¹H-NMR (CDCl₃; δ): 4.71 (s; 2H), 7.32-7.43 (m; 1H), 7.56 (d; 1H), 7.74 (dd; 1H), 7.76-7.87 (m; 1H), 8.18 (d; 1H)

c) 7-Fluoro-2-[4-(4-indolyl)-1-piperazinylmethyl]quinoline (Compound 46C)

Compound 46C was obtained in the same way as described for Compound 1C (procedure d) but using Compound 46B instead of 2-chloromethylquinoline. The crude was purified by flash chromatography eluting with petroleum ether—ethyl acetate 6:4 to give the title compound (41%).

¹H-NMR (CDCl₃; δ): 2.68-2.96 (m; 4H), 3.22-3.39 (m; 4H), 3.92 (s; 2H), 6.48-6.66 (m; 2H), 7.02-7.22 (m; 3H), 7.24-7.39 (m; 1H), 7.62-7.88 (m; 3H), 8.09-8.31 (m; 2H).

d) 1-(7-Fluoro-1,2,3,4-tetrahydroquinoline-2-ylmethyl)-4-(4-indolyl)piperazine (Compound 46D)

The title compound was prepared as described for Compound 1B (procedure e) starting from Compound 46C instead of Compound 1C. The crude was purified by flash chromatography eluting with petroleum ether—ethyl acetate 7:3 to give the title compound (45%).

¹H-NMR (CDCl₃; δ): 1.41-1.68 (m; 1H), 1.86-2.01 (m; 1H), 2.43-2.98 (m; 8H), 3.14-3.57 (m; 5H), 4.51-4.89 (br; 1H), 6.12-6.36 (m; 2H), 6.48-6.68 (m; 2H), 6.81-6.90 (m; 1H), 7.04-7.19 (m; 3H), 8.08-8.23 (br, 1H).

e) 1-(1-Cyclohexanecarbonyl-7-fluoro-1,2,3,4-tetrahydroguinoline-2-ylmethyl)-4-(4-indolyl)piperazine

The title compound was synthesised following the alternative acylation procedure described for the compound of Example 1 (step f) but starting from Compound 46D instead of Compound 1B. The crude was purified by flash chromatography eluting with petroleum ether-ethyl acetate 7:3 affording the title compound (57%).

¹H-NMR (CDCl₃; δ): 0.91-2.01 (m; 11H), 2.03-2.26 (m; 1H), 2.29-2.89 (m; 9H), 3.11-3.40 (m; 4H); 4.91-5.25 (m; 1H), 6.48-6.68 (m; 2H), 6.82-7.24 (m; 6H), 8.03-8.22 (br, 1H).

### EXAMPLE 47

1-(1-Cyclohexanecarbonyl-6-phenyl-1,2,3,4-tetrahydroquinoline-2-ylmethyl)-4-(4-indolyl)piperazine

To a solution of 0.053 g of the compound of Example 34 in 2.5 ml of anhydrous 1,2-DME was added 0.008 g of tetrakis(triphenylphosphine)-palladium(0), 0.018 g of 97% phenylboric acid and 0.7 ml of K₂CO₃ aq. saturated solution. The mixture was stirred for 12 h under nitrogen and then diluted with H₂O (20 ml) and extracted with EtOAc (3×10 ml). The organic layer was washed with brine, dried (anhydrous Na₂SO₄) and evaporated to dryness in vacuo. The residue was purified by flash chromatography (petroleum ether—EtOAc 1:1) affording 0.010 g (10%) of the title compound as a thick oil.

¹H-NMR (δ): 0.70-2.05 (m,l 1H), 2.05-2.30 (m, 1H), 2.30-2.90 (m, 9H), 3.05-3.35 (m, 4H), 4.90-5.35 (m, 1H), 6.45-6.70 (m, 2H), 7.00-7.25 (m, 4H), 7.30-7.55 (m, 5H), 7.55-7.70 (m, 2H), 8.15 (b, 1H)

### EXAMPLE 48

1-(1-Cyclohexanecarbonyl-2,3-dihydroindole-2-ylmethyl)-4-(4-indolyl)piperazine

a) 2-[4-(4-Indolyl)-1-piperazinyl]carbonyllindoline (Compound 48A)

The title compound was synthesised following the procedure described for Compound 1D (step f) but using 2-indolinecarboxylic acid instead of 1,2,3,4-tetrahydroquinoline-2-carboxylic acid. The crude solid residue was stirred with Et₂O (30 ml) for 0,5 h at r.t. to afford 0.65 g (93, 2%) of the title compound as an ivory solid.

¹H-NMR (δ): 3.05-3.45 (m, 5H), 3.45-3.65 (m, 1H), 3.65-4.00 (m, 8H), 3.05-4.40 (b, 1H), 4.60-4.80 (m, 1H), 6.50-6.70 (m, 2H), 6.70-6.95 (m, 2H), 6.95-7.25 (m, 5H), 8.20-8.50 (b, 1H).

b) 1-(2,3-Dihydroindole-2-ylmethyl)-4-(4-indolyl)piperazine (Compound 48B)

The title compound was synthesised following the procedure described for Compound 1B (step h) but using compound 48A instead of Compound 1D.

The residue was purified by flash chromatography (CHCl₃2 N methanolic ammonia 100:1) affording the title compound (11.8%) as a thick oil.

¹H-NMR (δ): 2.30-2.80 (m, 4H), 2.80-3.10 (m, 1H), 3.10-3.50 (m, 7H), 3.90-4.15 (m, 1H), 4.20-5.00 (b, 1H), 6.50-6.80 (m, 4H), 6.90-7.20 (m, 5H), 8.10 (b, 1H)

c) 1-(1-Cclohexanecarbonyl-2,3-dihydroindole-2-ylmethyl)-4-(4-indolyl)piperazine

The title compound was synthesised following the alternative acylation procedure described for the compound of Example 1 (step f) but starting from Compound 48B instead of Compound 1B. The crude was purified by flash chromatography eluting with petroleum ether-ethyl acetate 6:4 affording the title compound (53%).

¹H-NMR (δ):1.00-2.05 (m, 10H), 2.10-2.32 (m, 1H), 2.32-2.95 (m, 5H), 2.95-3.45 (m, 7H), 4.40-4.70 (m, 1H), 6.48-6.70 (m, 2H), 6.95-7.30 (m, 7H), 8.15 (b, 1H)

### EXAMPLE 49

1-(1-Cyclohexanecarbonyl-8-methoxy-1,2,3,4-tetrahydroquinoline-2-ylmethyl)-4-(4-indolyl)piperazine

a) 1-(8-Hydroxyquinoline-2-ylmethyl)-4-(4-indolyl)piperazine (Compound 49A)

The title compound was synthesised following the reductive amination procedure described for Compound 35B, but using 8-hydroxyquinolin-2-carbaldehyde (commercial) instead of compound 35A. The residue was purified by flash chromatography (petroleum ether—EtOAc-2.5 N methanolic ammonia 1:9:0.2) affording Compound 49A (74%).

¹H-NMR (δ):2.71-2.93 (m, 4H), 3.23-3.41 (m, 4H), 3.88-4.03 (m, 2H), 6.50-6.63 (m, 2H), 7.0-7.49 (m, 6H), 7.71 (d, 1H), 8.15 (d, 1H), 8.10-8.45 (bs, 1H)

b) 1-(8-Methoxyquinolin-2-ylmethyl)-4-(4-indolyl)piperazine (Compound 49B)

To a mixture of 0.08 g of freshly prepared sodium methoxide in 10 ml of anhydrous THF was added 0.50 g of compound 49A and the resulting solution was stirred for 0.5 hours at r.t. 0.19 ml of iodometane was added and the solution was stirred at reflux for 5 hours, cooled to r.t., poured into H₂O and extracted with EtOAc. The organic layer was washed with water, dried (Na₂SO₄) and evaporated to dryness in vacuo to afford the title compound (73%).

¹H-NMR (δ):2.73-2.90 (m, 4H), 3.21-3.38 (m, 4H), 4.00-4.14 (m, 2H), 6.50-6.65 (m, 2H), 7.00-7.14 (m, 4H), 7.35-7.48 (m, 2H), 7.83 (s, 1H), 8.12 (s, 1H), 8.22-8.34 (bs, 1H)

c) 1-(8-Methoxy-1,2,3,4-tetrahydroquinoline-2-ylmethyl)-4-(4-indolyl)piperazine (Compound 49C)

The title compound was synthesised as reported in Example 1 (step e) but using compound 49B instead of Compound 1C. The residue was purified by flash chromatography (petroleum ether—EtOAc-3:7) affording the title compound. (42%).

¹H-NMR (δ):1.50-1.74 (m, 1H), 1.88-2.05 (m, 11H), 2.42-3.05 (m, 8H), 3.21-3.58 (m, 5H), 3.82 (s, 3H), 5.00 (bs, 1H), 6.50-6.70 (m, 5H), 7.02-7.19 (m, 3H), 8.18 (bs, 1H)

d) 1-(1-Cyclohexanecarbonyl-8-methoxy-1,2,3,4-tetrahydroquinoline-2-ylmethyl)-4-(4-indolyl)piperazine

The title compound was synthesised following the alternative acylation procedure described for the compound of Example 1 (step f) but starting from Compound 49C instead of Compound 1B. The crude was purified by flash chromatography eluting with petroleum ether-ethyl acetate 7:3 affording the title compound (57%).

¹H-NMR (δ):0.75-1.99 (m, 15H), 2.12-3.60 (m, 14H), 4.99-5.24 (m, 1H), 6.43-6.61 (m, 2H), 6.39-6.91 (m, 2H), 7.02-7.21 (m, 4H), 8.20 (bs, 1H)

### EXAMPLE 50

1-(1-Cyclohexanecarbonyl-6-hydroxy-1,2,3,4-tetrahydroquinoline-2-ylmethyl)-4-(4-indolyl)piperazine

a) 6-Hydroxyquinaldine (Compound 50A)

A suspension of 6-methoxyquinaldine (1 g) in 48% HBr (20 ml) was refluxed for 8 h. Afterwards, the cooled mixture was poured into 100 ml of water, alkalinised with 32% NaOH and extracted with ethyl acetate (2×20 ml). The organic layer was dried on anhydrous sodium sulphate and evaporated to dryness. giving 0.83 g of the title compound which was used for the next step without further purification (91%).

¹H-NMR (δ): 1.31-2.05 (br 1H), 2.75 (s, 3H), 7.02-7.30 (m, 3H), 7.81-7.98 (m, 2H).

b) 6-tert-Butoxycarbonyloxyquinaldine (Compound 50B)

A solution of Compound 50A (0.83 g) and dimethylaminopyridine (0.64 g) in THF (40 ml) was cooled at 0-5° C.; then di-tert-butyldicarbonate (1.2 g) was added and the solution stirred at r.t. for 6 h. Afterwards, the solvent was evaporated and the crude purified by flash chromatography eluting with petroleum ether-EtOAc 6:4 affording 0.82 g (63%) of the title compound.

¹H-NMR (δ): 1.55 (s, 9H), 2.78 (s, 3H), 7.22-7.34 (m, 1H), 7.51-7.62 (m, 2H), 7.95-8.07 (m 2H).

c) 2-Bromomethyl-6-tert-butoxycarbonyloxyquinoline (Compound 50C)

The title compound was prepared as described for Compound 5A starting from Compound 50B (0.82 g) instead of 6-fluoro-2-methylquinoline. The crude was purified by flash chromatography eluting with petroleum ether-EtOAc 8:2 to give 0.57 g of the title compound (54%).

¹H-NMR (δ): 1.58 (s, 9H), 5.78 (s, 2H), 7.50-7.69 (m, 3H), 8.03-8.19 (m, 2H)

d) 6-tert-Butoxycarbonyloxy-2-[4-(4-indolyl)-1-piperazinylmethyl]quinoline (Compound 50D)

This compound was obtained in the same way as described for Compound 1C (procedure d) but using Compound 50C instead of 2-chloromethylquinoline. The crude was purified by flash chromatography eluting with methylene chloride-methanol 95:5 to give the title compound (37%).

¹H-NMR (δ): 1.36-1.71 (m, 9H), 2.71-3.00 (m, 4H), 3.23-3.42 (m, 4H), 3.86-4.02 (m, 2H), 6.49-6.66 (m, 2H), 7.00-7.21 (m, 3H), 7.48-7.71 (m, 3H), 7.98-8.36 (m, 3H).

e) 1-(6-tert-Butoxycarbonyloxy-1,2,3,4-tetrahydroquinoline-2-ylmethyl)-4-(4-indolyl)piperazine (Compound 50E)

The title compound was prepared as described for Compound 1B (procedure e) starting from Compound 50D instead of Compound 1C. The crude was purified by flash chromatography eluting with dichloromethane-methanol 97:3 to give the title compound (52%).

¹H-NMR (δ): 1.48-1.73 (m, 11H), 1.81-1.98 (m, 1H), 2.42-3.01 (m, 8H), 3.17-3.52 (m, 5H), 6.41-6.53 (m, 3H), 6.65-6.78 (m, 2H), 7.02-7.18 (m, 3H), 8.09-8.24 (br, 1H)

f) 1-(1-Cyclohexanecarbonyl-6-tert-butoxycarbonyloxy-1,2,3,4-tetrahydroquinoline-2-ylmethyl)-4-(4-indolyl)piperazine (Compound 50F)

The title compound was synthesised following the alternative acylation procedure described for the compound of Example 1 (step f) but starting from Compound 50E instead of Compound 1B. The crude was purified by flash chromatography eluting with petroleum ether-EtOAc 1:1 to give the title compound (47%).

¹H-NMR (δ): 0.81-2.00 (m, 20H), 2.01-2.23 (m, 1H), 2.24-2.82 (m, 9H), 3.07-3.34 (m, 4H), 4.98-5.27 (m, 1H), 6.42-6.61 (m, 2H), 6.92-7.15 (m, 6H), 8.08-8.27 (br, 1H).

g) 1-(1-Cyclohexanecarbonyl-6-hydroxy-1,2,3,4-tetrahydroquinoline-2-ylmethyl)-4-(4-indolyl)piperazine

To a cooled solution of Compound 50F (86 mg) in CH₂Cl₂ (4 ml), 0.5 ml of 3.6 N HCl in diethyl ether was added and the resulting mixture heated at reflux for 5h. Then the solvent was removed under vacuum, the residue alkalinised with NaHCO₃ and extracted with CH₂Cl₂ (2×20 ml). The combined organic layers were dried over Na₂SO₄ and evaporated to dryness. The crude was purified by flash chromatography eluting with CH₂Cl₂-2N NH₃ in MeOH 95:5 to give the title compound (57%).

¹H-NMR (δ): 0.81-1.98 (m, 12H), 1.99-2.20 (m, 1H), 2.22-2.51 (m, 4H), 2.52-2.78 (m, 5H), 3.10-3.31 (m, 4H), 5.05-5.25 (m, 1H), 6.45-6.65 (m, 2H), 6.67-6.75 (m, 2H), 6.92-7.11 (m, 4H), 8.05-8.21 (br, 1H).

### EXAMPLE 51

1-(6-Bromo-1-cyclohexanecarbonyl-1,2,3,4-tetrahydroquinoline-2-ylmethyl)-4-(4-fluoro-2-methoxyphenyl)piperazine

a) 6-Bromo-2-[4-(4-fluoro-2-methoxyphenyl)-1-piperazinyl]methyl]-1,2,3,4-tetrahydroquinoline (Compound 51A)

The title compound was synthesized following the procedure described for compound 34D but using 1-(4-fluoro-2-methoxyphenyl)piperazine as a starting material instead of 1-(4-indolyl)piperazine.

The residue was purified by flash chromatography (petroleum ether—EtOAc 70:30) to afford the title compound (44.4%) as a thick oil.

¹H-NMR (δ): 1.55 (s, 1H), 1.80-2.00 (m, 1H), 2.30-4.00 (m, 17H), 6.50-6.80 (m, 3H), 6.80-7.00 (m, 1H), 7.00-7.20 (m, 2H).

b) 1-(6-Bromo-1-cyclohexanecarbonyl-1,2,3,4-tetrahydroquinoline-2-ylmethyl)-4-(4-fluoro-2-methoxyphenyl)piperazine

The title compound was synthesised following the alternative acylation procedure described for the compound of Example 1 (step f) but starting from Compound 51A instead of Compound 1B. The residue was purified by flash chromatography (petroleum ether—EtOAc 80:20) affording the title compound (69%) as a thick oil.

¹H-NMR (δ): 0.85-2.00 (m, 11H), 2.00-2.80 (m, 8H), 2.80-3.70 (m, 6H), 3.90 (s, 3H), 4.80-5.20 (m, 1H), 6.45-6.65 (m, 2H), 6.70-7.15 (m, 2H), 7.30-7.50 (m, 2H),

### EXAMPLE 52

1-(1-Cyclohexanecarbonyl-8-hydroxy-1,2,3,4-tetrahydroquinoline-2-ylmethyl)-4-(4-indolyl)piperazine

a) 1-(8-Hydroxy-1,2,3,4-tetrahydroquinoline-2-ylmethyl)-4-(4-indolyl)piperazine (Compound 52A)

The title compound was synthesised as reported in Ex. 1 (step e) but using compound 49A instead of Compound 1C. The residue was purified by flash chromatography (petroleum ether—EtOAc 1:1) affording 0.15 g (30%) of the title compound.

¹H-NMR (δ): 1.50-2.05 (m, 3H), 2.41-3.09 (m, 8H), 3.12-3.70 (m, 6H), 6.30-6.82 (m, 4H), 7.02-7.24 (m, 4H), 8.15 (bs, 1H).

b) 1-(8-Cyclohexanecarbonyloxy-1-cyclohexanecarbonyl-1,2,3,4-tetrahydroguinoline-2-ylmethyl)-4-(4-indolyl)piperazine (Compound 52B)

A solution of 0.14 g of compound 52A, 0.20 ml of cyclohexanecarbonyl chloride and 0.41 ml of triethylamine in 5 ml of toluene was stirred at reflux for 0.5 hours, cooled to r.t. The mixture was extracted with EtOAc. The organic layer was washed with 1 N NaOH, with H₂O, dried (Na₂SO₄) and evaporated to dryness in vacuo. The crude was purified by flash chromatography (petroleum ether—EtOAc 6:4) to afford 0.17 g (77%) of the title compound.

¹H-NMR (δ): 0.78-2.05 (m, 24H), 2.05-2.90 (m, 8H), 2.97-3.38 (m, 4H), 4.94-5.07 (m, 1H), 6.45-6.62 (m, 2H), 6.85-7.24 (m, 6H), 8.15 (bs, 1H).

c) 1-(1-Cyclohexanecarbonyl-8-hydroxy-1,2,3,4-tetrahydroquinoline-2-ylmethyl)-4-(4-indolyl)piperazine

A mixture of 0.17 g of compound 52B, 0.075 g of lithium hydroxide monohydrate in 3 ml of THF and 0.2 ml of H₂O was stirred at r.t. for 12 hours, diluted with H₂O and extracted with EtOAc. The organic layer was washed with water, dried (Na₂SO₄) and evaporated to dryness in vacuo. The crude was purified by flash chromatography (petroleum ether—EtOAc 6:4) to afford 0.32 g (65%) of the title compound.

¹H-NMR (δ): 0.80-1.95 (m, 14H), 2.15-2.58 (m, 6H), 2.91-3.48 (m, 6H), 5.38-5.57 (m, 1H), 6.45-6.62 (m, 2H), 6.76 (d, 1H), 6.90 (d, 1H), 7.02-7.20 (m, 4H), 8.15 (bs, 1H).

### EXAMPLE 53

1-(1-Cyclohexanecarbonyl-6-methyl-1,2,3,4-tetrahydroquinoline-2-ylmethyl)-4-(4-fluoro-2-methoxyphenyl)piperazine

a) 1-(4-Fluoro-2-methoxyphenyl)-4-(6-methyl-1,2,3,4-tetrahydroquinoline-2-ylcarbonyl)piperazine (Compound 53A)

The title compound was synthesised following the procedure described for Compound 1D (step g) but using Compound 32B instead of 1,2,3,4-tetrahydroquinoline-2-carboxylic acid and 1-(4-fluoro-2-methoxyphenyl)piperazine instead of 1-(4-indolyl)piperazine. The residue was purified by flash chromatography eluting with petroleum ether—EtOAc 6:4 to give of the title compound (69%).

¹H-NMR (δ): 1.60-1.81 (m, 1H), 2.04-2.10 (m, 1H), 2.11 (s; 3H), 2.66-2.89 (m, 2H), 2.93-3.17 (m, 4H), 3.61-3.98 (m, 7H), 4.02-4.26 (m, 1H), 4.28-4.42 (br, 1H), 6.51-6.70 (m, 3H), 6.71-6.94 (m, 3H).

b) 1-(4-Fluoro-2-methoxy)-4-(6-methyl-1,2,3,4-tetrahydroquinoline-2-ylmethyl)piperazine (Compound 53B)

The title compound was synthesised following the procedure described for Compound 1B (step h) but using Compound 53A instead of Compound1D. The residue was purified by flash chromatography eluting with petroleum ether—EtOAc 7:3 to give the title compound (86.5%).

¹H-NMR (δ): 1.45-1.67 (m, 1H), 1.80-1.97 (m, 1H), 2.21 (s, 3H), 2.46 (d, 2H), 2.50-2.62 (m, 2H), 2.63-2.91 (m, 4H), 2.99-3.18 (m, 4H), 3.31-3.52 (m, 1H), 3.87 (s, 3H), 4.71-4.84 (br, 1H), 6.41-6.52 (m, 1H), 6.53-6.70 (m, 2H), 6.75-6.94 (m, 3H).

c) 1-(1-Cyclohexanecarbonyl-6-methyl-1,2,3,4-tetrahydroquinoline-2-ylmethyl)-4-(4-fluoro-2-methoxyphenyl)piperazine

The title compound was synthesised following the alternative acylation procedure described for the compound of Example 1 (step f) but starting from Compound 53B instead of Compound 1B. The crude was purified by flash chromatography eluting with petroleum ether-ethyl acetate 1:1 to give the title compound (80%).

¹H-NMR (δ):0.81-1.99; (m; 11H), 2.01-2.19 (m; 1H), 2.23-2.81 (m; 12H), 2.84-3.08 (m; 4H), 3.82 (s; 3H), 4.98-5.17 (m; 1H), 6.49-6.68 (m; 2H), 6.74-7.09 (m; 4H).

### EXAMPLE 54

1-[1-(3-Cyanominopropionyl)-1,2,3,4-tetrahydroquinoline-2-ylmethyl]-4-(4-indolyl)piperazine

A mixture of 0.021 g of cyanamide, 0.1 g of K₂CO₃ and 0.75 ml of DMF was stirred for 1.5 h at 80° C., cooled at r.t.; afterwards, 0.04 g of Compound 27A was added with stirring at 80° C. for further 6 h. After cooling at r.t., the reaction mixture was diluted with water (7 ml), strirred for 0.5 h and filtered recovering the precipitated solid. This was purified by flash chromatography (EtOAc2 N NH₃ sol. in methanol 96:4) to give 0.025 g (56%) of the title compound

¹H-NMR (δ): 1.25-1.65 (m, 1H), 2.00-2.85 (m, 11H), 2.85-3.65 (m, 6H), 5.10 (b, 1H), 5.70 (b, 1H), 6.40-6.55 (m, 2H), 6.90-7.25 (m, 7H), 8.10 (s, 1H)

### EXAMPLE 55

1-(5-Chloro-1-cyclohexanecarbonyl-1,2,3,4-tetrahydroquinoline-2-ylmethyl)-4-(4-fluoro-2-methoxyphenyl)piperazine

a) 1-Benzoyl-5-chloro-2-cyano-1,2-dihydroquinoline (Compound 55A)

The title compound was prepared as described for compound 32A, but starting from 5-chloroquinoline (WO 0144247) instead of 6-methylquinoline. (67%).

¹H-NMR (δ): 6.13-6.22 (m; 2H), 6.51 (d;1H), 6.95 (dd; 1H), 7.18-7.51 (m; 6H).

b) 5-Chloroquinoline-2-carboxylic Acid (Compound 55B)

Compound 55B was obtained in the same manner described for compound 32B, but using compound 55A instead of compound 32A. The crude was used without additional purification. (63%).

¹H-NMR (DMSO-d₆ δ): 6.15-7.01 (br; 1H), 7.60-7.81 (m; 2H), 7.92-8.18 (m; 2H), 8.48 (d; 1H)

c) 5-Chloro-1,2,3,4-tetrahydroquinoline-2-carboxylic acid (Compound 55C)

The title compound was prepared as described for Compound 1B (step e) starting from Compound 55B instead of Compound 1D. The crude was used without further purification to give the title compound (78%).

¹H-NMR (DMSO-d₆ δ): 1.87-2.20 (m; 2H), 2.67-2.88 (m; 2H), 3.99-4.18 (m; 1H), 5.12-5.82 (br; 2H), 6.40 (d; 1H), 6.71 (d; 1H), 6.97 (dd; 1H).

d) 1-(5-Chloro-1,2,3,4-tetrahydroquinoline-2-ylcarbonyl)-4-(4-fluoro-2-methoxyphenyl)piperazine (Compound 55D)

The title compound was synthesised following the procedure described for Compound 1D (step g) but using Compound 55C instead of 1,2,3,4-tetrahydroquinoline-2-carboxylic acid. The residue was purified by flash chromatography eluting with petroleum ether—EtOAc 6:4 to give the title compound (79%).

¹H-NMR (δ): 1.59-1.71 (m, 1H), 2.12-2.28 (m, 1H), 2.60-2.82 (m, 1H), 2.92-3.12 (m, 5H), 3.55-3.96 (m, 7H), 4.11-4.23 (m, 1H), 4.61-4.74 (br, 1H), 6.50-6.78 (m, 4H), 6.80-7.04 (m, 2H).

e) 1-(5-Chloro-1,2,3,4-tetrahydroquinoline-2-ylmethyl)-4-(4-fluoro-2-methoyphenyl)piperazine (Compound 55E)

The title compound was synthesised following the procedure described for Compound 1B (step h). The residue was purified by flash chromatography eluting with petroleum ether—EtOAc 1:1 to give the title compound (53%).

¹H-NMR (δ): 1.40-1.79 (m, 1H), 1.81-2.09 (m, 1H), 2.24-3.64 (m, 13H), 3.84 (s, 3H), 4.55-4.91 (br, 1H), 6.31-6.75 (m, 4H), 6.77-7.04 (m, 2H).

f) 1-(1-Cyclohexanecarbonyl-5-chloro-1,2,3,4-tetrahydroquinoline-2-ylmethyl)-4-(4-fluoro-2-methoxyphenyl)piperazine

The title compound was synthesised following the alternative acylation procedure described for the compound of Example 1 (step f) but starting from Compound 55E instead of Compound 1B. The crude was purified by flash chromatography eluting with petroleum ether—ethyl acetate 65:35 to give the title compound (67%).

¹H-NMR (δ): 0.76-3.49 (m, 25H), 3.80 (s, 3H), 4.90-5.34 (m, 1H), 6.47-6.63 (m, 2H), 6.80-6.91 (m, 1H), 7.08-7.32 (m, 3H).

### EXAMPLE 56

1-[5-Chloro-1-(4-methoxybenzoyl)-1,2,3,4-tetrahydroquinoline-2-ylmethyl]-4-(4-fluoro-2-methoxyphenyl)piperazine

The title compound was synthesised following the alternative acylation procedure described for the compound of Example 1 (step f) but starting from Compound 55E instead of Compound 1B, using 4-methoxybenzoyl chloride instead of cyclohexanecarbonyl chloride, toluene instead 1,2-dichlorometane and refluxing for 1 hour. The crude was purified by flash chromatography eluting with petroleum ether—ethyl acetate 6:4 to give the title compound (28%).

¹H-NMR (δ): 1.94-2.12 (m, 1H), 2.13-2.39 (m, 1H), 2.44-3.08 (m, 12H), 3.79, 3.88 (2s, 6H), 4.89-5.08 (br, 1H), 6.51-6.66 (m, 3H), 6.72-6.91 (m, 4H), 7.11 (d, 1H), 7.24-7.39 (m, 2H).

### EXAMPLE 57

1-(7-Chloro-1-cyclohexanecarbonyl-1,2,3,4-tetrahydroquinoline-2-ylmethyl)-4-(4-fluoro-2-methoxyphenyl)piperazine

a) 2-Bromomethyl-7-chloroquinoline (Compound 57A)

The title compound was prepared as described for Compound 5A but using 7-chloro-2-methylquinoline imstead of 6-fluoro-2-methylquinoline. The residue was purified by flash chromatography (petroleum ether—EtOAc gradient from 100:5 to 100:10) to afford Compound 57A (42%) as a white solid.

¹H-NMR (δ): 4.70 (s, 2H), 7.50(dd, 1H), 7.58 (d, 1H), 7.75 (d, 1H), 8.10 (d, 1H), 8.18 (d, 1H),

b) 7-Chloro-2-[4-(4-fluoro-2-methoxyphenyl)-1-piperazinylmethyl]quinoline (Compound 57B)

The title compound was prepared as described for Compound 1D (step d) but using Compound 57A instead of 2-chloromethylquinoline. The residue was purified by flash chromatography (petroleum ether—EtOAc 70:30) to afford Compound 57B (42.3%) as an orange solid.

¹H-NMR (δ): 2.65-2.85 (m, 4H), 2.90-3.20 (m, 4H), 3.80 (s, 3H), 3.90 (s, 3H), 6.45-6.65 (m, 2H), 6.75-6.95 (m, 1H), 7.45 (dd, 1H), 7.55-7.80 (m, 2H), 8.00-8.20 (m, 2H).

c) 1-(7-Chloro-1,2,3,4-tetrahydroquinoline-2-ylmethyl)-4-(4-fluoro-2-methoxyphenyl)piperazine (Compound 57C)

The title compound was synthesised following the procedure reported for Compound 1B (step e) starting from compound 57B instead Compound 1C.

The residue was purified by flash chromatography (petroleum ether—EtOAc 60:40) affording Compound 57C (42%) as an orange oil.

¹H-NMR (δ): 1.32-1.65 (m, 1H), 1.80-2.00 (m, 1H), 2.32-2.65 (m, 4H), 2.65-2.95 (m, 6.80-7.00 (m, 2H).

d) 1-(7-Chloro-1-cyclohexanecarbonyl-1,2,3,4-tetrahydroquinoline-2-ylmethyl)-4-(4-fluoro-2-methoxyphenyl)piperazine

The title compound was synthesised following the alternative acylation procedure described for the compound of Example 1 (step f) but starting from Compound 57C instead of Compound 1B. The crude was purified by flash chromatography eluting The residue was purified by flash chromatography (CHCl₃-2 N methanolic ammonia 100: 5) affording the title compound (52.1%) as a thick oil.

¹H-NMR (δ): 0.75-2.20 (m, 12H), 2.20-2.80 (m, 9H), 2.80-3.20 (m, 4H), 3.92 (s, 3H), 4.80-5.20 (m, 1H), 6.50-6.70 (m, 2H), 6.75-6.95 (m, 2H), 7.03-7.25 (m, 2H).

### EXAMPLE 58

3-Benzyl-1-(1-cyclohexanecarbonyl-1,2,3,4-tetrahydroquinoline-2-ylmethyl)piperidine (upper TLC Rf diastereomer)

The title compound was synthesised using the methodology described in Example 35 using Intermediate 35 B and 3-benzylpiperidine (WO 97/23458) instead of 1-(4-fluoro-2-methoxyphenyl)piperazine. Eluent: EtOAc—MeOH 95:5. Yield: 35%.

¹H-NMR (δ): 0.82-3.48 (m, 28H), 4.88-5.32 (m, 1H), 6.85-7.42 (m, 9H).

### EXAMPLE 59

3-Benzyl-1-(1-cyclohexanecarbonyl-1,2,3,4-tetrahydroquinoline-2-ylmethyl)piperidine (lower TLC Rf diastereomer)

The title compound was isolated as the lower Rf diastereomer during the purification of Compound 58.

Eluent: EtOAc-MeOH 95:5. Yield: 13%.

¹H-NMR (δ): 0.82-1.41 (m, 6H), 1.42-2.01 (m, 1H), 2.17-3.01 (m, 9H), 3.23-3.52 (m, 2H), 5.02-5.32 (m, 1H), 6.88-7.42 (m, 9H).

### EXAMPLE 60

The following compounds were synthesised using the methodology described in Example 35 but in a parallel synthesis fashion using dichloromethane instead of chloroform as a solvent and using the proper basic head instead of 1-(4-fluoro-2-methoxyphenyl)piperazine. After having alkalinised, diluted with CH₂Cl₂, the aqueous layer was removed by filtering on solid phase extraction (SPE) cartridges. The organic layer was evaporated to dryness in vacuo and the crude purified by flash chromatography using traditional techiques or parallel flash chromatography techniques (QUAD3™from Biotage) (eluent shown):

### EXAMPLE 60A

1-(4-Chloro-2-1-propoxyphenyl)-4-(1-cyclohexanecarbonyl-1,2,3,4-tetrahydroquinoline-2-ylmethyl)piperazine

From 1-(4-chloro-2-i-propoxyphenyl)piperazine (prepared by the method of Martin, G. E. et al., J. Med. Chem. 32, 1052-1056, (1989) starting from 4-chloro-2-hydroxynitrobenzene).

Flash chromatography: petroleum ether—EtOAc 7:3. Yield: 72.7%.

¹H-NMR (δ): 0.85-3.52 (m, 31H), 4.41-4.69 (m 1H), 5.05-5.23 (m, 1H), 6.80-6.95 (m, 3H), 7.05-7.15 (m, 4H).

### EXAMPLE 60A1:

1-(5-Chloro-2-fluorophenyl)-4-(1-cyclohexanecarbonyl-1,2,3,4-tetrahydroquinoline-2-ylmethyl)piperazine

From 1-(5-chloro-2-fluorophenyl)piperazine (WO 01/05765).

Flash chromatography eluent: petroleum ether—EtOAc 8:2. Yield: 50%.

¹H-NMR (δ): 0.79-1.99 (m, 11H), 2.01-2.18 (m, 1H), 2.20-2.81 (m, 9H), 2.91-3.18 (m, 4H), 4.98-5.21 (m, 1H), 6.77-7.01 (m, 3H), 7.02-7.25 (m, 4H).

### EXAMPLE 60A2

1-[4-(2,1,3-Benzothiadiazolyl)]-4-(1-cyclohexanecarbonyl-1,2,3,4-tetrahydroquinoline-2-ylmethyl)piperazine.

From 1-[4-(2,1,3-benzothiadiazolyl]piperazine (U.S. Pat. No. 4,831,031).

Flash chromatography eluent: petroleum ether—EtOAc 1:1. Yield: 55%.

¹H-NMR (δ): 0.75-1.88 (m, 111H), 1.99-2.08 (m, 1H), 2.25-3.01 (m, 9H), 3.31-3.82 (m, 4H), 5.00-5.37 (m, 1H), 6.72 (d, 1H), 7.09-7.32 (m, 4H); 7.44-7.64 (m, 2H).

### EXAMPLE 60A3

1-(1-Cyclohexanecarbonyl-1,2,3,4-tetrahydroquinoline-2-ylmethyl)-4-(2-ethoxy-4-fluorophenyl)piperazine

From 1-(2-ethoxy-4-fluorophenyl)piperazine (WO 96/17831).

Flash chromatography: petroleum ether—EtOAc 6:4. Yield: 44.8%.

¹H-NMR (δ): 0.85-1.87 (m, 13H), 1.88-2.21 (m, 2H), 2.22-2.81 (m, 8H), 2.82-3.21 (m, 4H), 4.08 (q, 2H), 4.95-5.23 (m, 1H), 6.48-6.61 (m, 3H), 6.73-6.91 (m, 1H), 7.03-7.30 (m, 4H).

### EXAMPLE 60A4

1-(1-Cyclohexanecarbonyl-1,2,3,4-tetrahydroquinoline-2-ylmethyl)-4-(2-methoxy-4-hydroxyphenyl)piperazine

From 1-(4-hydroxy-2-methoxyhenyl)piperazine (Eur. J. Med. Chem. 25, 291 (1990)).

Flash chromatography: petroleum ether—EtOAc 6:4. Yield: 58%.

¹H-NMR (δ): 0.85-1.87 (m, 13H), 1.88-2.21 (m, 2H), 2.22-2.81 (m, 8H), 2.82-3.21 (m, 4H), 4.08 (q, 2H), 4.95-5.23 (m, 1H), 6.48-6.61 (m, 3H), 6.73-6.91 (m, 1H), 7.03-7.30 (m, 4H).

### EXAMPLE 60A5

1-(1-Cyclohexanecarbonyl-1,2,3,4-tetrahydroquinoline-2-ylmethyl)-4-(7-methoxy-4-indolyl)piperazine.

From 1-(7-methoxy-4-indolyl)piperazine.

Flash chromatography: petroleum ether—EtOAc2 N methanolic ammonia 8:2:0.3. Yield:32.7%

¹H-NMR (δ): 0.80-2.80 (m, 21H), 3.00-3.20 (m, 4H), 3.90 (s, 3H), 5.10 (b, 1H), 6.40-6.55 (m, 3H), 7.00-7.30 (m, 5H), 8.45 (b, 1H).

The starting piperazine was obtained as following:

a) 4-Amino-7-methoxyindole (Compound 60A5-A)

A mixture of 0.06 g of 4-nitro-7-methoxyindole (N. Rouè et al, Heterocycles 43, 263-267), 0.003 g of 10% Pd-C and 8 ml of EtOH was hydrogenated in a Parr apparatus at 30 p.s.i. The catalyst was filtered off and the solution was evaporated to dryness in vacuo affording the title compound (0.04 g, 80%), which was used in the next step without further purification.

¹H-NMR (δ): 1.65-3.70 (b, 2H), 3.85 (s, 3H), 6.30 (d, 1H), 6.40-6.55 (m, 2H), 7.15 (dd, 1H), 8.35 (b, 1H)

b) 1-(7-Methoxy-4-indolyl)piperazine (Compound 60A5-B)

The title compound was prepared as described for Compound 35A13-B starting from Compound 60A5-A instead of Compound 35A13-A. The crude was purified by flash chromatography eluting with CH₂Cl₂-2 N methanolic ammonia 90:10 to give the title compound (23%).

¹H-NMR (δ): 2.65-3.60 (m, 9H), 3.90 (s, 3H), 6.40-6.60 (m, 3H), 7.15 (dd, 1H), 8.40 (b, 1H).

### EXAMPLE 60A6

1-(1-Cyclohexanecarbonyl-1,2,3,4-tetrahydroquinoline-2-ylmethyl)-4-(2-pyrazinyl)piperazine

From 1-(2-pyrazinyl)piperazine (commercial)

Flash chromatography: petroleum ether—EtOAc 7:3. Yield: 44%.

¹H-NMR (δ): 0.80-2.11 (m, 12H), 2.12-3.21 (m, 9H), 3.22-3.76 (m, 9H), 5.05-5.31 (m, 1H), 7.03-7.30 (m, 4H), 7.82 (s, 1H), 8.32 (dd, 2H).

### EXAMPLE 60A7

1-(2-Cyano-4-nitrophenyl)-4-(1-cyclohexanecarbonyl-1,2,3,4-tetrahydroquinoline-2-ylmethyl)piperazine

From 1-(2-cyano-4-nitrophenyl)piperazine (CA vol.97, 1982, 109953s)

Flash chromatography: petroleum ether—EtOAc 1:1. Yield: 64%.

¹H-NMR (δ): 0.75-2.01 (m, 11H), 2.11-2.89 (m, 10H), 3.40-3.59 (m, 4H), 5.01-5.21 (m, 1H), 6.94 (d, 1H), 7.03-7.27 (m, 4H), 8.24 (dd, 1H), 8.43 (d, 1H).

### EXAMPLE 60A8

1-[4-(2,1,3-Benzoxadiazolyl)]-4-(1-cyclohexanecarbonyl-1,2,3,4-tetrahydroquinoline-2-ylmethyl)piperazine.

From 1-[4-(2,1,3-Benzoxadiazolyl)]piperazine (EP 189612).

Flash chromatography: petroleum ether—EtOAc 1:1. Yield: 86%.

¹HNMR (δ): 0.81-2.01 (m,l 1H), 2.04-2.21 (m, 1H), 2.25-2.87 (m, 9H), 3.41-3.66 (m, 4H), 4.98-5.28 (m, 1H), 6.34 (d, 1H), 6.98-7.48 (m, 6H).

### EXAMPLE 60A9

1-(1-Cyclohexanecarbonyl-1,2,3,4-tetrahydroquinoline-2-ylmethyl)-4-(2-methoxy-5-trifluoromethylphenyl)piperazine.

From 1-(2-methoxy-5-trifluoromethylphenyl)piperazine (EP 156443) CA:104, P129918a.

Flash chromatography: petroleum ether—EtOAc 6:4. Yield: 86%.

¹H-NMR (δ): 0.84-2.21 (m, 12H), 2.28-2.87 (m, 9H), 3.92-3.26 (m, 4H), 3.91 (s, 3H), 4.99-3.31 (m, 1H), 6.88 (d, 1H), 7.04-7.35 (m, 6H).

### EXAMPLE 60A10

1-(1-Cyclohexanecarbonyl-1,2,3,4-tetrahydroquinoline-2-ylmethyl)-4-[1-(1,2,3,4-tetrahydronaphthyl)]piperazine.

From 1-(1,2,3,4-tetrahydronaphthyl)piperazine (J. Med. Chem., 40:952 (1997)).

Flash chromatography: petroleum ether—EtOAc 1:1. Yield: 45%.

¹H-NMR (δ): 0.81-2.17 (m, 16H), 2.20-2.92 (m, 15H), 3.65-4.92 (m, 1H), 4.89-5.21 (m, 1H), 7.02-7.28 (m, 7H), 7.57-7.77 (m, 1H).

### EXAMPLE 60A11

1-(7-Chloro-2,3-dihydro-1,4-benzodioxin-5-yl)-4-(1-cyclohexanecarbonyl-1,2,3,4-tetrahydroquinoline-2-ylmethyl)piperazine.

From 1-(7-chloro-2,3-dihydro-1,4-benzodioxin-5-yl)piperazine (Commercial).

Flash chromatography: CH₂Cl₂— EtOAc 7:3. Yield: 45%.

¹H-NMR (δ): 0.74-2.21 (m, 12H), 2.25-2.88 (m, 8H), 2.90-3.21 (m, 4H), 4.18-4.89 (m, 4H), 4.96-5.38 (m, 1H), 6.43-6.69 (m, 2H), 7.04-7.32 (m, 5H).

### EXAMPLE 60A12

1-(1-Cyclohexanecarbonyl-1,2,3,4-tetrahydroquinoline-2-ylmethyl)-4-(4-pyridinyl)piperazine.

From 1-(4-pyridinyl)piperazine (Regneir et al. Arzneim. Forsch. 24, 12, 1974).

Flash chromatography: CH₂Cl₂— MeOH 9:1. Yield: 79%.

¹H-NMR (δ): 0.71-2.02 (m, 11H), 2.04-2.81 (m, 10H), 3.35-3.61 (m, 4H), 5.02-5.21 (m, 1H), 6.75 (d, 2H), 6.97-7.31 (m, 4H), 8.17 (d, 2H).

### EXAMPLE 60A13

1-(1-Cyclohexanecarbonyl-1,2,3,4-tetrahydroquinoline-2-ylmethyl)-4-[4-(6,7-di-methoxyquinazolinyl)]piperazine

From 1-[4-(6,7-dimethoxyquinazolinyl)]piperazine (CA 70 68419 (1969)).

Flash chromatography: EtOAc—MeOH 98:2. Yield: 81%.

¹H-NMR(δ):0.75-1.89 (m, 11H), 1.91-2.12 (m, 11H9,2.19-3.17 (m, 9H), 3.79-4.24 (m, 10H), 7.07 (s, 1H), 7.14-7.39 (m, 4H), 7.43 (s, 1H), 8.63 (s, 1H).

### EXAMPLE 61

1-(1-Cyclohexanecarbonyl-6-nitro-1,2,3,4-tetrahydroquinoline-2-ylmethyl)-4-(4-fluoro-2-methoxyphenyl)piperazine

a) 1-Cyclohexanecarbonyl-6-nitro-1,2,3,4-tetrahydroquinoline-2-carboxylic acid methyl ester (Compound 61A)

Into a solution of methyl 1-cyclohexanecarbonyl-1,2,3,4-tetrahydroquinoline-2-carboxylate (2.6 g) in acetic anhydride (3.7 ml) a solution of 37% HNO₃ (0.94 ml) and Ac₂O was dropped. The resulting mixture was stirred at 60° C. for 4 h. Afterwards, the solution was poured into 200 ml of water and extracted with ethyl acetate (2×60 ml). The organic layer was dried on anhydrous sodium sulphate and evaporated to dryness. The crude was purified by flash chromatography eluting with petroleum ether—EtOAc 7:3 giving 1.8 g of the title compound (60%).

¹H-NMR (δ): 0.89-2.10 (m, 11H), 2.49-2.89 (m, 4H), 3.65 (s, 3H), 5.01-5.18 (m, 1H), 7.35 (dd, 1H), 8.03-8.22 (m, 2H).

b) 2-Hydroxymethyl-6-nitro-1,2,3,4-tetrahydroquinoline (Compound 61B)

To a solution of 0.8 g of Compound 61A in 15 ml of anhydrous THF stirred at 0-5° C. was added 1.2 ml of 2 M LiBH₄ in THF; then the mixture was stirred at r.t. for 6 h. Afterwards, the reaction was diluted with an aqueous saturated solution of ammonium chloride (20 ml), extracted with EtOAc (2×50 ml), dried on sodium sulphate and evaporated in vacuo. The residue was purified by flash chromatography eluting with CH₂Cl₂— MeOH 95:5 affording 0.3 g of the title compound (62%).

¹H-NMR (δ): 1.48-1.81 (m, 1H), 1.92-2.11 (m, 1H), 2.74-2.94 (m, 2H), 3.45-3.61 (m, 2H), 3.65-3.85 (m, 1H), 4.94-5.41 (bs, 1H), 6.48 (dd, 1H), 7.81-8.01 (m, 2H).

c) 2-Bromomethyl-6-nitro-1,2,3,4-tetrahydroquinoline (Compound 61C)

A solution of Compound 61B (0.3 g), triphenylphosphine (0.42 g) and CBr₄ (0.86 g) in CH₂Cl₂ (40 ml) was stirred at r.t. for 12 h. Then the solvent was evaporated and the crude purified by flash chromatography eluting with CH₂Cl₂— MeOH 95:5 giving 0.24 g of the title compound (60%).

¹H-NMR (δ): 1.65-1.83 (m, 1H), 1.99-2.19 (m, 1H), 2.74-2.94 (m, 2H), 3.18-3.42 (m, 2H), 3.55-3.74 (m, 1H), 6.47 (dd, 1H), 7.83-8.02 (m, 2H)

d) 1-(4-Fluoro-2-methoxyphenyl)-4-(6-nitro-1,2,3,4-tetrahydroquinoline-2-ylmethyl)piperazine (Compound 61D).

A mixture of Compound 61C (110 mg), 1-(4-fluoro-2-methoxyphenyl)piperazine (85 mg) and K₂CO₃ was heated for 30 min at 200° C. Afterwards, the reaction was diluted with H₂O (10 ml) and extracted with EtOAc (2×20 ml). The combined organic layers were dried over Na₂SO₄ and evaporated to dryness. The crude was purified by flash chromatography eluting with petroleum ether—EtOAc 1:1 affording 60 mg of the title compound (38%).

¹H-NMR (δ): 1.41-1.71 (m, 1H), 1.91-2.09 (m, 1H), 2.32-3.31 (m, 12H), 3.51-3.73 (m, 1H), 3.81 (s, 3H), 5.52-5.95 (br, 1H), 6.41-6.51 (m, 1H), 6.52-6.68 (m, 2H), 6.83-6.95 (m, 1H), 7.82-7.95 (m, 2H).

e) 1-(1-Cyclohexanecarbonyl-6-nitro-(1,2,3,4-tetrahydroquinoline-2-ylmethyl)-4-(4-indolyl)piperazine

The title compound was synthesised following the alternative acylation procedure described for the compound of Example 1 (step f) but starting from Compound 61D instead of Compound 1B. The crude was purified by flash chromatography eluting with petroleum ether—EtOAc 1:1 to give the title compound (47%).

¹H-NMR (δ): 0.70-3.12 (m, 23H), 3.13-3.41 (m, 2H), 3.82 (s, 3H), 4.82-5.03 (m, 1H), 6.42-6.71 (m, 1H), 6.72-6.98 (m, 2H), 7.29-7.41 (m, 1H), 8.03-8.21 (m, 2H).

### EXAMPLE 62

The following compounds were synthesised using the methodology described in Example 35 but in a parallel synthesis fashion using dichloromethane instead of chloroform as the solvent and using the proper basic head instead of 1-(4-fluoro-2-methoxyphenyl)piperazine. After having alkalinised, diluted with CH₂Cl₂, the aqueous layer was removed by filtering on Solid phase extraction (SPE) cartridges. The organic layer was evaporated to dryness in vacuo and the crude purified by flash chromatography using traditional techiques or parallel flash chromatography techniques (QUAD3™ from Biotage) (eluent shown):

### EXAMPLE 62A

1-(1-Cyclohexanecarbonyl-1,2,3,4-tetrahydroquinoline-2-ylmethyl)-4-(1-ethyl-4-indolyl)piperazine

From 1-(1-ethyl-4-indolyl)piperazine.

Flash chromatography: petroleum ether-EtOAc 8:2. Yield: 52%

¹H-NMR (δ): 0.80-1.88 (m, 14H), 1.89-2.11 (m, 1H), 2.21-2.95 (m, 9H), 2.96-3.42 (m, 4H), 4.11 (q, 2H), 4.99-5.30 (m, 1H), 6.35-6.61 (m, 2H), 6.95-7.32 (m, 7H).

The starting piperazine was obtained as following:

a) 1-tert-Butoxycarbonyl-4-(1-ethyl-4-indolyl)piperazine (Compound 62A-A)

The title compound was prepared as described in Example 35A25-A using ethyl bromide instead of acetyl chloride. Flash chromatography: petroleum ether—EtOAc 8:2. Yield: 71%.

¹H-NMR (δ): 1.30-1.70 (m, 3H), 1.50 (s, 9H), 2.95-3.40 (m, 4H), 3.45-3.90 (m, 4H), 4.20 (q, 2H), 6.40-7.25 (m, 2H), 0.62 (dd, 1H), 6.95-7.30 (m, 3H).

b) 1-(1-Ethyl-4-indolyl)piperazine (Compound 62A-B)

The title compound was prepared as described in Example 35A25-B using Compound 62A-A instead of Compound 35A25-B.

¹H-NMR (δ): 1.42 (t, 3H), 3.12-3.33 (m, 8H), 4.15 (q, 2H), 4.99-5.30 (m, 1H), 6.38 (dd, 1H), 6.62 (dd, 1H), 6.99-7.20 (m, 3H).

### EXAMPLE 62A1

1-(2-Bromo-5-methoxybenzyl)-4-(1-cyclohexanecarbonyl-1,2,3,4-tetrahydroquinoline-2-ylmethyl)piperazine

From 1-(2-bromo-5-methoxybenzyl)piperazine (CA2188484)).

Flash chromatography: petroleum ether—EtOAc 7:3. Yield: 63%.

¹H-NMR (δ): 0.78-1.99 (m, 11H), 2.00-2.21 (m, 1H), 2.22-2.95 (m, 13H), 3.45-3.62 (m, 2H), 3.81 (s, 3H), 4.92-5.21 (m, 1H), 6.61-6.75 (m, 1H), 6.98-7.24 (m, 5H), 7.31-7.48 (m, 1H).

### EXAMPLE 62A2

1-(4-Chloro-2-methoxyphenyl)-4-(1-cyclohexanecarbonyl-1,2,3,4-tetrahydroquinoline-2-ylmethyl)piperazine.

From 1-(4-chloro-2-methoxyphenyl)piperazine (U.S. Pat. No. 5,859,014).

Flash chromatography: petroleum ether—EtOAc 8:2. Yield: 65%.

¹H-NMR (δ): 0.79-3.42 (m, 25H), 3.87 (s 3H); 4.88-5.38 (m, 1H), 6.71-6.97 (m, 3H), 7.04-7.31 (m, 4H).

### EXAMPLE 62A3

1-(4-Chloro-2-ethoxyphenyl)-4-(1-cyclohexanecarbonyl-1,2,3,4-tetrahydroquinoline-2-ylmethyl)piperazine

From 1-(4-chloro-2-ethoxyphenyl)piperazine prepared by the method of Martin, G. E. et al., J. Med. Chem. 32, 1052-1056 (1989), starting from 4-chloro-2-hydroxynitrobenzene.

Flash chromatography: petroleum ether—EtOAc 8:2. Yield: 58%.

¹H-NMR (δ): 0.79-1.18 (m, 111H), 1.19-2.20 (m, 1H), 2.22-2.84 (m, 9H), 2.88-3.24 (m, 4H), 4.05 (q, 2H), 4.98-5.28 (m, 1H), 6.69-6.90 (m, 3H), 7.08-7.38 (m, 4H).

### EXAMPLE 62A4

1-(1-Cyclohexanecarbonyl-1,2,3,4-tetrahydroquinoline-2-ylmethyl)-4-(4-fluoro-2- -propoxyphenyl)piperazine

From 1-(4-fluoro-2-1-propoxyphenyl)piperazine (EP 102 9851, Yoshitomi, 2000).

Flash chromatography: petroleum ether—EtOAc 8:2. Yield: 58%.

¹H-NMR (δ): 0.78-1.92 (m, 16H), 1.93-2.17 (m 2H), 2.18-2.76 (m, 9H), 2.77-3.34 (m, 4H), 4.48-4.62 (m, 1H), 5.03-5.28 (m, 1H), 6.49.-6.63 (m, 2H), 6.78-6.84 (m, 1H), 7.05-7.30 (m, 4H).

### EXAMPLE 62A5

1-(1-Cyclohexanecarbonyl-1,2,3,4-tetrahydroquinoline-2-ylmethyl)-4-(7-methyl-4-indolyl)piperazine

From 1-(7-methyl-4-indolyl)piperazine.

Flash chromatography: petroleum ether—EtOAc 7:3. Yield: 54%.

¹H-NMR (δ): 0.81-1.82 (m, 11H), 1.83-2.04 (m, 1H), 2.34-2.99 (m, 12H), 3.00-3.42 (m, 4H), 5.01-5.31 (m, 1H), 6.41-6.61 (m, 2H), 6.83-7.93 (m, 1H), 7.05-7.34 (m, 5H), 8.06-8.27 (br, 1H).

The starting piperazine was obtained as following:

a) 4-Amino-7-methylindole (Compound 62A5-A)

The title compound was synthesised using the methodology described in Example 35A1 using 7-methyl-4-nitroindole (J. Bergman et al, Tetrahedron 46, 6085-6112 (1990)) instead of 2-methyl-4-nitroindole. Yield: 91%.

¹H-NMR (δ): 2.46 (s, 3H), 3,69-4.02 (br, 2H), 6.25 (dd, 1H), 6.38 (dd, 1H), 6.75 (dd, 1H), 7.14 (dd 1H), 8.01-8.13 (br, 1H).

b) 1-(7-Methyl-4-indolyl)piperazine (Compound 62A5-B)

The title compound was synthesised using the methodology described in Example 35A1 (step b) using Compound 62A5-A instead of Compound 35A1-A. The crude was purified by flash chromatography eluting with CH₂Cl₂-2 N methanolic ammonia 9:1 to give the title compound (60%).

¹H-NMR (δ): 2.47 (s, 3H), 2.99-3.35 (m, 9H), 6.45-6.52 (m 2H), 6.87 (dd, 1H), 7.21 (dd, 1H), 8.03-8.18 (br 1H).

### EXAMPLE 62A6

1-(1-Cyclohexanecarbonyl-1,2,3,4-tetrahydroquinoline-2-ylmethyl)-4-(2-methoxyphenoxy)piperidine

From 4-(2-methoxyphenoxy)piperidine. (GB 1280699)

Flash chromatography: petroleum ether—EtOAc 6:4. Yield: 48%.

¹H-NMR (δ): 0.75-3.43 (m, 25H), 3.81 (s, 3H), 4.08-4.32 (m, 1H), 5.06-5.22 (m, 1H), 6.81.7.00 (m, 4H), 7.11-7.28 (m, 4H).

### EXAMPLE 63

1-[6-(5-Acetyl-2-thienyl)-1-cyclohexanecarbonyl-1,2,3,4-tetrahydroquinoline-2-ylmethyl)-4-(4-fluoro-2-methoxyphenyl)piperazine

The title compound was synthesised following the procedure described for the compound of Example 47 but starting from the Compound of Example 51 instead of the compound of Example 34 and using 5-acetylthiophen-2-boronic acid instead of phenylboronic acid. Flash chromatography: petroleum ether—EtOAc 3:7. Yield: 48%.

¹H-NMR (δ): 0.70-2.25 (m, 12H), 2.55 (s, 3H), 2.25-3.20 (m, 13H), 3.80 (s, 3H), 4.85-5.40 (m, 1H), 6.50-6.70 (m, 2H), 6.80-6.95 (m, 1H), 7.05-7.40 (m, 2H), 7.40-7.60 (m, 2H), 7.68 (d, 1H).

### EXAMPLE 64

1-[1-Cyclohexanecarbonyl-6-(3,5-dimethylisoxazol-4-yl)-1,2,3,4-tetrahydroquinoline-2-ylmethyl]-4-(4-fluoro-2-methoxyphenyl)piperazine

The title compound was synthesised following the procedure described for the compound of Example 47 but starting from the Compound of Example 51 instead of the compound of Example 34 and using 3,5-dimethylisoxazole-4-boronic acid instead of phenylboronic acid. Yield: 48%.

¹H-NMR (δ):0.70-2.25 (m, 12H), 2.30 and 2.43 (2s, 6H), 2.25-3.20 (m, 13H), 3.80 (s, 3H), 4.80-5.40 (m, 1H), 6.50-6.70 (m, 2H), 6.75-6.95 (m, 1H), 7.00-7.20 (m, 3H).

### EXAMPLE 65

1-(1-Cyclohexanecarbonyl-7-methyl-1,2,3,4-tetrahydroquinoline-2-ylmethyl)-4-(4-fluoro-2-methoxyphenyl)piperazine

a) 1-(4-Fluoro-2-methoxyphenyl)-4-(7-methyl-1,2,3,4-tetrahydroquinoline-2-ylcarbonyl)piperazine (Compound 65A)

The title compound was synthesised following the procedure described for Compound 1D (step g) but using 7-methyl-1,2,3,4-tetrahydroquinoline-2-carboxylic acid (U.S. Pat. No. 4,461,896, 1984) instead of 1,2,3,4-tetrahydroquinoline-2-carboxylic acid and 1-(4-fluoro-2-methoxyphenyl)piperazine instead of 1-(4-indolyl)piperazine. The residue was purified by flash chromatography eluting with CH₂Cl₂-EtOAc 8:2 to giving the title compound (72%).

¹H-NMR (δ): 1.60-1.85 (m, 1H), 2.05-2.20 (m, 1H), 2.25 (s, 3H), 2.60-3.40 (m, 7H), 3.65-4.05 (m, 7H), 4.20-4.35 (m, 1H), 6.50-6.75 (m, 4H), 6.80-7.20 (m, 2H).

b) 1-(4-Fluoro-2-methoxyphenyl)-4-(7-methyl-1,2,3,4-tetrahydroquinoline-2-ylmethyl)piperazine (Compound 65B)

The title compound was synthesised following the procedure described for Compound 1B (step h) but using Compound 65A. The residue was purified by flash chromatography eluting with petroleum ether—EtOAc 5:5 to give the title compound (81%).

¹H-NMR (δ):1.40-1.75 (m, 1H), 1.75-2.00 (m, 1H), 2.20 (s, 3H), 2.35-3.80 (m, 13H), 3.85 (s, 3H), 4.60 (b, 1H), 6.30-6.50 (m, 2H), 6.50-6.70 (m, 2H), 6.80-6.95 (m, 2H).

c) 1-(1-Cyclohexanecarbonyl-7-methyl-1,2,3,4-tetrahydroquinoline-2-ylmethyl)-4-(4-fluoro-2-methoxyphenyl)piperazine

The title compound was synthesised following the alternative acylation procedure described for the compound of Example 1 (step f) but starting from Compound 65B instead of Compound 1B. Yield: 100%.

¹H-NMR (δ): 0.80-3.30 (m, 28H), 3.80 (s, 3H), 5.08 (b, 1H), 6.50-6.65 (m, 2H), 6.75-7.15 (m, 4H).

### EXAMPLE 66

Cis-1-(1-Cyclohexanecarbonyl-4-methyl-1,2,3,4-tetrahydroquinoline-2-ylmethyl)-4-(4-fluoro-2-methoxyphenyl)piperazine

a) cis-1-(4-Fluoro-2-methoxyphenyl)-4-(4-methyl-1,2,3,4-tetrahydroquinoline-2-ylcarbonyl)piperazine (Compound 66A)

The title compound was synthesised following the procedure described for Compound 1D (step g) but using cis-4-methyl-1,2,3,4-tetrahydroquinoline-2-carboxylic acid (obtained by hydrolysis with aq. NaOH in MeOH from the corresponding methyl ester (U.S. Pat. No. 5,616,586, (1997)) instead of 1,2,3,4-tetrahydroquinoline-2-carboxylic acid and 1-(4-fluoro-2-methoxyphenyl)piperazine instead of 1-(4-indolyl)piperazine. The residue was purified by flash chromatography eluting with petroleum ether—EtOAc 1:1 to giving the title compound (72%).

¹H-NMR (δ): 1.30-2.00 (m, 4H), 2.00-2.20 (m, 1H), 2.90-4.50 (m, 14H), 6.55-7.25 (m, 7H).

b) cis-1-(4-Fluoro-2-methoxyphenyl)-4-(4-methyl-1,2,3,4-tetrahydroquinoline-2-ylmethyl)piperazine (Compound 66B)

The title compound was synthesised following the procedure described for Compound 1B (step h) but using Compound 66A. The residue was purified by flash chromatography eluting with petroleum ether—EtOAc 6:4 to give the title compound (77%).

¹H-NMR (δ):1.20-2.00 (m, 5H), 2.30-3.75 (m, 12H), 3.85 (s, 3H), 4.60 (b, 1H), 6.45-6.70 (m, 4H), 6.85-7.00 (m, 2H), 7.10 (d, 1H).

c) cis-1-(1-Cyclohexanecarbonyl-4-methyl-1,2,3,4-tetrahydroquinoline-2-ylmethyl)-4-(4-fluoro-2-methoxyphenyl)piperazine

The title compound was synthesised following the alternative acylation procedure described for the compound of Example 1 (step f) but starting from Compound 66B instead of Compound 1B. Yield: 100%.

¹-NMR (δ): 0.80-3.30 (m, 27H), 3.80,(s, 3H), 5.10 (b, 1H), 6.50-6.65 (m; 2H), 6.75-6.90 (m, 1H), 7.00-7.35 (m, 4H).

### EXAMPLE 67

1-(1-Cyclohexanecarbonyl-8-methyl-1,2,3,4-tetrahydroquinoline-2-ylmethyl)-4-(4-fluoro-2-methoxyphenyl)piperazine

a) 2,8-Dimethylquinoline (Compound 67A)

The title compound was prepared as reported for Compound 25A but using 2-methylaniline instead of 4-trifluoromethylaniline. Flash chromatography: petroleum ether—EtOAc 92:8. Yield: 38%.

¹H-NMR (δ):2.80 (2×s, 6H), 7.19-7.39 (m, 2H), 7.50-7.63 (m, 2H), 8.05 (d, 1H)

b) 8-Methylquinoline-2-carbaldehyde (Compound 67B)

A suspension of SeO₂ (6.66 g) and of Compound 67A (3.87 g) in EtOH (40 ml) was refluxed for 24 h. The reaction mixture was cooled to r.t. The precipitated solid was filtered off, the solvent evaporated under vacuum and the crude was taken up with 2 N HCl (50 ml) and THF (200 ml) and stirred for 1 h. Afterwards, the mixture was diluted with water, alkalinised with NaHCO₃ and extracted with EtOAc. The organic layer was dried (Na₂SO₄) and evaporated to dryness affording the title compound used in the next step without further purification. Yield: 48%.

¹H-NMR (δ): 2.90 (s, 3H), 7.51-7.78 (m 3H), 8.02 (d, 1H), 8.28 (d, 1H), 10.25 (s, 1H).

c) 1-(4-Fluoro-2-methoxyphenyl)-4-(8-methylquinoline-2-ylmethyl)piperazine (Compound 67C)

The title compound was synthesised using the same methodology described in Example 35A using Compound 67B instead of Compound 35A-B and 1-(4-fluoro-2-methoxyphenyl)piperazine. The crude was purified by flash chromatography eluting with petroleum ether—EtOAc 7:3. Yield: 62%.

¹H-NMR (δ): 2.70-3.25 (m, 10H), 3.81 (s 3H), 4.02 (s, 2H), 6.50-6.68 (m, 2H), 6.80-6.94 (m, 1H), 7.32-7.72 (m, 4H), 8.10 (d, 1H).

d) 1-(4-Fluoro-2-methoxyphenyl)-4-(8-methyl-1,2,3,4-tetrahydroquinoline-2-ylmethyl)piperazine (Compound 67D)

The title compound was prepared as described for Compound 1B (procedure e) starting from Compound 67C instead of Compound 1C. The crude was purified by flash chromatography eluting with petroleum ether—EtOAc 7:3 to give the title compound (54%).

¹H-NMR (δ): 1.42-1.72 (m, 1H), 1.82-1.98 (m, 1H), 2.11 (s 3H), 2.35-3.20 (11H), 3.38-3.58 (m, 2H), 3.87 (s, 3H) 4.52-4.79 (m, 1H), 6.50-6.68 (m, 3H), 6.80-6.94 (m, 3H)

e) 1-(1-Cyclohexanecarbonyl-8-methyl-1,2,3,4-tetrahydroquinoline-2-ylmethyl)-4-(4-fluoro-2-methoxyphenyl)piperazine

The title compound was synthesised following the alternative acylation procedure described for the compound of Example 1 (step f) but starting from Compound 67D instead of Compound 1B stirring at reflux in toluene. The crude was purified by flash chromatography eluting with petroleum ether—EtOAc 6:4 to give the title compound (80%).

¹H-NMR (δ): 0.72-1.40 (m, 8H), 1.40-2.56 (m, 14H), 2.56-3.08 (m, 6H), 3.82 (s, 3H), 5.20-5.40 (m, 1H), 6.50-6.68 (m, 2H), 6.80-6.91 (m, 1H), 7.00-7.18 (m, 3H)

### EXAMPLE 68

### EXAMPLE 68A

1-(4-Indolyl)-4-[1-(2-pyrrolylcarbonyl)-1,2,3,4-tetrahydroquinoline-2-ylmethyl]piperazine

To a suspension of 2-pyrrolecarboxylic acid (0.109 g) in CH₂Cl₂ (1 ml) was added through a syringe 2 ml of a 2.5 M solution of oxalyl chloride in CH₂Cl₂ followed after 30 min. by 0.020 ml of DMF. After 18 h stirring at r.t., the solution was evaporated (t=40° C.; p=770 mbar). To ensure the complete removal of oxalyl chloride the procedure of evaporation was repeated twice after having added CH₂Cl₂.

The residue was dissolved in 1.85 ml of CH₂Cl₂ and added to a suspension of Compound 1B (0.084 g) and 4 eq. of polimer supported diisopropylethylamine. After 18 h stirring the the mixture was filtered to remove the PS-DIPEA and the solution so obtained was filtered on cationic exchange resin (Mega Bond Elut®, Bonded Phase SCX), washing with a solution of NH₃ in MeOH (about 3%), evaporating the desired eluted fraction. The residue was purified by flash chromatography eluting with CH₂Cl₂— MeOH 99.5:0.5 to 90:10 to give the title compound.

[M+H]⁺=440.3

¹H-NMR (DMSO; δ): 1.60-1.75 (m, 1H), 2.15-2.34 (m, 2H), 2.40-2.78 (m, 7H), 3.02-3.17 (m, 4H), 4.85-4.96 (m, 1H), 5.65-5.73 (m, 1H), 5.88-5.94 (br, 1H), 6.31-6.38 (br, 1H), 6.40-6.45 (m, 1H), 6.81-6.86 (br, 1H), 6.90-7.15 (m, 5H) 7.19-7.24 (br, 1H), 7.25-7.31 (br, 1H), 10.92-11.03 (br, 1H), 11.73 (br, 1H).

The following compounds were synthesized in the like manner using the proper carboxylic acid instead of 2-pyrrolecarboxylic acid.

When necessary, after the filtration on the resin a further purification by flash chromatography was carried out (eluent: CH₂Cl₂— MeOH gradient from 99.5:0.5 to 90:10). NMR spectra were registered at 400 MHz in DMSO.

### EXAMPLE 68A1

1-[1-(4-Dimethylaminobenzoyl)-1,2,3,4-tetrahydroquinoline-2-ylmethyl]-4-(4-indolyl)piperazine

[M+H]⁺=494.3

¹H-NMR (δ): 1.73-1.85 (m, 1H), 2.15-2.34 (m, 2H), 2.45-2.82 (m, 7H), 2.88 (m, 6H), 3.00-3.17 (m, 4H), 4.73-4.85 (m, 1H), 6.31-6.38 (br, 1H), 6.40 (d, 1H), 6.52 (d, 2H), 6.57-6.63 (m, 1H), 6.85-7.03 (m, 4H), 7.10 (d, 2H), 7.15-7.23 (br, 2H), 10.92-11.03 (br, 1H).

### EXAMPLE 68A2

1-(4-Indolyl)-4-[1-(2-phenylacetyl)-1,2,3,4-tetrahydroquinoline-2-ylmethyl]piperazine

[M+H]⁺=465.3

### EXAMPLE 68A3

1-[1-(4-Cyanobenzoyl)-1,2,3,4-tetrahydroquinoline-2-ylmethyl]-4-(4-indolyl)piperazine

[M+H]⁺=476.2

¹H-NMR (δ): 1.63-1.75 (m, 1H), 2.16-2.27 (m, 1H), 2.30-2.45 (m, 1H), 2.45-2.82 (m, 7H), 2.88 (m, 6H), 3.00-3.17 (m, 4H), 4.73-4.85 (m, 1H), 6.31-6.38 (br, 1H), 6.40 (d, 1H), 6.52 (d, 2H), 6.57-6.63 (m, 1H), 6.85-7.03 (m, 4H), 7.10 (d, 2H), 7.15-7.23 (br, 2H), 10.92-11.03 (br, 1H).

### EXAMPLE 68A4

1-[1-(2-Chlorobenzoyl)-1,2,3,4-tetrahydroquinoline-2-ylmethyl]-4-(4-indolyl)piperazine

[M+H]⁺=485.3

¹H-NMR (δ): 1.51-1.70 (m, 1H), 2.20-2.83 (m, 9H), 2.92-3.16 (m, 4H), 5.04-5.16 (m, 1H), 6.26-6.44 (m, 2H), 6.71-7.60 (m, 11H), 10.92-11.03 (br, 1H).

### EXAMPLE 68A5

1-[1-(4-Chlorobenzoyl)-1,2,3,4-tetrahydroquinoline-2-ylmethyl]-4-(4-indolyl)piperazine

[M+H]⁺=485.3

¹H-NMR (δ): 1.61-1.77 (m, 1H), 2.13-2.26 (m, 1H), 2.29-2.81 (m, 8H), 2.97-3.17, (m, 4H), 4.71-4.88 (m, 1H), 6.31-6.35 (m, 1H), 6.37-6.47 (m, 1H), 6.56-6.69 (m, 1H), 6.86-7.08 (m, 4H), 7.15-7.31 (m, 4H), 7.33-7.38 (m, 2H), 10.94-11.01 (br, 1H).

### EXAMPLE 68A6

1-[1-(3-Chlorobenzoyl)-1,2,3,4-tetrahydroquinoline-2-ylmethyl]-4-(4-indolyl)piperazine

[M+H]⁺=485.3

¹H-NMR (δ):1.65-1.76 (m, 1H), 2.17-2.30 (m, 1H), 2.32-2.84 (m, 8H), 3.01-3.16 (m, 4H), 4.74.-4.81 (m, 1H), 6.56-6.41 (m, 2H), 6.67-6.78 (m, 1H), 6.86-7.09 (m, 4H), 7.16-7.35 (m, 4H), 7.39-7.49 (m, 2H), 10.95-11.04 (br, 1H).

### EXAMPLE 68A7

1-[1-(5-Benzo[1,3]dioxolylcarbonyl)-1,2,3,4-tetrahydroquinoline-2-ylmethyl]-4-(4-indolyl)piperazine

[M+H]⁺=495.3

¹H-NMR (δ): 1.66-1.82 (m, 1H), 2.16-2.86 (m, 9H), 3.01-3.19 (m, 4H), 4.72-4.88 (m, 1H), 6.01 (s, 2H), 6.31-6.49 (m, 2H), 6.66-6.84 (m, 4H), 6.92-7.12 (m, 4H), 7.18-7.32 (m, 2H), 10.94-11.02 (br, 1H).

### EXAMPLE 68A8

1-(1-Cyclopropylcarbonyl-1,2,3,4-tetrahydroquinoline-2-ylmethyl)-4-(4-indolyl)piperazine

[M+H]⁺=415.3

¹H-NMR (δ): 0.76-1.05 (m, 4H), 1.45-1.63 (m, 1H), 1.77-1.88 (m, 1H), 2.11-2.20 (m, 1H), 2.24-2.79 (m, 8H), 2.97-1.13 (m, 4H), 4.79-4.96 (m, 1H), 6.31-6.49 (m, 2H), 6.86-7.40 (m, 7H), 10.91-11.03 (br, 1H).

### EXAMPLE 68A9

1-(4-Indolyl)-4-[1-(2-methylpropionyl)-1,2,3,4-tetrahydroquinoline-2-ylmethyl]piperazine

[M+H]⁺=417.3

¹H-NMR (δ): 1.14 (d, 6H), 1.37-1.56 (m, 1H), 1.2.07-2.17 (m, 1H), 2.24-2.71 (m, 8H), 2.96-3.15 (m, 5H), 4.84-4.98 (m, 1H), 6.32-6.44 (m, 2H), 6.91-7.06 (m, 2H), 7.12-7.32 (m, 5H), 0.91-11.03 (br, 1H).

### EXAMPLE 68A10

1-(4-Indolyl)-4-[1-(2-methoxyacetyl)-1,2,3,4-tetrahydroquinoline-2-ylmethyl]piperazine

[M+H]⁺=419.3

¹H-NMR (δ): 1.46-1.64 (m, 11H), 2.11-2.73 (m, 9H), 2.99-3.10 (m, 4H), 3.26 (s, 3H), 3.92 (s, 2H), 4.72-4.88 (m, 1H), 6.29-6.44 (m, 2H), 6.90-7.11 (m, 2H), 7.16-7.25 (m, 4H), 7.31-7.44 (m, 1H), 10.92-11.03 (br, 1H).

### EXAMPLE 68A11

1-(2-Cyclopropylacetyl-1,2,3,4-tetrahydroquinoline-2-ylmethyl)-4-(4-indolyl)piperazine

[M+H]⁺=429.3

¹H-NMR (δ): 0.21--0.04 (m, 1H), 0.03-0.11 (m, 1H), 0.25-0.45 (m, 2H), 0.84-0.95 (m, 1H), 1.41-1.58 (m, 1H), 2.06-2.35 (m, 4H), 2.42-2.70 (m, 7H), 3.00-3.14 (m, 4H), 4.81-5.02 (m, 11H), 6.31-6.49 (m, 2H), 6.88-7.02 (m, 2H), 7.11-7.34 (m, 5H), 10.91-11.00 (br, 1H).

### EXAMPLE 68A12

1-(4-Indolyl)-4-[1-(3-methylbutanoyl)-1,2,3,4-tetrahydroquinoline-2-ylmethyl]piperazine

[M+H]⁺=431.3

¹H-NMR (δ): 0.9 (s, 6H), 1.45-1.63 (m, 1H), 1.85-2.48 (m, 8H), 2.55-3.75 (m, 4H), 2.98-3.15 (m, 4H), 4.85-5.04 (m, 1H), 6.31-6.50 (m, 2H), 6.90-7.35 (m, 7H), 10.91-11.03 (br, 1H).

### EXAMPLE 68A13

1-(2-Furoyl-1,2,3,4-tetrahydroquinoline-2-ylmethyl)-4-(4-indolyl)piperazine

[M+H]⁺=441.3

¹H-NMR (δ): 1.66-1.82 (m, 1H), 2.20-2.33 (m, 2H), 2.51-2.80 (m, 7H), 2.90-3.15 (m, 4H), 4.75-4.88 (m, 1H), 6.23-6.61 (m, 4H), 6.81-7.15 (m, 5H), 7.17-7.27 (m, 2H), 7.60-6.70 (m, 1H), 10.94-11.02 (br, 1H).

### EXAMPLE 68A14

1-(4-Indolyl)-4-[1-(5-isoxazolylcarbonyl)-1,2,3,4-tetrahydroquinoline-2-ylmethyl]piperazine

[M+H]⁺=442.2

¹H-NMR (δ): 1.54-1.73 (m, 1H), 2.25-2.51 (m, 2H), 2.68-2.81 (m, 7H), 3.12-3.31 (m, 4H), 4.80-5.05 (m, 1H), 6.35-6.61 (m, 3H), 6.80-7.31 (m, 8H), 10.96-11.04 (br, 1H).

### EXAMPLE 68A15

1-(4-Indolyl)-4-[1-(3-tetrahydrofurancarbonyl)-1,2,3,4-tetrahydroquinoline-2-ylmethyl]piperazine

[M+H]⁺=445.3

### EXAMPLE 68A16

1-(4-Indolyl)-4-[1-(2-tetrahydrofurancarbonyl)-1,2,3,4-tetrahydroquinoline-2-ylmethyl]piperazine

[M+H]⁺=445.3

¹H-NMR (δ): 1.34-2.41 (m, 9H), 2.51-2.70 (m, 5H), 2.98-3.15 (m, 4H), 3.61-3.82 (m, 2H), 4.53-4.82 (m, 2H), 6.28-6.48 (m, 2H), 6.81-7.03 (m, 2H), 7.11-7.32 (m, 5H), 10.95-11.03 (br, 1H).

### EXAMPLE 68A17

1-[1-(3,3-Dimethylbutanoyl)-1,2,3,4-tetrahydroquinoline-2-ylmethyl]-4-(4-indolyl)piperazine

[M+H]⁺=445.4

¹H-NMR (δ): 0.85 (s, 9H), 1.39-1.52 (m, 1H), 2.01-2.12 (m, 1H), 2.20-2.31 (m, 3H), 2.35-2.48 (m, 2H), 2.51-2.63 (m, 5H), 2.98-3.12 (m, 4H), 4.81-4.97 (m, 1H), 6.28-6.39 (m, 2H), 6.83-7.03 (m, 2H), 7.07-7.31 (m, 5H), 10.89-11.01 (br, 1H).

### EXAMPLE 68A18

1-[1-(2-Acetoxyacetyl)-1,2,3,4-tetrahydroquinoline-2-ylmethyl]-4-(4-indolyl)piperazine

[M+H]⁺=447.3

¹H-NMR (δ): 1.41-1.54 (m, 1H), 2.01 (s, 3H), 2.20-2.45 (m, 3H), 2.48-2.52 (m, 1H), 2.55-2.69 (m, 5H), 3.01-3.17 (m, 4H), 4.68-4.85 (m, 1H), 4.98-5.03 (m, 2H), 6.26-6.45 (m, 2H), 6.85-7.01 (m, 2H), 7.04-7.46 (m, 5H), 10.91-11.03 (br, 1H).

### EXAMPLE 68A19

1-(4-Indolyl)-4-[1-(2-thienylcarbonyl)-1,2,3,4-tetrahydroquinoline-2-ylmethyl]piperazine

[M+H]⁺=457.2

¹H-NMR (δ): 1.56-1.75 (m, 1H), 2.12-2.25 (m, 1H), 2.27-2.48 (m, 1H), 2.51-2.58 (m, 1H), 2.60-2.77 (m, 6H), 3.02-3.19 (m, 4H), 4.81-4.94 (m, 1H), 6.26-6.48 (m, 2H), 6.81-7.11 (m, 6H), 7.12-7.38 (m, 3H), 7.61-7.70 (m, 1H), 10.98-11.05 (br, 1H).

### EXAMPLE 68A20

1-(4-Indolyl)-4-[1-(3-thienylcarbonyl)-1,2,3,4-tetrahydroquinoline-2-ylmethyl]piperazine

[M+H]⁺=457.2

¹H-NMR (δ): 1.58-1.76(m, 1H), 2.16-2.25 (m, 1H), 2.26-2.47 (m, 1H), 2.52-2.68 (m, 4H), 2.70-2.78 (m, 3H), 3.05-3.21 (m, 4H), 4.76-4.82 (m, 1H), 6.30-6.48 (m, 2H), 6.68-6.81 (m, 2H), 6.84-7.12 (m, 4H), 7.17-7.26 (m, 2H), 7.36-7.40 (m, 1H), 7.58-7.63 (m, 1H), 10.95-11.00 (br, 1H).

### EXAMPLE 68A21

1-[1-(2-Cyclohexylacetyl)-1,2,3,4-tetrahydroquinoline-2-ylmethyl]-4-(4-indolyl)piperazine

[M+H]⁺=471.3

### EXAMPLE 68A22

1-(4-Indolyl)-4-[1-(3-trifluoromethylbenzoyl)-1,2,3,4-tetrahydroquinoline-2-ylmethyl]piperazine

[M+H]⁺=519.3

¹H-NMR (δ): 1.68-1.78 (m, 1H), 2.38-2.50 (m, 1H), 2.60-2.82 (m, 8H), 3.02-3.17 (m, 4H), 4.75-4.80 (m, 1H), 6.25-6.48 (m, 2H), 6.78-7.16 (m, 3H), 7.19-7.27 (m, 2H), 7.48-7.68 (m, 3H), 7.70-7.77 (m, 2H), 8.16-8.24 (m, 1H), 10.93-11.02 (br, 1H).

### EXAMPLE 68A23

1-(4-Indolyl)-4-[1-(4-trifluoromethylbenzoyl)-1,2,3,4-tetrahydroquinoline-2-ylmethyl]piperazine

[M+H]⁺=519.3

¹H-NMR (δ): 1.60-1.78 (m, 1H), 2.18-2.35 (m, 1H), 2.40-2.51 (m, 1H), 2.61-2.71 (m, 4H), 2.76-2.81 (m, 3H), 3.03-3.17 (m, 4H), 4.77-4.91 (m, 1H), 6.26-6.50 (m, 2H), 6.87-7.13 (m, 5H), 7.18-7.26 (m, 2H), 7.49-7.52 (m, 2H), 7.65-7.73 (m, 2H), 10.95-11.02 (br, 1H).

### EXAMPLE 68A24

1-(4-Indolyl)-4-[1-(3-phenylpropionyl)-1,2,3,4-tetrahydroquinoline-2-ylmethyl]piperazine

[M+H]⁺=479.3

¹H-NMR (δ): 1.35-1.53 (m, 1H), 2.05-2.37 (m, 4H), 2.51-2.69 (m, 5H), 2.71-2.90 (m, 4H), 2.93-3.15 (m, 4H), 4.73-4.97, (m, 1H), 6.31-6.48 (m, 2H), 6.90-7.35 (m, 12H), 10.90-11.00 (br, 1H).

### EXAMPLE 68A25

1-(4-Indolyl)-4-[1-(2-methoxybenzoyl)-1,2,3,4-tetrahydroquinoline-2-ylmethyl]piperazine

[M+H]⁺=481.2

### EXAMPLE 68A26

1-(4-Indolyl)-4-[1-(4-methoxybenzoyl)-1,2,3,4-tetrahydroquinoline-2-ylmethyl]piperazine

[M+H]⁺=481.2

¹H-NMR (δ): 1.68-1.87 (m, 1H), 2.17-2.25 (m, 1H), 2.27-2.41 (m, 1H), 2.53-2.69(m, 4H), 2.71-2.77 (in, 3H), 3.04-3.14 (m, 4H), 3.68 (s, 3H), 4.75-4.85 (m, 1H), 6.28-6.49 (m, 2H), 6.58-6.76 (m, 1H), 6.77-6.80 (m, 2H), 6.81-6.89 (m, 2H), 6.90-7.00(m, 2H), 7.19-7.26 (m, 4H), 10.98-11.05 (br; 1H).

### EXAMPLE 68A27

1-[1-[2-(4-Fluorophenyl)acetyl]-1,2,3,4-tetrahydroquinoline-2-ylmethyl]-4-(4-indolyl)piperazine

[M+H]⁺=483.3

¹H-NMR (δ): 1.35-1.51 (m, 1H), 2.07-2.39 (m, 3H), 2.52-2.69 (m, 4H), 2.98-3.17 (m, 4H), 3.57 (s, 2H), 3.59-3.83 (m, 2H), 4.79-4.93 (m, 1H), 6.27-6.43 (m, 2H), 6.87-7.36 (m, 11H), 10.93-11.01 (br, 1H).

### EXAMPLE 68A28

1-[1-(2,6-Difluorobenzoyl)-1,2,3,4-tetrahydroquinoline-2-ylmethyl]-4-(4-indolyl)piperazine

[M+H]⁺=487.2

¹H-NMR (δ): 1.47-1.62 (m, 1H), 2.20-2.28 (m, 1H), 2.29-2.48 (m, 1H), 2.53-2.68 (m, 3H), 2.70-2.77 (m, 4H), 3.03-3.15 (m, 4H), 5.02-5.11 (m, 1H), 6.30-6.45 (m, 2H), 6.75-6.81 (m, 1H), 6.82-7.11 (m, 4H), 7.12-7.31 (m, 5H), 10.94-11.00 (br, 1H).

### EXAMPLE 68A29

1-(4-Indolyl)-4-[1-[2-(2-methoxyphenyl)acetyl]-1,2,3,4-tetrahydroquinoline-2-ylmethyl]piperazine

[M+H]⁺=495.2

¹H-NMR (δ): 1.45-1.58 (m, 1H), 2.15-2.31 (m, 2H), 2.52-2.73 (m, 3H), 3.03-3.15 (m, 4H), 3.61-3.73 (m, 4H), 3.74 (s, 2H), 4.81-4.50 (m, 1H), 6.29-.6.48 (m, 2H), 6.76-7.04 (m, 5H), 7.10-7.28 (m, 6H), 10.94-10.99(br, 1H).

### EXAMPLE 68A30

1-[1-[2-(4-Chlorophenyl)acetyl]-1,2,3,4-tetrahydroquinoline-2-ylmethyl]-4-(4-indolyl)piperazine

[M+H]⁺=499.3

### EXAMPLE 68A31

1-(4-Indolyl)-4-[1-[2-(4-trifluoromethylphenyl)acetyl]-1,2,3,4-tetrahydroquinoline]-2-ylmethyl]piperazine

[M+H]⁺=533.3

### EXAMPLE 68A32

1-(4-Indolyl)-4-[1-(2-pyridylcarbonyl)-1,2,3,4-tetrahydroquinoline-2-ylmethyl]piperazine

[M+H]⁺=452.3

### EXAMPLE 68A33

1-(4-Indolyl)-4-[1-(3-pyridylcarbonyl)-1,2,3,4-tetrahydroquinoline-2-ylmethyl]piperazine

[M+H]⁺=452.3

### EXAMPLE 68A34

1-(4-Indolyl)-4-[1-(4-pyridylcarbonyl)-1,2,3,4-tetrahydroquinoline-2-ylmethyl]piperazine

[M+H]⁺=452.3

### EXAMPLE 68A35

1-[1-(3,5-Dimethyl-4-isoxazolylcarbonyl)-1,2,3,4-tetrahydroquinoline-2-ylmethyl]-4-(4-indolyl)piperazine

[M+H]⁺=470.3

### EXAMPLE 68A36

1-[1-(2,2-Dimethylpropionyl)-1,2,3,4-tetrahydroquinoline-2-ylmethyl]-4-(4-indolyl)piperazine

[M+H]⁺=431.3

¹H-NMR (δ): 1.21 (s, 9H), 1.43-1.50 (m, 1H), 2.20-2.28 (m, 1H), 2.31-2.41 (m, 2H), 2.42-2.51 (m, 2H), 2.60-2.73 (m, 4H), 3.03-3.17 (m, 4H), 4.95-5.01 (m, 1H), 6.25-6.48 (m, 2H), 6.82-7.02 (m, 3H), 7.18-7.28 (m, 4H), 10.95-11.01 (br, 1H).

### EXAMPLE 68A37

1-(4-Indolyl)-4-[1-[2-(3-pyridyl)acetyl]-1,2,3,4-tetrahydroquinoline-2-ylmethyl]piperazine

[M+H]⁺=466.3

### EXAMPLE 68A38

1-[1-(1-Acetylpiperidine-4-carbonyl)-1,2,3,4-tetrahydroquinoline-2-ylmethyl]-4-(4-indolyl)piperazine

[M+H]⁺=500.3

### EXAMPLE 68A39

1-(4-Indolyl)-4-[1-[2-(2-thienylacetyl)]-1,2,3,4-tetrahydroquinoline-2-ylmethyl]piperazine

[M+H]⁺=471.2

### EXAMPLE 68A40

1-(4-Indolyl)-4-[1-[2-(3-thienylacetyl)]-1,2,3,4-tetrahydroquinoline-2-ylmethyl]piperazine

[M+H]⁺=471.3

### EXAMPLE 68A41

1-(4-Indolyl)-4-[1-[3-(3-pyridylpropionyl)]-1,2,3,4-tetrahydroquinoline-2-ylmethyl]piperazine

[M+H]⁺=480.3

### EXAMPLE 68A42

1-(4-Indolyl)-4-[1-(2-phenoxyacetyl)-1,2,3,4-tetrahydroquinoline-2-ylmethyl]piperazine

[M+H]⁺=481.3

### EXAMPLE 68A43

1-(4-Indolyl)-4-[1-[2-(4-methoxyphenylacetyl)]-1,2,3,4-tetrahydroquinoline-2-ylmethyl]piperazine

[M+H]⁺=495.3

### EXAMPLE 68A44

1-(4-Indolyl)-4-[1-(2-phenylmethoxyacetyl)-1,2,3,4-tetrahydroquinoline-2-ylmethyl]piperazine

[M+H]⁺=495.3

### EXAMPLE 68A45

1-[1-[2-(2-Chlorophenylacetyl)]-1,2,3,4-tetrahydroquinoline-2-ylmethyl]-4-(4-indolyl)piperazine

[M+H]⁺=499.2

### EXAMPLE 68A46

1-[1-[2-(N-Benzoyl-N-methylamino)acetyl]-1,2,3,4-tetrahydroquinoline-2-ylmethyl]-4-(4-indolyl)piperazine

[M+H]⁺=522.3

### EXAMPLE 68A47

1-(4-Indolyl)-4-[1-(1-methyl-3-indolylcarbonyl)-1,2,3,4-tetrahydroquinoline-2-ylmethyl]piperazine

[M+H]⁺=504.3

### EXAMPLE 68A48

1-[1-[2-(4-Dimethylaminophenylacetyl)]-1,2,3,4-tetrahydroquinoline-2-ylmethyl]-4-(4-indolyl)piperazine

[M+H]⁺=508.3

### EXAMPLE 68A49

1-[1-[2-(5-Benzo[1,3]dioxolyl)acetyl]-1,2,3,4-tetrahydroquinoline-2-ylmethyl]-4-(4-indolyl)piperazine

[M+H]⁺=509.3

### EXAMPLE 68A50

1-[1-[2-(2-Chlorophenoxy)acetyl]-1,2,3,4-tetrahydroquinoline-2-ylmethyl]-4-(4-indolyl)piperazine

[M+H]⁺=515.3

### EXAMPLE 68A51

1-[1-[2-(4-Chlorophenoxy)acetyl]-1,2,3,4-tetrahydroquinoline-2-ylmethyl]-4-(4-indolyl)piperazine

[M+H]⁺=515.3

### EXAMPLE 68A52

1-(4-Indolyl)-4-[1-[2-(4-morpholinyl)pyridine-5-carbonyl]-1,2,3,4-tetrahydroquinoline-2-ylmethyl]piperazine

[M+H]⁺=537.3

### EXAMPLE 68A53

1-(4-Indolyl)-4-[1-(5-methylisoxazole-4-carbonyl)-1,2,3,4-tetrahydroquinoline-2-ylmethyl]piperazine

[M+H]⁺=456.2

### EXAMPLE 68A54

1-(4-Indolyl)-4-[1-[2-(5-oxopyrrolidin-2yl)acetyl]-1,2,3,4-tetrahydroquinoline-2-ylmethyl]piperazine

[M+H]⁺=472.3

### EXAMPLE 68A55

1-(4-Indolyl)-4-[1-(2-methyl-5-piperidin-1-ylsulfonylfuran-3-carbonyl)-1,2,3,4-tetrahydroquinoline-2-ylmethyl]piperazine

[M+H]⁺=602.38

### EXAMPLE 68A56

1-(4-Indolyl)-4-[1-(2-methyl-5-morpholin-4-ylsulfonylfuran-3-carbonyl)-1,2,3,4-tetrahydroquinoline-2-ylmethyl]piperazine

[M+H]⁺=604.45

### EXAMPLE 68A57

1-(4-Indolyl)-4-[1-[1-(4-methylphenylsulphonyl)pyrrole-3-carbonyl]-1,2,3,4-tetrahydroquinoline-2-ylmethyl]piperazine

[M+H]⁺=594.26

### EXAMPLE 68A58

1-(4-Indolyl)-4-[1-(2-methyl-5-dimethylaminolsulfonylfuran-3-carbonyl)-1,2,3,4-tetrahydroquinoline-2-ylmethyl]piperazine

[M+H]⁺=562

### EXAMPLE 68A59

1-(4-Indolyl)-4-[1-(2-methyl-5-pyrrolidin-1-ylsulfonylfuran-3-carbonyl)-1,2,3,4-tetrahydroquinoline-2-ylmethyl]piperazine

[M+H]⁺=588

### EXAMPLE 68A60

1-[(1-Adamantylcarbonyl)-1,2,3,4-tetrahydroquinoline-2-ylmethyl]-4-(4-indolyl)piperazine

[M+H]⁺=509.19

### EXAMPLE 68A61

1-(4-Indolyl)-4-[1-(3-phenoxypropionyl)-1,2,3,4-tetrahydroquinoline-2-ylmethyl]piperazine

[M+H]⁺=495.18

### EXAMPLE 68A62

1-(4-Indolyl)-1-[1-(4-phenoxybutanoyl)-1,2,3,4-tetrahydroquinoline-2-ylmethyl]piperazine

[M+H]⁺=509.19

### EXAMPLE 68A63

1-(4-Indolyl)-4-[1-[2-(2-fluorophenylacetyl)]-1,2,3,4-tetrahydroquinoline-2-ylmethyl]piperazine

[M+H]⁺=483.15

### EXAMPLE 68A64

1-(4-Indolyl)-4-[1-[2-(2-trifluoromethylphenylacetyl)]-1,2,3,4-tetrahydroquinoline-2-ylmethyl]piperazine

[M+H]⁺=533.16

### EXAMPLE 68A65

1-[1-(2-Bicyclo[2.2.2]octylcarbonyl)-1,2,3,4-tetrahydroquinoline-2-ylmethyl]-4-(4-indolyl)piperazine

[M+H]⁺=483.21

### EXAMPLE 68A66

1-(4-Indolyl)-1-[1-(4-phenylbutanoyl)-1,2,3,4-tetrahydroquinoline-2-ylmethyl]piperazine

[M+H]⁺=493.2

### EXAMPLE 68A67

1-(4-Indolyl)-4-[1-(3-methoxybenzoyl)-1,2,3,4-tetrahydroquinoline-2-ylmethyl]piperazine

[M+H]⁺=481.16

### EXAMPLE 68A68

1-[1-(4-Hydroxybenzoyl)-1,2,3,4-tetrahydroquinoline-2-ylmethyl]-4-(4-indolyl)piperazine

[M+H]⁺=467.16

### EXAMPLE 68A69

1-[1-(4-Ethoxybenzoyl)-1,2,3,4-tetrahydroquinoline-2-ylmethyl]-4-(4-indolyl)piperazine

[M+H]⁺=495.18

### EXAMPLE 68A70

1-(4-Indolyl)-4-[1-(4-methylbenzoyl)-1,2,3,4-tetrahydroquinoline-2-ylmethyl]piperazine

[M+H]⁺=465.19

### EXAMPLE 68A71

1-[1-[2-(N-Benzyl-N-methanesulphonylamino)acetyl]-1,2,3,4-tetrahydroquinoline-2-ylmethyl]-4-(4-indolyl)piperazine

[M+H]⁺=572.13

### EXAMPLE 68A72

1-(4-Indolyl)-4-[1-[(2S)-5-oxopyrrolidine-2-ylcarbonyl]-1,2,3,4-tetrahydroquinoline-2-ylmethyl]piperazine

[M+H]⁺=458.2

### EXAMPLE 68A73

1-(4-Indolyl)-4-[1-[(2R)-5-oxopyrrolidine-2-ylcarbonyl]-1,2,3,4-tetrahydroquinoline-2-ylmethyl]piperazine

[M+H]⁺=458

### EXAMPLE 69

General Method:

To a solution of Compound 1B was added a solution of the proper amine (1 eq.) in anhydrous THF (5 ml); then via a stringe was added, under magnetic stirring at r.t. in a nitrogen atmosphere, the isocyanate as it was or disssolved in about 1 ml of anhydrous THF. The reaction mixture was reacted at r.t. or at reflux for 20-50 h, then was filtered on a cationic resin (Mega Bond Elut®, Bonded Phase SCX) washing with CH₂Cl₂— MeOH 1:1 to remove the not alkaline impurities followed by a 3% solution of NH₃ in MeOH to recover the title products. When necessary a further purification by flash chromatography was carried out (eluent: CH₂Cl₂— MeOH gradient from 99.5:0.5 to 90:10).

The following compounds were synthesised according to the above procedure:

### EXAMPLE 69A

1-(4-Indolyl)-4-[1-(2-methylphenylaminocarbonyl)-1,2,3,4-tetrahydroquinoline-2-ylmethyl]piperazine

[M+H]⁺=480.2

### EXAMPLE 69A1

1-(4-Indolyl)-4-[1-(3-methylphenylaminocarbonyl)-1,2,3,4-tetrahydroquinoline-2-ylmethyl]piperazine

[M+H]⁺=480.2

### EXAMPLE 69A2

1-[1-(4-Fluorophenylaminocarbonyl)-1,2,3,4-tetrahydroquinoline-2-ylmethyl)-4-(4-indolyl)piperazine

[M+H]⁺=484.3

### EXAMPLE 69A3

1-(4-Indolyl)-4-[1-(4-methylphenylaminocarbonyl)-1,2,3,4-tetrahydroquinoline-2-ylmethyl]piperazine

[M+H]⁺=480.3

### EXAMPLE 69A4

1-(4-Indolyl)-4-[1-[(1S)-1-phenylethylaminocarbonyl]-1,2,3,4-tetrahydroquinoline-2-ylmethyl]piperazine

[M+H]⁺=494.4

### EXAMPLE 69A5

1-(4-Indolyl)-1-[1-[(1R)-1-phenylethylaminocarbonyl]-1,2,3,4-tetrahydroquinoline-2-ylmethyl)piperazine (diastereoisomer A)

[M+H]⁺=494.4

### EXAMPLE 69A6

1-(4-Indolyl)-1-[1-[(1R)-1-phenylethylaminocarbonyl]-1,2,3,4-tetrahydroquinoline-2-ylmethyl]piperazine (1:1 diastereoisomers mixture)

[M+H]⁺=494.3

### EXAMPLE 69A7

1-(4-Indolyl)-4-[1-(1-methylethylaminocarbonyl)-1,2,3,4-tetrahydroquinoline-2-ylmethyl]piperazine

[M+H]⁺=432.3

### EXAMPLE 69A8

1-(4-Indolyl)-4-[1-methylaminothiocarbonyl-1,2,3,4-tetrahydroquinoline-2-ylmethyl)piperazine

[M+H]⁺=420.2

### EXAMPLE 69A9

1-(1-Benzylaminocarbonyl-1,2,3,4-tetrahydroquinoline-2-ylmethyl)-4-(4-indolyl)piperazine

[M+H]⁺=480.3

### EXAMPLE 69A10

1(4-Indolyl)-4-[1-(2-phenylethylaminocarbonyl)-1,2,3,4-tetrahydroquinoline-2-ylmethyl]piperazine

[M+H]⁺=494.3

### EXAMPLE 69A11

1-(4-Indolyl)-4-(1-pentylaminocarbonyl-1,2,3,4-tetrahydroquinoline-2-ylmethyl]piperazine

[M+H]⁺=460.3

### EXAMPLE 69A12

1-[1-(1,1-Dimethylethylaminocarbonyl)-1,2,3,4-tetrahydroquinoline-2-ylmethyl]-1-(4-indolyl)piperazine

[M+H]⁺=446.3

### EXAMPLE 69A13

1(4-Indolyl)-4-[1-(4-methoxyphenylaminocarbonyl)-1,2,3,4-tetrahydroquinoline-2-ylmethyl]piperazine

[M+H]⁺=496.18

### EXAMPLE 69A14

1(4-Indolyl)-4-[1-(4-methoxyphenylmethylaminocarbonyl)-1,2,3,4-tetrahydroquinoline-2-ylmethyl]piperazine

[M+H]⁺=510.18

### EXAMPLE 70

The sulphonyl chloride was dissolved in 1.85 ml of CH₂Cl₂ and added to a suspension of Compound 1B and 4 eq. of polimer supported diisopropylethylamine (PS-DIPEA). After 18 h stirring, the mixture was filtered to remove the PS-DIPEA and the solution so obtained was filtered on cationic exchange resin (Mega Bond Elut®, Bonded Phase SCX), washing with a solution of NH₃ in MeOH (about 3%), evaporating the desired eluted fraction. The residue was purified by flash chromatography eluting with CH₂Cl₂— MeOH gradient from 99.5:0.5 to 90:10 to give the title compound.

The following compounds were synthesised according to the above procedure:

### EXAMPLE 70A

1-(4-Indolyl)-1-[1-phenylsulphonyl-1,2,3,4-tetrahydroquinoline-2-ylmethyl]piperazine

[M+H]⁺=487.2

### EXAMPLE 70A1

1-(4-Indolyl)-4-[1-(2-thienylsulphonyl)-1,2,3,4-tetrahydroquinoline-2-ylmethyl]piperazine

[M+H]⁺=493.7

### EXAMPLE 70A2

1(4-Indolyl)-4-[1-(4-methoxyphenylsulphonyl)-1,2,3,4-tetrahydroquinoline-2-ylmethyl]piperazine

[M+H]⁺=517.12

### EXAMPLE 70A3

1(4-Indolyl)-4-[1-(4-methylphenylsulphonyl)-1,2,3,4-tetrahydroquinoline-2-ylmethyl]piperazine

[M+H]⁺=501.14

### EXAMPLE 70A4

1-[1-(4-Fluorophenylsulphonyl)-1,2,3,4-tetrahydroquinoline-2-ylmethyl]-4-(4-indolyl)piperazine

[M+H]⁺=505.11

### EXAMPLE 70A5

1-[1-(4-Cyanophenylsulphonyl)-1,2,3,4-tetrahydroquinoline-2-ylmethyl]-4-(4-indolyl)piperazine

[M+H]⁺=512.16

### EXAMPLE 70A6

1-(4-Indolyl)-4-(1-phenylmethylsulphonyl-1,2,3,4-tetrahydroquinoline-2-ylmethyl)piperazine

[M+H]⁺=501.2

### EXAMPLE 71

A slightly different procedure (versus the acylation method reported in Example 78A) was used to acylate Compound 1B with acids containing the tert-butoxycarbonyl moiety.

To a solution of compound 1B (100 mg, 1 eq.), of the proper acid (1.5 eq.) and of 4-DMAP (10.5 mg, 0.3 eq.) in anhydrous CH₂Cl₂ (5 ml) was added DCC (870 μl of a 1 M solution, 3 eq.) via a syringe, stirring at r.t. under nitrogen atmosphere. After at least 18 h, the dicyclohexylurea by-product precipitated. The reaction mixture was dissolved in MeOH and the solution was eluted through a cationic resin (Mega Bond Elut®, Bonded Phase SCX) washing with a solution of NH₃ in MeOH (about 3%). When necessary a further purification was carried out by flash chromatography (CH₂Cl₂— MeOH gradient from 99.5:0.5 to 90:10).

By the above procedure the following compounds of Example 71 were synthesized:

### EXAMPLE 71A

1-[1-(2-tert-Butoxycarbonylamino)acetyl-1,2,3,4-tetrahydroquinoline-2-ylmethyl]-4-(4-indolyl)piperazine

[M+H]⁺=504.5

### EXAMPLE 71A1

1-[1-(3-tert-Butoxycarbonylamino)propionyl-1,2,3,4-tetrahydroquinoline-2-ylmethyl]-4-(4-indolyl)piperazine

[M+H^{]}=518.3

### EXAMPLE 71A2

1-[1-[2-(1-tert-Butoxycarbonylindol-3-yl)acetyl]-1,2,3,4-tetrahydroquinoline-2-ylmethyl]-4-(4-indolyl]piperazine

[M+H]⁺=604.45

### EXAMPLE 71A3

1-[1-[4-(1-tert-Butoxycarbonylpiperidino)carbonyl]-1,2,3,4-tetrahydroquinoline-2-ylmethyl]-4-(4-indolyl)piperazine

[M+H]⁺=558.42

### EXAMPLE 71A4

1-[1-(1-tert-Butoxycarbonyl-(2R)-pyrrolidin-2yl)carbonyl-1,2,3,4-tetrahydroquinoline-2-ylmethyl]-4-(4-indolyl)piperazine

[M+H]⁺=544.31

### EXAMPLE 71A5

1-[1-(1-tert-Butoxycarbonyl-(2S)-pyrrolidin-2yl)carbonyl-1,2,3,4-tetrahydroquinoline-2-ylmethyl]-4-(4-indolyl)piperazine

[M+H]⁺=544.44

### EXAMPLE 72

General Method:

A solution of the proper amine (1.2 eq.) in MeOH (1.5 ml) and a solution of Compound 35A-B (100 mg, 1 eq.) in MeOH (1.5 ml) were sequentially added via a syringe to PS-NEt₃CNBH₃ (317 mg, 2 eq., 2.32 mmol/g) under nitrogen atmosphere. After stirring for 18 h at r.t., the PS-NEt₃CNBH₃ was removed by filtration and the filtrate was purified on a cationic exchange resin (Mega Bond Elut®, Bonded Phase SCX) washing with CH₂Cl₂— MeOH 1:1 to remove the not alkaline impurities; then with a 3% solution of NH₃ in MeOH to yield the wished products. The solvent was evaporated and the residue dissolved in CH₂Cl₂ (5 ml) and taken up with PS-NCO (250 mg, 1.51 mmol/g). After 2 h the resin was removed by filtration and the solvent evaporated. When necessary a further purification was carried out by flash chromatography (CH₂Cl₂— MeOH gradient from 99:1 to 90:10).

By the above procedure the following compounds of Example 72 were synthesized:

### EXAMPLE 72A

1-(1-Cyclohexanecarbonyl-1,2,3,4-tetrahydroquinolin-2-ylmethyl)-2-(1-hydroxy-1-phenylmethyl)piperidine

[M+H]⁺=447.38

### EXAMPLE 72A1

1-(1-Cyclohexanecarbonyl-1,2,3,4-tetrahydroquinolin-2-ylmethyl)-4-(2,6-dimethylphenyl)piperidine

[M+H]⁺=445.37

### EXAMPLE 72A2

2-(4-Chlorophenyl)-1-(1-cyclohexanecarbonyl-1,2,3,4-tetrahydroquinolin-2-ylmethyl)piperidine

[M+H]⁺=452.33

### EXAMPLE 72A3

1-(1-Cyclohexanecarbonyl-1,2,3,4-tetrahydroquinolin-2-ylmethyl)-4-phenylpiperidine

[M+H]⁺=417.21

### EXAMPLE 72A4

4-Benzoyl-11-(1-Cyclohexanecarbonyl-1,2,3,4-tetrahydroquinolin-2-ylmethyl)piperidine

[M+H]⁺=445.37

### EXAMPLE 72A5

1-(1-Cyclohexanecarbonyl-1,2,3,4-tetrahydroquinolin-2-ylmethyl)-4-(2-nitrophenyl)piperazine

[M+H]⁺=463.34

### EXAMPLE 72A6

1-(1-Cyclohexanecarbonyl-1,2,3,4-tetrahydroquinolin-2-ylmethyl)-4-(3-trifluoromethylpyridin-2-yl)piperazine

[M+H]⁺=487.23

### EXAMPLE 72A7

1-(1-Cyclohexanecarbonyl-1,2,3,4-tetrahydroquinolin-2-ylmethyl)-4-(4-fluoro-2-nitrophenyl)piperazine

[M+H]⁺=481.31

### EXAMPLE 72A8

1-(1-Cyclohexanecarbonyl-1,2,3,4-tetrahydroquinolin-2-ylmethyl)-4-(2,3-dichlorophenyl)piperazine

[M+H]⁺=487.23

### EXAMPLE 72A9

1-(1-Cyclohexanecarbonyl-1,2,3,4-tetrahydroquinolin-2-ylmethyl)-4-(3-cyanopyridin-2-yl)piperazine

[M+H]⁺=444.33

### EXAMPLE 72A10

1-(1-Cyclohexanecarbonyl-1,2,3,4-tetrahydroquinolin-2-ylmethyl)-4-(3,4-dichlorophenyl)piperazine

[M+H]⁺=486.25

### EXAMPLE 72A11

1-(1-Cyclohexanecarbonyl-1,2,3,4-tetrahydroquinolin-2-ylmethyl)-4-(2-ethylphenyl)piperazine

[M+H]⁺=446.38

### EXAMPLE 72A12

1-(4-Chloro-3-trifluoromethylphenyl)-4-(1-cyclohexanecarbonyl-1,2,3,4-tetrahydroquinolin-2-ylmethyl)piperazine

[M+H]⁺=520.32

### EXAMPLE 72A13

1-(2-Cyanophenyl)-4-(1-cyclohexanecarbonyl-1,2,3,4-tetrahydroquinolin-2-ylmethyl)piperazine

[M+H]⁺=443.35

### EXAMPLE 72A14

1-(1-Cyclohexanecarbonyl-1,2,3,4-tetrahydroquinolin-2-ylmethyl)-4-(2,4-dimethoxyphenyl)piperazine

[M+H]⁺=478.38

### EXAMPLE 72A15

1-(1-Cyclohexanecarbonyl-1,2,3,4-tetrahydroquinolin-2-ylmethyl)-4-(2-methylthiophenyl)piperazine

[M+H]⁺=464.35

### EXAMPLE 72A16

1-(1-Cyclohexanecarbonyl-1,2,3,4-tetrahydroquinolin-2-ylmethyl)-4-(3,4-dimethoxyphenyl)piperazine

[M+H]⁺=478.39

### EXAMPLE 72A17

1-(1-Cyclohexanecarbonyl-1,2,3,4-tetrahydroquinolin-2-ylmethyl)-4-(3-hydroxyphenyl)piperazine

[M+H]⁺=434.32

### EXAMPLE 72A18

1-(1-Cyclohexanecarbonyl-1,2,3,4-tetrahydroquinolin-2-ylmethyl)-4-(2,4-difluorophenyl)piperazine

[M+H]⁺=454.3

### EXAMPLE 72A19

1-(5-Chloro-2-methoxyphenyl)-4-(1-cyclohexanecarbonyl-1,2,3,4-tetrahydroquinolin-2-ylmethyl)piperazine

[M+H]⁺=482.29

### EXAMPLE 72A20

1-(1-Cyclohexanecarbonyl-1,2,3,4-tetrahydroquinolin-2-ylmethyl)-4-(6-methylpyridin-2-yl)piperazine

[M+H]⁺=432.37

### EXAMPLE 72A21

1-(1-Cyclohexanecarbonyl-1,2,3,4-tetrahydroquinolin-2-ylmethyl)-4-(4-methylphenyl)piperazine

[M+H]⁺=432.36

### EXAMPLE 72A22

1-(1-Cyclohexanecarbonyl-1,2,3,4-tetrahydroquinolin-2-ylmethyl)-4-(4-trifluoromethylphenyl)piperazine

[M+H]⁺=486.32

### EXAMPLE 72A23

1-(3-Chlorophenyl)-4-(1-cyclohexanecarbonyl-1,2,3,4-tetrahydroquinolin-2-ylmethyl)piperazine

[M+H]⁺=452.26

### EXAMPLE 72A24

1-(2-Chlorophenyl)-4-(1-cyclohexanecarbonyl-1,2,3,4-tetrahydroquinolin-2-ylmethyl)piperazine

[M+H]⁺=452.26

### EXAMPLE 72A25

1-(1-Cyclohexanecarbonyl-1,2,3,4-tetrahydroquinolin-2-ylmethyl)-4-(3-methoxyphenyl)piperazine

[M+H]⁺=448.36

### EXAMPLE 72A26

1-(1-Cyclohexanecarbonyl-1,2,3,4-tetrahydroquinolin-2-ylmethyl)-4-(4-fluorophenyl)piperazine

[M+H]⁺=436.2

### EXAMPLE 72A27

1-(4-Acetylphenyl)-4-(1-cyclohexanecarbonyl-1,2,3,4-tetrahydroquinolin-2-ylmethyl)piperazine

[M+H]⁺=460.2

### EXAMPLE 72A28

1-(1-Cyclohexanecarbonyl-1,2,3,4-tetrahydroquinolin-2-ylmethyl)-4-(4-hydroxyphenyl)piperazine

[M+H]⁺=434.03

### EXAMPLE 72A29

1-(5-Chloro-2-methylphenyl)-4-(1-cyclohexanecarbonyl-1,2,3,4-tetrahydroquinolin-2-ylmethyl)piperazine

[M+H]⁺=466.2

### EXAMPLE 72A30

1-(1-Cyclohexanecarbonyl-1,2,3,4-tetrahydroquinolin-2-ylmethyl)-4-(3-methylphenyl)piperazine

[M+H]⁺=432

### EXAMPLE 72A31

1-(1-Cyclohexanecarbonyl-1,2,3,4-tetrahydroquinolin-2-ylmethyl)-4-(2-nitro-4-trifluoromethylphenyl)piperazine

[M+H]⁺=531

### EXAMPLE 72A32

1-(1-Cyclohexanecarbonyl-1,2,3,4-tetrahydroquinolin-2-ylmethyl)-4-(5-trifluoromethylpyridin-2-yl)piperazine

[M+H]⁺=487

### EXAMPLE 72A33

1-(1-Cyclohexanecarbonyl-1,2,3,4-tetrahydroquinolin-2-ylmethyl)-4-(3-chloro-5-trifluoromethylpyridin-2-yl)piperazine

[M+H]⁺=521

### EXAMPLE 72A34

1-(5-Cyanopyridin-2-yl)-4-(1-cyclohexanecarbonyl-1,2,3,4-tetrahydroquinolin-2-ylmethyl)piperazine

[M+H]⁺=444

### EXAMPLE 72A35

1-(1-Cyclohexanecarbonyl-1,2,3,4-tetrahydroquinolin-2-ylmethyl)-4-(2-methylquinolin-4-yl)piperazine

[M+H]⁺=483.05

### EXAMPLE 72A36

1-(1-Cyclohexanecarbonyl-1,2,3,4-tetrahydroquinolin-2-ylmethyl)-4-(phenylmethyl)piperidine

[M+H]⁺=431

### EXAMPLE 72A37

1-(Benzofuran-3-yl)-4-(1-Cyclohexanecarbonyl-1,2,3,4-tetrahydroquinolin-2-ylmethyl)piperidine

[M+H]⁺=457

### EXAMPLE 72A38

1-(1-Cyclohexanecarbonyl-1,2,3,4-tetrahydroquinolin-2-ylmethyl)-4-(4-fluorophenyl)piperidine

[M+H]⁺=435.2

### EXAMPLE 72A39

1-(1-Cyclohexanecarbonyl-1,2,3,4-tetrahydroquinolin-2-ylmethyl)-4-(3-fluorophenyl)piperidine

[M+H]⁺=435.2

### EXAMPLE 72A40

1-(1-Cyclohexanecarbonyl-1,2,3,4-tetrahydroquinolin-2-!ylmethyl)-4-(2-fluorophenyl)piperidine

[M+H]⁺=435.2

### EXAMPLE 72A41

4-(4-Chlorophenyl)-1-(1-cyclohexanecarbonyl-1,2,3,4-tetrahydroquinolin-2-ylmethyl)piperidine

[M+H]⁺=451.2

### EXAMPLE 72A42

1-(1-Cyclohexanecarbonyl-1,2,3,4-tetrahydroquinolin-2-ylmethyl)-4-(4-trifluoromethylphenyl)piperidine

[M+H]⁺=485.2

### EXAMPLE 72A43

1-(1-Cyclohexanecarbonyl-1,2,3,4-tetrahydroquinolin-2-ylmethyl)-4-(3-trifluoromethylphenyl)piperidine

[M+H]⁺=485

### EXAMPLE 72A44

1-(1-Cyclohexanecarbonyl-1,2,3,4-tetrahydroquinolin-2-ylmethyl)-4-[5-(2-furyl)-2H-pyrazol-3-yl]piperidine

[M+H]⁺=473

### EXAMPLE 72A45

1-(1-Cyclohexanecarbonyl-1,2,3,4-tetrahydroquinolin-2-ylmethyl)-4-(4-methoxyphenyl)piperidine

[M+H]⁺=447

### EXAMPLE 72A46

1-(1-Cyclohexanecarbonyl-1,2,3,4-tetrahydroquinolin-2-ylmethyl)-4-(3-methoxyphenyl)piperidine

[M+H]⁺=447

### EXAMPLE 72A47

1-(1-Cyclohexanecarbonyl-1,2,3,4-tetrahydroquinolin-2-ylmethyl)-4-(4-fluorobenzoyl)piperidine

[M+H]⁺=463

### EXAMPLE 72A48

1-(1-Cyclohexanecarbonyl-1,2,3,4-tetrahydroquinolin-2-ylmethyl)-4-(4-phenylmethylaminocarbonylamino)piperidine

[M+H]⁺=489.2

### EXAMPLE 72A49

1-(1-Cyclohexanecarbonyl-1,2,3,4-tetrahydroquinolin-2-ylmethyl)-2-(4-methoxyphenylmethyl)piperidine

[M+H]⁺=461

### EXAMPLE 72A50

1-(1-Cyclohexanecarbonyl-1,2,3,4-tetrahydroquinolin-2-ylmethyl)-2-(4-fluorophenylmethyl)piperidine

[M+H]⁺=449

### EXAMPLE 72A51

1-(1-Cyclohexanecarbonyl-1,2,3,4-tetrahydroquinolin-2-ylmethyl)-4-(2,3-dichlorophenyl)piperidine

[M+H]⁺=485.2

### EXAMPLE 72A52

1-(1-Cyclohexanecarbonyl-1,2,3,4-tetrahydroquinolin-2-ylmethyl)-4-(2,6-difluorophenyl)piperidine

[M+H]⁺=453.2

### EXAMPLE 72A53

4-(2-Chloro-6-fluorophenyl)-1-(1-cyclohexanecarbonyl-1,2,3,4-tetrahydroquinolin-2-ylmethyl)piperidine

[M+H]⁺=469.2

### EXAMPLE 72A54

1-(1-Cyclohexanecarbonyl-1,2,3,4-tetrahydroquinolin-2-ylmethyl)-4-(2,5-dimethylphenyl)piperidine

[M+H]⁺=445.2

### EXAMPLE 72A55

1-(1-Cyclohexanecarbonyl-1,2,3,4-tetrahydroquinolin-2-ylmethyl)-4-(3-methylphenyl)piperidine

[M+H]⁺=431.2

### EXAMPLE 72A56

1-(1-Cyclohexanecarbonyl-1,2,3,4-tetrahydroquinolin-2-ylmethyl)-4-(2-methylphenyl)piperidine

[M+H]⁺=431.2

### EXAMPLE 72A57

1-(1-Cyclohexanecarbonyl-1,2,3,4-tetrahydroquinolin-2-ylmethyl)-4-(3-fluoro-2-methylphenyl)piperidine

[M+H]⁺=449.2

### EXAMPLE 72A58

1-(1-Cyclohexanecarbonyl-1,2,3,4-tetrahydroquinolin-2-ylmethyl)-4-(3,5-difluorophenyl)piperidine

[M+H]⁺=453.2

### EXAMPLE 72A59

1-(1-Cyclohexanecarbonyl-1,2,3,4-tetrahydroquinolin-2-ylmethyl)-4-(2,5-difluorophenyl)piperidine

[M+H]⁺=453.2

### EXAMPLE 72A60

1-(1-Cyclohexanecarbonyl-1,2,3,4-tetrahydroquinolin-2-ylmethyl)-4-(3,5-dimethylphenyl)piperidine

[M+H]⁺=445.2

### EXAMPLE 72A61

4-(3-Bromophenyl)-1-(1-cyclohexanecarbonyl-1,2,3,4-tetrahydroquinolin-2-ylmethyl)piperidine

[M+H]⁺=495.1

### EXAMPLE 72A62

4-(2-Bromophenyl)-1-(1-cyclohexanecarbonyl-1,2,3,4-tetrahydroquinolin-2-ylmethyl)piperidine

[M+H]⁺=495.1

### EXAMPLE 72A63

4-(4-Butoxyphenyl)-1-(1-cyclohexanecarbonyl-1,2,3,4-tetrahydroquinolin-2-ylmethyl)piperidine

[M+H]⁺=489.2

### EXAMPLE 72A64

1-(1-Cyclohexanecarbonyl-1,2,3,4-tetrahydroquinolin-2-ylmethyl)-4-(2-fluorophenyl)piperazine

[M+H]⁺=436.2

### EXAMPLE 72A65

1-(1-Cyclohexanecarbonyl-1,2,3,4-tetrahydroquinolin-2-ylmethyl)-4-(6-chloropyridin-2-yl)piperazine

[M+H]⁺=453.1

### EXAMPLE 72A66

1-(1-Cyclohexanecarbonyl-1,2,3,4-tetrahydroquinolin-2-ylmethyl)-4-(3,5-dichloropyridin-4-yl)piperazine

[M+H]⁺=487.1

### EXAMPLE 72A67

1-(4-Aminophenyl)-4-(1-cyclohexanecarbonyl-1,2,3,4-tetrahydroquinolin-2-ylmethyl)piperazine

[M+H]⁺=433.2

### EXAMPLE 72A68

4-(3-Chlorophenyl)-1-(1-cyclohexanecarbonyl-1,2,3,4-tetrahydroquinolin-2-ylmethyl)piperidine

[M+H]⁺=451.2

### EXAMPLE 72A69

4-(2-Chlorophenyl)-1-(1-cyclohexanecarbonyl-1,2,3,4-tetrahydroquinolin-2-ylmethyl)piperidine

[M+H]⁺=451.2

### EXAMPLE 72A70

1-(1-Cyclohexanecarbonyl-1,2,3,4-tetrahydroquinolin-2-ylmethyl)-2-(pyridin-2-yl)-pyrrolidine

[M+H]⁺=404.1

### EXAMPLE 72A71

1-(1-Cyclohexanecarbonyl-1,2,3,4-tetrahydroquinolin-2-ylmethyl)-2-(2-thienyl)pyrrolidine

[M+H]⁺=409.1

### EXAMPLE 72A72

1-(1-Cyclohexanecarbonyl-1,2,3,4-tetrahydroquinolin-2-ylmethyl)-2-phenylpyrrolidine

[M+H]⁺=403.2

### EXAMPLE 72A73

1-(1-Cyclohexanecarbonyl-1,2,3,4-tetrahydroquinolin-2-ylmethyl)-2-(2,4-dimethoxy)phenylpyrrolidine

M+H]⁺=463.2

### EXAMPLE 72A74

1(1-Cyclohexanecarbonyl-1,2,3,4-tetrahydroquinolin-2-ylmethyl)-2-(pyridin-3-yl)-pyrrolidine

[M+H]⁺=404.1

### EXAMPLE 72A75

1-(1-Cyclohexanecarbonyl-1,2,3,4-tetrahydroquinolin-2-ylmethyl)-2-phenylmethylpyrrolidine

[M+H]⁺=417.2

### EXAMPLE 72A76

1-(1-Cyclohexanecarbonyl-1,2,3,4-tetrahydroquinolin-2-ylmethyl)-4-(3,5-dimethoxyphenyl)piperazine

[M+H]⁺=478.2

### EXAMPLE 72A77

1-(1-Cyclohexanecarbonyl-1,2,3,4-tetrahydroquinolin-2-ylmethyl)-2-(2-furyl)pyrrolidine

[M+H]⁺=393.1

### EXAMPLE 72A78

1-(1-Cyclohexanecarbonyl-1,2,3,4-tetrahydroquinolin-2-ylmethyl)-2-(4-methoxyphenylmethyl)pyrrolidine

[M+H]⁺=447.2

### EXAMPLE 72A79

1-(1-Cyclohexanecarbonyl-1,2,3,4-tetrahydroquinolin-2-ylmethyl)-2-(4-chlorophenyl)pyrrolidine

[M+H]⁺=437.2

### EXAMPLE 72A80

1-(1-Cyclohexanecarbonyl-1,2,3,4-tetrahydroquinolin-2-ylmethyl)-2-(2-indolyl)pyrrolidine

[M+H]⁺=442.2

### EXAMPLE 72A81

1-(1-Cyclohexanecarbonyl-1,2,3,4-tetrahydroquinolin-2-ylmethyl)-4-(2-trifluoromethylphenyl)piperidine

[M+H]⁺=485.2

### EXAMPLE 72A82

N-(1-Cyclohexanecarbonyl-1,2,3,4-tetrahydroquinolin-2-ylmethyl)-2-(2-methoxyphenoxy)ethylmethylamine

[M+H]⁺=437.1

### EXAMPLE 72A83

N-(1-Cyclohexanecarbonyl-1,2,3,4-tetrahydroquinolin-2-ylmethyl)-N-(2,3-dihydrobenzo[1,4]dioxin-2-ylmethyl)methylamine

[M+H]⁺=435.2

### EXAMPLE 72A84

N-(1-Cyclohexanecarbonyl-1,2,3,4-tetrahydroquinolin-2-ylmethyl)-2-(2-chlorophenoxy)ethylmethylamine

[M+H]⁺=441.1

### EXAMPLE 72A85

N-(1-Cyclohexanecarbonyl-1,2,3,4-tetrahydroquinolin-2-ylmethyl)-2-[2-(2,2,2-trifluoroethoxy)phenoxy]ethylmethylamine

[M+H]⁺=505.1

### EXAMPLE 72A86

N-(1-Cyclohexanecarbonyl-1,2,3,4-tetrahydroquinolin-2-ylmethyl)-2-[4-(1H)-indolyloxy]ethylmethylamine

[M+H]⁺=446.2

### EXAMPLE 72A87

N-(1-Cyclohexanecarbonyl-1,2,3,4-tetrahydroquinolin-2-ylmethyl)-2-(8-quinolyloxy)ethylmethylamine

[M+H]⁺=458.1

### EXAMPLE 72A88

1-(4-Chloro-2-nitrophenyl)-4-(1-cyclohexanecarbonyl-1,2,3,4-tetrahydroquinolin-2-ylmethyl)piperazine

[M+H]⁺=498.08

### EXAMPLE 72A89

1-(1-Cyclohexanecarbonyl-1,2,3,4-tetrahydroquinolin-2-ylmethyl)-4-(2-trifluoromethylquinolin-4-yl)piperazine

[M+H]⁺=537.21

### EXAMPLE 72A90

4-(1-Benzimidazolyl)-1-(1-cCyclohexanecarbonyl-1,2,3,4-tetrahydroquinolin-2-ylmethyl)piperidine

[M+H]⁺=457.16

### EXAMPLE 72A91

1-(1-Cyclohexanecarbonyl-1,2,3,4-tetrahydroquinolin-2-ylmethyl)-2-(4-methoxyphenyl)piperidine

[M+H]⁺=447.19

### EXAMPLE 72A92

1-(1-Cyclohexanecarbonyl-1,2,3,4-tetrahydroquinolin-2-ylmethyl)-4-(2,6-dichlorophenyl)piperidine

[M+H]⁺=486.09

### EXAMPLE 72A93

1-(1-Cyclohexanecarbonyl-1,2,3,4-tetrahydroquinolin-2-ylmethyl)-4-(2-pyridyl)piperazine

[M+H]⁺=419.3

### EXAMPLE 72A94

1-(1-Cyclohexanecarbonyl-1,2,3,4-tetrahydroquinolin-2-ylmethyl)-4-(4-quinolyl)piperazine

[M+H]⁺=469.35

### EXAMPLE 72A95

1-(1-Cyclohexanecarbonyl-1,2,3,4-tetrahydroquinolin-2-ylmethyl)-4-(7-methoxy-4-quinolyl)piperazine

[M+H]⁺=499.38

### EXAMPLE 73

The Compounds of this Example were prepared from Compound 53B instead of Compound 1B following the acylation/sulphonylation procedures described in Example 68A, 69, 70.

### EXAMPLE 73A

1-(4-Fluoro-2-methoxyphenyl)-4-[1-(2-methylphenylaminocarbonyl)-1,2,3,4-tetrahydroquinolin-6-methyl-2-ylmethyl]piperazine

[M+H]⁺=503.24

### EXAMPLE 73A1

1-(4-Fluoro-2-methoxyphenyl)-4-[1-(4-methylphenylaminocarbonyl)-1,2,3,4-tetrahydroquinolin-6-methyl-2-ylmethyl]piperazine

[M+H]⁺=503.24

### EXAMPLE 73A2

1-[1-(1,1-Dimethylethylaminocarbonyl)-1,2,3,4-tetrahydroquinolin-6-methyl-2-ylmethyl]-4-(4-fluoro-2-methoxyphenyl)piperazine

[M+H]⁺=469.2

### EXAMPLE 73A3

1-(4-Fluoro-2-methoxyphenyl)-4-(1-pentylaminocarbonyl-1,2,3,4-tetrahydroquinolin-6-methyl-2-ylmethyl)piperazine

[M+H]⁺=483.21

### EXAMPLE 73A4

1-(4-Fluoro-2-methoxyphenyl)-4-[1-(2-thienylacetyl)-1,2,3,4-tetrahydroquinolin-6-methyl-2-ylmethyl]piperazine

[M+H]⁺=494.12

### EXAMPLE 73A5

1-(4-Fluoro-2-methoxyphenyl)-4-[1-(3-thienylacetyl)-1,2,3,4-tetrahydroquinolin-6-methyl-2-ylmethyl]piperazine

[M+H]⁺=494.13

### EXAMPLE 73A6

1-(1-Cyclohexylacetyl-1,2,3,4-tetrahydroquinolin-6-methyl-2-ylmethyl)-4-(4-fluoro-2-methoxyphenyl)piperazine

[M+H]⁺=494.13

### EXAMPLE 73A7

1-[1-(4-Cyanobenzoyl)-1,2,3,4-tetrahydroquinolin-6-methyl-2-ylmethyl]-4-(4-fluoro-2-methoxyphenyl)piperazine

[M+H]⁺=499.17

### EXAMPLE 73A8

1-(4-Fluoro-2-methoxyphenyl)-4-[1-(4-methoxybenzoyl)-1,2,3,4-tetrahydroquinolin-6-methyl-2-ylmethyl)piperazine

[M+H]⁺=504.16

### EXAMPLE 73A9

1-[1-(4-Chlorobenzoyl)-1,2,3,4-tetrahydroquinolin-6-methyl-2-ylmethyl]-4-(4-fluoro-2-methoxyphenyl)piperazine

[M+H]⁺=508.13

### EXAMPLE 73A10

1-[1-(Benzo[1,3]dioxol-5-ylcarbonyl)-1,2,3,4-tetrahydroquinolin-6-methyl-2-ylmethyl]-4-(4-fluoro-2-methoxyphenyl)piperazine

[M+H]⁺=518.17

### EXAMPLE 73A11

1-[1-[2-(2-Chlorophenyl)acetyl]-1,2,3,4-tetrahydroquinolin-6-methyl-2-ylmethyl]-4-(4-fluoro-2-methoxyphenyl)piperazine

[M+H]⁺=522.15

### EXAMPLE 73A12

1-(4-Fluoro-2-methoxyphenyl)-4-[1-(1-methyl-1H-indol-3-ylcarbonyl)-1,2,3,4-tetrahydroquinolin-6-methyl-2-ylmethyl]piperazine

[M+H]⁺=527.20

### EXAMPLE 73A13

1-(1-Cycloheptanecarbonyl-1,2,3,4-tetrahydroquinolin-6-methyl-2-ylmethyl)-4-(4-fluoro-2-methoxyphenyl)piperazine

[M+H]⁺=494.25

### EXAMPLE 73A14

1-(4-Fluoro-2-methoxyphenyl)-4-(1-phenylsulphonyl-1,2,3,4-tetrahydroquinolin-6-methyl-2-ylmethyl)piperazine

[M+H]⁺=510.16

### EXAMPLE 73A15

1-(4-Fluoro-2-methoxyphenyl)-4-(1-phenylmethylsulphonyl-1,2,3,4-tetrahydroquinolin-6-methyl-2-ylmethyl)piperazine

[M+H]⁺=524.2

### EXAMPLE 73A16

1-(4-Fluoro-2-methoxyphenyl)-4-[1-(4-methoxyphenylsulphonyl)-1,2,3,4-tetrahydroquinolin-6-methyl-2-ylmethyl]piperazine

[M+H]⁺=540.19

### EXAMPLE 73A17

1-(4-Fluoro-2-methoxyphenyl)-4-[1-(4-methylphenylsulphonyl)-1,2,3,4-tetrahydroquinolin-6-methyl-2-ylmethyl]piperazine

[M+H]⁺=524.19

### EXAMPLE 73A18

1-[1-(4-Cyanophenylsulphonyl)-6-methyl-1,2,3,4-tetrahydroquinolin-2-ylmethyl]-4-(4-fluoro-2-methoxyphenyl)piperazine

[M+H]⁺=535.16

### EXAMPLE 73A19

1-[1-(4-Fluorophenylsulphonyl)-6-methyl-1,2,3,4-tetrahydroquinolin-2-ylmethyl]-4-(4-fluoro-2-methoxyphenyl)piperazine

[M+H]⁺=528.16

### EXAMPLE 74

The Compounds of this Example were prepared from Compound 74A-B instead of Compound 1B following the acylation/sulphonylation procedures described in Example 68A, 69, 70.

### EXAMPLE 74A

1-(4-Fluoro-2-methoxyphenyl)-4-[1-(3-methylbutanoyl)-1,2,3,4-tetrahydroquinolin-2-ylmethyl]piperazine

a) 2-F4-(4-Fluoro-2-methoxy)phenyl-1-piperazinylmethyl]quinoline (Compound 74A-A)

Compound 74A-A was obtained in the same way as described for Compound 1C (procedure d) but using 1-(4-fluoro-2-methoxy)phenylpiperazine instead of 1-(4-indolyl)piperazine. The crude was purified by flash chromatography eluting with ethyl acetate to give the title compound (82%).

¹H-NMR (δ): 2.71-3.39 (m; 8H), 3.85 (s; 3H), 3.93-4.29 (m; 1H), 6.51-6.72 (m; 2H); 6.80-6.97 (m; 1H), 7.48-7.62 (m; 1H), 7.65-7.96 (m; 3H), 8.10 (d; 1H), 8.21 (d; 1H).

b) 1-(4-Fluoro-2-methoxyphenyl)-4-(1,2,3,4-tetrahydroquinoline-2-ylmethyl)piperazine (Compound 74A-B)

The title compound was prepared as described for Compound 1B (procedure e) starting from Compound 74A-A instead of Compound 1C. The crude was purified by flash chromatography eluting with petroleum ether-ethyl acetate 7:3 to give the title compound (74%).

¹H-NMR (δ): 1.49-1.70 (m; 1H), 1.77-2.01 (m; 1H), 2.31-3.27 (m; 12H), 3.36-3.62 (m; 1H), 3.82 (s, 3H), 4.57-4.89 (br; 1H), 6.48-6.71 (m; 4H), 6.81-7.04 (m; 3H).

c) 1-(4-Fluoro-2-methoxyphenyl)-4-[1-(3-methylbutanoyl)-1,2,3,4-etrahydroquinolin-2-ylmethyl]piperazine

[M+H]⁺=440.28

### EXAMPLE 74A1

1-(3,3-Dimethylbutanoyl-1,2,3,4-tetrahydroquinolin-2-ylmethyl)-4-(4-fluoro-2-methoxyhenyl)piperazine

[M+H]⁺=454.33

### EXAMPLE 74A2

1-(1-Cyclopropylacetyl-1,2,3,4-tetrahydroquinolin-2-ylmethyl)-4-(4-fluoro-2-methoxyhenyl)piperazine

[M+H]⁺=438.27

### EXAMPLE 74A3

1-(4-Fluoro-2-methoxyphenyl)-4-[1-(2-pyrrolylcarbonyl)-1,2,3,4-tetrahydroquinolin-2-ylmethyl]piperazine

[M+H]⁺=449.24

### EXAMPLE 74A4

1-(4-Fluoro-2-methoxyphenyl)-4-[1-(2-furoyl)-1,2,3,4-tetrahydroquinolin-2-ylmethyl]piperazine

[M+H]⁺=450.24

### EXAMPLE 74A5

1-(4-Fluoro-2-methoxyphenyl)-4-[1-(4-methoxybenzoyl)-1,2,3,4-tetrahydroquinolin-2-ylmethy]piperazine

[M+H]⁺=490.28

### EXAMPLE 74A6

1-[1-(4-Cyanobenzoyl)-1,2,3,4-tetrahydroquinolin-2-ylmethy]-4-(4-fluoro-2-methoxyphenyl)piperazine

[M+H]⁺=485.26

### EXAMPLE 74A7

1-[1-(1-Acetylpiperidin-4-ylcarbonyl)-1,2,3,4-tetrahydroquinolin-2-ylmethy]-4-(4-fluoro-2-methoxyphenyl)piperazine

[M+H]⁺=509.35

### EXAMPLE 74A8

1-(4-Fluoro-2-methoxyphenyl)-4-[1-(2-thienylacetyl)-1,2,3,4-tetrahydroquinolin-2-ylmethyl]piperazine

[M+H]⁺=480.3

### EXAMPLE 74A9

1-(4-Fluoro-2-methoxyphenyl)-4-[1-(3-thienylacetyl)-1,2,3,4-tetrahydroquinolin-2-ylmethyl]piperazine

[M+H]⁺=480.3

### EXAMPLE 74A10

1-(1-Cyclobutylcarbonyl-1,2,3,4-tetrahydroquinolin-2-ylmethy)-4-(4-fluoro-2-methoxyphenyl)piperazine

[M+H]⁺=438.33

### EXAMPLE 74A11

1-(4-Fluoro-2-methoxyphenyl)-4-[1-(4-phenoxybutanoyl)-1,2,3,4-tetrahydroquinolin-2-ylmethyl]piperazine

[M+H]⁺=518.37

### EXAMPLE 74A12

1-(1-Benzenesulphonyl-1,2,3,4-tetrahydroquinolin-2-ylmethy)-4-(4-fluoro-2-methoxyphenyl)piperazine

[M+H]⁺=496.17

### EXAMPLE 74A13

1-(4-Fluoro-2-methoxyphenyl)-4-(1-phenylmethylsulphonyl-1,2,3,4-tetrahydroquinolin-2-ylmethyl]piperazine

[M+H]⁺=510.18

### EXAMPLE 74A14

1-(4-Fluoro-2-methoxyphenyl)-4-[1-(2-methylphenylaminocarbonyl)-1,2,3,4-tetrahydroquinolin-2-ylmethyl]piperazine

[M+H]⁺=489.23

### EXAMPLE 74A15

1-[1-(1,1-Dimethyethylaminocarbonyl)-1,2,3,4-tetrahydroquinolin-2-ylmethy]-4-(4-fluoro-2-methoxyphenyl)piperazine

[M+H]⁺=455.25

### EXAMPLE 75

The Compounds of this Example were prepared from Compound 21B instead of Compound 1B following the acylation/sulphonylation procedures described in Example 68A, 69, 70.

### EXAMPLE 75A

1-(1-Isoquinolinyl)-4-[1-(3-methylbutanoyl)-1,2,3,4-tetrahydroquinolin-2-ylmethyl]piperazine

[M+H]⁺=443.29

### EXAMPLE 75A1

1-[1-(3,3-Dimethylbutanoyl)-1,2,3,4-tetrahydroquinolin-2-ylmethyl]-4-(1-isoquinolinyl)piperazine

[M+H]⁺=457.37

### EXAMPLE 75A2

1-[1-(2-Cyclopropylacetyl)-1,2,3,4-tetrahydroquinolin-2-ylmethyl]-4-(1-isoquinolinyl)piperazine

[M+H]⁺=441.37

### EXAMPLE 75A3

1-(1-Isoquinolinyl)-4-[1-(2-pyrrolylcarbonyl)-1,2,3,4-tetrahydroquinolin-2-ylmethyl]piperazine

[M+H]⁺=452.35

### EXAMPLE 75A4

1-[1-(2-Furancarbonyl)-1,2,3,4-tetrahydroquinolin-2-ylmethyl]-4-(1-isoquinolinyl)piperazine

[M+H]⁺=453.34

### EXAMPLE 75A5

1-(1-Isoquinolinyl)-4-[1-(4-methoxybenzoyl)-1,2,3,4-tetrahydroquinolin-2-ylmethyl]piperazine

[M+H]⁺=493.39

### EXAMPLE 75A6

1-[1-(4-Cyanobenzoyl)-1,2,3,4-tetrahydroquinolin-2-ylmethyl]-4-(1-isoquinolinyl)piperazine

[M+H]⁺=488.37

### EXAMPLE 75A7

1-[1-(1-Acetyl-4-piperidinecarbonyl)-1,2,3,4-tetrahydroquinolin-2-ylmethyl]-4-(1-isoquinolinyl)piperazine

[M+H]⁺=512.43

### EXAMPLE 75A8

1-(1-Isoquinolinyl)-4-[1-(2-thienyacetyl)-1,2,3,4-tetrahydroquinolin-2-ylmethyl]piperazine

[M+H]⁺=483.34

### EXAMPLE 75A9

1-(1-Isoquinolinyl)-4-[1-(3-thienyacetyl)-1,2,3,4-tetrahydroquinolin-2-ylmethyl]piperazine

[M+H]⁺=483.34

### EXAMPLE 75A10

1-(1-Cyclobutanecarbonyl-1,2,3,4-tetrahydroquinolin-2-ylmethyl)-4-(1-isoquinolinyl)piperazine

[M+H]⁺=441.37

### EXAMPLE 75A11

1-(1-Isoquinolinyl)-4-[1-(4-phenoxybutanoyl)-1,2,3,4-tetrahydroquinolin-2-ylmethyl]piperazine

[M+H]⁺=521.41

### EXAMPLE 75A12

1-(1-Benzenesulphonyl-1,2,3,4-tetrahydroquinolin-2-ylmethyl)-4-(1-isoquinolinyl)piperazine

[M+H]⁺=499.21

### EXAMPLE 75A13

1-(1-Isoquinolinyl)-4-(1-phenylmethylsulphonyl-1,2,3,4-tetrahydroquinolin-2-ylmethyl)piperazine

[M+H]⁺=513.22

### EXAMPLE 75A14

1-(1-Isoquinolinyl)-4-[1-(2-methylphenylaminocarbonyl)-1,2,3,4-tetrahydroquinolin-2-ylmethyl)piperazine

[M+H]⁺=492.27

### EXAMPLE 75A15

1-(1-Isoquinolinyl)-4-[1-(1,1-Dimethyethylaminocarbonyl)-1,2,3,4-tetrahydroquinolin-2-ylmethyl]piperazine

[M+H]⁺=458.29

### EXAMPLE 76

The Compounds of this Example were prepared from Compound 76A-B instead of Compound 1B following the acylation/sulphonylation procedures described in Example 68A, 69, 70.

### EXAMPLE 76A

1-(2,3-Dihydrobenzo[1,4]dioxin-5-yl)-4-[1-(3-methylbutanoyl)-1,2,3,4-tetrahydroquinolin-2-ylmethyl]piperazine

a) 1-(2,3-Dihydrobenzo[1,4]dioxin-5-yl)-4-(2-quinolinylmethyl)piperazine (Compound 76A-A)

Compound 74A-A was obtained in the same way as described for Compound 1C (procedure d) but using 1-(2,3-dihydrobenzo[1,4]dioxin-5-yl)piperazine instead of 1-(4-indolyl)piperazine. The crude was purified by flash chromatography eluting with CHCl₃-2 N methanolic ammonia 100:1 to give the title compound (82%).

¹H-NMR (δ): 2.77-3.02 (m, 4H); 3.11-3.34 (m, 4H); 3.93-4.12 (m, 2H), 4.18-4.37 (m, 4H); 6.48-6.68 (m, 2H); 6.79 (t, 1H); 7.47-7.62 (m, 1H); 7.64-7.87 (m, 3H); 8.07-8.28 (m, 2H).

b) 1-(2,3-Dihydrobenzo[1,4]dioxin-5-yl)-4-(1,2,3,4-tetrahydroquinoline-2-ylmethyl)piperazine (Compound 76A-B)

The title compound was prepared as described for Compound 1B (procedure e) starting from Compound 76A-A instead of Compound 1C. The crude was purified by flash chromatography eluting with petroleum ether-ethyl acetate 7:3 to give the title compound (41.2%).

¹H-NMR (δ):1.41-1.74 (m, 1H); 1.82-2.01 (m, 1H); 2.33-3.91 (m, 13H); 4.18-4.38 (m) 4H); 4.59-4.99 (br, 1H); 6.47-6.68 (m, 4H11, 6.81 (t, 1H); 6.87-7.07 (m, 2H).

c) 1-(2,3-Dihydrobenzo[1,4]dioxin-5-y)-4-[1-(3-methylbutanoyl)-1,2,3,4-tetrahydroquinolin-2-ylmethyl]piperazine

[M+H]⁺=450.29

### EXAMPLE 76A1

4-[1-(2-Cyclopropylacetyl)-1,2,3,4-tetrahydroquinolin-2-ylmethyl]-1-(2,3-dihydrobenzo[1,4]dioxin-5-yl)piperazine

[M+H]⁺=448.25

### EXAMPLE 76A2

1-(2,3-Dihydrobenzo[1,4]dioxin-5-yl)-4-[1-(2-pyrrolylcarbonyl)-1,2,3,4-tetrahydroquinolin-2-ylmethyl]piperazine

[M+H]⁺=459.22

### EXAMPLE 76A3

1-(2,3-Dihydrobenzo[1,4]dioxin-5-yl)-4-[1-(2-furanecarbonyl)-1,2,3,4-tetrahydroquinolin-2-ylmethyl]piperazine

[M+H]⁺=460.21

### EXAMPLE 76A4

1-(2,3-Dihydrobenzo[1,4]dioxin-5-yl)-4-[1-(4-methoxybenzoyl)-1,2,3,4-tetrahydroquinolin-2-ylmethyl]piperazine

[M+H]⁺=500.26

### EXAMPLE 76A5

1-[1-(4-Cyanobenzoyl)-1,2,3,4-tetrahydroquinolin-2-ylmethyl]-4-(2,3-dihydrobenzo[1,4]dioxin-5-yl)piperazine

[M+H]⁺=495.24

### EXAMPLE 76A6

1-(2,3-Dihydrobenzo[1,4]dioxin-5-yl)-4-[1-[2-(2-thienyl)acetyl]-1,2,3,4-tetrahydroquinolin-2-ylmethyl]piperazine

[M+H]⁺=490.22

### EXAMPLE 76A7

1-(2,3-Dihydrobenzo[1,4]dioxin-5-yl)-4-[1-[2-(3-thienyl)acetyl]-1,2,3,4-tetrahydroquinolin-2-ylmethyl]piperazine

[M+H]⁺=490.22

### EXAMPLE 76A8

1-(1-Cyclobutanecarbonyl)-1,2,3,4-tetrahydroquinolin-2-ylmethyl]-4-(2,3-dihydrobenzo[1,4]dioxin-5-yl)piperazine

[M+H]⁺=448.25

### EXAMPLE 76A9

1-(2,3-Dihydrobenzo[1,4]dioxin-5-yl)-4-[1-(4-phenoxybutanoyl-1,2,3,4-tetrahydroquinolin-2-ylmethyl]piperazine

[M+H]⁺=528.29

### EXAMPLE 76A10

1-(1-Benzenesulphonyl-1,2,3,4-tetrahydroquinolin-2-ylmethyl)-4-(2,3-dihydrobenzo[1,4]dioxin-5-yl)piperazine

[M+H]⁺=506.21

### EXAMPLE 76A11

1-(2,3-Dihydrobenzo[1,4]dioxin-5-yl)-4-(1-phenylmethanesulphonyl-1,2,3,4-tetrahydroquinolin-2-ylmethyl)piperazine

[M+H]⁺=520.22

### EXAMPLE 76A12

1-(2,3-Dihydrobenzo[1,4]dioxin-5-yl)-4-[1-(2-methylphenylaminocarbonyl)-1,2,3,4-tetrahydroquinolin-2-ylmethyl]piperazine

[M+H]⁺=499.25

### EXAMPLE 76A13

1-(2,3-Dihydrobenzo[1,4]dioxin-5-yl)-4-[1-(1,1-dimethylethylaminocarbonyl)-1,2,3,4-tetrahydroquinolin-2-ylmethyl]piperazine

[M+H]⁺=465.26

### EXAMPLE 76A14

1-(2,3-Dihydrobenzo[1,4]dioxin-5-yl)-4-[1-(3,3-dimethylbutanoyl)-1,2,3,4-tetrahydroquinolin-2-ylmethyl]piperazine

[M+H]⁺=464.31

### EXAMPLE 76A15

1-[1-(1-acetyl-4-piperidinecarbonyl)-1,2,3,4-tetrahydroquinolin-2-ylmethyl]-4-(2,3-dihydrobenzo[1,4]dioxin-5-yl)piperazine

[M+H]⁺=519.31

### EXAMPLE 76A16

1-(2,3-Dihydrobenzo[1,4]dioxin-5-yl)-4-[1-(2,2-dimethylbutanoyl)-1,2,3,4-tetrahydroquinolin-2-ylmethyl]piperazine

[M+H]⁺=464.42

### EXAMPLE 76A17

1-(2,3-Dihydrobenzo[1,4]dioxin-5-yl)-4-[1-(1-trifluoromethylcyclopropanecarbonyl)-1,2,3,4-tetrahydroquinolin-2-ylmethyl]piperazine

[M+H]⁺=502.27

### EXAMPLE 76A18

1-[1-(1-Bicyclo[2.2.2]oct-2-ylcarbonyl)-1,2,3,4-tetrahydroquinolin-2-ylmethyl]-4-(2,3-dihydrobenzo[1,4]dioxin-5-yl)piperazine

[M+H]⁺=502.40

### EXAMPLE 76A19

1-[1-(1-Cyclopentanecarbonyl)-1,2,3,4-tetrahydroquinolin-2-ylmethyl]-4-(2,3-dihydrobenzo[1,4]dioxin-5-yl)piperazine

[M+H]⁺=462.33

### EXAMPLE 76A20

1-(2,3-Dihydrobenzo[1,4]dioxin-5-yl)-4-[1-(4-fluorobenzoyl)-1,2,3,4-tetrahydroquinolin-2-ylmethyl]piperazine

[M+H]⁺=488.30

### EXAMPLE 76A21

1-[1-(Benzo[1,3]dioxol-5-ylcarbonyl)-1,2,3,4-tetrahydroquinolin-2-ylmethyl]-4-(2,3-dihydrobenzo[1,4]dioxin-5-yl)piperazine

[M+H]⁺=514.29

### EXAMPLE 76A22

1-(2,3-Dihydrobenzo[1,4]dioxin-5-yl)-4-[1-(3-phenylpropenoyl)-1,2,3,4-tetrahydroquinolin-2-ylmethyl]piperazine

[M+H]⁺=496.31

### EXAMPLE 76A23

1-(2,3-Dihydrobenzo[1,4]dioxin-5-yl)-4-[1-[3-(4-fluorophenyl)propenoyl]-1,2,3,4-tetrahydroquinolin-2-ylmethyl]piperazine

[M+H]⁺=514.34

### EXAMPLE 76A24

1-(2,3-Dihydrobenzo[1,4]dioxin-5-yl)-4-[1-(3-methyl-2-thiophenecarbonyl)-1,2,3,4-tetrahydroquinolin-2-ylmethyl]piperazine

[M+H]⁺=490.28

### EXAMPLE 76A25

1-(2,3-Dihydrobenzo[1,4]dioxin-5-yl)-4-[1-(3-furanecarbonyl)-1,2,3,4-tetrahydroquinolin-2-ylmethyl]piperazine

[M+H]⁺=460.28

### EXAMPLE 76A26

1-(2,3-Dihydrobenzo[1,4]dioxin-5-yl)-4-[1-(2-methyl-3-furanecarbonyl)-1,2,3,4-tetrahydroquinolin-2-ylmethyl]piperazine

[M+H]⁺=474.29

### EXAMPLE 76A27

1-(2,3-Dihydrobenzo[1,4]dioxin-5-yl)-4-[1-[5-methyl-4-(1,2,4-triazol-1-ylmethyl)-1-furanecarbonyl)-1,2,3,4-tetrahydroquinolin-2-ylmethyl]piperazine

[M+H]⁺=555.31

### EXAMPLE 76A28

1-(2,3-Dihydrobenzo[1,4]dioxin-5-yl)-4-[1-(5-methyl-4-isoxazolecarbonyl)-1,2,3,4-tetrahydroquinolin-2-ylmethyl]piperazine

[M+H]⁺=475.28

### EXAMPLE 76A29

1-[1-(4-Acetyl-2-pyrrolecarbonyl)-1,2,3,4-tetrahydroquinolin-2-ylmethyl]-4-(2,3-dihydrobenzo[1,4]dioxin-5-yl)piperazine

[M+H]⁺=501.32

### EXAMPLE 77

The Compounds of this Example were prepared from Compound 77A-B instead of Compound 1B following the acylation/sulphonylation procedures described in Example 68A, 69, 70.

### EXAMPLE 77A

N-[2-(2-Methoxyphenoxy)ethyl]-N-[1-(3-methylbutanoyl)-1,2,3,4-tetrahydroquinolin-2-ylmethyl]methylamine

a) N-[2-(2-Methoxyphenoxy)ethyl]-N-(2-quinolylmethyl)methylamine (Compound 77A-A)

Compound 77A-A was obtained in the same way as described for Compound 1C (procedure d) but using N-[2-(2-methoxyphenoxy)ethyl]methylamine instead of 1-(4-indolyl)piperazine. The crude was purified by flash chromatography eluting with ethyl acetate—MeOH 100:4 to give the title compound (77%).

¹H-NMR (δ): 2.50 (s, 3H), 3.00-3.22 (m, 2H), 3.85 (s; 3H), 4.00-4.20 (m, 2H), 4.21-4.40 (m, 2H), 6.80-7.00 (m; 4H); 7.55 (dd, 1H), 7.65-7.85 (m, 3H), 8.00-8.20 (m, 2H).

b) N-[2-(2-Methoxyphenoxy)ethyl]-N-(1,2,3,4-tetrahydroquinoline-2-ylmethyl)methylamine (Compound 77A-B)

The title compound was prepared as described for Compound 1B (procedure e) starting from Compound 77A-A instead of Compound 1C. The crude was purified by flash chromatography eluting with CH₂Cl₂-2 N methanolic ammonia 98.5:1.5 to give the title compound (67%).

¹H-NMR (δ): 1.45-1.75 (m, 1H), 1.80-2.00 (m, 1H), 2.30-3.15 (m, 9H), 3.30-3.60 (m, 1H), 3.85 (s; 3H), 4.10-4.25 (m, 2H), 4.50-5.10 (b, 1H), 6.45.-6.60 (m, 2H), 6.80-7.00 (m; 6H);

### EXAMPLE 77A

N-[2-(2-Methoxyphenoxy)ethyl]-N-[1-(3-methylbutanoyl)-1,2,3,4-tetrahydroquinolin-2-ylmethyl]methyl amine

[M+H]⁺=411.30

### EXAMPLE 77A1

N-[1-(3,3-Dimethylbutanoyl)-1,2,3,4-tetrahydroquinolin-2-ylmethyl]-N-[2-(2-methoxyphenoxy)ethyl]methylamine

[M+H]⁺=425.31

### EXAMPLE 77A2

N-[1-(2-Cyclopropylacetyl)-1,2,3,4-tetrahydroquinolin-2-ylmethyl]-N-[2-(2-methoxyphenoxy)ethyl]methylamine

[M+H]⁺=409.29

### EXAMPLE 77A3

N-[2-(2-Methoxyphenoxy)ethyl]-N-[1-(2-pyrrolylcarbonyl)-1,2,3,4-tetrahydroquinolin-2-ylmethyl]methylamine

[M+H]⁺=420.22

### EXAMPLE 77A4

N-[1-(2-Furoyl)-1,2,3,4-tetrahydroquinolin-2-ylmethyl]-N-[2-(2-methoxyphenoxy)ethyl]methylamine

[M+H]⁺=421.24

### EXAMPLE 77A5

N-[1-(4-Methoxybenzoyl)-1,2,3,4-tetrahydroquinolin-2-ylmethyl]-N-[2-(2-methoxyphenoxy)ethyl]methylamine

[M+H]⁺=461.27

### EXAMPLE 77A6

N-[1-(1-Acetyl-4-piperidinecarbonyl)-1,2,3,4-tetrahydroquinolin-2-ylmethyl]-N-[2-(2-methoxyphenoxy)ethyl]methylamine

[M+H]⁺=480.28

### EXAMPLE 77A7

N-[2-(2-Methoxyphenoxy)ethyl]-N-[1-[2-(2-thienyl)acetyl]-1,2,3,4-tetrahydroquinolin-2-ylmethyl]methylamine

[M+H]⁺=451.22

### EXAMPLE 77A8

N-[2-(2-Methoxyphenoxy)ethyl]-N-[1-[2-(3-thienyl)acetyl]-1,2,3,4-tetrahydroquinolin-2-ylmethyl]methylamine

[M+H]⁺=451.22

### EXAMPLE 77A9

N-[(1-Cyclobutanecarbonyl)-1,2,3,4-tetrahydroquinolin-2-ylmethyl]-N-[2-(2-methoxyphenoxy)ethyl]methylanine

[M+H]⁺=409.29

### EXAMPLE 77A10

N-[2-(2-Methoxyphenoxy)ethyl]-N-[1-(4-phenoxybutanoyl-1,2,3,4-tetrahydroquinolin-2-ylmethyl]methylamine

[M+H]⁺=489.31

### EXAMPLE 77A11

N-(1-Benzenesulphonyl-1,2,3,4-tetrahydroquinolin-2-ylmethyl)-N-[2-(2-methoxyphenoxy)ethyl]methylamine

[M+H]⁺=467.28

### EXAMPLE 77A12

N-[2-(2-Methoxyphenoxy)ethyl]-N-(1-phenylmethanesulphonyl-1,2,3,4-tetrahydroquinolin-2-ylmethyl)methylamine

[M+H]⁺=481.25

### EXAMPLE 77A13

N-[2-(2-Methoxyphenoxy)ethyl]-N-[1-(2-methylphenylaminocarbonyl)-1,2,3,4-tetrahydroquinolin-2-ylmethyl]methylamine

[M+H]⁺=460.28

### EXAMPLE 77A14

N-[1-(1,1-Dimethylethyl)-1,2,3,4-tetrahydroquinolin-2-ylmethyl]-N-[2-(2-methoxyphenoxy)ethyl]methylamine

[M+H]⁺=426.30

### EXAMPLE 78

1-(1-Cyclohexanecarbonyl-5-fluoro-1,2,3,4-tetrahydroquinoline-2-ylmethyl)-4-(4-fluoro-2-methoxyphenyl)piperazine.

a) 1-Benzoyl-2-cyano-5-fluoro-1,2-dihydroquinoline (Compound 78A)

The title compound was prepared as described for compound 32A, but starting from 5-fluoroquinoline (WO 0144247) instead of 6-methylquinoline. (46%)

¹H-NMR (δ): 6.13-6.22 (m; 1H), 6.41 (d; 1H), 6.79-7.02 (m; 2H), 7.18-7.37 (m; 7H).

b) 5-Fluoroquinoline-2-carboxylic acid (Compound 78B)

Compound 78B was obtained in the same manner as described for compound 32B, but using compound 78A instead of compound 32A. The crude was used in the next step without additional purification. (54%).

¹H-NMR (DMSO-d₆, δ): 6.15-7.17 (br; 1H), 7.29-7.48 (m; 1H), 7.61-7.80 (m; 1H), 7.90 (d; 1H), 8.04 (d; 1H), 8.87 (d; 1H).

c) 5-Fluoro-1,2,3,4-tetrahydroquinoline-2-carboxylic acid (Compound 78C)

The title compound was prepared as described for Compound 1B (step e) starting from Compound 78B instead of Compound 1D. The crude was treated with 37% HCl and taken up with MeCN (5 ml) to afford, after cooling to 0° C. and filtration, Compound 78C (62%).

¹H-NMR (DMSO-d₆ δ): 1.87-2.08 (m; 2H), 2.34-2.52 (m; 1H), 2.61-2.78 (m; 1H), 3.86-4.06 (m; 1H), 6.30 (t; 1H), 6.39 (d; 1H), 6.77-6.97 (m; 1H), 6.99-7.62 (br, 1H)

d) 1-(4-Fluoro-2-methoxyphenyl)-4-(5-fluoro-1,2,3,4-tetrahydroquinoline-2-ylcarbonyl)piperazine (Compound 78D)

The title compound was synthesised following the procedure described for Compound 1D (step g) but using Compound 78C instead of 1,2,3,4-tetrahydroquinoline-2-carboxylic acid. The residue was purified by flash chromatography eluting with petroleum ether—EtOAc 6:4 to give the title compound (48%).

¹H-NMR (δ): 1.59-1.70 (m, 1H), 2.12-2.31 (m, 1H), 2.54-2.85 (m, 1H), 2.89-3.17 (m, 5H), 3.61-3.98 (m, 7H), 4.11-4.79 (m, 1H), 4.61-4.74 (br, 1H), 6.31-6.50 (m, 2H), 6.55-6.73 (m, 2H), 6.79-7.05 (m, 2H).

e) 1-(4-Fluoro-2-methoyphenyl)-4-(5-fluoro-1,2,3,4-tetrahydroquinoline-2-ylmethyl)piperazine (Compound 78E)

The title compound was synthesised following the procedure described for Compound 1B (step h), but using Compound 78D instead of Compound 1D. The residue was purified by flash chromatography eluting with petroleum ether—EtOAc 55:45 to give the title compound (73%).

¹H-NMR (δ): 1.40-1.67 (m, 1H), 1.81-2.04 (m, 1H), 2.39-2.94 (m, 8H), 3.32-3.52 (m, 1H), 3.84 (s, 3H), 4.70-4.91 (br, 1H), 6.24-6.41 (m, 2H), 6.54-6.68 (m, 2H), 6.70-6.99 (m, 2H).

f) 1-(1-Cyclohexanecarbonyl-5-fluoro-1,2,3,4-tetrahydroquinoline-2-ylmethyl)-4-(4-fluoro-2-methoxyphenyl)piperazine

The title compound was synthesised following the alternative acylation procedure described for the compound of Example 1 (step f) but starting from Compound 78E instead of Compound 1B. The crude was purified by flash chromatography eluting with petroleum ether—ethyl acetate 7:3 to give the title compound (88%).

¹H-NMR (δ): 0.77-2.02 (m, 11H), 2.08-2.48 (m, 4H), 2.51-3.06 (m, 10H), 3.81 (s, 3H), 4.96-5.17 (m, 1H), 6.51-6.67 (m, 2H), 6.74-7.03 (m, 2H), 7.09-7.19 (m, 2H).

### EXAMPLE 79

1-(1-Cyclohexanecarbonyl-1,2,3,4-tetrahydroquinoline-2-ylmethyl)-4-(2-triluoromethanesulphonyloxyphenyl)piperazine.

To a solution of compound 35A10 (0.2 g) in 1,2-DCE (10 mL) was added N-phenyltriflimide (0.75 g) and the solution was refluxed for 18 h, cooled to r.t. and washed with H₂O. The organic phase was dried (Na₂SO₄), filtered and evaporated at reduced pressure. The crude was purified by flash chromatography eluting with petroleum ether—ethyl acetate 7:3 affording 0.14 g (54%) of the title compound

¹H-NMR (δ): 0.78-2.20 (m, 13H), 2.21-3.80 (m, 8H), 2.80-3.08 (m, 4H), 5.00-5.20 (m, 1H), 7.00-7.52 (m, 8H)

### EXAMPLE 80

1-(1-Cyclohexanecarbonyl-1,2,3,4-tetrahydroquinoline-2-ylmethyl)-4-(1-isopropyl-4-indolyl)piperazine

The title compound was synthesised using the methodology described in Example 35 using Compound 35A-B and 1-(1-isopropyl-4-indolyl)piperazine instead of 1-(4-fluoro-2-methoxyphenyl)piperazine. Eluent: petroleum ether—EtOAc 7:3. Yield: 66%.

¹H-NMR (δ): 0.82-1.87 (m, 16H), 1.88-2.04 (m, 1H), 2.05-2.23 (m, 1H), 2.31-2.76 (m, 9H), 3.11-3.30 (m, 4H), 4.51-4.70 (m, 1H), 5.02-5.23 (m, 1H), 6.41-6.60 (m., 1H), 6.95-7.22 (m, 7H).

The starting piperazine was prepared as following:

a) 1-(1-Isopropyl-4-indolyl)piperazine (Compound 80A)

The starting piperazine was prepared as described in Example 35A25-A and B using isopropylbromide instead of acetyl chloride in step a. Yield: 75% overall.

¹H-NMR (δ): 1.51 (d, 6H), 2.78-3.42 (m, 9H), 4.52-4.76 (m, 1H), 6.48 (dd, 1H), 6.61 (dd, 1H), 7.02-7.21 (m, 3H).

### EXAMPLE 81

1-(6-tert-Butoxycarbonylamino-1-cyclohexanecarbonyl-1,2,3,4-tetrahydroquinoline-2-ylmethyl)-4-(4-fluoro-2-methoxyphenyl)piperazine

a) 6-tert-Butoxycarbonylaminoquinaldine (Compound 81A)

The title compound was synthesised using the methodology described for Compound 50B but using 6-aminoquinaldine instead of Compound 50A.

The crude was purified by flash chromatography eluting with CH₂Cl₂— MeOH 9:1 to give the title compound (85%).

¹H-NMR (δ): 1.53 (s, 9H), 2.74 (s, 3H), 6.63-6.75 (br, 1H), 7.23 (dd, 1H), 7.42 (dd, 1H), 7.84-8.09 (m, 3H).

b) 6-tert-Butoxycarbonylaminoquinoline-2-carbaldehyde (Compound 81B)

To a suspension of SeO₂ (0.33 g) in dioxane (10 ml) a solution of Compound 81A (0.5 g) in 10 ml of the same solvent was added dropwise and the resulting mixture refluxed for 4 h. Afterwards, the precipitated solid was filtered off, the solvent evaporated under vacuum and the crude used for the next step without further purification. (95%).

¹H-NMR (DMSO-d₆, δ): 1.50 (s, 9H), 7.78 (dd, 1H), 7.89 (dd, 1H), 8.11 (dd, 1H), 8.32 (dd, 1H), 8.42 (dd, 1H), 9.90 (s, 1H), 10.03 (s, 1H).

c) 1-(6-tert-Butoxycarbonylaminoquinoline-2-ylmethyl)-4-(4-fluoro-2-methoxyphenyl)piperazine (Compound 81C)

The title compound was synthesised using the same methodology described in Example 35A using Compound 82B instead of Compound 35A-B and 1-(4-fluoro-2-methoxyphenyl)piperazine. The crude was purified by flash chromatography eluting with CH₂Cl₂— MeOH 95:5 to give the title compound (57%).

¹H-NMR (δ): 1.50 (s, 9H), 2.82-3.31 (m, 8H), 3.75 (s, 3H), 3.78-4.21 (m, 2H), 6.50-6.62 (m, 2H), 6.63-6.78 (m, 1H), 6.80-6.92 (m, 1H), 7.51 (dd, 1H), 7.70-7.89 (m, 1H), 7.91-8.21 (m, 3H).

d) 1-(6-tert-Butoxycarbonylamino-1,2,3,4-tetrahydroquinoline-2-ylmethyl)-4-(4-fluoro-2-methoxyphenyl)piperazine (Compound 81D)

The title compound was prepared as described for Compound 1B (procedure e) starting from Compound 81C instead of Compound 1C. The crude was purified by flash chromatography eluting with petroleum ether—EtOAc 1:1 to give the title compound (65%).

¹H-NMR (δ): 1.40-1.68 (m, 10H), 1.81-1.99 (m, 1H), 2.42-2.61 (m, 3H), 2.62-2.87 (m, 4H), 2.88-3.15 (m, 4H), 3.31-3.51 (m, 1H), 3.83 (s, 3H), 6.08-6.20 (m, 1H), 6.42 (dd, 1H), 6.47-6.55 (m, 2H), 6.81-6.95 (m, 2H), 7.02-7.08 (br, 1H).

f) 1-(6-tert-Butoxycarbonylamino-1-cyclohexanecarbonyl-1,2,3,4-tetrahydroquinoline-2-ylmethyl)-4-(4-fluoro-2-methoxyphenyl)piperazine

The title compound was synthesised following the alternative acylation procedure described for the compound of Example 1 (step f) but starting from Compound 81D instead of Compound 1B. The crude was purified by flash chromatography eluting with petroleum ether—EtOAc 6:4 to give the title compound (80%).

¹H-NMR (δ): 0.78-1.95 (m, 20H), 1.96-2.19 (m, 1H), 2.20-2.75 (m, 9H), 2.76-3.08 (m, 4H), 3.82 (s, 3H), 5.02-5.22 (m, 1H), 6.40-6.51 (m, 1H), 6.52-6.68 (m, 2H), 6.75-6.85 (m, 1H), 6.97-7.19 (m, 2H), 7.30-7.41 (m, 1H).

### EXAMPLE 82

1-(1-Cyclohexanecarbonyl-1,2,3,4-tetrahydroquinoline-2-ylmethyl)-4-(1-methoxymethyl-4-indolyl)piperazine

The title compound was synthesised using the methodology described in Example 35 using Intermediate 35A-B and 1-(1-methoxymethyl-4-indolyl)piperazine instead of 1-(4-fluoro-2-methoxyphenyl)piperazine.

Eluent: petroleum ether-EtOAc 1:1. Yield: 63%.

¹H-NMR (δ): 0.81-1.97 (m, 11H), 1.98-2.14 (m, 1H), 2.25-2.78 (m, 9H), 3.10-3.28 (m, 7H), 5.03-5.27 (m, 1H), 5.43 (s, 2H), 6.41-6.73 (m., 2H), 7.03-7.29 (m, 7H).

The starting piperazine was prepared as following:

a) 1-(1-Methoxymethyl-4-indolyl)piperazine (Compound 82A)

The starting piperazine was prepared as described in Example 35A25 A and B but using methoxymethyl chloride instead of acetyl chloride in step A.

¹H-NMR (δ): 2.02-2.11 (br, 1H), 3.07-3.17 (m, 4H), 3.18-3.37 (m, 7H), 5.42 (s, 2H), 6.45 (dd, 1H), 6.65 (dd, 1H), 7.02-7.23 (m, 3H).

### EXAMPLE 83

1-(1-Cyclohexanecarbonyl-2-methyl-1,2,3,4-tetrahydroquinoline-2-ylmethyl)-4-(4-fluoro-2-methoxyphenyl)piperazine

a) 2-Bromomethyl-2-methyl-1,2,3,4-tetrahydroquinoline (Compound 83A)

A mixture of 0.24 g of 2-hydroxymethyl-2-methyl-1,2,3,4-tetrahydroquinoline (Bioorg. Med. Chem. Lett. 5, 1527-1532, (1995)), 0.55 g of CBr₄, 0.44 g of Ph₃P and 8 ml of CH₂Cl₂ was stirred at r.t. for 2 h. After evaporation to dryness, the residue was purified by flash chromatography (petroleum ether—ethyl acetate 95:5) to give the title compound (0.225 g, 69%).

¹H-NMR (δ):1.35 (s, 3H), 1.70-1.90 (m, 1H), 1.90-2.10 (m, 1H), 2.70-2.85 (m, 2H), 3.45 (d, 2H), 4.00-5.85 (b, 1H), 6.55-6.80 (m, 2H), 6.95-7.10 (m, 2H)

b) 1-(2-Methyl-1,2,3,4-tetrahydroquinoline-2-ylmethyl)-4-(4-fluoro-2-methoxyphenyl)piperazine (Compound 83B)

The title compound was synthesized following the procedure described for compound 34D but using as a starting material compound 83A instead of compound 34C.

The residue was purified by flash chromatography (petroleum ether—EtOAc 6:4) to afford the title compound (74%).

¹H-NMR (δ): 1.20 (s, 3H), 1.40-1.90 (m, 2H), 2.30-3.50 (m, 11H), 3.50-3.90 (m, 4H), 4.15 (b, 0.6H), 6.25 (b, 0.4H), 6.40-7.10 (m, 7H)

c) 1-(1-Cyclohexanecarbonyl-2-methyl-1,2,3,4-tetrahydroquinoline-2-ylmethyl)-4-(4-fluoro-2-methoxyphenyl)piperazine

The title compound was synthesised following the alternative acylation procedure described for the compound of Example 1 (step f) but starting from Compound 83B instead of Compound 1B. The residue was purified by flash chromatography eluting with toluene—EtOAc 7:3 then with CH₂Cl₂ affording the title compond (12%)

¹H-NMR (δ): 0.80-3.00 (m, 28H), 3.80 (s, 3H), 6.50-6.65 (m, 2H), 6.65-6.85 (m, 2H), 7.00-7.20 (m, 3H)

### EXAMPLE 84

1-(1-Cyclopentylcarbonyl-6-methyl-1,2,3,4-tetrahydroquinoline-2-ylmethyl]-4-(4-fluoro-2-methoxyphenyl)piperazine

The title compound was synthesised following the alternative acylation procedure described for the compound of Example 1 (step f) but starting from Compound 53B instead of Compound 1B and using cyclopentanecarbonyl chloride instead of cyclohexanecarbonyl chloride. The crude was purified by flash chromatography eluting with petroleum ether—ethyl acetate 6:4 to afford the title compound (78%).

¹H-NMR (δ):1.31-2.11 (m, 10H); 2.15-3.48 (m, 16H), 3.82 (s, 3H), 5.03-5.26 (m, 1H), 6.50-6.68 (m, 2H), 6.77-6.98 (m, 1H), 6.99-7.21 (m, 3H).

### EXAMPLE 85

1-(6-Amino-1-cyclohexanecarbonyl-1,2,3,4-tetrahydroquinoline-2-ylmethyl)-4-(4-fluoro-2-methoxyphenyl)piperazine

To a cooled solution of the compound of Example 81 (0.24 g) in MeOH (10 ml) 1.2 mL of 1.8 N HCl in diethyl ether was added and the resulting mixture heated at reflux for 5 h. Then the solvent was removed under vacuum, the residue alkalinised with aq. NaHCO₃ and extracted with CH₂Cl₂ (2×20 ml). The combined organic layers were dried over Na₂SO₄ and evaporated to dryness. The crude was purified by flash chromatography eluting with CH₂Cl₂₋ 2 N methanolic ammonia 95:5 to give the title compound (72%).

¹H-NMR (δ): 0.82-3.35 (m, 26H), 3.52-3.85 (m, 1H), 3.83 (s, 3H), 5.07-5.29 (m, 1H), 6.45-6.68 (m, 3H), 6.81-7.25 (m, 3H).

### EXAMPLE 86

### EXAMPLE 86A

1-(1-Cyclohexanecarbonyl-1,2,3,4-tetrahydroquinoline-2-ylmethyl)-4-(2-methoxy-4-methylphenyl)piperazine

The title compound and the following were prepared using the method reported for Example 35A.

¹H-NMR (δ): 0.80-3.60 (m, 28H), 3.80 (s, 3H), 5.10 (b, 1H), 6.60-6.85 (m, 3H), 7.10-7.25 (m, 4H)

### EXAMPLE 86A1

1-(Benzo[1,3]dioxol-5-ylmethyl)-4-(1-cyclohexanecarbonyl-1,2,3,4-tetrahydroquinoline-2-ylmethyl)piperazine

¹H-NMR (δ): 0.80-2.80 (m, 25H), 3.45 (s, 2H), 5.05 (b, 1H), 5.90 (s, 2H), 6.70-6.90 (m, 3H), 6.95-7.25 (m, 4H)

### EXAMPLE 86A2

N-(1-Cyclohexanecarbonyl-1,2,3,4-tetrahydroquinoline-2-ylmethyl)-N-[2-(2-methoxyphenoxy)]ethylamine

From 2-(2-methoxyphenoxy)ethylamine (commercially available).

¹H-NMR (δ): 0.78-2.78 (m, 18H), 2.99-3.18 (m, 2H), 2.82 (s, 3H), 4.06-4.15 (m, 2H), 5.00-5.20 (m, 1H), 6.80-6.99 (m, 4H), 7.13-7.23 (m, 4H)

### EXAMPLE 86A3

N-(1-Cyclohexanecarbonyl-1,2,3,4-tetrahydroquinoline-2-ylmethyl)-N-[3-(2-methoxyphenoxy)propylmethylamine]

From 3-(2-methoxyphenoxy)propylmethylamine (U.S. Pat. No. 3,221,054, 1965).

¹H-NMR (CDCl₃): 0.80-2.15 (m, 15H); 2.20-2.80 (m, 9H); 3.83 (s, 3H); 4.05 (t, 2H); 5,00 (b, 1H); 6.82-6.98 (m, 4H); 7.00-7.14 (m, 1H); 7.14-7.25 (m, 3H).

### EXAMPLE 86A4

4-Cyano-1-(1-cyclohexanecarbonyl-1,2,3,4-tetrahydroquinoline-2-ylmethyl)-4-phenylpiperidine

From 4-cyano-4-phenylpiperidine (commercially available.)

¹H-NMR (δ): 0.78-3.22 (m, 25H), 5.00-5.20 (m, 1H), 6.93-7.60 (m, 9H).

### EXAMPLE 86A5

4-Benzyl-1-(1-cyclohexanecarbonyl-1,2,3,4-tetrahydroquinoline-2-ylmethyl)-4-hydroxypiperidine

From 4-benzyl-4-hydroxypiperidine (commercially available).

¹H-NMR (δ): 0.78-2.12 (m, 19H), 2.20-2.91 (m, 9H), 5.00-5.20 (m, 1H), 6.93-7.48 (m, 9H)

### EXAMPLE 86A6

4-Benzyloxy-1-(1-cyclohexanecarbonyl-1,2,3,4-tetrahydroquinoline-2-ylmethyl)piperidine

From 4-benzyloxypiperidine (EP 0015817, 1980).

¹H-NMR (δ): 0.80-3.50 (m, 26H); 4.50 (s, 2H); 5.05 (b, 1H); 7.10-7.46 (m, 9H).

### EXAMPLE 86A7

4-Benzoylamino-1-(1-cyclohexanecarbonyl-1,2,3,4-tetrahydroquinoline-2-ylmethyl)piperidine

From N-piperidin-4-ylbenzamide (commercially available).

¹H-NMR (δ): 0.76-4.30 (m, 26H); 5.15 (b, 1H); 7.10-7.55 (m, 9H); 7.75 (b, 1H).

### EXAMPLE 86A8

4-Acetyl-1-(1-cyclohexanecarbonyl-1,2,3,4-tetrahydroquinoline-2-ylmethyl)-4-phenylpiperidine

From 4-acetyl-4-phenylpiperidine (commercially available).

¹H-NMR (δ): 0.75-2.90 (m, 25H); 1.92 (s, 3H); 4.85-5.25(m, 1H); 6.95-7.50(m, 9H).

### EXAMPLE 86A9

1-(Benzo[1,3]dioxol-4-yl)-4-(1-cyclohexanecarbonyl-1,2,3,4-tetrahydroquinoline-2-ylmethyl)piperazine

From 1-(4-benzo[1,3]dioxolyl)piperazine (U.S. Pat. No. 5,424,313)

¹H-NMR (δ): 0.70-2.10 (m, 12H); 2.20-3.50 (m, 13H); 4.80-5.15 (m, 1H); 5.90 (s, 2H); 6.35-6.60 (m, 2H); 6.78 (ddd, 1H); 7.00-7.30 (m, 4H).

### EXAMPLE 86A10

N-[2-(3-Chlorophenoxy)ethyl]-N-(1-cyclohexanecarbonyl-1,2,3,4-tetrahydroquinoline-2-yl)methylamine

From 2-(3-Chlorophenoxy)ethylmethylamine (CA53:11401 i)

¹H-NMR (δ): 0.80-2.05 (m, 12H); 2.05-3.10 (m, 10H); 3.65-4.35 (m, 2H); 4.85-5.25 (m, 1H); 6.70 (dd, 1H); 6.80 (m, 1H); 6.92 (dd, 1H); 7.05-7.30 (m, 5H).

### EXAMPLE 86A11

N-[2-(4-Chlorophenoxy)ethyl]-N-(1-cyclohexanecarbonyl-1,2,3,4-tetrahydroquinoline-2-yl)methylamine

From 2-(4-chlorophenoxy)ethylmethylamine (commercially available).

¹H-NMR (δ): 0.70-2.03 (m, 12H); 2.03-3.15 (m, 10H); 3.60-4.30 (m, 2H); 4.80-5.25 (m, 1H); 6.65-6.85 (m, 2H); 7.00-7.30 (m, 6H).

### EXAMPLE 86A12

N-Benzyl-N-(1-cyclohexanecarbonyl-1,2,3,4-tetrahydroquinoline-2-ylmethyl)-N-[2-(2-methoxyphenoxy)ethyl]amine

From N-2-(2-methoxyphenoxy)ethyl-N-benzylethylamine (EP1142873, 2001).

¹H-NMR (δ): 0.75-2.15 (m, 12H); 2.15-2.80 (m, 5H); 2.80-3.00 (m, 2H); 3.75 (s, 5H); 3.85-4.00 (m, 2H); 4.90-5.20 (m, 1H); 6.60-7.50 (m, 13H).

### EXAMPLE 86A13

N-[(1-Cyclohexanecarbonyl-1,2,3,4-tetrahydroquinoline-2-yl)methyl]-N-(3-phenylpropyl)methylamine

From N-(3-phenylpropyl)methylamine (commercially available).

H-NMR (δ): 0.75-2.91 (m, 25H); 4.81-5.38 (m, 1H); 6.99-7.41 (m, 9H).

### EXAMPLE 86A14

1-[4-Cyano-2-trifluoromethoxyphenyl)-4-(1-cyclohexanecarbonyl-1,2,3,4-tetrahydroquinoline-2-ylmethyl)piperazine

From 1-(4-Cyano-2-trifluoromethoxyphenyl)piperazine

¹H-NMR (δ): 0.71-2.24 (m, 12H); 2.27-4.42 (m, 13H); 4.92-5.47 (m, 1H); 6.88-7.98 (m, 7H).

The starting piperazine was prepared as following:

4-Cyano-2-trifluoromethoxyphenylbromobenzene (Compound 86A14-A)

To a solution of 4-amino-3-trifluoromethoxybenzonitrile (0.5 g) in H₂O (1.5 mL) and 48% HBr (1.5 mL), cooled at 0° C., NaNO₂ (0.18 g) in H₂O (1.5 ml) was added. The mixture was stirred at 0° C. for 30′, poured into a suspension of CuBr (0.42 g) in 48% HBr (3 mL) and then heated at 75° C. for 1 h and at r.t. overnight. H₂O (10 mL) was added and the solution was extracted with CH₂Cl₂ (3×15 ml). The organic phase was dried (Na₂SO₄), filtered and evaporated at reduced pressure. The crude was purified by flash chromatography eluting with petroleum ether—-ethyl acetate 9:1 affording 0.39 g (60%) of the title compound.

¹H-NMR (δ): 7.49 (dd, 1H); 7.61 (d, 1H); 7.86 (d, 1H).

b) 1-(4-Cyano-2-trifluoromethoxyphenyl)piperazine (Compound 86A14-B)

A mixture of compound 86A14-A (0.39 g), piperazine anhydrous (0.76 g), sodium tert-butylate (0.21 g), BINAP (0.055 g) and tris(dibenzylideneacetone)dipalladium chloroform adduct (0.031 g) in toluene was refluxed under N₂ atmosphere for 4 h. After cooling at r.t., the insoluble were filtered on celite, washed with toluene and the filtrate was evaporated at reduced pressure. The crude was purified by flash chromatography eluting with EtOAc—2 N methanolic ammonia 9:1 to give 0.24 (59%) of the title compound.

¹H-NMR (δ): 2.91-3.11 (m, 4H); 3.15-3.31 (m, 4H); 7.01 (d, 1H); 7.47 (d, 1H), 7.52 (dd, 1H)

### EXAMPLE 86A15

1-(1-Cyclohexanecarbonyl-1,2,3,4-tetrahydroquinoline-2-ylmethyl)-4-(4-fluoro-2-methoxyphenylamino)piperidine

From 4-(4-fluoro-2-methoxyphenylamino)piperidine

¹H-NMR (δ): 0.82-2.93 (m, 25H); 3.10-3.29 (m, 1H); 3.61-4.11 (br, 4H); 4.88-5.16 (m; 1H); 6.43-6.62 (m, 3H); 7.02-7.25 (m, 3H).

The starting piperidine was prepared as following:

a) 1-tert-Butoxycarbonyl-4-(4-fluoro-2-methoxyphenylamino)piperidine (Compound 86A15-A)

The title compound was prepared as described for the Compound of Example 35A but reacting 1-tert-butoxycarbonyl-4-oxopiperidine with 2-methoxy-4-fluoroaniline. Purification by flash chromatography (Petr. Ether—EtOAc 9:1)afforded compound 86A15-A (96%).

¹H-NMR (δ): 1.10-1.65 (m, 11H); 1.90-2.15 (m, 2H); 2.80-3.15 (m, 2H); 3.25-3.50 (m, 1H); 3.60-4.25 (m, 6H); 6.40-6.80 (m, 3H).

b) 4-(4-Fluoro-2-methoxyphenylamino)piperidine HCl (Compound 86A15-B)

To a solution of compound 86A15-A (1.55 g) in MeOH (10 mL) was added 1.8 N HCl in Et₂O (70 mL) and the mixture stirred at r.t. for 2.5 h. The precipitated solid was filtered, washed with acetone to give 1.42 g (100%) of the title compound.

¹H-NMR (δ):1.70-2.20 (m, 4H); 2.75-3.05 (m, 2H); 3.20-3.40 (m, 2H); 3.40-3.75 (m, 2H); 3.85 (s, 3H); 3.90 (s, 1H); 6.85 (ddd, 1H); 7.10 (dd, 1H); 7.30 (ddd, 1H);

### EXAMPLE 86A16

1-(3H-Benzotriazol-4-yl)-4-(1-cyclohexanecarbonyl-1,2,3,4-tetrahydroquinoline-2-ylmethyl)piperazine

From 1-(3H-Benzotriazol-4-yl)piperazine (U.S. Pat. No. 4,459,296)

¹H-NMR (δ): 0.85-2.03 (m, 11H), 2.06-2.24 (m, 1H); 2.29-2.90 (m, 9H); 3.53-3.81 (m, 4H); 5.01-5.29 (m, 1H); 6.65 (d, 1H); 7.05-7.40 (m, 7H).

### EXAMPLE 86A17

1-(1H-Benzoimidazol-4-yl)-4-(1-cyclohexanecarbonyl-1,2,3,4-tetrahydroquinoline-2-ylmethyl)piperazine

From 1-(1H-Benzoimidazol-4-yl)piperazine (U.S. Pat. No. 4,459,296)

H¹-NMR (δ): 0.75-2.03 (m, 11H); 2.09-2.25 (m, 1H); 2.28-2.89 (m, 9H); 2.99-3.18 (m, 1H); 3.36-3.78 8m, 3H); 4.97-5.26 (m; 1H); 6.62 (d, 1H), 7.01-7.32 (m; 6H); 7.97 (s; 1H); 9.05-9.42 (br, 1H)

### EXAMPLE 86A18

N-(1-Cyclohexanecarbonyl-1,2,3,4-tetrahydroquinoline-2-ylmethyl)-N-[2-(2-pyridyloxy)ethylamine]

From N-[2-(2-pyridyloxy)ethylamine] U.S. Pat. No. 4,011,324

¹H-NMR (δ): 0.73-2.00 (m, 10H), 2.05-2.79 (m, 7H), 2.91-3.13 (m, 2H), 4.32 (d, 2H), 4.83-5.08 (m, 1H), 6.70 (d, 1H), 6.80-6.91 (m, 1H), 7.02-7.38 (m, 4H), 7.52 (dt, 1H), 8.05-8.18 (m, 1H)

### EXAMPLE 86A19

N-(1-Cyclohexanecarbonyl-1,2,3,4-tetrahydroquinoline-2-ylmethyl)-N-[2-(2-pyridyloxy)ethylmethylamine]

From N-[2-(2-pyridyloxy)ethylmethylamine] U.S. Pat. No. 4,011,324 using N-nethyletanolamine instead of ethanolamine.

¹H-NMR (δ): 0.73-2.05 (m, 10H), 2.05-3.09 (m, 12H), 4.20-4.48 (m, 2H), 4.83-5.08 (m, 1H), 6.65 (d, 1H), 6.80-6.91 (m, 1H), 7.02-7.38 (m, 4H), 7.52 (dt, 1H), 8.05-8.18 (m, 1H)

### EXAMPLE 86A20

N-(1-Cyclohexanecarbonyl-1,2,3,4-tetrahydroquinoline-2-ylmethyl)-N-[3-(2-methoxyphenyl)propylmethylamine]

3-(2-methoxyphenyl)propylmethylamine (CA52:10919i)

¹H-NMR (CDCl₃): 0.80-2.15 (m, 14H); 2.20-2.80 (m, 12H); 3.76 (s, 3H); 4.96 (b, 1H); 6.75-6.95 (m, 2H); 7.05-7.25 (m, 6H).

### EXAMPLE 86A21

1-(4-Benzoyl)-4-(1-cyclohexanecarbonyl-1,2,3,4-tetrahydroquinoline-2-ylmethyl)piperazine

From 1-benzoylpiperazine (Ber. B66, 113-116, 1933).

¹H-NMR (δ): 0.75-2.0 (m, 12H); 2.0-2.8 (m, 8H); 3.2-3.8 (m, 5H); 5.0-5.3 (m, 1H); 7.0-7.5 (m 9H).

### EXAMPLE 86A22

1-(1-Cyclohexanecarbonyl-1,2,3,4-tetrahydroquinoline-2-ylmethyl)-3-oxo-4-phenylpiperazine

From 1-phenyl-2-oxopiperazine (Tetrahedron Letters, 39 (1998) 7459-7462.)

¹H-NMR (δ): 0.75-2.0 (m, 11H); 2.0-2.2 (m, 1H); 2.2-2.8 (m, 6H); 3.0-3.2 (m, 1H); 3.2-3.3 (s, 2H); 3.5-3.8 (m, 2H); 5.0-5.2 (m, 1H); 7.1-7.6 (m, 9H).

### EXAMPLE 87

1-(4-Fluoro-2-methoxyphenyl)-4-(1-hexanoyl-1,2,3,4-tetrahydroquinoline-2-ylmethyl)piperazine

The title compound was synthesised following the alternative acylation procedure described for the compound of Example 1 (step f) but starting from Compound 74A-B instead of Compound 1B and using hexanoyl chloride instead of cyclohexanecarbonyl chloride. The crude was purified by flash chromatography eluting with petroleum ether-ethyl acetate 65:35 to give the title compound (75%).

¹H-NMR (δ): 0.71-1.79 (m, 10H), 2.03-2.79 (m, 14H), 2.82-3.10 (m, 4H), 3.32 (s, 3H), 4.96-5.28 (m, 1H); 6.48-6.67 (m, 2H), 6.75-6.91 (m, 1H), 7.04-7.25 (m, 4H).

### EXAMPLE 88

1-(2,3-Dihydrobenzo[1,4]dioxin-5-yl)-4-(1-pentafluoropropionyl-1,2,3,4-tetrahydroquinolin-2-ylmethyl)piperazine

The title compound was synthesised following the alternative acylation procedure described for the compound of Example 1 (step f) but starting from Compound 76A-B instead of Compound 1B and using pentafluoropropionyl anhydride instead of cyclohexanecarbonyl chloride. The crude was purified by flash chromatography eluting with petroleum ether-ethyl acetate 8:2 to give the title compound (70%).

¹H-NMR (δ):1,40-1,70 (m, 1H), 2.00-2.85 (m, 9H), 2.90-3.20 (M, 4H), 4.15-4.40 (m, 4H), 4.80-5.25 (m, 1H), 6.45-6.65 (m, 2H), 6.75 (ddd, 1H), 7.10-7.50 (m, 4H).

### EXAMPLE 89

1-(2,3-Dihydrobenzo[1,4]dioxin-5-yl)-4-[1-(3,3,3-trifluoropropionyl)-1,2,3,4-tetrahydroquinolin-2-ylmethyl)piperazine

The title compound was synthesised following the alternative acylation procedure described for the compound of Example 1 (step f) but starting from Compound 76A-B instead of Compound 1B and using 3,3,3-trifluoropropionyl chloride instead of cyclohexanecarbonyl chloride. The crude was purified by flash chromatography eluting with petroleum ether-ethyl acetate 7:3 to give the title compound (64%).

¹H-NMR (δ):1.35-1.60 (m, 1H), 2.00-2.20 (m, 1H), 2.20-2.80 (m, 8H), 2.90-3.10 (m, 4H), 3.10-3.55 (m, 2H), 4.15-4.35 (m, 4H), 5.00-5.25 (m, 1H), 6.40-6.60 (m, 2H), 6.75 (ddd, 1H), 6.95-7.15 (m, 1H), 7.15-7.35 (m, 3H).

### EXAMPLE 90

(Z)-1-(2,3-Dihydrobenzo[1,4]dioxin-5-yl)-4-[1-(4-hydroxycyclohexanecarbonyl)-1,2,3,4-tetrahydroquinolin-2-ylmethyl]piperazine

a) (Z)-1-(2,3-Dihydrobenzo[1,4]dioxin-5-yl)-4-[1-(4-diphenyltertbutylsilyloxycyclohexanecarbonyl)-1,2,3,4-tetrahydroquinoline-2-ylmethyl]piperazine (Compound 90A)

The title compound was synthesised following the acylation procedure described for the compound of Example 1 (step f) starting from Compound 76A-B but using cis-4-diphenyltertbutylsilyloxycyclohexanecarbonyl chloride, (prepared as described in EP 352909), instead of cyclohexanecarbonyl chloride. The title product was used without further purification in the next step.

b) (Z)-1-(2,3-Dihydrobenzo[1,4]dioxin-5-yl)-4-[1-(4-hydroxycyclohexanecarbonyl)-1,2,3,4-tetrahydroquinolin-2-ylmethyl]piperazine

To a solution of compound 90A (0.3 g) in 30 ml of MeOH was added 0.5 mL of 1:8 N HCl in isopropanol and the resulting suspension stirred at 50° C. for 12 h. Afterwards the solvent was evaporated under vacuum; the residue was dissolved in CH₂Cl₂ (10 ml) and 1N NaOH (10 mL). The organic layer was separated and washed with H₂O (10 ml). The organic layer was dried (Na₂SO₄) and the crude was purified by flash chromatography (CH₂Cl₂— MeOH 9:1) to give the title compound (83%).

### EXAMPLE 91

1-(1-Cyclohexanecarbonyl-6-fluoro-1,2,3,4-tetrahydroquinoline-2-ylmethyl)-4-(4-fluoro-2-methoxyphenyl)piperazine

a) 6-Fluoroquinoline-2-carbaldehyde (Compound 91A)

To a suspension of selenium dioxide (0.84 g;) in dioxane (20 mL) a solution of 6-fluoro quinaldine (0.81 g;) in dioxane (20 mL) was added and the resulting mixture refluxed for 4 h. After cooling to r.t., the inorganic salts were filtered off and the solution was evaporated to dryness under reduced pressure affording 0.87 g (99%) of the title compound.

H¹-NMR (δ): 7.48-7.69 (m, 2H); 8.06 (d, 1H); 8.20-8.34 (m, 2H); 10.21 (s; 1H)

b) 6-Fluoro-2-[4-(4-fluoro-2-methoxyphenyl)piperazin-1-ylmethyl]quinoline (Compound 91B)

The title compound was synthesised following the reductive amination procedure described for Compound 35B, but using Compound 91A instead of compound 35A. The residue was purified by flash chromatography (petroleum ether—EtOAc 1:1) affording Compound 91B (56%).

H¹-NMR (δ): 2.68-2.87 (m, 4H); 2.99-3.18 (m, 4H); 3.77-3.96 (m, 5H), 6.50-6.69 (m, 2H); 6.78-6.94 (m, 1H); 7.37-7.58 (m, 2H); 7.21 (d, 1H); 8.01-8.18 (m, 2H)

c) 6-Fluoro-2-[4-(4-fluoro-2-methoxyphenyl)piperazin-1-ylmethyl]-1,2,3,4-tetrahydroquinoline (Compound 91C)

The title compound was synthesised as reported in Example. 1 (step e) but using compound 91B instead of Compound 1C. The residue was used in the next step without futher purification. (86%).

H¹-NMR (δ): 1.41-1.70 (m, 1H); 1.79-2.01 (m, 1H); 2.34-2.97 (m, 8H); 2.99-3.28 (m, 4H); 3.31-3.52 (m, 1H); 3.89 (s, 3H); 4.52-4.82 (br, 1H); 6.38-6.97 (m, 6H).

d) 1-(1-Cyclohexanecarbonyl-6-fluoro-1,2,3,4-tetrahydroquinoline-2-ylmethyl)-4-(4-fluoro-2-methoxyphenyl)piperazine

The title compound was synthesised following the alternative acylation procedure described for the compound of Example 1 (step f) but starting from Compound 91C instead of Compound 1B. The crude was purified by flash chromatography eluting with petroleum ether-ethyl acetate 6:4 to give the title compound (85%).

H¹-NMR (δ): 0.82-2.99 (m, 11H); 2.01-2.18 (m, 1H); 2.21-2.81 (m, 9H); 2.86-3.11 (m, 4H); 3.85 (s, 3H); 4.96-5.26 (m, 1H), 6.51-6.68 (m, 2H), 6.76-6.99 (m, 3H); 7.01-7.17 (m, 1H).

### EXAMPLE 92

### Radioligand Binding to Recombinant 5-HT_{1A} Receptors

A. Method:

Genomic clone G-21 coding for the human 5HT_{1A}-serotonergic receptor was stably transfected in a human cell line (HeLa). HeLa cells were grown as monolayers in Dulbecco's modified Eagle medium (DMEM), containing 10% foetal bovine serum, gentamicin (10 mg/ml) and 5% carbon dioxide, at 37° C. The cells were detached from the growth flask at 95% confluence by a cell scraper and were lysed in cold 5 mM Tris and 5 mM EDTA buffer (pH 7.4). The homogenates were centrifuged at 40000×g×20 minutes and the pellets were resuspended in a small volume of cold 5 mM Tris and 5 mM EDTA buffer (pH 7.4) and immediately frozen and stored at −70° C. until use. On the day of experiment, the cell membranes were resuspended in incubation buffer: 50 mM Tris HCl (pH 7.4), 2.5 mM MgCl₂, 10 mM pargyline (Fargin et al., Nature 335, 358-360, 1988). The membranes were incubated in a final volume of 1 ml for 30 minutes at 30° C. with 1 nM [³H]8-OH-DPAT, in the absence or presence of the test compounds. Non-specific binding was determined in the presence of 10 μM 5-HT. Incubation was stopped by addition of cold Tris-HC 1 buffer and rapid filtration through a 0.2%-polyethyleneimine-pretreated Whatman-GF/B or Schleicher-&-Schuell-GF52 filter.

B. Results

The affinity of the tested compounds was evaluated as inhibition of specific binding of the radioligand to 5-HT_{1A} receptors (IC₅₀) by using the non-linear curve-fitting program Allfit (De Lean et al., Am. J. Physiol. 235, E97-E102 (1978). The IC₅₀ value was converted to an affinity constant (Ki) by the equation of Cheng & Prusoff (Cheng Y. C., Prusoff W. H., Biochem. Pharmacol. 22, 3099-3108 (1973)).

The results reported in Tables 1a-f show that the compounds of the invention tested have a high affinity for the 5-HT_{1A} receptor.

TABLE 1a
Binding affinity for 5HT_{1A} receptors
Data are expressed as Ki (nM)

| Compound | Affinity |
|---|---|
| 1 | 3.3 |
| 1 (+) | 0.2 |
| 2 | 0.3 |
| 3 | 8.4 |
| 4 | 0.7 |
| 5 | 10.7 |
| 7 | 31.2 |
| 8 | 10.3 |
| 9 | 9.6 |
| 11 | 40.8 |
| 12 | 47.5 |
| 14 | 82.0 |
| 15 | 8.9 |
| 18 | 2.8 |
| 20 | 38.6 |
| 24A | 12.9 |
| 24A1 | 2.6 |
| 24A3 | 5.4 |
| 24A4 | 9.3 |
| 26 | 47.9 |
| 31 | 12.8 |
| 32 | 2.8 |
| 33 | 47.7 |
| 34 | 18.9 |
| 35A9 | 39.6 |
| 35A12 | 9.5 |
| 35A13 | 4.0 |
| 35A18 | 3.0 |
| 35A19 | 5.2 |
| 35A21 | 18.2 |
| 35A23 | 3.1 |
| 35A24 | 11.6 |
| 35A25 | 10.1 |
| 35A26 | 3.1 |
| 35A29 | 31.1 |
| 35A32 | 20.0 |
| 35A34 | 12.3 |
| 35A35 | 6.2 |
| 35A36 | 2.4 |

TABLE 1b
Binding affinity for 5HT_{1A} receptors
Data are expressed as Ki (nM)

| Compound | Affinity |
|---|---|
| 36 | 15.6 |
| 38 | 17.3 |
| 43 | 5.0 |
| 44 | 30.0 |
| 46 | 11.2 |
| 48 | 17.7 |
| 50 | 41.0 |
| 53 | 24.2 |
| 54 | 51.0 |
| 55 | 2.4 |
| 56 | 3.6 |
| 57 | 2.3 |
| 59 | 45.7 |
| 60A | 10.1 |
| 60A1 | 8.1 |
| 60A4 | 18.5 |
| 60A8 | 12.2 |
| 60A9 | 6.5 |
| 60A10 | 31.6 |
| 60A11 | 10.6 |
| 62A | 43.4 |
| 62A2 | 15.6 |
| 62A3 | 5.7 |
| 62A5 | 31.2 |
| 63 | 42.9 |
| 64 | 23.9 |
| 67 | 29.4 |
| 68A | 9.2 |
| 68A1 | 23.9 |
| 68A2 | 13.9 |
| 68A3 | 7.1 |
| 68A4 | 4.7 |
| 68A5 | 4.0 |
| 68A6 | 14.2 |
| 68A9 | 24.6 |
| 68A11 | 6.8 |
| 68A12 | 2.6 |
| 68A13 | 5.3 |

TABLE 1c
Binding affinity for 5HT_{1A} receptors
Data are expressed as Ki (nM)

| Compound | Affinity |
|---|---|
| 68A19 | 4.1 |
| 68A20 | 30.7 |
| 68A23 | 42.8 |
| 68A24 | 32.9 |
| 68A28 | 34.4 |
| 68A30 | 23.8 |
| 68A31 | 41.8 |
| 68A32 | 19.4 |
| 68A33 | 13.3 |
| 68A34 | 24.9 |
| 68A36 | 10.7 |
| 68A38 | 24.4 |
| 68A39 | 6.8 |
| 68A40 | 7.5 |
| 68A41 | 49.1 |
| 68A42 | 19.8 |
| 68A43 | 42.7 |
| 68A44 | 20.7 |
| 68A45 | 8.6 |
| 68A46 | 34.9 |
| 68A47 | 17.1 |
| 68A49 | 18.7 |
| 68A50 | 27.3 |
| 68A51 | 15.6 |
| 68A52 | 18.3 |
| 68A54 | 41.4 |
| 68A55 | 14.3 |
| 68A56 | 26.8 |
| 68A57 | 33.0 |
| 68A59 | 46.5 |
| 68A61 | 19.7 |
| 68A63 | 8.7 |
| 68A64 | 13.4 |
| 68A66 | 19.6 |
| 68A67 | 9.1 |
| 68A68 | 7.5 |
| 68A69 | 4.2 |
| 68A70 | 5.3 |

TABLE 1d
Binding affinity for 5HT_{1A} receptors
Data are expressed as Ki (nM)

| Compound | Affinity |
|---|---|
| 68A71 | 23.5 |
| 68A73 | 17.9 |
| 68A74 | 41.4 |
| 69A | 7.3 |
| 69A1 | 17.5 |
| 69A2 | 24.0 |
| 69A3 | 9.3 |
| 69A5 | 15.7 |
| 69A7 | 36.0 |
| 69A8 | 39.5 |
| 69A9 | 25.5 |
| 69A10 | 13.5 |
| 69A11 | 10.4 |
| 69A12 | 2.1 |
| 69A13 | 21.3 |
| 70A | 7.8 |
| 70A1 | 18.8 |
| 70A2 | 18.8 |
| 70A3 | 15.2 |
| 70A4 | 20.9 |
| 70A5 | 40.3 |
| 70A6 | 4.7 |
| 71A | 35.7 |
| 71A1 | 15.2 |
| 71A2 | 92.0 |
| 71A3 | 40.6 |
| 71A4 | 45.9 |
| 71A5 | 45.0 |
| 72A3 | 47.2 |
| 72A6 | 3.2 |
| 72A7 | 4.6 |
| 72A8 | 31.6 |
| 72A11 | 7.7 |
| 72A13 | 7.8 |
| 72A17 | 38.5 |
| 72A18 | 11 |
| 72A20 | 5.4 |
| 72A23 | 24.6 |

TABLE 1e
Binding affinity for 5HT_{1A} receptors
Data are expressed as Ki (nM)

| Compound | Affinity |
|---|---|
| 72A25 | 15.3 |
| 72A26 | 19.6 |
| 72A29 | 28.1 |
| 72A30 | 10.9 |
| 72A39 | 39.1 |
| 72A40 | 19.8 |
| 72A51 | 15.0 |
| 72A53 | 6.1 |
| 72A56 | 10.1 |
| 72A57 | 6.4 |
| 72A65 | 15.5 |
| 72A66 | 10.2 |
| 72A68 | 38.6 |
| 72A69 | 3.9 |
| 72A81 | 25.5 |
| 72A84 | 15.7 |
| 72A85 | 3.6 |
| 72A86 | 4.3 |
| 72A87 | 3.9 |
| 72A89 | 34.7 |
| 72A93 | 7.8 |
| 72A94 | 10.0 |
| 73A6 | 18.1 |
| 73A13 | 8.1 |
| 74A | 17.6 |
| 74A1 | 2.0 |
| 74A5 | 2.0 |
| 74A8 | 5.9 |
| 74A9 | 38.3 |
| 75A | 3.2 |
| 75A2 | 10.1 |
| 75A3 | 14.0 |
| 75A4 | 14.6 |
| 75A5 | 2.6 |
| 75A6 | 4.3 |
| 75A7 | 5.4 |

TABLE 1f
Binding affinity for 5HT_{1A} receptors
Data are expressed as Ki (nM)

| Compound | Affinity |
|---|---|
| 75A8 | 17.0 |
| 75A9 | 16.1 |
| 75A10 | 26.4 |
| 75A11 | 17.1 |
| 75A15 | 27.7 |
| 76A1 | 5.4 |
| 76A2 | 5.1 |
| 76A3 | 5.0 |
| 76A4 | 3.9 |
| 76A5 | 12.4 |
| 76A6 | 5.5 |
| 76A7 | 8.5 |
| 76A8 | 19.2 |
| 76A10 | 21.5 |
| 76A11 | 4.5 |
| 76A12 | 8.0 |
| 76A13 | 2.3 |
| 76A15 | 8.1 |
| 77A1 | 24.2 |
| 77A5 | 13.1 |
| 79 | 4.5 |
| 85 | 41.2 |
| 86A | 18.2 |

### EXAMPLE 93

### Effects on Rhythmic Bladder-Voiding Contractions Induced by Bladder Filling in Anaesthetised Rats

A. Method:

Female Sprague-Dawley rats weighing 225-275 g (Crl: CDo Br, Charles River Italia) were used. The animals were housed with free access to food and water and maintained on a forced 12-hour alternating light-dark cycle at 22-24° C. for at least one week, except during the experiment. The activity on rhythmic bladder voiding contractions was evaluated according to the method of Dray (Dray J., Pharmacol. Methods, 13:157, 1985), with some modifications as in Guarneri (Guarneri, Pharmacol. Res. 27:173, 1993). Briefly, the rats were anaesthetised by subcutaneous injection of 1.25 g/kg (5 ml/kg) urethane, after which the urinary bladder was catheterised via the urethra using PE 50 polyethylene tubing filled with physiological saline. The catheter was tied in place with a ligature around the external urethral orifice and was connected to conventional pressure transducers (Statham P23 ID/P23 XL). The intravesical pressure was displayed continuously on a chart recorder (Battaglia Rangoni KV 135 with DCI/TI amplifier). The bladder was then filled via the recording catheter by incremental volumes of warm (37° C.) saline until reflex bladder-voiding contractions occurred (usually 0.8-1.5 ml). For intravenous injection of bioactive compounds, PE 50 polyethylene tubing filled with physiological saline was inserted into the jugular vein.

From the cystometrogram, the number of contractions recorded 15 minutes before (basal values) and after treatment, as well as the mean amplitude of these contractions (mean height of the peaks in mmHg), was evaluated.

Since most compounds produced an effect that was relatively rapid in onset and led to a complete cessation of bladder contractions, bioactivity was conveniently estimated by measuring the duration of bladder quiescence (i.e. the length of the time during which no contractions occurred). The number of tested animals showing a reduction in the number of contractions higher than 30% of that observed in the basal period was recorded too.

To compare the potency of the tested compounds for inhibiting the bladder voiding contractions, equieffective doses which resulted in the disappearance of contractions for a time of 10 minutes (ED₁₀ₘᵢₙ) were computed by means of linear regression using the least square method. The extrapolated doses which induced a reduction in the number of contractions greater than 30% in 50% of the treated rats (ED₅₀) were evaluated by the method of Bliss (Bliss C. I., Quart J. Pharm. Pharmacol. 11, 192-216, 1938).

B. Results

The rapid distension of the urinary bladder in urethane-anaesthetised rats produced a series of rhythmic bladder-voiding contractions whose characteristics have been described (Maggi et al., Brain Res. 380:83, 1986; Maggi et al., J. Pharmacol. Exp. Ther., 230: 500, 1984). The frequency of these contractions is related to the sensory afferent arm of reflex micturition and to the integrity of the micturition centre, while their amplitude depends on the function of the reflex efferent arm. In this model system, compounds that act mainly on the central nervous system (such as morphine) cause a block in voiding contraction, whereas drugs that act at the level of the detrusor muscle, such as oxybutynin, lower the amplitude of the bladder contractions.

The results obtained after administration of prior-art compounds and compounds of the invention are shown in Table 2.

The compounds of the invention were clearly superior to the reference standards, particularly with regard to the ED₅₀ values which are indicators of urinary frequency.

Oxybutynin only decreased the amplitude of the contractions in a dose-related manner, with an ED₅₀ value (the extrapolated dose inducing a 30% reduction of amplitude of the contractions in 50% of treated rats) of 240 μg/kg. That is, oxybutynin does not cause cessation of bladder contractions. This amplitude-reduction effect characteristic of oxybutynin, which can potentially cause lower bladder contractility and the undesirable retention of residual urine in the bladder after micturition, is not a characteristic of the compounds of the invention.

TABLE 2
Effects on rhythmic bladder-voiding contractions after
intravenous administration
Data represent the ED_{10 min values (the extrapolated dose inducing}
10 minutes of disappearance of the contractions), the ED₅₀ values
(the extrapolated doses inducing a reduction of the number of
contractions >30% in 50% of treated rats) (frequency), and the ED₅₀
values (the extrapolated doses inducing a 30% reduction of amplitude
of the contractions in 50% of treated rats) (amplitude).

| | | ED₅₀ frequency | ED₅₀ amplitude |
|---|---|---|---|
| Compound | ED_{10 min} (μg/kg) | (μg/kg) | (μg/kg) |
| Ex. 1 | 107 | 64 | n.a. |
| Ex. (+) − 1 | 72 | 24 | n.a. |
| Ex. 2 | 383 | 172 | n.a. |
| Ex. 3 | 80 | 20 | n.a. |
| Flavoxate | >10000 | 2648 | n.a. |
| Oxybutynin | 7770 | >10000 | 240 |

n.a.—not active; no significant reduction of the height of the peaks

### EXAMPLE 94

### Effect on Cystometric Parameters in Conscious Rats After Oral Administration

A. Method:

Male Sprague-Dawley rats [Crl: CD° (SD) BR] of 300-400 g supplied by Charles River Italia were used. The animals were housed with free access to food and water and maintained on a forced 12-hour-light/12-hour-dark cycle at 22-24° C. of temperature, except during the experiment. To quantify urodynamic parameters in conscious rats, cystometrographic studies were performed according to the procedure previously reported (Guarneri et al., Pharmacol. Res. 24: 175, 1991).

Briefly, the rats were anaesthetised by intraperitoneal administration of 3 ml/kg of Equithensin solution (pentobarbital 30 mg/kg and chloral hydrate 125 mg/kg) and placed in a supine position. An approximately-10-mm-long midline incision was made in the shaved and cleaned abdominal wall. The urinary bladder was gently freed from adhering tissues, emptied and then cannulated via an incision in the bladder body, using a polyethylene cannula (0.58-mm internal diameter, 0.96-mm external diameter) which was permanently sutured with silk thread. The cannula was exteriorised through a subcutaneous tunnel in the retroscapular area, where it was connected to a plastic adapter in order to avoid the risk of removal by the animal. For drug testing, the rats were utilised one day after implantation.

On the day of the experiment, the rats were placed in modified Bollman cages, i.e. restraining cages that were large enough to permit the rats to adopt a normal crouched posture, but narrow enough to prevent turning around. After a stabilisation period of about 20 minutes, the free tip of the bladder cannula was connected through a T-shaped tube to a pressure transducer (Statham P23XL) and to a peristaltic pump (Gilson minipuls 2) for continues infusion of a warm (37° C.) saline solution into the urinary bladder, at a constant rate of 0.1 ml/minute. The intraluminal-pressure signal during infusion of saline into the bladder was continuously recorded on a polygraph (Rectigraph-8K San-ei with BM614/2 amplifier from Biomedica Mangoni) and, from the cystometrogram, two urodynamic parameters were evaluated: bladder volume capacity (BVC) and micturition pressure (MP). BVC (in ml) is defined as the volume of saline infused into the bladder necessary to induce detrusor contraction followed by micturition. MP (in mmHg) is defined as the maximal intravesical pressure caused by contraction during micturition. Basal BVC and MP values were evaluated as mean of the values observed in the cystometrograms recorded in an initial period of 30-60 minutes. At this point in the assay, the infusion was interrupted and the test compounds were administered orally by a stomach tube. The bladder infusion restarted and changes in BVC and MP were evaluated from the mean values obtained in the cystometrograms observed during 1, 2, 3, 4 and 5 hours after treatment. The compounds were administered in a volume of 2 ml/kg and groups of control animals received the same amount of vehicle (0.5% methocel in water) orally.

Statistical Analysis

All data were expressed as mean±standard error. Time-course data were analysed by S.A.S./STAT software, version 6.12. The difference between vehicle and active-treatment effect was evaluated by: General Linear Model Procedure—Repeated Measures Analysis of Variance: Univariate test of Hypotheses for within Subject Effects—Analysis of Variance of Contrast Variables. In the figures, data were reported as % changes versus the basal values.

B. Results:

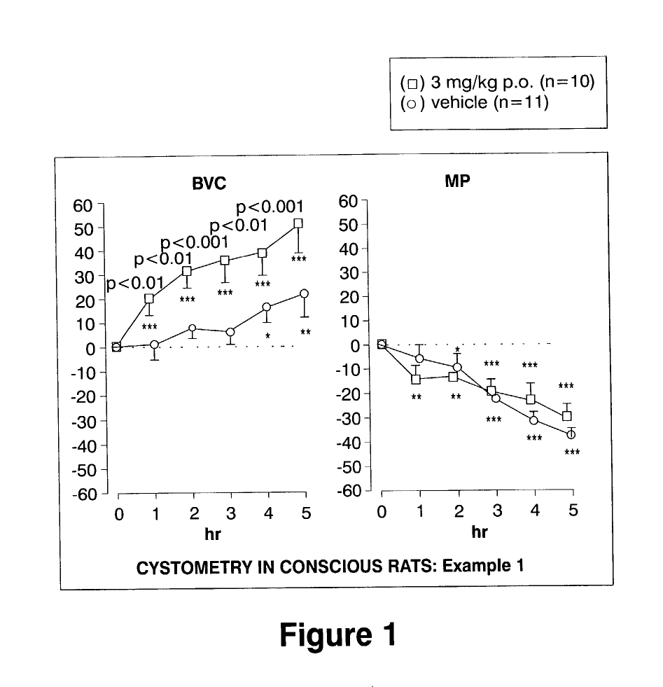

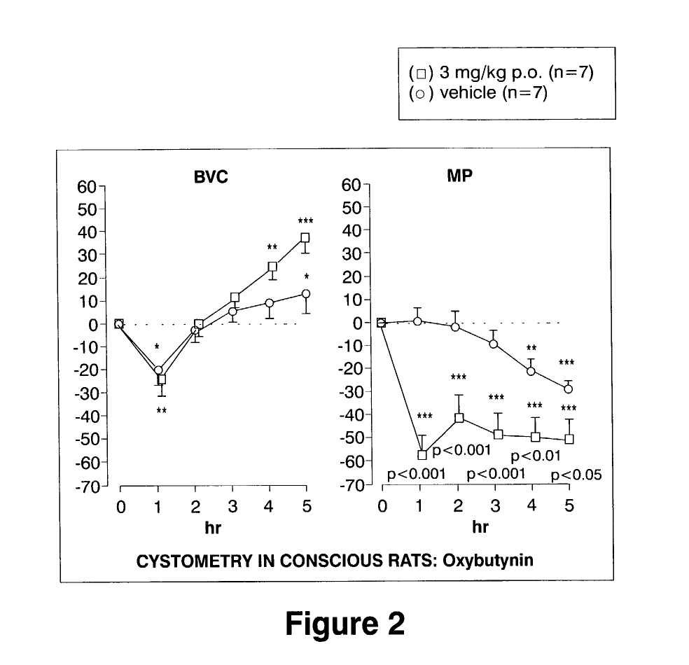

The time course of the effects of the administered doses of the tested compounds is shown in FIGS. 1 and 2. The compound of Example 1 administered at 3 mg/kg p.o. proved effective in increasing the bladder volume capacity without significant effects on micturition pressure (FIG. 1).

On the contrary, the oral administration of 3 mg/kg of oxybutynin induced a slight, not-significant increase of BVC in comparison with the control group. Moreover this dose greatly reduced the micturition pressure values and the differences from control animals were at all times statistically significant (FIG. 2).

### EXAMPLE 95

### Inhibition of Stereotypy (Rhythmic Forepaw Treading) Induced by 8-OH-DPAT in Rats (Post-Synaptic Antagonism)

A. Method:

The inhibitory effect of 5-HT_{1A}-receptor antagonists on stereotyped forepaw treading induced in rats by subcutaneous injection of 8-OH-DPAT was evaluated by the method of Tricklebank (Tricklebanket al., Eur. J. Pharmacol., 117: 15, 1985) with minor modifications as described below.

Male Sprague-Dawley rats [Crl: CD° (SD) BR] weighing 150-175 g from Charles River Italia were used. The animals were housed with free access to food and water and maintained on a forced 12-hour-light/12-hour-dark cycle at 22-24° C. of temperature. On the day of the experiment, the rats were placed singly in clear plastic containers, 10-15 minutes before administration of the vehicle or compounds to be tested. For evaluation of antagonistic activity after oral administration, the compounds were administered 0.5, 1 and 4 hours before induction of stereotypy by 8-OH-DPAT (1 mg/kg subcutaneously). Observation sessions lasted 30 seconds and began 3 minutes after 8-OH-DPAT treatment and were repeated every 3 minutes over a period of 15 minutes.

The appearance of the symptom induced by postsynaptic stimulation of 5-HT_{1A} receptors was noted, and the intensity was scored using an intensity scale in which: 0=absent, 1=equivocal, 2=present and 3=intense. Behavioural scores for treated rats were accumulated throughout the observation time (5 observation periods) and expressed as mean values of 4 rats/dose. Change in mean values of treated animals in comparison with control (vehicle) group, expressed as per-cent inhibition, was used in order to quantify the antagonistic activity.

B. Results:

The results are shown in Table 3. These results demonstrate that the compound of Example 1, after oral administration of a dose of 1 mg/kg, exhibits significant and long-lasting post-synaptic 5-HT_{1A}-receptor antagonist activity. The results obtained after oral administration of 1 mg/kg of Example 1 enantiomers are reported in Table 3 too. The (+)-enantiomer exhibits significant and complete inhibition of the symptom up to 4 hours after administration, whereas the same dose of the (−)-enantiomer proved devoid of activity. Also the other compounds tested, after oral administration of a dose of 10 mg/kg, exhibited significant and long-lasting post-synaptic 5-HT_{1A}-receptor antagonist activity.

TABLE 3
Inhibition of forepaw treading induced by 8-OH-DPAT in rats
(post-synaptic antagonism)

| | Dose | % Inhibition of forepaw treading | | |
|---|---|---|---|---|
| Compound | (mg/kg p.o.) | 0.5 h | 1 h | 4 h |
| Ex. 1 | 1 | 91 | 91 | 96 |
| Ex.(+) 1 | 1 | 100 | 100 | 98 |
| Ex.(−) 1 | 1 | 19 | 5 | 9 |
| 18 | 10 | 98 | 92 | 92 |
| 24A1 | 10 | 100 | 100 | 100 |
| 24A3 | 10 | 100 | 93 | 100 |
| 35A13 | 10 | n.t. | 95 | 100 |

n.t.—not tested

We claim:

1. A method of reducing the frequency of urinary bladder contractions in a mammal in need of such treatment comprising administering to said mammal an effective amount of a compound of formula I 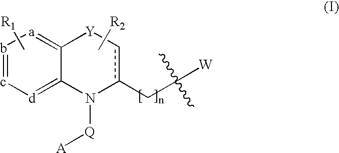
where:
W represents group 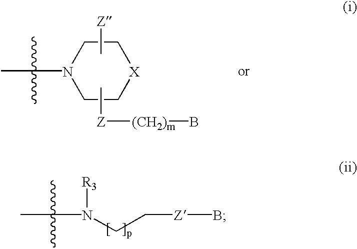
R₁ is one or more substituents selected from the group consisting of hydrogen, halogen, hydroxyl, (C₁-C₇)-alkyl, substituted (C₁-C₇)-alkyl, (C₁-C₇)-alkoxyl, substituted (C₁-C₇)-alkoxyl, nitro, aryl, substituted aryl, heterocycle, substituted heterocycle, (C₂-C₅)-alkenyl, substituted (C₂-C₅)-alkenyl, amino, (C₁-C₅)-alkylamino, di-[(C₁-C₅)]-alkylamino, cyano, —SR₃, —C(O)R₃, —C(O)NR₃R₃, —NR₃C(O)R₃, —NR₃SO₂R₃, —NR₃C(O)OR₃ and —N(H)C(O)N(H)R₃, where R₃ is independently selected from the group consisting of hydrogen, (C₁-C₇)-alkyl, substituted (C₁-C₇)-alkyl, cycloalkyl, substituted cycloalkyl, aryl, substituted aryl, heterocycle and substituted heterocycle;
R₂ is one or two substituents selected from the group consisting of hydrogen, halogen, oxo, (C₁-C₇)-alkyl, substituted (C₁-C₇)-alkyl, (C₂-C₅)-alkenyl and substituted (C₂-C₅)-alkenyl groups;
Y represents a CH, CH₂, CR₂, CHR₂ group or a bond;
Q represents a carbonyl, thiocarbonyl or sulfonyl group;
A represents a (C₁-C₇)-alkyl, substituted (C₁-C₇)-alkyl, cycloalkyl, substituted cycloalkyl, (C₂-C₅)-alkenyl, substituted (C₂-C₅)-alkenyl, cycloalkenyl, substituted cycloalkenyl, aryl, substituted aryl, heterocycle, substituted heterocycle, (C₁-C₅)-alkylamino, substituted (C₁-C₅)-alkylamino, di-[(C₁-C₅)]-alkylamino, substituted di-[(C₁-C₅)]-alkylamino, cyclic amino, substituted cyclic amino, arylamino, substituted arylamino, arylalkylamino or substituted arylalkylamino group;
n is independently 1 or 2;
m is independently 0, 1 or 2;
p is independently 1, 2 or 3;
a, b, c and d are independently a carbon or nitrogen atom, or CH, CH₂ or NH group, with the proviso that no more than two of a, b, c and d may simultaneously be nitrogen atoms, NH groups or a combination of nitrogen atoms and NH groups;
X represents a bond, CH, CH₂, SO or SO₂ group or a carbon, nitrogen or sulphur atom, with the provisos that when X is a nitrogen atom or CH group, the -Z-(CH₂)ₘ—B group is bound to said nitrogen atom or CH group, and when X is a carbon atom Z″ is not a hydrogen atom or oxo group and the -Z-(CH₂)ₘ—B and Z″ groups are bound to said carbon;
Z represents a bond, an oxygen or sulphur atom or —CH(OH)—, —C(O)—NR₃C(O)—, —NR₃—C(O)—NR₃—, or —NR₃— group;
Z′ represents a bond or an oxygen or sulphur atom;
Z″ represents a hydrogen atom or hydroxyl, oxo, alkylcarbonyl or cyano group,
B represents a monocyclic aryl, substituted monocyclic aryl, bicyclic aryl, substituted bicyclic aryl, monocyclic heterocycle, substituted monocyclic heterocycle, bicyclic heterocycle or substituted bicyclic heterocycle; [symbol]
represents a single or double bond and, when Y═CH, the double bond is shifted so as to contain it; or
an enantiomer, diastereomer, N-oxide, crystalline form, hydrate, solvate, pharmaceutically acceptable salt, prodrug or active metabolite thereof, in combination with a muscarinic receptor antagonist.

2. The method of claim 1 wherein said mammal is a human.

3. The method of claim 1 wherein said muscarinic receptor antagonist is selected from the group consisting of oxybutynin, tolterodine, darifenacin, and temiverine.

4. A method of treating neuromuscular dysfunction of the lower urinary tract in a mammal in need of such treatment, comprising administering to said mammal an effective amount of a compound of formula I 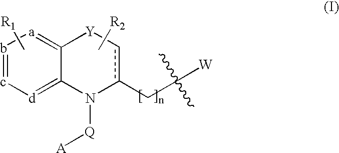
where:
W represents group 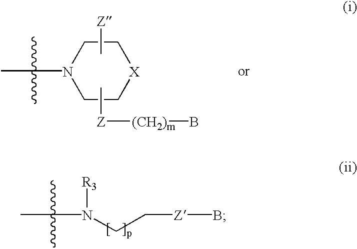
R₁ is one or more substituents selected from the group consisting of hydrogen, halogen, hydroxyl, (C₁-C₇)-alkyl, substituted (C₁-C₇)-alkyl, (C₁-C₇)-alkoxyl, substituted (C₁-C₇)-alkoxyl, nitro, aryl, substituted aryl, heterocycle, substituted heterocycle, (C₂-C₅)-alkenyl, substituted (C₂-C₅)-alkenyl, amino, (C₁-C₅)-alkylamino, di-[(C₁-C₅)]-alkylamino, cyano, —SR₃, —C(O)R₃, —C(O)NR₃R₃, —NR₃C(O)R₃, —NR₃SO₂R₃, —NR₃C(O)OR₃ and —N(H)C(O)N(H)R₃, where R₃ is independently selected from the group consisting of hydrogen, (C₁-C₇)-alkyl, substituted (C₁-C₇)-alkyl, cycloalkyl, substituted cycloalkyl, aryl, substituted aryl, heterocycle and substituted heterocycle;
R₂ is one or two substituents selected from the group consisting of hydrogen, halogen, oxo, (C₁-C₇)-alkyl, substituted (C₁-C₇)-alkyl, (C₂-C₅)-alkenyl and substituted (C₂-C₅)-alkenyl groups;
Y represents a CH, CH₂, CR₂, CHR₂ group or a bond;
Q represents a carbonyl, thiocarbonyl or sulfonyl group;
A represents a (C₁-C₇)-alkyl, substituted (C₁-C₇)-alkyl, cycloalkyl, substituted cycloalkyl, (C₂-C₅)-alkenyl, substituted (C₂-C₅)-alkenyl, cycloalkenyl, substituted cycloalkenyl, aryl, substituted aryl, heterocycle, substituted heterocycle, (C₁-C₅)-alkylamino, substituted (C₁-C₅)-alkylamino, di-[(C₁-C₅)]-alkylamino, substituted di-[(C₁-C₅)]-alkylamino, cyclic amino, substituted cyclic amino, arylamino, substituted arylamino, arylalkylamino or substituted arylalkylamino group;
n is independently 1 or 2;
m is independently 0, 1 or 2;
p is independently 1, 2 or 3;
a, b, c and d are independently a carbon or nitrogen atom, or CH, CH₂ or NH group, with the proviso that no more than two of a, b, c and d may simultaneously be nitrogen atoms, NH groups or a combination of nitrogen atoms and NH groups;
X represents a bond, CH, CH₂, SO or SO₂ group or a carbon, nitrogen or sulphur atom, with the provisos that when X is a nitrogen atom or CH group, the -Z-(CH₂)ₘ—B group is bound to said nitrogen atom or CH group, and when X is a carbon atom Z″ is not a hydrogen atom or oxo group and the -Z-(CH₂)ₘ—B and Z″ groups are bound to said carbon;
Z represents a bond, an oxygen or sulphur atom or —CH(OH)—, —C(O)—NR₃C(O)—, —NR₃—C(O)—NR₃—, or —NR₃— group;
Z′ represents a bond or an oxygen or sulphur atom;
Z″ represents a hydrogen atom or hydroxyl, oxo, alkylcarbonyl or cyano group,
B represents a monocyclic aryl, substituted monocyclic aryl, bicyclic aryl, substituted bicyclic aryl, monocyclic heterocycle, substituted monocyclic heterocycle, bicyclic heterocycle or substituted bicyclic heterocycle; [symbol]
represents a single or double bond and, when Y═CH, the double bond is shifted so as to contain it; or
an enantiomer, diastereomer, N-oxide, crystalline form, hydrate, solvate, pharmaceutically acceptable salt, prodrug or active metabolite thereof,
in combination with a muscarinic receptor antagonist.

5. The method of claim 4 wherein said mammal is a human.

6. The method of claim 4 wherein said muscarinic receptor antagonist is selected from the group consisting of oxybutynin, tolterodine, darifenacin, and temiverine.

7. The method of claim 4 wherein administration of said compound ameliorates a condition or symptom selected from the group consisting of urinary urgency, overactive bladder, increased urinary frequency, incontinence, mixed incontinence, urine leakage, enuresis, dysuria, urinary hesitancy and difficulty in emptying the urinary bladder.

8. A method for treating disorders of the central nervous system caused by serotonergic dysfunction, comprising administering an effective amount of a compound of formula I 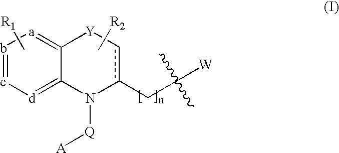
where:
W represents group 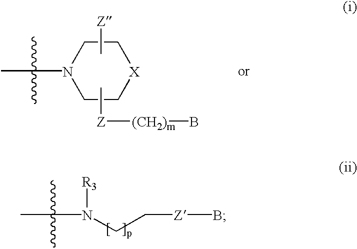
R₁ is one or more substituents selected from the group consisting of hydrogen, halogen, hydroxyl, (C₁-C₇)-alkyl, substituted (C₁-C₇)-alkyl, (C₁-C₇)-alkoxyl, substituted (C₁-C₇)-alkoxyl, nitro, aryl, substituted aryl, heterocycle, substituted heterocycle, (C₂-C₅)-alkenyl, substituted (C₂-C₅)-alkenyl, amino, (C₂-C₅)-alkylamino, di-[(C₁-C₅)]-alkylamino, cyano, —SR₃, —C(O)R₃, —C(O)NR₃R₃, —NR₃C(O)R₃, —NR₃SO₂R₃, —NR₃C(O)OR₃ and —N(H)C(O)N(H)R₃, where R₃ is independently selected from the group consisting of hydrogen, (C₁-C₇)-alkyl, substituted (C₁-C₇)-alkyl, cycloalkyl, substituted cycloalkyl, aryl, substituted aryl, heterocycle and substituted heterocycle;
R₂ is one or two substituents selected from the group consisting of hydrogen, halogen, oxo, (C₁-C₇)-alkyl, substituted (C₁-C₇)-alkyl, (C₂-C₅)-alkenyl and substituted (C₂-C₅)-alkenyl groups;
Y represents a CH, CH₂, CR₂, CRH₂ group or a bond;
Q represents a carbonyl, thiocarbonyl or sulfonyl group;
A represents a (C₁-C₇)-alkyl, substituted (C₁-C₇)-alkyl, cycloalkyl, substituted cycloalkyl, (C₂-C₅)-alkenyl, substituted (C₂-C₅)-alkenyl, cycloalkenyl, substituted cycloalkenyl, aryl, substituted aryl, heterocycle, substituted heterocycle, (C₁-C₅)-alkylamino, substituted (C₁-C₅)-alkylamino, di-[(C₁-C₅)]-alkylamino, substituted di-[(C₁-C₅)]-alkylamino, cyclic amino, substituted cyclic amino, arylamino, substituted arylamino, arylalkylamino or substituted arylalkylamino group;
n is independently 1 or 2;
m is independently 0, 1 or 2;
p is independently 1, 2 or 3;
a, b, c and d are independently a carbon or nitrogen atom, or CH, CH₂ or NH group, with the proviso that no more than two of a, b, c and d may simultaneously be nitrogen atoms, NH groups or a combination of nitrogen atoms and NH groups;
X represents a bond, CH, CH₂, SO or SO₂ group or a carbon, nitrogen or sulphur atom, with the provisos that when X is a nitrogen atom or CH group, the -Z-(CH₂)ₘ—B group is bound to said nitrogen atom or CH group, and when X is a carbon atom Z″ is not a hydrogen atom or oxo group and the -Z-(CH₂)ₘ—B and Z″ groups are bound to said carbon;
Z represents a bond, an oxygen or sulphur atom or —CH(OH)—, —C(O)—NR₃C(O)—, —NR₃—C(O)—NR₃—, or —NR₃— group;
Z′ represents a bond or an oxygen or sulphur atom;
Z″ represents a hydrogen atom or hydroxyl, oxo, alkylcarbonyl or cyano group,
B represents a monocyclic aryl, substituted monocyclic aryl, bicyclic aryl, substituted bicyclic aryl, monocyclic heterocycle, substituted monocyclic heterocycle, bicyclic heterocycle or substituted bicyclic heterocycle; [symbol]
represents a single or double bond and, when Y═CH, the double bond is shifted so as to contain it; or
an enantiomer, diastereomer, N-oxide, crystalline form, hydrate, solvate, pharmaceutically acceptable salt, prodrug or active metabolite thereof;
in combination with a muscarinic receptor antagonist.

9. The method of claim 8 wherein said compound of formula I is delivered via an extracorporeal route.

10. The method of claim 8 wherein said muscarinic receptor antagonist is selected from the group consisting of oxybutynin, tolterodine, darifenacin, and temiverine

11. The method of claim 8 wherein said mammal is a human.

12. The method of claim 8 wherein said disorder of the central nervous system is selected from the group consisting of anxiety, depression, hypertension, sleep/wake cycle disorders, feeding behavior, sexual dysfunction and cognition disorders.

13. A method for reducing the activity of a 5HT_{1A} receptor comprising exposing said 5HT_{1A} receptor to an activity-lowering amount of a 5HT_{1A} receptor antagonist of formula I 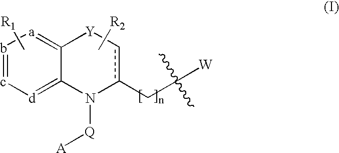
where:
W represents group 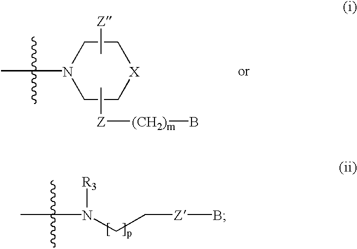
R₁ is one or more substituents selected from the group consisting of hydrogen, halogen, hydroxyl, (C₁-C₇)-alkyl, substituted (C₁-C₇)-alkyl, (C₁-C₇)-alkoxyl, substituted (C₁-C₇)-alkoxyl, nitro, aryl, substituted aryl, heterocycle, substituted heterocycle, (C₂-C₅)-alkenyl, substituted (C₂-C₅)-alkenyl, amino, (C₁-C₅)-alkylamino, di-[(C₁-C₅)]-alkylamino, cyano, —SR₃, —C(O)R₃, —C(O)NR₃R₃, —NR₃C(O)R₃, —NR₃SO₂R₃, —NR₃C(O)OR₃ and —N(H)C(O)N(H)R₃, where R₃ is independently selected from the group consisting of hydrogen, (C₁-C₇)-alkyl, substituted (C₁-C₇)-alkyl, cycloalkyl, substituted cycloalkyl, aryl, substituted aryl, heterocycle and substituted heterocycle;
R₂ is one or two substituents selected from the group consisting of hydrogen, halogen, oxo, (C₁-C₇)-alkyl, substituted (C₁-C₇)-alkyl, (C₂-C₅)-alkenyl and substituted (C₂-C₅)-alkenyl groups;
Y represents a CH, CH₂, CR₂, CHR₂ group or a bond;
Q represents a carbonyl, thiocarbonyl or sulfonyl group;
A represents a (C₁-C₇)-alkyl, substituted (C₁-C₇)-alkyl, cycloalkyl, substituted cycloalkyl, (C₂-C₅)-alkenyl, substituted (C₂-C₅)-alkenyl, cycloalkenyl, substituted cycloalkenyl, aryl, substituted aryl, heterocycle, substituted heterocycle, (C₁-C₅)-alkylamino, substituted (C₁-C₅)-alkylamino, di-[(C₁-C₅)]-alkylamino, substituted di-[(C₁-C₅)]-alkylamino, cyclic amino, substituted cyclic amino, arylamino, substituted arylamino, arylalkylamino or substituted arylalkylamino group;
n is independently 1 or 2;
m is independently 0, 1 or 2; p is independently 1, 2 or 3;
a, b, c and d are independently a carbon or nitrogen atom, or CH, CH₂ or NH group, with the proviso that no more than two of a, b, c and d may simultaneously be nitrogen atoms, NH groups or a combination of nitrogen atoms and NH groups;
X represents a bond, CH, CH₂, SO or SO₂ group or a carbon, nitrogen or sulphur atom, with the provisos that when X is a nitrogen atom or CH group, the -Z-(CH₂)ₘ—B group is bound to said nitrogen atom or CH group, and when X is a carbon atom Z″ is not a hydrogen atom or oxo group and the -Z-(CH₂)ₘ—B and Z″ groups are bound to said carbon;
Z represents a bond, an oxygen or sulphur atom or —CH(OH)—, —C(O)—NR₃C(O)—, —NR₃—C(O)—NR₃—, or —NR₃— group;
Z′ represents a bond or an oxygen or sulphur atom;
Z″ represents a hydrogen atom or hydroxyl, oxo, alkylcarbonyl or cyano group,
B represents a monocyclic aryl, substituted monocyclic aryl, bicyclic aryl, substituted bicyclic aryl, monocyclic heterocycle, substituted monocyclic heterocycle, bicyclic heterocycle or substituted bicyclic heterocycle; [symbol]
represents a single or double bond and, when Y═CH, the double bond is shifted so as to contain it; or
an enantiomer, diastereomer, N-oxide, crystalline form, hydrate, solvate, pharmaceutically acceptable salt, prodrug or active metabolite thereof;
in combination with muscarinic receptor antagonist.

14. The method of claim 13 wherein said muscarinic receptor antagonist is selected from the group consisting of oxybutynin, tolterodine, darifenacin, and temiverine.

15. A method of reducing the frequency of urinary bladder contractions in a mammal in need of such treatment comprising administering to said mammal an effective amount of a compound of formula I 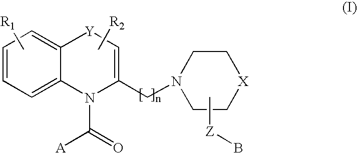
where:
R₁ is one or more substituents selected from the group consisting of hydrogen, halogen, hydroxyl, (C₁-C₇)-alkyl, substituted (C₁-C₇)-alkyl, (C₁-C₇)-alkoxyl, substituted (C₁-C₇)-alkoxyl, nitro, (C₅-C₁₂)-aryl, substituted (C₅-C₁₂)-aryl, heterocycle with 5-12 atoms in the ring, substituted heterocycle with 5-12 atoms in the ring, (C₂-C₅)-alkenyl, substituted (C₂-C₅)-alkenyl, amino, (C₁-C₄)-alkylamino, di-[(C₁-C₄)-alkyl]amino, cyano, —SR₃, —C(O)R₃, —C(O)NR₃R₃, —NR₃C(O)R₃, —NR₃SO₂R₃ and —N(H)C(O)N(H)R₃;
R₃ is independently selected from the group consisting of hydrogen, (C₁-C₇)-alkyl, substituted (C₁-C₇)-alkyl, (C₃-C₇)-cycloalkyl, substituted (C₃-C₇)-cycloalkyl, (C₅-C₁₂)-aryl, substituted (C₅-C₁₂)-aryl, heterocycle with 5-12 atoms in the ring, and substituted heterocycle with 5-12 atoms in the ring;
R₂ is one or two substituents selected from the group consisting of hydrogen, (C₁-C₇)-alkyl, substituted (C₁-C₇)-alkyl, (C₂-C₅)-alkenyl and substituted (C₂-C₅)-alkenyl;
Y represents a CH or CH₂ group or a bond;
A represents a (C₁-C₇)-alkyl, (C₃-C₇)-cycloalkyl, (C₅-C₇)-cycloalkenyl, (C₅-C₁₂)-aryl, heterocycle with 5-12 atoms in the ring, (C₁-C₄)-alkylamino, di-[(C₁-C₄)-alkyl]amino, cyclic amino containing 4-6 carbon atoms and optionally one or more heteroatoms such as O, N or S, (C₅-C₁₂)-arylamino, (C₅-C₁₂)-aryl-(C₁-C₄)-alkylamino, each optionally substituted;
n is independently 1 or 2;
X represents a CH, CH₂, SO or SO₂ group or a nitrogen or sulphur atom and, when X represents a nitrogen atom or a CH group, the Z-B group of formula I is bound to said nitrogen or CH;
Z represents a valence bond, an oxygen or sulphur atom or a CH₂, CH₂CH₂ or CO group;
B represents a monocyclic (C₅-C₇)-aryl, bicyclic (C₉-C₁₂)-aryl, monocyclic heterocycle with 5-7 atoms in the ring, bicyclic heterocycle with 9-12 atoms in the ring, and each of these rings can be optionally substituted; [symbol]
represents a single or double bond and, when Y═CH, the double bond is shifted so as to contain it; or
an enantiomer, diastereomer, N-oxide, crystalline form, hydrate, solvate, pharmaceutically acceptable salt, prodrug or active metabolite of such a compound,
in combination with a muscarinic receptor antagonist.

16. The method of claim 15 wherein said mammal is a human.

17. The method of claim 15 wherein said muscarinic receptor antagonist is selected from the group consisting of oxybutynin, tolterodine, darifenacin, and temiverine.

18. A method of treating neuromuscular dysfunction of the lower urinary tract in a mammal in need of such treatment, comprising administering to said mammal an effective amount of a compound of formula I 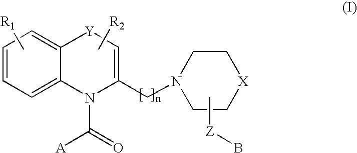
where:
R₁ is one or more substituents selected from the group consisting of hydrogen, halogen, hydroxyl, (C₁-C₇)-alkyl, substituted (C₁-C₇)-alkyl, (C₁-C₇)-alkoxyl, substituted (C₁-C₇)-alkoxyl, nitro, (C₅-C₁₂)-aryl, substituted (C₅-C₁₂)-aryl, heterocycle with 5-12 atoms in the ring, substituted heterocycle with 5-12 atoms in the ring, (C₂-C₅)-alkenyl, substituted (C₂-C₅)-alkenyl, amino, (C₁-C₄)-alkylamino, di-[(C₁-C₄)-alkyl]amino, cyano, —SR₃, —C(O)R₃, —C(O)NR₃R₃, —NR₃C(O)R₃, —NR₃SO₂R₃ and —N(H)C(O)N(H)R₃;
R₃ is independently selected from the group consisting of hydrogen, (C₁-C₇)-alkyl, substituted (C₁-C₇)-alkyl, (C₃-C₇)-cycloalkyl, substituted (C₃-C₇)-cycloalkyl, (C₅-C₁₂)-aryl, substituted (C₅-C₁₂)-aryl, heterocycle with 5-12 atoms in the ring, and substituted heterocycle with 5-12 atoms in the ring;
R₂ is one or two substituents selected from the group consisting of hydrogen, (C₁-C₇)-alkyl, substituted (C₁-C₇)-alkyl, (C₂-C₅)-alkenyl and substituted (C₂-C₅)-alkenyl;
Y represents a CH or CH₂ group or a bond;
A represents a (C₁-C₇)-alkyl, (C₃-C₇)-cycloalkyl, (C₅-C₇)-cycloalkenyl, (C₅-C₁₂)-aryl, heterocycle with 5-12 atoms in the ring, (C₁-C₄)-alkylamino, di-[(C₁-C₄)-alkyl]amino, cyclic amino containing 4-6 carbon atoms and optionally one or more heteroatoms such as O, N or S, (C₅-C₁₂)-arylamino, (C₅-C₁₂)-aryl-(C₁-C₄)-alkylamino, each optionally substituted;
n is independently 1 or 2;
X represents a CH, CH₂, SO or SO₂ group or a nitrogen or sulphur atom and, when X represents a nitrogen atom or a CH group, the Z-B group of formula I is bound to said nitrogen or CH;
Z represents a valence bond, an oxygen or sulphur atom or a CH₂, CH₂CH₂ or CO group;
B represents a monocyclic (C₅-C₇)-aryl, bicyclic (C₉-C₁₂)-aryl, monocyclic heterocycle with 5-7 atoms in the ring, bicyclic heterocycle with 9-12 atoms in the ring, and each of these rings can be optionally substituted; [symbol]
represents a single or double bond and, when Y═CH, the double bond is shifted so as to contain it; or
an enantiomer, diastereomer, N-oxide, crystalline form, hydrate, solvate, pharmaceutically acceptable salt, prodrug or active metabolite of such a compound,
in combination with a muscarinic receptor antagonist.

19. The method of claim 18 wherein said mammal is a human.

20. The method of claim 18 wherein said muscarinic receptor antagonist is selected from the group consisting of oxybutynin, tolterodine, darifenacin, and temiverine.

21. The method of claim 18 wherein administration of said compound ameliorates a condition or symptom selected from the group consisting of urinary urgency, overactive bladder, increased urinary frequency, incontinence, mixed incontinence, urine leakage, enuresis, dysuria, urinary hesitancy and difficulty in emptying the urinary bladder.

22. A method for treating disorders of the central nervous system caused by serotonergic dysfunction, comprising administering an effective amount of a compound of formula I 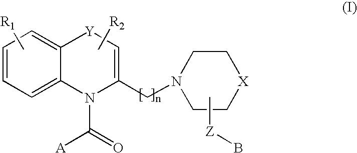
where:
R₁ is one or more substituents selected from the group consisting of hydrogen, halogen, hydroxyl, (C₁-C₇)-alkyl, substituted (C₁-C₇)-alkyl, (C₁-C₇)-alkoxyl, substituted (C₁-C₇)-alkoxyl, nitro, (C₅-C₁₂)-aryl, substituted (C₅-C₁₂)-aryl, heterocycle with 5-12 atoms in the ring, substituted heterocycle with 5-12 atoms in the ring, (C₂-C₅)-alkenyl, substituted (C₂-C₅)-alkenyl, amino, (C₁-C₄)-alkylamino, di-[(C₁-C₄)-alkyl]amino, cyano, —SR₃, —C(O)R₃, —C(O)NR₃R₃, —NR₃C(O)R₃, —NR₃SO₂R₃ and —N(H)C(O)N(H)R₃;
R₃ is independently selected from the group consisting of hydrogen, (C₁-C₇)-alkyl, substituted (C₁-C₇)-alkyl, (C₃-C₇)-cycloalkyl, substituted (C₃-C₇)-cycloalkyl, (C₅-C₁₂)-aryl, substituted (C₅-C₁₂)-aryl, heterocycle with 5-12 atoms in the ring, and substituted heterocycle with 5-12 atoms in the ring;
R₂ is one or two substituents selected from the group consisting of hydrogen, (C₁-C₇)-alkyl, substituted (C₁-C₇)-alkyl, (C₂-C₅)-alkenyl and substituted (C₂-C₅)-alkenyl;
Y represents a CH or CH₂ group or a bond;
A represents a (C₁-C₇)-alkyl, (C₃-C₇)-cycloalkyl, (C₅-C₇)-cycloalkenyl, (C₅-C₁₂)-aryl, heterocycle with 5-12 atoms in the ring, (C₁-C₄)-alkylamino, di-[(C₁-C₄)-alkyl]amino, cyclic amino containing 4-6 carbon atoms and optionally one or more heteroatoms such as O, N or S, (C₅-C₁₂)-arylamino, (C₅-C₁₂)-aryl-(C₁-C₄)-alkylamino, each optionally substituted;
n is independently 1 or 2;
X represents a CH, CH₂, SO or SO₂ group or a nitrogen or sulphur atom and, when X represents a nitrogen atom or a CH group, the Z-B group of formula I is bound to said nitrogen or CH;
Z represents a valence bond, an oxygen or sulphur atom or a CH₂, CH₂CH₂ or CO group;
B represents a monocyclic (C₅-C₇)-aryl, bicyclic (C₉-C₁₂)-aryl, monocyclic heterocycle with 5-7 atoms in the ring, bicyclic heterocycle with 9-12 atoms in the ring, and each of these rings can be optionally substituted; [symbol]
represents a single or double bond and, when Y═CH, the double bond is shifted so as to contain it; or
an enantiomer, diastereomer, N-oxide, crystalline form, hydrate, solvate, pharmaceutically acceptable salt, prodrug or active metabolite of such a compound,
in combination with a muscarinic receptor antagonist.

23. The method of claim 22 wherein said compound of formula I is delivered via an extracorporeal route.

24. The method of claim 22 wherein said muscarinic receptor antagonist is selected from the group consisting of oxybutynin, tolterodine, darifenacin, and temiverine

25. The method of claim 22 wherein said mammal is a human.

26. The method of claim 22 wherein said disorder of the central nervous system is selected from the group consisting of anxiety, depression, hypertension, sleep/wake cycle disorders, feeding behavior, sexual dysfunction and cognition disorders.

27. A method for reducing the activity of a 5HT₁ A receptor comprising exposing said 5HT_{1A} receptor to an activity-lowering amount of a 5HT_{1A} receptor antagonist of formula I 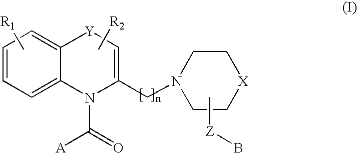
where:
R₁ is one or more substituents selected from the group consisting of hydrogen, halogen, hydroxyl, (C₁-C₇)-alkyl, substituted (C₁-C₇)-alkyl, (C₁-C₇)-alkoxyl, substituted (C₁-C₇)-alkoxyl, nitro, (C₅-C₁₂)-aryl, substituted (C₅-C₁₂)-aryl, heterocycle with 5-12 atoms in the ring, substituted heterocycle with 5-12 atoms in the ring, (C₂-C₅)-alkenyl, substituted (C₂-C₅)-alkenyl, amino, (C₁-C₄)-alkylamino, di-[(C₁-C₄)-alkyl]amino, cyano, —SR₃, —C(O)R₃, —C(O)NR₃R₃, —NR₃C(O)R₃, —NR₃SO₂R₃ and —N(H)C(O)N(H)R₃;
R₃ is independently selected from the group consisting of hydrogen, (C₁-C₇)-alkyl, substituted (C₁-C₇)-alkyl, (C₃-C₇)-cycloalkyl, substituted (C₃-C₇)-cycloalkyl, (C₅-C₁₂)-aryl, substituted (C₅-C₁₂)-aryl, heterocycle with 5-12 atoms in the ring, and substituted heterocycle with 5-12 atoms in the ring;
R₂ is one or two substituents selected from the group consisting of hydrogen, (C₁-C₇)-alkyl, substituted (C₁-C₇)-alkyl, (C₂-C₅)-alkenyl and substituted (C₂-C₅)-alkenyl;
Y represents a CH or CH₂ group or a bond;
A represents a (C₁-C₇)-alkyl, (C₃-C₇)-cycloalkyl, (C₅-C₇)-cycloalkenyl, (C₅-C₁₂)-aryl, heterocycle with 5-12 atoms in the ring, (C₁-C₄)-alkylamino, di-[(C₁-C₄)-alkyl]amino, cyclic amino containing 4-6 carbon atoms and optionally one or more heteroatoms such as O, N or S, (C₅-C₁₂)-arylamino, (C₅-C₁₂)-aryl-(C₁-C₄)-alkylamino, each optionally substituted;
n is independently 1 or 2;
X represents a CH, CH₂, SO or SO₂ group or a nitrogen or sulphur atom and, when X represents a nitrogen atom or a CH group, the Z-B group of formula I is bound to said nitrogen or CH;
Z represents a valence bond, an oxygen or sulphur atom or a CH₂, CH₂CH₂ or CO group;
B represents a monocyclic (C₅-C₇)-aryl, bicyclic (C₉-C₁₂)-aryl, monocyclic heterocycle with 5-7 atoms in the ring, bicyclic heterocycle with 9-12 atoms in the ring, and each of these rings can be optionally substituted; [symbol]
represents a single or double bond and, when Y═CH, the double bond is shifted so as to contain it; or
an enantiomer, diastereomer, N-oxide, crystalline form, hydrate, solvate, pharmaceutically acceptable salt, prodrug or active metabolite of such a compound,
in combination with a muscarinic receptor antagonist.

28. The method of claim 27 wherein said muscarinic receptor antagonist is selected from the group consisting of oxybutynin, tolterodine, darifenacin, and temiverine.
